(19) United States
(12) Patent Application Publication
(10) Pub. No.: US 2016/0287696 A1
(43) Pub. Date: Oct. 6, 2016

(54) PAN POLLEN IMMUNOGENS AND METHODS AND USES FOR IMMUNE RESPONSE MODULATION

(71) Applicants: LA JOLLA INSTITUTE FOR ALLERGY AND IMMUNOLOGY, La Jolla, CA (US); ALK-ABELLÓ A/S, Hørsholm (DK)

(72) Inventors: Bjoern Peters, La Jolla, CA (US); Alessandro Sette, La Jolla, CA (US); Jason Greenbaum, San Diego, CA (US); Ilka Hoof, Frederiksberg (DK); Lars Harder Christensen, Allerød (DK)

(73) Assignees: La Jolla Institute For Allergy And Immunology, La Jolla, CA (US); ALK-Abelló A/S, Hørsholm (DK)

(21) Appl. No.: 15/037,825

(22) PCT Filed: Nov. 20, 2014
(86) PCT No.: PCT/US2014/066577

§ 371 Date: May 19, 2016

Related U.S. Application Data
(60) Provisional application No. 61/906,821, filed on Nov. 20, 2013.
(60) Provisional application No. 61/946,370, filed on Feb. 28, 2014.

(51) Int. Cl. A61K 39/36; C07K 14/415

(52) U.S. Cl. CPC A61K 39/36; C07K 14/415; A61K 2039/545

(57) ABSTRACT

The invention relates to pan pollen immunogens such as polypeptides, proteins and peptides, and methods and uses of such immunogens for modulating or relieving an immune response in a subject. For example, the immunogens can be used for treating a subject for an allergic immune response or inducing or promoting immunological tolerance to the immunogen or a pollen allergen in a subject.

### GOVERNMENT SUPPORT

This invention was made with government support under contract NIH-NIAIDHHSN272200700048C awarded by the National Institutes of Health. The government has certain rights in the invention.

### FIELD OF THE INVENTION

The invention relates to pan pollen immunogens such as polypeptides, proteins and peptides, and methods and uses of such immunogens for modulating or relieving an immune response in a subject, such as treating a subject for an allergic immune response or inducing or promoting immunological tolerance to the immunogen or a pollen allergen in a subject.

### INTRODUCTION

Patients with pollen allergies are typically poly-sensitized as evidenced by positive RAST- and/or skin prick tests to multiple pollen allergens, like grass, weed and tree pollen allergens. However, today it is not possible to treat multisensitized patients with one immunotherapeutic product. Although several investigators have suggested that immunotherapy with a single grass species such as Timothy grass is sufficient to also treat allergies to other grass pollens due to observed cross-reactivity at the IgE level, it has not been suggested to treat multiple pollen allergies with one single immunogen.

It is firmly established that allergen-specific T-cells play an important role in allergic inflammation and that induction of antigen specific T regulatory cells (Tregs) or elimination of allergen-specific T helper type 2 cells (Th2) might be a prerequisite for the induction of specific tolerance. Yet, cross-reactivity among multiple pollen families at the T-cell level is less explored.

Allergen-specific immunotherapy (SIT) is a hyposensitizing immunotherapy introduced in clinical medicine almost a century ago for the treatment of an allergic immune response using the allergens that the subject is sensitized to. An allergic immune response may be mediated by activated allergen-specific Th2 cells, which produce cytokines such as IL-4, IL-5, and IL-13. In healthy individuals, the allergen-specific T-cell response is mediated predominantly by Th1 cells. SIT may reduce the ratio of Th2:Th1 cells and may alter the cytokine profile, reducing the production of IL-4, IL-5, and IL-13 and increasing the production of IFN-gamma in response to major allergens or allergen extracts.

Despite its efficacy, SIT has several limitations, including safety concerns about giving patients allergenic substances. Because most SIT regimens involve the administration of whole, unfractionated, allergen extracts, adverse IgE-mediated events are a considerable risk. Significant efforts have been devoted to developing approaches to modulate allergen-specific T-cell responses without inducing IgE-meditated, immediate-type reactions. These approaches include developing hypoallergens that do not contain IgE-binding epitopes, allergens that are coupled to adjuvants and carriers of bacterial or viral origin or peptides that contain dominant T-cell epitopes and do not react with IgE in allergic individuals.

It was recently shown that a large fraction of Timothy Grass-specific T cells target epitopes contained in novel Timothy Grass antigens (NTGA). NTGA's are unrelated to the known allergens of Timothy grass, which mainly are identified based on their high IgE reactivity. International patent application, WO2013/119863 A1, relates to novel antigens (NTGA's) derived from Timothy grass pollen.

It has also recently been shown and described in International patent application WO2012/049310 that an immunogen derived from an allergenic pollen source is able to reduce an allergic immune response caused by an unrelated allergen via bystander suppression.

As disclosed herein, immunogens related to recently detected immunogens of Timothy grass pollen (NTGA's) share high sequence conservation/homology to polypeptides identified in several different pollen families and are broadly reactive. Such immunogens have potential therapeutical utilization against immune responses triggered by pollen of a broad array of pollen families.

### SUMMARY

Disclosed herein are immunogens, also named pan-pollen immunogens, derived from previously detected NTGA's. A pan-pollen immunogen consists of or contain as part of its sequence an amino acid sequence that is conserved across polypeptides detected in a grass pollen and at least one non-grass pollen species, e.g. the non-grass pollen species Ambrosia psilostachya (Amb p), Ambrosia artemisiifolia, (Amb a), Plantago lanceolate (Pla I), Quercus alba (Que a), Betula verrucosa, (Bet v), Fraxinus Excelsior (Fra e) and Olea Europaea, (Ole e). In some embodiments, the immunogens may contain conserved subsequences, e.g. T cell epitope-containing subsequences of previously detected NTGA's, which T cell epitope-containing subsequence is conserved across polypeptides detected in a grass pollen and at least one non-grass pollen species. These are herein named PG+ sequences or PG+ peptides and have less than 3 mismatches to 15 contiguous amino acids of polypeptides detected in a grass pollen species and a non-grass pollen species described herein. Table 1 shows examples on such conserved subsequences (PG+ peptides) derived from previously detected NTGA's. In other embodiments, the immunogens may be larger amino acid sequences containing one or more conserved subsequences of Table 1, for example a wild type sequence of an NTGA. Table 2 shows examples on wild type polypeptides found in Phl p grass pollen, which contain one or more PG+ sequences of Table 1. Still other PG+ containing sequences or sequences with less than 3 mismatches to a PG+ peptide may be found in polypeptides found in non-grass pollen species, e.g. of the plant genera Ambrosia, Quercus and Betula (Table 4). Disclosed herein are also longer conserved regions or stretches that may derive from a wild type polypeptide described herein. A conserved region was defined as the region resulting from merging overlapping conserved 15mer peptides in a Phl p sequence. Table 3 shows conserved regions that are conserved across polypeptides found in grass-, weed- and tree pollen species (herein named GWT sequences). Such GWT sequences may be an immunogen in itself, or may give rise to additional immunogens comprising the entire conserved regions or subsequences thereof.

In certain embodiments, an immunogen may contain at least one T cell epitope as may be determined by the T cell response observed against immunogens of Tables 1, 2, 3, or 4 in cultured PBMC's obtained from grass pollen allergic donors or alternatively from ragweed, oak and/or birch pollen allergic donors. Furthermore, it was found that a T cell response of grass allergic donors to an immunogen of the invention may be cross reactive to non-grass pollen species, thereby indicating that grass pollen immunogens and its conserved homolog in non-grass pollen families share T cell epitopes. It was in general demonstrated (tendency) that T cells previously stimulated with a PG+ peptide produced a T cell response in response to different non-grass pollen extracts when the mismatch of the PG+ peptide compared to a subsequence of a polypeptide in the non-grass pollen extract was less than 3 mismatches (Table 10, FIG. 1). Therefore, in certain embodiments, the immunogens may contain at least one PG+ peptide disclosed in Table 10, e.g. a PG+ peptide with SEG ID NO: 246, 258 and 315. That is not to exclude that an immunogen may contain another peptide disclosed in Table 10.

Therefore, the invention relates in a first aspect to a method for relieving an allergic immune response against a pollen allergen, wherein the allergen is not a grass pollen allergen, in a subject in need thereof, comprising administering an effective amount of an immunogenic molecule, wherein said molecule comprises or consists of

a) a polypeptide, which includes at least one amino acid sequence with 0, 1 or 2 mismatches compared to a sequence selected from any one of SEQ ID NOs: 1-397 set out in Table 1;

b) a polypeptide comprising an amino acid sequence having at least 65% sequence similarity or identity to a sequence selected from any one of SEQ ID NOs: 398-443 set out in Table 2;

c) a polypeptide comprising an amino acid sequence having at least 65% sequence similarity or identity to a sequence selected from any one of SEQ ID NOs: 444-664 set out in Table 3; or

d) a polypeptide comprising an amino acid sequence having at least 65% sequence similarity or identity to a subsequence of at least 15 contiguous amino acid residues of a sequence selected from any of SEQ ID NOs: 444-664 set out in Table 3.

SEQ ID NOs: 1-397 as set out in Table 1 refers to PG+ peptides, which 15mer amino acid sequence contain less than 3 mismatches to a corresponding sequence identified in a non-grass pollen species, for example across a sequence identified in one or more of the species Amb p, Pla I, Ole e, Fra e, Que a and Bet v.

SEQ ID NOs: 398-443 as set out in Table 2 refers to wild type sequences of NTGAs identified by combined transcriptomic and Mass Spectrometry analysis, which contain one or more PG+ peptides.

SEQ ID NOs: 444-664 as set out in Table 3 refers to conserved regions (GWT) that are conserved across polypeptides identified in Phl p pollen (NTGA's) and polypeptides identified in weed pollen (Amb a and/or Amb p) and tree pollen (Que a and/or Bet v).

Below is shown embodiments specifically related to each of the pan-pollen immunogens identified. For example in embodiment F, a polypeptide relates to NTGA 6, and a polypeptide of option a) includes at least one amino acid sequence with 0, 1 or 2 mismatches compared to a sequence selected from any one of SEQ ID NOs: 52-74; the polypeptide of option b) comprises an amino acid sequence having at least 65% sequence similarity or identity to SEQ ID NOs: 403, the polypeptide of option c) comprises an amino acid sequence having at least 65% sequence similarity or identity to a sequence selected from any one of SEQ ID NOs: 474-479 and polypeptide of option d) comprises an amino acid sequence having at least 65% sequence similarity or identity to a subsequence of at least 15 contiguous amino acid residues of a sequence selected from any of SEQ ID NOs: 474-479 set out in Table 3. Other embodiments (A to AK) may be constructed the same way using the list below:

| | | Polypeptide | Polypeptide | Polypeptide |
|---|---|---|---|---|
| | | option a) | option b) | option c |
| | | PG + | Wild type | and d) GWT |
| | NTGA | Sequence | sequences | sequence |
| Embodiments: | No: | of Table 1: | of Table 2: | of Table 3: |
| Embodiment A | 1 | 1-7 | 398 | 444-449 |
| Embodiment B | 2 | 18-32 | 399 | 450-456 |
| Embodiment C | 3 | 33 | 400 | 457-459 |
| Embodiment D | 4 | 34-45 | 401 | 460-465 |
| Embodiment E | 5/64 | 46-51 | 402 | 466-473 |
| Embodiment F | 6 | 52-74 | 403 | 474-479 |
| Embodiment G | 7 | 75-83 | 404 | 480-485 |
| Embodiment H | 9 | 84-88 | 406 | 486-496 |
| Embodiment I | 10 | 89-91 | 407 | 497-506 |
| Embodiment J | 11 | 92-98 | 408 | 507-515 |
| Embodiment K | 13 | 99-113 | 409 | 516-525 |
| Embodiment L | 19 | 119-123 | 410 | 526-528 |
| Embodiment M | 20 | 124-131 | 411 | 529-530 |
| Embodiment N | 22 | 137-142 | 412 | 531 |
| Embodiment O | 24 | 143-153 | 413 | 532-537 |
| Embodiment P | 26 | 154-161 | 414 | 538-545 |
| Embodiment Q | 27 | 162-166 | 415 | 540-553 |
| Embodiment R | 29 | 168-175 | 416 | 554-561 |
| Embodiment S | 30 | 176-193 | 417 | 532-574 |
| Embodiment T | 34 | 202-211 | 419 | 575-584 |
| Embodiment U | 39/59 | 223-229, | 420 | 585-592 |
| | | 270-277 | | |
| Embodiment V | 43 | 238 | 421-423 | 593 |
| Embodiment X | 47 | 240-242 | 424-425 | 594-598 |
| Embodiment Y | 49/54 | 244-247, | 426-428 | 599-601, |
| | | 257-260 | | 606-613 |
| Embodiment Z | 53 | 252-256 | 431 | |
| Embodiment AA | 56 | 262-265 | 432 | 614-620 |
| Embodiment AB | 62 | 283 | 433 | 621-625 |
| Embodiment AC | 65 | 286-289 | 434 | |
| Embodiment AD | 73 | 308-311 | 435 | 626-632 |
| Embodiment AE | 76 | 312-319 | 436 | 633-640 |
| Embodiment AF | 77 | 320-337 | 437 | 641-648 |
| Embodiment AG | 86/51 | 357-370, | 438-439 | 602-605, |
| | | 249-251 | | 649-658 |
| Embodiment AH | 87 | 371 | 440 | 659-663 |
| Embodiment AI | 89 | 373-393, | 441 | |
| | | 394-396 | | |
| Embodiment AJ | 90 | 394-396 | | |
| Embodiment AK | 91 | 397 | 442-443 | 664 |

In other embodiments, a polypeptide of option a) includes one or more PG+ peptides from different NTGA's, so as to construct polypeptides with desirable properties. For example one polypeptide of option a) may contain as part of its sequence an amino acid sequence of one or more PG+ peptides selected from any one of SEQ ID NOs 1-397. In particularly, a polypeptide of option a) may include one or more immunodominant PG+ peptides, like those recognized by at least 3 subjects in a population of 20 subjects, e.g. one or more sequences selected from any one of SEQ ID NOs: 23, 24, 32, 57, 59, 60, 64, 65, 67, 68, 74, 75, 76, 78, 83, 143, 148, 244, 246, 258, 387, 391, 393 and 397, or a sequence with 0, 1 or 2 mismatches compared to the SEQ ID NOs: 23, 24, 32, 57, 59, 60, 64, 65, 67, 68, 74, 75, 76, 78, 83, 143, 148, 244, 246, 258, 387, 391, 393 and 397.

Accordingly, a polypeptide of option c) and d) may also comprise GWT sequences or portions thereof, respectively, that derive from different NTGA's to construct polypeptides with desirable properties, for example high conservation throughout the entire sequence of the polypeptide.

The invention also relates to a molecule for use as a medicament, in particularly for use in relieving an allergic immune response against a pollen allergen other than a grass pollen allergen in a subject, wherein said molecule comprises or consists of

a) a polypeptide, which includes at least one amino acid sequence with 0, 1 or 2 mismatches compared to a sequence selected from any one of SEQ ID NOs: 1-397;

b) a polypeptide comprising an amino acid sequence (being of the same length as) and having at least 65% sequence similarity or identity to a sequence selected from any one of SEQ ID NOs: 398-443;

c) a polypeptide comprising an amino acid sequence having at least 65% sequence similarity or identity to a sequence selected from any one of SEQ ID NOs: 444-664; or

d) a polypeptide comprising an amino acid sequence having at least 65% sequence similarity or identity to a subsequence of at least 15 contiguous amino acid residues of a sequence selected from any of SEQ ID NOs: 444-664.

The invention also relates to the use of a molecule as a medicament, e.g. for the use of a molecule for the preparation of a medicament for relieving an allergic immune response against a pollen allergen other than a grass pollen allergen in a subject, wherein said molecule comprises or consists of

a) a polypeptide, which includes at least one amino acid sequence with 0, 1 or 2 mismatches compared to a sequence selected from any one of SEQ ID NOs: 1-397;

b) a polypeptide comprising an amino acid sequence (being of the same length as) and having at least 65% sequence similarity or identity to a sequence selected from any one of SEQ ID NOs: 398-443;

c) a polypeptide comprising an amino acid sequence having at least 65% sequence similarity or identity to a sequence selected from any one of SEQ ID NOs: 444-664; or

d) a polypeptide comprising an amino acid sequence having at least 65% sequence similarity or identity to a subsequence of at least 15 contiguous amino acid residues of a sequence selected from any of SEQ ID NOs: 444-664.

The invention relates in a further aspect to an immunogenic molecule, e.g. a molecule comprising of or consisting of

b) a polypeptide having at least 65% sequence similarity or identity to a sequence selected from any one of SEQ ID NOs: 398-443; or

c) a polypeptide having at least 65% sequence similarity or identity to a sequence selected from any one of SEQ ID NOs: SEQ ID NOs: 444-664.

For example, an immunogenic molecule may contain a conserved sequence of NTGA 6 (embodiment F) of the above table. Thus, in one particular aspect, a molecule comprises or consists of b) a polypeptide having at least 65% sequence similarity or identity to SEQ ID NOs: 403; or comprises or consists of c) a polypeptide having at least 65% sequence similarity or identity to a sequence selected from any one of SEQ ID NOs: 474-479. Other embodiments (A to AK) may be constructed the same way using the list above.

Also provided are cells expressing an immunogen described herein. In various embodiments, a cell expresses an immunogen. In certain aspects, a cell is a eukaryotic or prokaryotic cell and may be a mammalian, insect, fungal or bacterium cell.

An immunogen of the present invention is suitable as a reagent, for example in immunotherapy against various pollen allergies including a pollen allergy, which is not grass pollen allergy in a subject.

In other embodiments, there are provided nucleic acid molecules encoding a polypeptide of option a), b), c) or d) or a molecule comprising a polypeptide of option a), b), c) or d).

In additional aspects, there are provided compositions, for example pharmaceutical compositions comprising an immunogenic molecule of the invention. In one embodiment, a pharmaceutical composition is suitable for immunotherapy (e.g., treatment, desensitization, tolerance induction, bystander suppression). In certain embodiments, a pharmaceutical composition is a vaccine, i.e. suitable formulated for the purpose of vaccination.

### BRIEF DESCRIPTION OF THE DRAWINGS

FIG. 1: Conservation in transcriptome predicts peptide cross-reactivity. For each peptide, TG allergic donors were selected that reacted to the peptide after expanding PBMCs in vitro with TG extract. PBMCs were stimulated with individual peptides for 14 days and IL-5 responses were measured by ELISPOT to i) the peptide itself, ii) TG extract, iii) non-TG extracts (e.g. Amb a, Que, Ole e, Bet v, Cyn d), iv) pools of pre-defined peptide pools (P20 and P19) that did or did not contain the peptide as relevant and irrelevant controls. T cell cultures that did not induce a robust response (>=200 SFC) to the peptide itself were excluded. Reponses to extracts and peptide pools are expressed as the relative fraction of the response to the peptide itself, and capped at 100%.

FIG. 2: Sensitization pattern of an immunogen of the invention (NTGA 86/51): It is shown that the in vitro T-cell response towards NTGA 86/51 is much weaker compared to the response to allergen Phl p 5.

FIGS. 3A-C: Tolerance induction investigated in mice. Figures show that prophylactic sublingual immunotherapy treatment (SLIT) with NTGA 86/51 in mice is capable of inducing tolerance towards the immunogen itself (3A) as well as towards Phl p extract (3B), as shown by the ability of NTGA 86/51 to reduce the proliferation of cells of splenocytes from treated mice compared to buffer (sham) treated mice. In addition, it was shown that NTGA 6 is capable of inducing tolerance towards itself (3C) as observed by its ability to reduce proliferation of cells of splenocytes.

FIGS. 4A and 4B: Bystander tolerance induction investigated in mice. As shown in FIG. 4A, prophylactic SLIT treatment with NTGA 86/51 is capable of inducing direct tolerance (towards NTGA 86/51 itself), as demonstrated by reduced proliferation of splenocytes of NTGA 86/51-treated mice compared to buffer treated mice. Furthermore, FIG. 4B shows that SLIT treatment with OVA is also able to downregulate the NTGA 86/51 specific in vitro response, demonstrating bystander tolerance induction by OVA. Likewise, SLIT treatment with NTGA 86/51 is also able to induce bystander tolerance, as demonstrated by the decreased OVA-specific in vitro proliferation of splenocytes from NTGA 86/51-SLIT treated mice compared to buffer treated mice.

### DETAILED DESCRIPTION

Definitions

The following terms and phrases shall have the following meaning:

The term “a” or “an” refers to an indefinite number and shall not only be interpreted as “one” but also may be interpreted to mean “some”, “several” or one or more.

The term “conserved sequence” is in the present context meant to include that a given sequence contains at least 15 contiguous amino acids within the sequence that has less than 3 mismatches compared to another sequence of 15 amino acid residues. Longer stretches of conserved sequences may contain several numbers of stretches of at least 15 contiguous amino acids having less than 3 mismatches compared to another sequence of 15 amino acids.

In the present context, e.g. for the purpose of detecting a conserved sequence, the term “mismatch” is meant to include any substitution of an amino acid residue within the 15mer peptide.

The term “sensitized to” is generally meant to encompass that the subject has been exposed to an immunogen, e.g. an allergen or an antigen, in a manner that the individual's adaptive immune system displays memory to the immunogen, for example that the immunogen has induced detectable IgE antibodies against the immunogen and thus qualifies as an IgE-reactive antigen (allergen) and/or that T-cells stimulated in vitro are able to proliferate under the presence of the immunogen or fragments of the immunogen (e.g. linear peptides).

The term “allergic immune response” is meant to encompass a hypersensitivity immune response, e.g. type 1 immune response, such as typically an immune response that is associated with the production of IgE antibodies (i.e. IgE-mediated immune response) and/or production of cytokines usually produced by Th2 cells. An allergic immune response may be associated with an allergic disease, for example atopic dermatitis, urticaria, contact dermatitis, allergic conjunctivitis, allergic rhinitis, allergic asthma, anaphylaxis, food allergy and hay fever.

The term “grass pollen” is meant to designate pollen of the plant family Poaceae, for example pollen of the plant genus Anthoxanthum, Cynodon, Dactylis, Festuca, Holcus, Hordeum, Lolium, Oryza, Paspalum, Phalaris, Phleum, Poa, Secale, Sorghum, Triticum and Zea.

As used herein, an “immunogen” refers to a substance, including but not limited to a protein, polypeptide or peptide that modifies, e.g. elicits, induces, stimulates, promotes enhances or decreases, reduces, inhibits, suppresses, relieves an immune response when administered to a subject. For example, an immunogen may induce tolerance to itself in a subject. An immune response elicited by an immunogen may include, but is not limited to, a B cell or a T cell response. An immune response can include a cellular response with a particular pattern of lymphokine/cytokine production (e.g., Th1, Th2), a humoral response (e.g., antibody production, like IgE, IgG or IgA), or a combination thereof, to a particular immunogen. Particular immunogens are antigens and allergens.

The term “an antigen” refers to a particular substance to which an immunoglobulin (Ig) isotype may be produced in response to the substance. For example, an “IgG antigen” refers to an antigen that induces an IgG antibody response. Likewise, an “IgE antigen” refers to an antigen that induces an IgE antibody response (and thus qualifies as an allergen); an “IgA antigen” refers to a substance that induces an IgA antibody response, and so forth. In certain embodiments, such an immunoglobulin (Ig) isotype produced in response to an antigen may also elicit production of other isotypes. For example, an IgG antigen may induce an IgG antibody response in combination with one more of an IgE, IgA, IgM or IgD antibody response. Accordingly, in certain embodiments, an IgG antigen may induce an IgG antibody response without inducing an IgE, IgA, IgM or IgD antibody response.

The term “allergen” refers to a particular type of a substance that can elicit production of IgE antibodies, such as in predisposed subjects. For example, if a subject previously exposed to an allergen (i.e. is sensitized or is hypersensitive) comes into contact with the allergen again, allergic asthma may develop due to a Th2 response characterized by an increased production of type 2 cytokines (e.g., IL-4, IL-5, IL-9, and/or IL-13) secreted by CD4+ T lymphocytes

The term “subject” is meant to designate a mammal having an adaptive immune system, such as a human, a domestic animal such as a dog, a cat, a horse or cattle.

The term “immunotherapy” is meant to encompass treatment of a disease by inducing, enhancing, or suppressing an immune response. Typically, the therapeutically active agent is an immunogen, particularly an antigen, more particularly an allergen. An immunogen may be a protein or a fragment thereof (e.g. immunogenic peptide). Immunotherapy in connection with allergy usually encompasses repeated administration of a sufficient dose of the immunogen/antigen/allergen/ usually in microgram quantities, over a prolonged period of time, usually for more than 3 months, 6 months, 1 year, such as 2 or 3 years, during which period the immunogen may be administered daily or less frequent, such as several times a week, weekly, bi-weekly, or monthly, every second month or quarterly. Immunotherapy can be effected by specific immunotherapy or may be effected by bystander tolerance induction.

The term “specific immunotherapy” in connection with allergy is meant to designate that immunotherapy is conducted with the administration of an immunogen to which the subject is sensitized to, particularly an immunogen to which the patient has raised specific IgE antibodies to, e.g. major allergens.

As used herein, the term “immunological tolerance” refers to a) a decreased or reduced level of a specific immunological response (thought to be mediated at least in part by antigen-specific effector T lymphocytes, B lymphocytes, antibody, a combination); b) a delay in the onset or progression of a specific immunological response; or c) a reduced risk of the onset or progression of a specific immunological response to an immunogen, such as an antigen or an allergen. “Specific” immunological tolerance occurs when tolerance is preferentially invoked against certain immunogens in comparison with other immunogens. Tolerance is an active immunogen dependent process and differs from non-specific immunosuppression and immunodeficiency.

The term “bystander tolerance induction” in connection with allergy is meant to encompass that immunotherapy is conducted with the administration of an immunogen that elicits, induces, stimulates, promotes enhances or decreases, reduces, inhibits, suppresses, relieves an immune response against another unrelated immunogen, for example an allergen, e.g. major allergens of pollen. For example, an immunogen may induce immunological tolerance to itself, and may be able to reactivate T regulatory cells specific to the immunogen to down-regulate an immune response caused by another unrelated immunogen, e.g. an allergen. Thus, an immunogen may induce immunological tolerance to an unrelated antigen, e.g. an allergen including a pollen allergen described herein.

The term “treatment” refers to any type of treatment that conveys a benefit to a subject afflicted with allergy, including improvement in the condition of the subject (e.g., in one or more symptoms), delay in the onset of symptoms, slowing the progression of symptoms, or induce disease modification etc. Typical symptoms of an allergic reaction are nasal symptoms in the form of itchy nose, sneezing, runny nose, blocked nose; conjunctival symptoms in the form of itchy eyes, red eyes, watery eyes; and respiratory symptoms in the form of decreased lung function. The treatment may also give the benefit that the patient needs less concomitant treatment with corticosteroids or H1 antihistamines to suppress the clinical symptoms. As used herein, “treatment” is not necessarily meant to imply cure or complete abolition of symptoms, but refers to any type of treatment that imparts a benefit to a patient. Treatment may be initiated before the subject becomes sensitized to a protein. This may be realized by initiating immunotherapy before the subject has raised detectable serum IgE antibodies capable of binding specifically to the sensitizing protein or before any other biochemical marker indicative of an allergic immune response can be detected in biological samples isolated from the individual. Furthermore, treatment may be initiated before the subject has evolved clinical symptoms of the allergic disease, such as symptoms of allergic rhinitis, allergic asthma or atopic dermatitis.

The phrase “therapeutically sufficient amount” or “sufficient amount” is meant to designate an amount effective to reduce, suppress, relieve or eliminate an allergic immune response, e.g. an amount sufficient to achieve the desirable reduction in clinical relevant symptoms or manifestations of the allergic immune response. For example, a therapeutically sufficient amount may be the accumulated dose of a polypeptide, a set of polypeptides administered during a course of immunotherapy in order to achieve the intended effect or it may be the maximal dose tolerated within a given period. The total dose or accumulated dose may be divided into single doses administered daily, twice a week or more, weekly, every second or fourth week or monthly depending on the route of administration and the pharmaceutical formulation used. The total dose or accumulated dose may vary. It is expected that a single dose is in the microgram range, such as in the range of 5 to 500 microgram dependent on the nature of the polypeptide.

The term “patient responding to therapy,” such as “immunotherapy” is meant to designate that the patient has improvement in the symptoms of the allergic immune response caused by a pollen allergen. Symptoms may be the clinically symptoms of allergic rhinitis, allergic asthma allergic conjunctivitis, atopic dermatitis, food allergy and/or hay fever. Typically, the symptoms are the same as experienced with a flu/cold, sneezing, itching, congestion, coughing, feeling of fatigue, sleepiness and body aches. For example nasal symptoms in the form of itchy nose, sneezing, runny nose, blocked nose; conjunctival symptoms in the form of itchy eyes, red eyes, watery eyes; and respiratory symptoms in the form of decreased lung function. A responder may also be evaluated by monitoring the patient's reduced need for concomitant treatment with corticosteroids or H1 antihistamines to suppress the clinical symptoms. Symptoms may be subjectively scored or in accordance with official guidelines used in clinical trials of SIT.

The term “adjuvant” refers to a substance that enhances the immune response to an immunogen. Depending on the nature of the adjuvant, it can promote either a cell-mediated immune response, humoral immune response or a mixture of the two.

As used herein an “epitope” refers to a region or part of an immunogen that elicits an immune response when administered to a subject. In particular embodiments, an epitope is a T cell epitope, i.e., an epitope that elicits, stimulates, induces, promotes, increases or enhances a T cell activity, function or response. An immunogen can be analyzed to determine whether it include at least one T cell epitope using any number of assays (e.g. T cell proliferation assays, lymphokine secretion assays, T cell non-responsiveness studies, etc.). In the context of the present invention, a T-cell epitope refers to an epitope that are MHC Class II binders (i.e. HLA-II binders), for example HLA-II binders shown in Table 9.

As used herein, the term “immune response” includes T cell (cellular) mediated and/or B cell (humoral) mediated immune responses, or both cellular and humoral responses. Exemplary immune responses include T cell responses, e.g., lymphokine production, cytokine production and cellular cytotoxicity. T-cell responses include Th1 and/or Th2 responses. In addition, the term immune response includes responses that are indirectly affected by T cell activation, e.g., antibody production (humoral responses) and activation of cytokine responsive cells, e.g., eosinophils, macrophages. Immune cells involved in the immune response include lymphocytes, such as T cells (CD4+, CD8+, Th1 and Th2 cells, memory T cells) and B cells; antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer (NK) cells; myeloid cells, such as macrophages, eosinophils, mast cells, basophils, and granulocytes.

The term “subsequence” or “stretch” means a fragment or part of a longer molecule, e.g. of a full length molecule (e.g. wild type proteins of Tables 2 and 4) or a conserved region thereof (e.g. GWT sequences of Table 3). A subsequence or portion therefore consists of one or more amino acids less than the wild type polypeptide or a conserved region thereof.

As disclosed herein, some immunogens (NTGA's) recently detected in Timothy grass pollen share substantial identity and similarity with immunogens detected in at least weed or tree pollen. Thus, such immunogens can be used to broadly treat a subject with or at risk of developing an allergic immune response to a pollen allergen of a variety of pollen plant families, or broadly induce or promote tolerance of a subject to a pollen allergen of a variety of pollen plant families and may include promoting or inducing tolerance to the immunogen itself.

Thus, by the present invention it is now possible to relieve an immune response of a multisensitized subject caused by pollen allergens of different plant families by administering an immunogen described herein. Likewise, it is also now possible to treat subjects with different pollen allergies using the same immunogen or set of immunogens.

In certain embodiments, the immunogen is a molecule comprising or consisting of a) a polypeptide, which includes at least one amino acid sequence with 0, 1 or 2 mismatches compared to a sequence selected from any one of SEQ ID NOs: 1-397 set out in Table 1 (PG+ peptides). The immunogen may contain at least one T cell epitope optionally a Th-2 cell epitope. Thus, in some embodiments, the polypeptide of option a) includes at least one amino acid sequence with 0, 1 or 2 mismatches to a sequence selected from any one of SEQ ID NOs: 4, 8, 9, 10, 14, 18, 19, 20, 21, 22, 23, 24, 25, 26, 27, 28, 29, 30, 31, 32, 34, 35, 38, 40, 52, 53, 54, 55, 56, 57, 58, 59, 60, 62, 63, 64, 65, 66, 67, 68, 69, 70, 71, 72, 73, 74, 75, 436, 77, 78, 79, 80, 81, 82, 83, 85, 87, 88, 89, 90, 91, 92, 93, 94, 95, 96, 114, 115, 130, 131, 137, 138, 141, 142, 143, 144, 145, 146, 147, 148, 149, 150, 151, 152, 153, 158, 162, 163, 164, 165, 166, 169, 184, 196, 197, 199, 200, 204, 210, 211, 212, 213, 225, 226, 230, 231, 235, 241, 244, 245, 246, 247, 249, 250, 252, 255, 256, 257, 258, 260, 264, 272, 274, 275, 276, 277, 283, 284, 286, 287, 299, 303, 312, 314, 315, 317, 318, 326, 327, 332, 333, 334, 335, 336, 338, 339, 340, 343, 344, 345, 346, 347, 348, 349, 352, 353, 355, 370, 372, 374, 375, 376, 384, 385, 386, 387, 388, 389, 390, 391, 393, 394, 395, 396 and 397.

In methods and uses described herein, one may consider using an immunogen recognized by a greater number of individuals, for example a polypeptide of option a) that includes at least one amino acid sequence with 0, 1 or 2 mismatches to a sequence selected from any one of SEQ ID NOs: 18, 22, 23, 24, 25, 26, 28, 30, 32, 52, 53, 57, 58, 59, 60, 64, 65, 66, 67, 68, 70, 72, 73, 74, 75, 76, 78, 80, 82, 83, 85, 87, 91, 93, 95, 115, 141, 143, 145, 146, 147, 148, 152, 164, 245, 246, 258, 275, 315, 376, 385, 386, 387, 388, 389, 391, 393, 394, 395, 396 and 397. For example, the immunogen may be recognized by at least 3 subjects in a population of 20 subjects, e.g. wherein the polypeptide of option a) includes at least one amino acid sequence with 0, 1 or 2 mismatches to a sequence selected from any one of SEQ ID NOs: 23, 24, 32, 57, 59, 60, 64, 65, 67, 68, 74, 75, 76, 78, 83, 143, 148, 244, 246, 258, 387, 391, 393 and 397.

In some embodiments, the number of amino acid mismatches is 0 or 1, for example the immunogen may be a molecule comprising or consisting of a) a polypeptide, which includes at least one amino acid sequence with 0 or 1 mismatches compared to a sequence selected from any one of SEQ ID NOs: 10, 13, 21, 23, 28, 32, 36, 51, 63, 80, 81, 99, 100, 109, 110, 111, 120, 121, 122, 125, 135, 137, 139, 140, 149, 156, 158, 160, 161, 164, 184, 197, 198, 199, 200, 207, 230, 231, 233, 246, 260, 305, 339, 340, 359, 360, 361, 367, 368, 369, 370 and 395.

In certain embodiments, the immunogen is a molecule comprising at least one of the PG+ peptides of Table 1, e.g. a wild type protein found in pollen of the genus Phleum (e.g. Pleum Pratense). Therefore, an immunogen molecule of the invention, may consist of or comprise a polypeptide of option b) comprising an amino acid sequence having at least 65% sequence similarity or identity to a sequence selected from any one of SEQ ID NOs: 398-443 set out in Table 2 (including NTGA's 1, 2, 3, 4, 6, 7, 9, 10, 11, 13, 19, 20, 22, 24, 26, 27, 29, 30, 32, 34, 43, 44, 47, 53, 56, 62, 65, 73, 76, 77, 87, 89, 91, 5/64, 39/59, 49/54 and 86/51. A polypeptide of option b) may contain at least one T cell epitope, for example NTGA's 1, 2, 4, 6, 7, 9, 10, 11, 20, 22, 24, 26, 27, 29, 30, 32, 34, 47, 49, 51, 53, 56, 62, 65, 76, 77, 86, 89, 91, 5/64, 39/59, 49/54, and 86/51. Thus, in some embodiments, a polypeptide of option b) comprises an amino acid sequence with at least 65% similarity or identity to a sequence selected from any of SEQ ID NOs: 398, 399, 401, 403, 404, 406, 407, 408, 411, 412, 413, 414, 415, 416, 417, 418, 419, 424, 429 431, 432, 433, 434, 436, 437, 441, 443, 402, 420, 426 and 438-439.

In methods and uses described herein, one may consider using an immunogen containing many PG+ peptides, such as at least five PG+ peptides of Table 1 (NTGA's 1, 2, 4, 6, 7, 13, 19, 20, 22, 24, 26, 27, 30, 32, 34, 76, 77, 89, 5/64, 39/59, 49/54, 86/51). Thus, in some embodiments the polypeptide of option b) comprises an amino acid sequence with at least 65% similarity or identity to a sequence selected from any one of SEQ ID NOs: 398, 399, 401, 403, 404, 409, 410, 411, 412, 413, 414, 415, 417, 418, 419, 436, 437, 441, 402, 420, 426, and 438-439 set out in Table 2.

An immunogen may contain at least eight PG+ peptides of Table 1 (NTGA's 1, 2, 4, 6, 7, 13, 24, 30, 34, 76, 77, 89, 5/64, 39/59, 49/54, 86/51). Thus, in some embodiments the polypeptide of option b) comprises an amino acid sequence with at least 65% similarity or identity to a sequence selected from any one of SEQ ID NOs: 398, 399, 401, 403, 404, 409, 413, 417, 419, 436, 437, 441, 402, 420, 426, 438-439 set out in Table 2.

In other embodiments, one may consider using an immunogen with the potential to produce or induce a T cell response in a greater fraction of the population, for example NTGA's numbered 2, 6, 7, 9, 10, 11, 22, 24, 27, 49/54, 39/59, 76, 89, 91. Thus, a polypeptide of option b) may comprise an amino acid sequence with at least 65% similarity or identity to a sequence selected from any of SEQ ID NOs: 399, 403, 404, 406, 407, 408, 412, 413, 415, 426, 420, 436, 441 and 443. In some embodiments, the polypeptide is recognized by at least 3 subjects of a population of 20 subjects, for example a polypeptide of option b) may comprise an amino acid sequence with at least 65% similarity or identity to a sequence selected from any of SEQ ID NOs: 399, 403, 404, 413, 426, 441 and 443 (NTGA's 2, 6, 7, 49/54, 89 and 91).

As mentioned, methods and uses described herein relate to relieving an allergic immune response against a pollen allergen, which is not a grass pollen allergen, for example not a grass pollen allergen of the plant family Poales. The plant family Poales typically encompasses plant genera from any of Anthoxanthum, Conydon, Dactylis, Lollium, Phleum or Poa. In a particular embodiment, the allergic immune response is not against a grass pollen allergen of the plant genus Phleum, e.g. Phleum Pratense.

An immunogen of the present invention is conserved across a grass pollen (for example of at least grass pollen of Phleum Pratense (Phl p)) and at least one non-grass pollen species. Therefore, immunogens of the present invention may be used in relieving an allergic immune response against a non-grass pollen allergen. For example, an immunogen of the present invention may be used in relieving an allergic immune response against a pollen allergen of a plant family from any of Asteraceae, Betulaceae, Fagaceae, Oleaceae, and Plantaginaceae, for example of a plant genus selected any of Ambrosia, Artemisia, Helianthus, Alnus, Betula, Carpinus, Castanea, Corylus, Ostrya, Ostryopsis, Fagus, Quercus, Fraxinus, Ligustrum, Lilac, or Plantago provided that the immunogen identified in Phl p pollen is conserved to an immunogen of the particular selected non-grass pollen species. As shown, herein many immunogens are conserved across the plant genera Ambrosia, Betula, Fraxinus, Quercus, or Plantago. Thus, an immunogen of the present invention may be used in relieving an allergic immune response against a pollen allergen of a plant genus selected from any of Ambrosia, Betula, Fraxinus, Quercus and/or Plantago.

Advantageously, the methods and uses described herein, comprises relieving an allergic immune response against pollen allergens of different pollen families, for example at least pollen allergens of weed and tree pollen. This is not meant to exclude that an immunogen of the present invention may in addition be used to treat an allergic immune response against a grass pollen allergen, for example against a grass pollen allergen of a plant genus selected from any of Anthoxanthum, Conydon, Dactylis, Lollium, Phleum or Poa, in particularly of the plant genus Phleum.

In particular embodiments, the immunogenic molecule consists of or comprises an amino acid sequence conserved across a polypeptide found in a grass pollen and a weed pollen and therefore is eligible for being used as a reagent in relieving at least an allergic immune response against a weed pollen allergen of the genus Ambrosia in a subject, e.g. in a subject at least sensitized to a weed pollen allergen of the genus Ambrosia and optionally also sensitized to a grass pollen allergen. For example, the immunogen may consist of or comprise a polypeptide of option a) that includes at least one amino acid sequence with 0, 1 or 2 mismatches to a sequence selected from any one of SEQ ID NOs: 2, 3, 4, 5, 6, 7, 8, 9, 10, 11, 12, 13, 14, 15, 20, 21, 22, 23, 24, 25, 26, 27, 28, 29, 30, 31, 32, 33, 34, 35, 36, 37, 38, 39, 40, 42, 43, 44, 45, 46, 48, 49, 50, 51, 53, 54, 55, 56, 58, 59, 60, 61, 62, 63, 64, 65, 66, 67, 68, 69, 70, 7375, 76, 77, 78, 79, 80, 81, 83, 84, 85, 86, 87, 95, 97, 98, 99, 100, 101, 102, 103, 104, 105, 106, 107, 109, 110, 111, 114, 115, 116, 118, 120, 121, 122, 123, 125, 126, 127, 128, 129, 131, 132, 133, 134, 135, 136, 137, 138, 139, 140, 141, 142, 145, 146, 147, 149, 150, 151, 152, 153, 154, 155, 156, 157, 158, 159, 160, 161, 162, 163, 164, 166, 167, 169, 170, 171, 172, 175, 179, 180, 181, 182, 184, 186, 187, 189, 190, 191, 192, 193, 196, 197, 198, 199, 200, 201, 202, 203, 204, 205, 206, 207, 209, 210, 211, 212, 214, 215, 216, 217, 218, 223, 224, 225, 226, 227, 228, 229, 230, 231, 232, 233, 234, 235, 236, 237, 239, 242, 244, 245, 246, 247, 249, 251, 256, 257, 258, 259, 260, 264, 265, 266, 267, 268, 269, 271, 273, 275, 276, 277, 278, 280, 281, 282, 283, 284, 291, 292, 294, 296, 298, 299, 300, 301, 302, 304, 305, 306, 308, 309, 311, 325, 326, 327, 328, 329, 330, 331, 333, 336, 337, 339, 340, 341, 343, 344, 345, 348, 351, 352, 353, 354, 355, 357, 359, 360, 361, 362, 363, 364, 366, 367, 368, 369, 370, 371, 381, 394, 395, 396 and 397, including SEQ ID NOs with proven T cell response reactivity (SEQ ID NOs: 4, 8, 9, 10, 14, 20, 21, 22, 23, 24, 25, 26, 27, 28, 29, 30, 31, 32, 34, 35, 38, 40, 53, 54, 55, 56, 58, 59, 60, 62, 63, 64, 65, 66, 67, 68, 69, 70, 73, 75, 76, 77, 78, 79, 80, 81, 83, 85, 87, 95, 114, 115, 131, 137, 138, 141, 142, 145, 146, 147, 149, 150151, 152, 153, 158, 162, 163, 164, 166, 169, 184, 196, 197, 199, 200, 204, 210, 211, 212, 225, 226, 230, 231, 235, 244, 245, 246, 247, 249, 256, 257, 258, 260, 264, 275, 276, 277, 283, 284, 299, 326, 327, 333, 336, 339, 340, 343, 344, 345, 348, 352, 353, 355, 370, 394, 395, 396 and 397).

In some embodiments thereof, the immunogen is a molecule containing at least 5 PG+ peptides with conservation across a grass pollen and a weed pollen, for example a molecule consisting of or comprising a polypeptide of option b) comprising an amino acid sequence with at least 65% similarity or identity to a sequence selected from any of SEQ ID NOs: 398, 399, 401, 403, 404, 411, 412, 413, 414, 416, 417, 418, 419, 437, 402, 420, 426, 438-439 (NTGA's 1, 2, 4, 6, 7, 20, 22, 24, 26, 29, 30, 32, 34, 77, 5/64, 39/59, 49/54 and 86/51)

In some embodiments thereof, the immunogen is a molecule containing at least 8 PG+ peptides with conservation across a grass pollen and in a weed pollen, for example a molecule consisting of or comprising a polypeptide of option b) comprising an amino acid sequence with at least 65% similarity or identity to a sequence selected from any of SEQ ID NOs: 398, 399, 401, 403, 404, 409, 413, 414, 417,419, 437, 402, 420, 426, 438-439. (NTGA's 1, 2, 4, 6, 7, 24, 26, 30, 34, 77, 5/64, 39/59, 49/54 and 86/51).

In other particular embodiments, the immunogen consists of or comprises an amino acid sequence conserved across polypeptides found in a grass pollen and a tree pollen and therefore is eligible for being used as a reagent in relieving at least an allergic immune response against a tree pollen allergen of the plant genus Quercus or Betula in a subject, e.g. in a subject at least sensitized to a tree pollen allergen of the genus Quercus or Betula and optionally also sensitized to a grass pollen allergen. For example, the immunogen may consist of or comprises a polypeptide of option a) that includes at least one amino acid sequence with 0, 1 or 2 mismatches to a sequence selected from any one of SEQ ID NOs: 1, 2, 3, 4, 5, 6, 7, 8, 9, 10, 11, 12, 13, 14, 15, 18, 19, 20, 21, 23, 24, 25, 26, 27, 28, 29, 30, 31, 32, 33, 34, 35, 36, 37, 39, 40, 41, 42, 43, 44, 45, 46, 47, 48, 49, 50, 51, 53, 55, 56, 58, 59, 60, 61, 62, 63, 64, 65, 66, 67, 68, 6970, 71, 72, 73, 74, 75, 76, 77, 78, 79, 80, 81, 82, 83, 84, 85, 88, 89, 90, 91, 92, 95, 97, 98, 99, 100, 101, 103, 104, 105, 106, 107, 108, 109, 110, 111, 112, 113, 114, 115, 117, 119, 120, 121, 122, 123, 124, 125, 126, 128, 129, 130, 131, 132, 133, 134, 135, 136, 137, 138, 139, 140, 141, 143, 145, 146, 147, 148, 149, 150, 151, 152, 153, 154, 155, 156, 158, 159, 160, 161, 162, 163, 164, 165, 166, 169, 172, 176, 178, 179, 180, 181, 182, 184, 186, 187, 189, 190, 191, 192, 193, 194, 195, 196, 197, 198, 199, 200, 201, 202, 203, 204, 205, 206, 207, 208, 209, 210, 212, 214, 215, 216, 217, 218, 219, 220, 222, 223, 224, 226, 228, 229, 230, 231, 232, 233, 234, 235, 236, 237, 238, 239, 241, 242, 244, 245, 246, 247, 248, 249, 250, 251, 253, 254, 255, 256, 257, 258, 259, 260, 261, 263, 264, 266, 267, 268, 269, 270, 271, 272, 273, 274, 276, 277, 278, 280, 281, 283, 284, 285, 286, 287, 288, 290, 292, 294, 295, 296, 297, 298, 299, 300, 301, 302, 304, 305, 306, 308, 310, 311, 312, 313, 314, 315, 316, 317, 318, 319, 320, 321, 322, 323, 324, 325, 327, 328, 329, 330, 331, 333, 336, 337, 338, 339, 340, 341, 343, 344, 345, 346, 347, 348, 349, 350, 351, 352, 353, 354, 355, 357, 358, 359, 360, 361, 363, 364, 365, 366, 367, 368, 369, 370, 371, 372, 373, 374, 376, 377, 378, 379, 380, 381, 382, 383, 384, 385, 386, 388, 389, 390, 391, 392, 393, 394, 395, 396 and 397, including SEQ ID NOs with proven T cell response reactivity (SEQ ID NOs: 4, 8, 9, 10, 14, 18, 19, 20, 21, 23, 24, 25, 26, 27, 28, 29, 30, 31, 32, 34, 35, 40, 53, 55, 56, 58, 59, 60, 62, 63, 64, 65, 66, 67, 68, 69, 70, 71, 72, 73, 74, 75, 76, 77, 78, 79, 80, 81, 82, 83, 85, 88, 89, 90, 91, 92, 95, 114, 115, 130, 131, 137138, 141, 143, 145, 146, 147, 148, 149, 150, 151, 152, 153, 158, 162, 163, 164, 165, 166, 169, 184, 196, 197, 199, 200, 204, 210, 212, 226, 230, 231, 235, 241, 244, 245, 246, 247, 249, 250, 255, 256, 257, 258, 260, 264, 272, 274, 276, 277, 283, 284, 286, 287, 299, 312, 314, 315, 317, 318, 327, 333, 336, 338, 339, 340, 343, 344, 345, 346, 347, 348, 349, 352, 353, 355, 370, 372, 374, 376, 384, 385, 386, 388, 389, 390, 391, 393, 394, 395, 396 and 397).

In some embodiments thereof, the immunogen is a molecule containing at least 5 PG+ peptides with conservation across a grass pollen and a tree pollen, for example a molecule consisting of or comprising a polypeptide of option b) comprising an amino acid sequence with at least 65% similarity or identity to a sequence selected from any of SEQ ID NOs: 398, 399, 401, 403, 404, 409, 410, 411, 412, 413, 414, 415, 417, 418, 419, 436, 437, 441, 402, 420, 426, 438-439 (NTGA's 1, 2, 4, 6, 7, 13, 19, 20, 22, 24, 26, 27, 30, 32, 34, 76, 77, 89, 5/64, 39/59, 49/54, 86/51.)

In some embodiments thereof, the immunogen is a molecule containing at least 8 PG+ peptides with conservation across a grass pollen and a tree pollen, for example a molecule consisting of or comprising a polypeptide of option b) comprising an amino acid sequence with at least 65% similarity or identity to a sequence selected from any of SEQ ID NOs: 398, 399, 401, 403, 404, 409, 413, 417, 419, 436, 437, 441, 402, 420, 426, 438-439 (NTGA's 1, 2, 4, 6, 7, 13, 24, 30, 34, 76, 77, 89, 5/64, 39/59, 49/54 and 86/51).

In other particular embodiments, the immunogen consists of or comprises an amino acid sequence conserved across polypeptides found in a grass pollen, a weed pollen and a tree pollen and therefore is eligible for being used as a reagent in relieving at least an allergic immune response against a weed pollen allergen of the genus Ambrosia and/or a tree pollen allergen of the plant genus Quercus or Betula in a subject, e.g. in a subject at least sensitized to a weed pollen allergen of the plant genus Ambrosia, and/or a tree pollen allergen of the genus Quercus or Betula and optionally also sensitized to a grass pollen allergen. For example, the immunogen may consist of or comprising a polypeptide of option a) that includes at least one amino acid sequence with 0, 1 or 2 mismatches to a sequence selected from any one of SEQ ID NOs: 2, 3, 4, 5, 6, 7, 8, 9, 10, 11, 12, 13, 14, 15, 20, 21, 23, 24, 25, 26, 27, 28, 29, 30, 31, 32, 33, 34, 35, 36, 37, 39, 40, 42, 43, 44, 45, 46, 48, 49, 50, 51, 53, 55, 56, 58, 59, 60, 61, 62, 63, 64, 65, 66, 67, 68, 69, 70, 73, 75, 76, 7778, 79, 80, 81, 83, 84, 85, 95, 97, 98, 99, 100, 101, 103, 104, 105, 106, 107, 109, 110, 111, 114, 115, 120, 121, 122, 123, 125, 126, 128, 129, 131, 132, 133, 134, 135, 136, 137, 138, 139, 140, 141, 145, 146, 147, 149, 150, 151, 152, 153, 154, 155, 156, 158, 159, 160, 161, 162, 163, 164, 166, 169, 172, 179, 180, 181, 182, 184, 186, 187, 189, 190, 191, 192, 193, 196, 197, 198, 199, 200, 201, 202, 203, 204, 205, 206, 207, 209, 210, 212, 214, 215, 216, 217, 218, 223, 224, 226, 228, 229, 230, 231, 232, 233, 234, 235, 236, 237, 239, 242, 244, 245, 246, 247, 249, 251, 256, 257, 258, 259, 260, 264, 266, 267, 268, 269, 271, 273, 276, 277, 278, 280, 281, 283, 284, 292, 294, 296, 298, 299, 300, 301, 302, 304, 305, 306, 308, 311, 325, 327, 328, 329, 330, 331, 333, 336, 337, 339, 340, 341, 343, 344, 345, 348, 351, 352, 353, 354, 355, 357, 359, 360, 361, 363, 364, 366, 367, 368, 369, 370, 371, 381, 394, 395, 396 and 397, including SEQ ID NOs with proven T cell response reactivity (SEQ ID NOs: 4, 8, 9, 10, 14, 20, 21, 23, 24, 25, 26, 27, 28, 29, 30, 31, 32, 34, 35, 40, 53, 55, 56, 58, 59, 60, 62, 63, 64, 65, 66, 67, 68, 69, 70, 73, 75, 76, 77, 78, 79, 80, 81, 83, 85, 95, 114, 115, 131, 137, 138, 141, 145, 146, 147, 149, 150, 151, 152, 153, 158, 162, 163, 164, 166, 169, 184, 196, 197, 199, 200, 204, 210, 212, 226, 230, 231, 235, 244, 245, 246, 247, 249, 256, 257, 258, 260, 264, 276, 277, 283, 284, 299, 327, 333, 336, 339, 340, 343, 344, 345, 348, 352, 353, 355, 370, 394, 395, 396 and 397).

In some embodiments thereof, the immunogen is a molecule containing at least 5 PG+ peptides with conservation across a grass pollen, a weed pollen and a tree pollen, for example a molecule consisting of or comprising a polypeptide of option b) comprising an amino acid sequence with at least 65% similarity or identity to a sequence selected from any of SEQ ID NOs: 398, 399, 401, 403, 404, 409, 411, 412, 413, 414, 417, 418, 419, 437, 420, 426, 438-439 (NTGA's 1, 2, 4, 6, 7,13, 20, 22, 24, 26, 30, 32, 34, 77, 39/59, 49/54 and 86/51).

In some embodiments thereof, the immunogen is a molecule containing at least 8 PG+ peptides with conservation across a grass pollen, a weed pollen and a tree pollen, for example a molecule consisting of or comprising a polypeptide of option b) comprising an amino acid sequence with at least 65% similarity or identity to a sequence selected from any of SEQ ID NOs: 398, 399, 401, 403, 404, 409, 413, 417, 419, 420, 426, 438-439 (NTGA's 1, 2, 4, 6, 7, 13, 24, 30, 34, 39/59, 49/54, 86/51).

In still some embodiments thereof, the immunogen comprises conserved regions (GWT) conserved across polypeptides identified in a grass, a weed and a tree pollen. Thus, in some embodiments the immunogen is a molecule consisting of or comprising a polypeptide of option c) comprising an amino acid sequence having at least 65% sequence similarity or identity to a sequence selected from any one of SEQ ID NOs: 444-449, 450-456, 457-459, 460-465, 466-473, 474-479, 480-485, 486-496, 497-506, 507-515, 516-525, 526-528, 529-530, 531, 532-537, 538-545, 540-553, 554-561, 532-574, 575-584, 585-592, 593, 594-598, 599-601, 606-613, 614-620, 621-625, 626-632, 633-640, 641-648, 602-605, 649-658, 659-663 and 664 as set out in Table 3. GWT sequences of Table 3 is contained in NTGA's 1, 2, 3, 4, 5/64, 6, 7, 9, 10, 11, 13, 19, 20, 22, 24, 26, 27, 29, 30, 34, 39, 51, 43, 47, 49/54, 56, 62, 73, 76, 77, 86/51, 87 and 91, respectively. As may be observed from Table 3, the GWT sequences of NTGA's 19, 20, 26, 30, 77 and 91 include longer conserved stretches covering a considerable portion of the wild type sequence. For example, NTGA 91 is highly conserved across the wild type sequences found in pollen of at least the genera Phleum, Ambrosia and Quercus.

In still other particular embodiments, the immunogen consists of or comprises an amino acid sequence conserved across polypeptide identified in the plant genera Ambrosia, Plantago, Fraxinus, Olea and Quercus and therefore is eligible for being used as a reagent in relieving at least an allergic immune response against a pollen allergen of the plant genera Ambrosia, Plantago, Fraxinus, Olea and Quercus in a subject, e.g. in a subject at least sensitized to a pollen allergen of the plant genera Ambrosia, Plantago, Fraxinus, Olea and Quercus and optionally also sensitized to a grass pollen allergen. For example, the immunogen may consist of or comprising a polypeptide of option a) that includes at least one amino acid sequence with 0, 1 or 2 mismatches to a sequence selected from any one of SEQ ID NOs: 2, 4, 5, 6, 7, 8, 9, 10, 11, 12, 13, 15, 20, 21, 23, 25, 26, 27, 28, 31, 32, 34, 35, 36, 37, 39, 40, 42, 43, 44, 49, 50, 51, 53, 56, 59, 60, 61, 63, 64, 67, 68, 69, 70, 75, 76, 77, 79, 80, 81, 84, 85, 95, 97, 98, 99, 100, 101, 103, 104, 105, 107109, 110, 111, 114, 115, 120, 121, 122, 123, 125, 126, 128, 129, 131, 132, 133, 134, 135, 136, 137, 138, 139, 140, 141, 145, 146, 147, 149, 150, 151, 152, 153, 154, 155, 156, 158, 159, 160, 161, 163, 164, 166, 169, 172, 179, 180, 181, 182, 184, 186, 187, 189, 190, 191, 192, 193, 196, 197, 198, 199, 200, 201, 202, 203, 204, 205, 206, 207, 209, 212, 214, 215, 216, 217, 223, 226, 228, 230, 231, 232, 233, 234, 235, 236, 237, 239, 244, 245, 246, 247, 249, 251, 256, 257, 258, 260, 264, 266, 267, 268, 269, 273, 277, 278, 284, 292, 294, 298, 299, 300, 301, 302, 304, 305, 306, 311, 325, 327, 329, 330, 331, 333, 336, 337, 339, 340, 341, 348, 351, 352, 353, 354, 355, 357, 359, 360, 361, 363, 364, 366, 367, 368, 369, 370, 371, 394, 395, 396 and 397, including SEQ ID NOs with proven T cell response reactivity (SEQ ID NOs: 4, 8, 9, 10, 20, 21, 23, 25, 26, 27, 28, 31, 32, 34, 35, 40, 53, 56, 59, 60, 63, 64, 67, 68, 69, 70, 75, 76, 77, 79, 80, 81, 85, 95, 114, 115, 131, 137, 138, 141, 145, 146, 147, 149, 150, 151, 152, 153, 158, 163, 164, 166, 169, 184, 196, 197, 199, 200, 204, 212, 226, 230231, 235, 244, 245, 246, 247, 249, 256, 257, 258, 260, 264, 277, 284, 299, 327, 333, 336, 339, 340, 348, 352, 353, 355, 370, 394, 395, 396 and 397).

In some embodiments thereof, the immunogen is a molecule containing at least 5 PG+ peptides with conservation across across the plant genera Ambrosia, Plantago, Fraxinus, Olea and Quercus, for example a molecule consisting of or comprising a polypeptide of option b) comprising an amino acid sequence with at least 65% similarity or identity to a sequence selected from any of SEQ ID NOs: 398, 399, 401, 403, 404, 409, 411, 412, 413, 414, 417, 418, 419, 437,420, 426 and 438-439 (NTGA's 1, 2, 4, 6, 7,13, 20, 22, 24, 26, 30, 32, 34, 77, 39/59, 49/54 and 86/51)

In some embodiments thereof, the immunogen is a molecule containing at least 8 PG+ peptides with conservation across across the plant genera Ambrosia, Plantago, Fraxinus, Olea and Quercus, for example a molecule consisting of or comprising a polypeptide of option b) comprising an amino acid sequence with at least 65% similarity or identity to a sequence selected from any of SEQ ID NOs: of 398, 399, 401, 403, 409, 413, 417, 420, 426 and 438-439. (NTGA's 1, 2, 4, 6, 13, 24, 30, 39/59, 49/54 and 86/51).

In still other particular embodiments, the immunogen consists of or comprises amino acid sequences conserved across polypeptides identified in the plant genera Ambrosia, Plantago, Fraxinus, Olea, Quercus and Betula and therefore is eligible for being used as a reagent in relieving at least an allergic immune response against a pollen allergen of the plant genera Ambrosia, Plantago, Fraxinus, Olea, Quercus and Betula in a subject, e.g. in a subject at least sensitized to a pollen allergen of the plant genera Ambrosia, Plantago, Fraxinus, Olea, Quercus and Betula and optionally also sensitized to a grass pollen allergen. For example, the immunogen may consist of or comprising a polypeptide of option a) that includes at least one amino acid sequence with 0, 1 or 2 mismatches to a sequence selected from any one of SEQ ID NOs: 4, 5, 6, 7, 8, 9, 10, 11, 12, 13, 15, 20, 21, 23, 25, 26, 27, 28, 31, 32, 34, 35, 36, 37, 39, 40, 42, 43, 49, 50, 51, 53, 56, 59, 60, 61, 63, 64, 67, 68, 69, 70, 75, 76, 77, 79, 80, 81, 84, 85, 95, 98, 99, 100, 101, 103, 105, 107, 109, 110, 111, 114, 115, 120, 121, 122, 123, 125, 126, 129, 131, 135, 137, 138, 139, 140, 145, 146, 147, 149, 150, 151, 152, 153, 154, 155, 156, 158, 159, 160, 161, 163, 164, 166, 172, 179, 180, 181, 182, 184, 186, 189, 190, 191, 192, 193, 196, 197, 198, 199, 200, 202, 203, 204, 205, 206, 207, 209, 212, 214, 215, 216, 217, 223, 226, 228, 230, 231, 232, 233, 234, 235, 236, 237, 239, 251, 264, 266, 273, 277, 278, 284, 292, 294, 299, 300, 304, 305, 306, 325, 327, 329, 330, 331, 333, 336, 339, 340, 341, 348, 351, 352, 353, 354, 355, 357, 359, 360, 361, 363, 364, 366, 367, 368, 369, 370, 371, 394, 395, 396 and 397, including SEQ ID NOs with proven T cell response reactivity (SEQ ID NOs: 4, 8, 9, 10, 20, 21, 23, 25, 26, 27, 28, 31, 32, 34, 35, 40, 53, 56, 59, 60, 63, 64, 67, 68, 69, 70, 75, 76, 77, 79, 80, 81, 85, 95, 114, 115, 131, 137, 138, 145, 146, 147, 149, 150, 151, 152, 153, 158, 163, 164, 166, 184, 196, 197, 199, 200, 204, 212, 226, 230, 231, 235, 264, 277, 284, 299, 327, 333, 336, 339, 340, 348, 352, 353, 355, 370, 394, 395, 396 and 397).

In some embodiments thereof, the immunogen is a molecule containing at least 5 PG+ peptides with conservation across across the plant genera Ambrosia, Plantago, Fraxinus, Olea and Quercus and Betula, for example a molecule consisting of or comprising a polypeptide of option b) comprising an amino acid sequence with at least 65% similarity or identity to a sequence selected from any of SEQ ID NOs: 398, 399, 401, 403, 404, 409, 411, 412, 413, 414, 417, 418, 419, 437, 420, 426 and 438-439 (NTGA's 1, 2, 4, 6, 7, 13, 20, 22, 24, 26, 30, 32, 34, 77, 39/59, 49/54 and 86/51.)

In some embodiments thereof, the immunogen is a molecule containing at least 8 PG+ peptides with conservation across across the plant genera Ambrosia, Plantago, Fraxinus, Olea and Quercus and Betula, for example a molecule consisting of or comprising a polypeptide of option b) comprising an amino acid sequence with at least 65% similarity or identity to a sequence selected from any of SEQ ID NOs: of 398, 399, 401, 403, 409, 413, 417, 420, 426 and438-439. (NTGA's 1, 2, 4, 6, 13, 24, 30, 39/59, 49/54 and 86/51.)

As mentioned, an immunogen of the invention may relieve an allergic immune response to a pollen allergen. Immunogens eligible for relieving an allergic immune response to an allergen unrelated to the immunogen is thought, at least in part, to be mediated via bystander tolerance induction, which mechanism requires, at least in part, co-existence of the immune response triggering allergen and the unrelated immunogen at the target organ.

Therefore, a polypeptide of option a), b), c) or d) may be derived from a wild type protein that co-releases/co-elutes with the pollen allergen that the subject is sensitized to and to which allergen the allergic immune response is sought relieved. In the present context, where multiple pollen allergies should be treated using one immunogen or a set of immungens, the wild type sequence of a polypeptide may be able to be “co-released” from multiple different pollen species.

In the present context, the term “co-release” or “co-elute” refers to an immunogen that starts release from a hydrated pollen within a period overlapping with a major allergen to which the allergic immune response is sought relieved. As major allergens start release from pollen within few minutes after hydration of pollen and continues to be released within the next 30 or 60 minutes, the term “co-release” or “co-elute” may refers to that an immunogen of the invention starts being released from pollen within 30 minutes after hydration of the pollen.

For example, a polypeptide of option a), option b), option c) or option d) may be derived from a polypeptide that co-releases with a major allergen from grass pollen of the genera Phleum and at least from a weed pollen of the genera Ambrosia.

Thus, in some embodiments, a polypeptide of option b) comprises an amino acid sequence with at least 65% similarity or identity to a sequence selected from any of SEQ ID NOs: 398, 401, 402, 403, 404, 413, 414, 416, 417, 420, 424-425, 438-439 and 442-443 (NTGA's 1, 4, 6, 7, 24, 26, 29, 30, 39, 47, 51, 59, 64, 86, 91, 5/64, 39/59 and 51/86 that starts release within 30 minutes after hydration from both grass and weed pollen); or a polypeptide of option a) that includes at least one amino acid sequence with 0, 1 or 2 mismatches to a sequence selected from any one of SEQ ID NOs:1-7, 34-45, 46-51, 52-74, 75-83, 143-153, 154-161, 168-175, 176-193, 223-229, 270-277, 240-242, 357-370,249-251 and 397; or a polypeptide of option c) comprises an amino acid sequence having at least 65% sequence similarity or identity to a sequence selected from any one of SEQ ID NOs: 444-449, 460-465, 466-473, 474-479, 480-485, 532-537, 538-545, 554-561, 532-574, 585-592, 594-598, 602-605, 649-658 and 664; or a polypeptide of option d) comprises an amino acid sequence having at least 65% sequence similarity or identity to a subsequence of at least 15 contiguous amino acid residues of a sequence selected from any of SEQ ID NOs: 444-449, 460-465, 466-473, 474-479, 480-485, 532-537, 538-545, 554-561, 532-574, 585-592, 594-598, 602-605, 649-658 and 664.

Furthermore, a polypeptide of option a), option b), option c) or option d) may be derived from a polypeptide that co-releases with a major allergen from grass pollen of the genera Phleum, and least from a tree pollen of the genera Quercus and/or betula.

In some embodiments, the polypeptide of option b) comprises an amino acid sequence with at least 65% similarity or identity to a sequence selected from any of SEQ ID NOs: 413, 416, 432 and 442-443 (NTGA's 24, 29, 56, 91 that starts release within 30 minutes after hydration from both grass and tree pollen (Que a); or a polypeptide of option a) includes at least one amino acid sequence with 0, 1 or 2 mismatches to a sequence selected from any one of SEQ ID NOs: 143-153, 168-175, 262-265 and 397; or a polypeptide of option c) comprises an amino acid sequence having at least 65% sequence similarity or identity to a sequence selected from any one of SEQ ID NOs: 532-537, 554-561, 614-620, 664; or a polypeptide of option d) comprises an amino acid sequence having at least 65% sequence similarity or identity to a subsequence of at least 15 contiguous amino acid residues of a sequence selected from any of SEQ ID NOs: 532-537, 554-561, 614-620 and 664.

Furthermore, a polypeptide of option a), option b), option c) or option d) may be derived from a polypeptide that co-releases with a major allergen from grass pollen of the genera Phleum, at least from a weed pollen of the genera Ambrosia and from a tree pollen of the genera Quercus and/or Betula.

In some embodiments, the polypeptide of option b) comprises an amino acid sequence with at least 65% similarity or identity to a sequence selected from any of SEQ ID NOs: 413, 416 and 442-443 (NTGA's 24, 29 and 91 that starts release within 30 minutes after hydration from both grass, weed (Amb a) and tree pollen (Que a) or a polypeptide of option a) includes at least one amino acid sequence with 0, 1 or 2 mismatches to a sequence selected from any one of SEQ ID NOs: 143-153, 168-175 and 397; or a polypeptide of option c) comprises an amino acid sequence having at least 65% sequence similarity or identity to a sequence selected from any one of SEQ ID NOs: 532-537, 554-561 and 664; or a polypeptide of option d) comprises an amino acid sequence having at least 65% sequence similarity or identity to a subsequence of at least 15 contiguous amino acid residues of a sequence selected from any of SEQ ID NOs: 532-537, 554-561 and 664.

It should be understood that an immunogen of the present invention may contain a PG+ peptides (with less than 1 to 3 mismatches) or a GWT sequence of Table 3. Examples are wild type sequences found in Phleum pollen as set out in Table 2, but other examples are wild type sequences found in other non-grass pollen, for example, a wild type sequence present in, based upon or derived from a pollen of a plant family from any of Asteraceae, Betulaceae, Fagaceae, Oleaceae, or Plantaginaceae, e.g. the plant genera Ambrosia, Artemisia, Helianthus, Alnus, Betula, Carpinus, Castanea, Corylus, Ostrya, Ostryopsis, Fagus, Quercus, Fraxinus, Ligustrum, Lilac, Olea or Plantago. Exemplary polypeptides are set out in Table 4. Thus a polypeptide of option b) may comprise an amino acid sequence having at least 65% similarity or identity to a sequence selected from any of SEQ ID NOs: 665-1109.

In specific embodiments of the invention, the polypeptide relates to NTGA 6, e.g. a polypeptide of option a) includes at least one amino acid sequence with 0, 1 or 2 mismatches compared to a sequence selected from any one of SEQ ID NOs: 52-74; the polypeptide of option b) comprises an amino acid sequence having at least 65% sequence similarity or identity to SEQ ID NOs: 403 or a homolog thereof in another pollen species, e.g. SEQ ID NOs: 704, 705, 706, 707, 708, 709, 711, 712, 713, 714, 715, 717, 718, 719, 720, 722, 723, 725; the polypeptide of option c) comprises an amino acid sequence having at least 65% sequence similarity or identity to a sequence selected from any one of SEQ ID NOs: 474-479 and polypeptide of option d) comprises an amino acid sequence having at least 65% sequence similarity or identity to a subsequence of at least 15 contiguous amino acid residues of a sequence selected from any of SEQ ID NOs: 474-479.

In specific embodiments of the invention, the polypeptide relates to NTGA 24, e.g. a polypeptide of option a) includes at least one amino acid sequence with 0, 1 or 2 mismatches compared to a sequence selected from any one of SEQ ID NOs: 143-153; the polypeptide of option b) comprises an amino acid sequence having at least 65% sequence similarity or identity to SEQ ID NOs: 413 or a homolog thereof in another pollen species, e.g. SEQ ID NOs: 808, 809, 810, 811, 812; the polypeptide of option c) comprises an amino acid sequence having at least 65% sequence similarity or identity to a sequence selected from any one of SEQ ID NOs: 532-537 and polypeptide of option d) comprises an amino acid sequence having at least 65% sequence similarity or identity to a subsequence of at least 15 contiguous amino acid residues of a sequence selected from any of SEQ ID NOs: 532-537.

In specific embodiments of the invention, the polypeptide relates to NTGA 29, e.g. a polypeptide of option a) includes at least one amino acid sequence with 0, 1 or 2 mismatches compared to a sequence selected from any one of SEQ ID NOs: 168-175; the polypeptide of option b) comprises an amino acid sequence having at least 65% sequence similarity or identity to SEQ ID NOs: 416 or a homolog thereof in another pollen species, e.g. SEQ ID NOs: 820, 821, 822, 823, 824, 825; the polypeptide of option c) comprises an amino acid sequence having at least 65% sequence similarity or identity to a sequence selected from any one of SEQ ID NOs: 554-561 and polypeptide of option d) comprises an amino acid sequence having at least 65% sequence similarity or identity to a subsequence of at least 15 contiguous amino acid residues of a sequence selected from any of SEQ ID NOs: 554-561.

In specific embodiments of the invention, the polypeptide relates to NTGA 39/59, e.g. a polypeptide of option a) includes at least one amino acid sequence with 0, 1 or 2 mismatches compared to a sequence selected from any one of SEQ ID NOs: 223-229, 270-277; the polypeptide of option b) comprises an amino acid sequence having at least 65% sequence similarity or identity to SEQ ID NOs: 420 or a homolog thereof in another pollen species, e.g. SEQ ID NOs: 865, 866, 867, 869, 870, 871; the polypeptide of option c) comprises an amino acid sequence having at least 65% sequence similarity or identity to a sequence selected from any one of SEQ ID NOs: 585-592 and polypeptide of option d) comprises an amino acid sequence having at least 65% sequence similarity or identity to a subsequence of at least 15 contiguous amino acid residues of a sequence selected from any of SEQ ID NOs: 585-592.

In specific embodiments of the invention, the polypeptide relates to NTGA 86/51, e.g. a polypeptide of option a) includes at least one amino acid sequence with 0, 1 or 2 mismatches compared to a sequence selected from any one of SEQ ID NOs: 357-370, 249-251; the polypeptide of option b) comprises an amino acid sequence having at least 65% sequence similarity or identity to SEQ ID NOs: 438-439 or a homolog thereof in another pollen species, e.g. SEQ ID NOs: 1025, 1026, 1027, 1029, 1030, 1031, 1032, 1033, 1034, 1035, 1036, 1037, 1040, 1041, 1042, 1043, 1044, 1046, 1048, 1049, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074; the polypeptide of option c) comprises an amino acid sequence having at least 65% sequence similarity or identity to a sequence selected from any one of SEQ ID NOs: 602-605, 649-658 and polypeptide of option d) comprises an amino acid sequence having at least 65% sequence similarity or identity to a subsequence of at least 15 contiguous amino acid residues of a sequence selected from any of SEQ ID NOs: 602-605, 649-658.

In specific embodiments of the invention, the polypeptide relates to NTGA 91, e.g. a polypeptide of option a) includes at least one amino acid sequence with 0, 1 or 2 mismatches compared to a sequence selected from any one of SEQ ID NOs: 397; the polypeptide of option b) comprises an amino acid sequence having at least 65% sequence similarity or identity to SEQ ID NOs: 442-443 or a homolog thereof in another pollen species, e.g. SEQ ID NOs: 1104, 1105, 1106, 1107, 1108, 1109; the polypeptide of option c) comprises an amino acid sequence having at least 65% sequence similarity or identity to a sequence selected from any one of SEQ ID NOs: 664 and polypeptide of option d) comprises an amino acid sequence having at least 65% sequence similarity or identity to a subsequence of at least 15 contiguous amino acid residues of SEQ ID NOs: 664.

In specific embodiments of the invention, the polypeptide relates to NTGA 1, e.g. a polypeptide of option a) includes at least one amino acid sequence with 0, 1 or 2 mismatches compared to a sequence selected from any one of SEQ ID NOs: 1-7; the polypeptide of option b) comprises an amino acid sequence having at least 65% sequence similarity or identity to SEQ ID NOs: 398 or a homolog thereof in another pollen species, e.g. SEQ ID NOs: 665, 666, 667, 668, 669;the polypeptide of option c) comprises an amino acid sequence having at least 65% sequence similarity or identity to a sequence selected from any one of SEQ ID NOs: 444-449 and polypeptide of option d) comprises an amino acid sequence having at least 65% sequence similarity or identity to a subsequence of at least 15 contiguous amino acid residues of a sequence selected from any of SEQ ID NOs: 444-449.

As mentioned a polypeptide defined herein may comprise one or more PG+ peptide sequences or a corresponding sequence with 1 or 2 mismatches compared to the PG+ peptide. In certain embodiments, a polypeptide of option a) comprises two or more PG+ peptides, e.g. 2-25 PG+ peptides defined herein, e.g. 3-25, 4-25, 5-25, 6-25, 7-25 PG+ peptides, such as 2-20, 3-20, 4-20, 5-20, 6-20 PG+ peptides or a corresponding sequence with 1 or 2 mismatches compared to the PG+ peptide. For example, a polypeptide of option a) may include one or more immunodominant PG+ peptides, like those recognized by at least 3 subjects in a population of 20 subjects, e.g. a polypeptide of option a) may include one or more sequences selected from any one of SEQ ID NOs: 23, 24, 32, 57, 59, 60, 64, 65, 67, 68, 74, 75, 76, 78, 83, 143, 148, 244, 246, 258, 387, 391, 393 and 397, or a sequence with 0, 1 or 2 mismatches compared to the SEQ ID NOs: 23, 24, 32, 57, 59, 60, 64, 65, 67, 68, 74, 75, 76, 78, 83, 143, 148, 244, 246, 258, 387, 391, 393 and 397.

Likewise a polypeptide may comprise several stretches of conserved regions of Table 3 from different NTGA's or a subsequence thereof. For example, a polypeptide may comprise. 2-25 conserved regions set out in of Table 1 or 3, e.g. 3-25, 4-25, 5-25, 6-25, 7-25 conserved regions set out in of Table 1 or 3, such as 2-20, 3-20, 4-20, 5-20, 6 conserved regions set out in of Table 1 or 3, for example conserved sequences deriving from immunogens able to start release within 30 minutes after hydration. For example a polypeptide may comprise one or more conserved sequences of NTGAs shown to be released from pollen (Table 6).

Thus, in some embodiments, a polypeptide of a polypeptide of option c) comprises one or more amino acid sequences selected from any one of SEQ ID NOs: 444-449, 460-465, 466-473, 474-479, 480-485, 532-537, 538-545, 554-561, 532-574, 585-592, 594-598, 602-605, 649-658 and 664 or an amino sequences having at least 65% sequence similarity or identity to the SEQ ID NOs selected, in particularly, a polypeptide of option c) comprises one or more amino acid sequences selected from any one of SEQ ID NOs: 532-537, 554-561, 614-620, 664 or an amino sequences having at least 65% sequence similarity or identity to the SEQ ID NOs selected.

In still some embodiments, a polypeptide of option d) comprises one or more amino acid sequences having at least 65% sequence similarity or identity to a subsequence of at least 15 contiguous amino acid residues of a sequence selected from any of SEQ ID NOs: 444-449, 460-465, 466-473, 474-479, 480-485, 532-537, 538-545, 554-561, 532-574, 585-592, 594-598, 602-605, 649-658 and 664, in particularly a polypeptide of option d) comprises one or more amino acid sequences having at least 65% sequence similarity or identity to a subsequence of at least 15 contiguous amino acid residues of a sequence selected from any of SEQ ID NOs: 532-537, 554-561, 614-620 and 664.

In still some embodiments, a polypeptide of option a) may include one or more sequences selected from any one of SEQ ID NOs: 1-7, 34-45, 46-51, 52-74, 75-83, 143-153, 154-161, 168-175, 176-193, 223-229, 270-277, 240-242, 357-370,249-251 and 397, or a sequence with 0, 1 or 2 mismatches compared to the SEQ ID NOs: 1-7, 34-45, 46-51, 52-74, 75-83, 143-153, 154-161, 168-175, 176-193, 223-229, 270-277, 240-242, 357-370,249-251 and 397, in particularly a polypeptide of option a) may include one or more sequences selected from any one of SEQ ID NOs: 143-153, 168-175, 262-265 and 39, or a sequence with 0, 1 or 2 mismatches compared to the SEQ ID NOs: 143-153, 168-175, 262-265 and 397.

In certain embodiments, the immunogen is a molecule comprising or consisting of a polypeptide, which includes at least one amino acid sequence with 0, 1 or 2 mismatches compared to a sequence selected from any one of SEQ ID NOs: 246, 258 and 315 that are described in both Table 1 and Table 10. Furthermore, an immunogen of the present may contain other peptides set out in Table 10, where it can be demonstrated that the peptide is conserved with a corresponding sequence in a non-grass pollen species. Thus, an immunogen may be a molecule comprising or consisting of a polypeptide, which includes at least one amino acid sequence with 0, 1 or 2 mismatches compared to a sequence selected from any one of SEQ ID NOs: 1110-1177 set out in Table 10. The immunogen may contain at least one T cell epitope, optionally a Th-2 cell epitope.

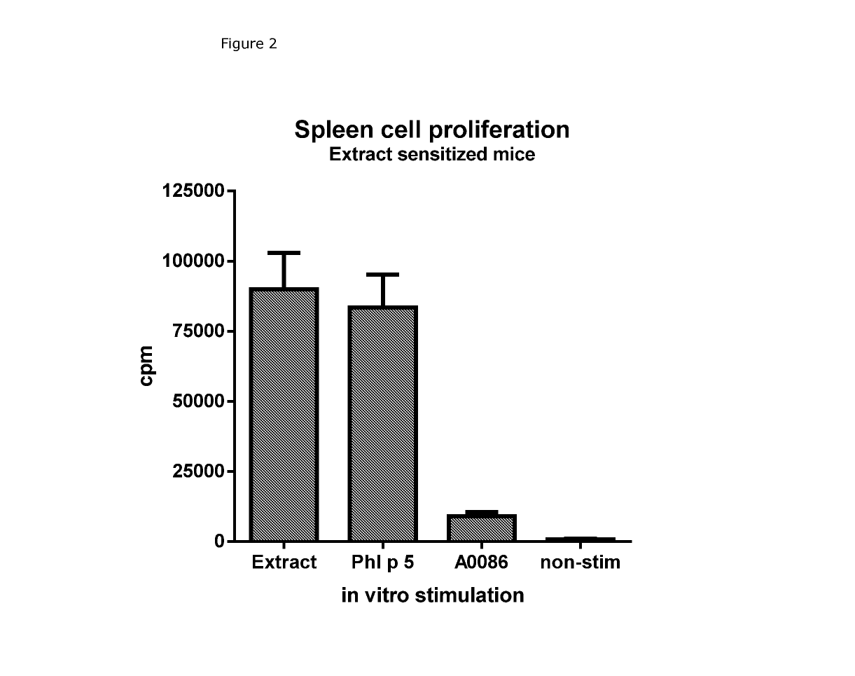

In some embodiments, an immunogen of the present invention is an IgE reactive molecule, e.g. able to bind to IgE antibodies specific for the immunogen. However, IgE reactivity towards an immunogen of the invention may only be conferred by a low fraction of an allergic population. Thus, an immunogen of the invention do not fall under the usual definitions of a major allergen. In some embodiments, the immunogen is able to react with, bind to or induce IgG antibodies in a subject, at least in detectable levels. In still other embodiments, the immunogen does not react with, bind to or induce IgG antibodies, at least in detectable levels. As demonstrated herein, an immunogen of the invention seems to be less immunogenic than a major allergen (FIG. 2), but still able to induce tolerance towards an unrelated immunogen (i.e. pollen allergen).

As mentioned, a subject eligible for being treated with an immunogen of the invention may also be sensitized to a grass pollen allergen, for example a grass pollen allergen of a plant genus selected from any of Anthoxanthum, Conydon, Phleum and Poa.

As disclosed herein, immunogens of the present invention may be found in various pollen families and share high identity and similarity with a wild type immunogen in non-grass pollen families and in other grass pollen families than of the genus Phleum. For example, a polypeptide of option b) comprises an amino acid sequence having at least 70% similarity or identity to a sequence selected from any one of SEQ ID NOs: 398-443, for example at least 75%, 80%, 85%, 90%, 91%, 92%, 93%, 94%, 95%, 96%, 97%, 98%, 99% similarity or identity. Examples on wild type immunogens with high identity and similarity to the wild type NTGA's are shown in Table 4. Here is disclosed wild type proteins found in other pollen species and which shares PG+ peptides or GWT regions with the NTGA's disclosed herein.

For example, wild type sequences comparable to NTGA 6 are found in at least Amb a, Amb p, Ant o, Bet v, Cyn d, Fra e, Lol p, Ole e, Pla I, Poa p, and Que a and comprises SEQ ID NOs: 704, 705, 706, 707, 708, 709, 710, 711, 712, 713, 714, 715, 716, 717, 718, 719, 720, 721, 722, 723, 724 and 725.

It follows that a polypeptide of option b) may comprise an amino acid sequence having at least 70% similarity or identity to a sequence selected from any one of SEQ ID NOs: 665-1109, for example at least 75%, 80%, 85%, 90%, 91%, 92%, 93%, 94%, 95%, 96%, 97%, 98%, 99% similarity or identity.

Furthermore, a polypeptide of option c) comprises an amino acid sequence having at least 70% similarity or identity to a sequence selected from any one of GWT sequences of Table 3 (SEQ ID NOs: 444-664), for example at least 75%, 80%, 85%, 90%, 91%, 92%, 93%, 94%, 95%, 96%, 97%, 98%, 99% similarity or identity. In certain embodiments thereof, it may be considered to utilize a polypeptide comprising an amino acid sequence having at least 85% similarity or identity to a sequence selected from any one of GWT sequences of Table 2. Furthermore, a polypeptide of option d) comprises an amino acid sequence having at least 70% sequence similarity or identity to a subsequence of at least 13, 14, 15 or 16 contiguous amino acid residues of a sequence selected from any of SEQ ID NOs: 444-664, for example at least 75%, 80%, 85%, 90%, 91%, 92%, 93%, 94%, 95%, 96%, 97%, 98%, 99% sequence similarity or identity to a subsequence of at least 13, 14, 15, or 16 contiguous amino acid residues of a sequence selected from any of SEQ ID NOs: 443-664. In certain embodiments thereof, it may be considered to utilize a polypeptide comprising an amino acid sequence having at least 85% sequence similarity or identity to a subsequence of at least 13, 14, 15 or 16 contiguous amino acid residues of any one GWT sequences of Table 2.

A subsequence may contain a T cell epitope, such as a Th2 cell epitope. A subsequence or a polypeptide described herein may have HLA Class II binding properties. HLA Class II binding can be predicted using NetMHClIpan-3.0 tool (Karosiene, Edita, Michael Rasmussen, Thomas Blicher, Ole Lund, Soren Buus, and Morten Nielsen. “NetMHClIpan-3.0, a Common Pan-specific MHC Class II Prediction Method Including All Three Human MHC Class II Isotypes, HLA-DR, HLA-DP and HLA-DQ.” Immunogenetics) available at the internet site <URL: http://www.cbs.dtu.dk/services/NetMHClIpan-3.0>.

A polypeptide of option a) may have different lengths according to the desirable use, for example of about 15-800 or more amino acid residues in length, for example 15-750, 15-700, 15-650, 15-600, 15-500 or more amino acid residues, for example 15-20, 15-25, 15-30, 20-25, 25-30, 30-35, 35-40, 45-50, 50-60, 60-70, 70-80, 90-100, 100-125, 125-150, 150-175, 175-200, 200-250, 250-300, 300-350, 350-400, 400-450, 450-500, 500-550, 550-600, 600-650, 650-700, 700-800 or more amino acid residues. One may consider utilizing short linear peptides, which when administered to a subject need not to be processed by an antigen presenting cells to interact with a relevant T cell receptor, but rather freely loaded onto a MHC class II molecule to interact with the relevant T cell receptor. Thus, in some embodiments, a polypeptide of option a) and a polypeptide of option d) has a length in the range of 15 to 30 amino acid residues, for example 15 to 25 amino acid residues. In other embodiments, a polypeptide of option a) is a longer polypeptide which comprises a secondary or tertiary structure, e.g. folded. Thus, in other embodiments, a polypeptide of option a) has a length in the range of 30 to 500 amino acid residues or more.

Polypeptides of option b) or c) may have the same length as the wild type sequence of the NTGA of Table 2, GWT sequence of Table 3, or the homolog of Table 4, respectively or may be shorter or longer. It is considered that the length of the amino acid sequence of a polypeptide of option b) is no more than 800 amino acid residues, for example no more than 750, 700, 650, 600, 550, 500 or 450 amino acid residues. Also it may be considered that the length of a polypeptide of option b) has an amino acid sequence length that is 80% to 120% of the length of any one of SEQ ID NOs: 398-443 and a polypeptide of option d) has an amino acid sequence length that is 80% to 120% of the length of any one of SEQ ID NOs: 444-664.

The term “identity” and “identical” and grammatical variations thereof, as used herein, mean that two or more referenced entities are the same (e.g., amino acid sequences). Thus, where two polypeptides are identical, they have the same amino acid sequence. The identity can be over a defined area (region or domain) of the sequence, e.g. over the sequence length of a sequence disclosed in Tables 1, 2, 3 or 4 or over a portion thereof e.g. at least 15 contiguous amino acid residues. Moreover, the identity can be over the length of the sequence overlapping the two polypeptides, when aligned with best fit with gaps permitted.

For example, to determine whether a polypeptide has at least 65% similarity or identity to a sequence set out in Tables 2, 3 and 4, the polypeptide may be aligned with a sequence of Table 2, 3 or 4 and the percent identity be calculated with reference to a sequence of Table 2, 3 and 4.

Identity can be determined by comparing each position in aligned sequences. A degree of identity between amino acid sequences is a function of the number of identical or matching amino acids at positions shared by the sequences, i.e. over a specified region. Optimal alignment of sequences for comparisons of identity may be conducted using a variety of algorithms, as are known in the art, including the Clustal Omega program available at http://www.ebi.ac.uk/Tools/msa/clustalo/, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerized implementations of these algorithms (such as GAP, BESTFIT, FASTA and TFASTA in the Wisconsin Genetics

Software Package, Genetics Computer Group, Madison, WI, U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at htt://www.ncbi.nlm.nih.gov/). Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch -2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

A polypeptide sequence is a “homologue” of, or is “homologous” to, another sequence if the two sequences have substantial identity over a specified region and a functional activity of the sequences is preserved or conserved, at least in part (as used herein, the term ‘homologous’ does not infer nor exclude evolutionary relatedness).

Examples of “homologous polypeptides” of the invention include polypeptides found in non-Timothy grass pollen and with high identity to the NTGA's disclosed in Table 2. For example, a homologous polypeptide may be found in pollen of plant families selected among Asteraceae, Betulaceae, Fagaceae, Oleaceae, or Plantaginaceae, e.g. the plant genera Ambrosia, Artemisia, Helianthus, Alnus, Betula, Carpinus, Castanea, Corylus, Ostrya, Ostryopsis, Fagus, Quercus, Fraxinus, Ligustrum, Lilac, Olea or Plantago.

Two polypeptide sequences are considered to be substantially identical if, when optimally aligned (with gaps permitted), they share at least about 65%, 70%, 75%, 80%, 85%, 90%, 95%, 96%, 97%, 98%, 99%, etc. identify over a specific region), for example, over all or a part of any amino acid sequence in Tables 1, 2, and 3, or if the sequences share defined functional motifs (e.g., epitopes). In particular aspects, the length of the sequence sharing the percent identity is at least 15, 16, 17, 18, 19, 20, etc. contiguous amino acids, e.g. more than 25, 30, 35, 40, 45 or 50 or more contiguous amino acids, including the entire length of a reference sequence of Tables 2, 3 or 4.

An “unrelated” or “non-homologous” sequence is considered to share less than 30% identity. More particularly, it may shares less than about 25% identity, with a polypeptide of the invention over a specified region of homology.

An amino acid sequence set out in any of Tables 2, 3 and 4 may contain modifications resulting in greater or less activity or function, such as ability to elicit, stimulate, induce, promote, increase, enhance, activate, modulate, inhibit, decreases, suppress, or reduce an immune response (e.g. a T cell response) or elicit, stimulate, induce, promote, increase or enhance immunological tolerance (desensitize) to an immunogen of the invention or a pollen allergen.

A modification includes deletions, including truncations and fragments; insertions and additions, substitutions, for example conservative substitutions, site-directed mutants and allelic variants.

Non-limiting examples of modifications include one or more amino acid substitutions (e.g., 1, 2, 3, 4, 5, 6, 7, 8, 9, 10, 11, 12, 13, 14, 15, 16, 17, 18, 19, 20-25, 25-30, 30-50, 50-100 or more residues), additions and insertions (e.g., 1, 2, 3, 4, 5, 6, 7, 8, 9, 10, 11, 12, 13, 14, 15 or more residues) and deletions (e.g., 1, 2, 3, 4, 5, 6, 7, 8, 9, 10, 11, 12, 13, 14, 15, 16, 17, 18, 19, 20-25, 25-30, 30-50, 50-100 or more) of a sequence set out in Tables 1, 2, 3 and 4.

The term “similarity” and “similar” and grammatical variations thereof, as used herein, mean that two or more referenced amino acid sequences contains a limited number of conservative amino acid substitutions of the amino acid sequence. A variety of criteria can be used to indicate whether amino acids at a particular position in a polypeptide are similar. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues have the same charge, or are both hydrophilic or hydrophobic. For example, a conservative amino acid substitution is one in which an amino acid residue is replaced with an amino acid residue having a similar side chain, which include amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, histidine); nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like. Proline, which is considered more difficult to classify, shares properties with amino acids that have aliphatic side chains (e.g., Leu, Val, Ile, and Ala). In certain circumstances, substitution of glutamine for glutamic acid or asparagine for aspartic acid may be considered a similar substitution in that glutamine and asparagine are amide derivatives of glutamic acid and aspartic acid, respectively. Conservative changes can also include the substitution of a chemically derivatized moiety for a non-derivatized residue, for example, by reaction of a functional side group of an amino acid. Variants and derivatives of polypeptides include forms having a limited number of one or more substituted residues.

As mentioned, a polypeptide of option a), b), c) and d) may be longer than the reference sequence set out in Tables 1, 2, 3 and 4.

An addition can be one or more additional amino acid residues. For example, a polypeptide of option a) may contain amino acid residues in addition to the 15 amino acid residues of the PG+ peptide, and optionally, the additional amino acid residues may be identical to those present in the wild type NTGA from which the PG+ peptide derives from. Thus, in some embodiments, the polypeptide of option a) comprises one or more amino acid residues in addition to the 15 contiguous amino acids (PG+ peptide) set out in Table 1, wherein the additional amino acid residue(s) is/are selected from an amino acid residue or an amino acid sequence within the wild type protein of which the PG+ peptide is a part of (e.g. wild type sequences of Tables 2 or 4 or a GWT sequence of Table 3). For example, the wild type amino acid residue or wild type amino acid sequence to be added may be adjacent to, subtended, comprised within, overlapping with or is a part of the PG+ peptide sequence, when present in its natural biological context within the wild type protein. An illustrative example is a PG+ peptide of NTGA 6 as set out in Table 1 that may be extended with amino acid residues from NTGA 6 set out in Table 2, or a homolog thereof set out in Table 3, such as amino acid residues adjacent to the PG+ sequence when aligned with NTGA 6 or the homolog thereof.

Likewise, a polypeptide of option c) may contain additional amino acid residues in addition to the GWT sequence set out in Table 3. Thus, a polypeptide of option c) may comprise one or more amino acid residues in addition to the GWT sequence set out in Table 3, wherein the additional amino acid residue(s) is/are selected from an amino acid residue or an amino acid sequence within the wild type protein of which the GWT sequence is a part of (e.g. a wild type protein of Tables 2 or 4). An illustrative example is a GWT sequence of NTGA 6 as set out in Table 2 that may be extended with amino acid residues from NTGA 6 set out in Table 2, or a homolog thereof set out in Table 3, such as amino acid residues adjacent to the GWT sequence when aligned with the corresponding wild type protein, NTGA 6 or a homolog thereof of Table 4.

The additional amino acid residues may be added to the N- and/or C- terminal end of a sequence set out in Tables 1, 2, 3 and 4, such as additional amino acids selected from amino acids flanking the N- and/or C- terminal ends when sequence is aligned with the source protein it is present in, based upon or derived from. Thus, where a sequence derives from NTGA 6, the additional amino acids may be the amino acids flanking the N- and/or C-terminal ends of the sequence when aligned to NTGA 6.

In one embodiment, a polypeptide of option a), b), c) or d) is derivatized. Specific non-limiting examples of derivatization are covalent or non-covalent attachment of another molecule. Specific examples include glycosylation, acetylation, phosphorylation, amidation, formylation, ubiquitination, and derivatization by protecting/blocking groups and any of numerous chemical modifications.

In particular embodiments, a derivative is a fusion (chimeric) sequence, an amino acid sequence having one or more molecules not normally present in the wild type sequence covalently attached to the sequence. The term “chimeric” and grammatical variations thereof, when used in reference to a sequence, means that the sequence contains one or more portions that are derived from, obtained or isolated from, or based upon other physical or chemical entities. For example, a chimera of two or more different polypeptides may have one part a polypeptide, and a second part of the chimera may be from a different sequence, or unrelated protein sequence.

Another particular example of a derivatized polypeptide is one in which a second heterologous sequence, i.e., heterologous functional domain is attached (covalent or non-covalent binding) that confers a distinct or complementary function. Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral), a metal (gold, silver), and radioisotope. For example, a tag such as T7 or polyhistidine can be attached in order to facilitate purification or detection of a protein, peptide, etc. For example, a 6-HIS tag may be added to the C- or N-terminal end of a polypeptide of option a), b), c) or d), e.g. the 6-HIS sequence GHHHHHHGSGMLDI, which optionally may remain in the immunogen when administered to a subject. Thus, a polypeptide linked to a Tag containing histidines may easily be purified by use of a HIS tag affinity column).

Accordingly, there are provided polypeptides linked to a heterologous domain, wherein the heterologous functional domain confers a distinct function on the polypeptide.

In some embodiments, the polypeptide is derivatized for example to improve solubility, stability, bioavailability or biological activity. For example, tagged polypeptides and fusion proteins; and modifications, including peptides having one or more non-amino acyl groups (q.v., sugar, lipid, etc.) covalently linked to the polypeptide and post-translational modifications.

Linkers, such as amino acid or peptidomimetic sequences may be inserted between the sequence and the addition (e.g., heterologous functional domain) so that the two entities maintain, at least in part, a distinct function or activity. Linkers may have one or more properties that include a flexible conformation, an inability to form an ordered secondary structure or a hydrophobic or charged character, which could promote or interact with either domain. Amino acids typically found in flexible protein regions include Gly, Asn and Ser. Other near neutral amino acids, such as Thr and Ala, may also be used in the linker sequence. The length of the linker sequence may vary without significantly affecting a function or activity of the fusion protein (see, e.g., U.S. Pat. No. 6,087,329). Linkers further include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Further non-limiting examples of derivatives are detectable labels. Thus, in another embodiment, the invention provides polypeptides that are detectably labeled. Specific examples of detectable labels include fluorophores, chromophores, radioactive isotopes (e.g., S³⁵, P³², I¹²⁵), electron-dense reagents, enzymes, ligands and receptors. Enzymes are typically detected by their activity. For example, horseradish peroxidase is usually detected by its ability to convert a substrate such as 3,3-′,5,5-′-tetramethylbenzidine (TMB) to a blue pigment, which can be quantified.

Modified polypeptides also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

A polypeptide of the invention may be modified to avoid oxidation, improve solubility in aqueous solution, avoid aggregation, overcome synthesis problems etc. For example the polypeptide amino acid sequence may include the following modifications:
a glutamate residue present at the N- terminus of a peptide replaced with pyroglutamate;
addition of one or more lysine amino residue(s) at the N- or C-terminus of the peptide;
addition of one or more arginine amino residue(s) at the N- or C-terminus of the peptide;
one or more modifications selected from the following: (a) any cysteine residues in the wild type sequence of the peptide are replaced with serine or 2-aminobutyric acid; (b) hydrophobic residues in the up to three amino acids at the N or C terminus of the wild type sequence of the peptide are deleted; (c) any two consecutive amino acids comprising the sequence Asp-Gly in the up to four amino acids at the N or C terminus of the wild type sequence of the peptide are deleted; and/or (d) one or more positively charged residues are added at the N- and/or C-terminus.

In particular, a polypeptide may comprise one, two or more lysine or arginine amino acid residue(s) added to the N- or C-terminus of the peptide to be modified, which may improve the aqueous solubility.

In particular, a polypeptide of the invention may comprise one or more cysteine residues that are substituted with amino acid residues less prone to oxidation, e.g. serine or arginine.

Polypeptides may be provided in the form of a salt, for example as a pharmaceutically acceptable and/or a physiologically acceptable salt. For example, the salt may be an acid addition salt with an inorganic acid, an acid addition salt with an organic acid, a salt with a basic inorganic acid, a salt with a basic organic acid, a salt with an acidic or basic amino acid or a mixture thereof. In particular embodiments of the invention a salt, such as a pharmaceutically acceptable salt, is an acetate salt.

The invention provides polypeptides and molecules in isolated and/or purified form.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g. polypeptides or molecules as defined herein) can also be “substantially pure” or “purified” when free of most or all of the materials with which it typically associates with in nature. Thus, an isolated polypeptide that also is substantially pure or purified does not include polypeptides or polynucleotides present among millions of other sequences, such as polypeptide of an peptide library or nucleic acids in a genomic or cDNA library, for example.

A “substantially pure” or “purified” composition can be combined with one or more other molecules. Thus, “substantially pure” or “purified” does not exclude combinations of compositions, such as combinations of polypeptides other antigens, agents, drugs or therapies.

Polypeptides can be prepared recombinantly, chemically synthesized, isolated from a biological material or source, and optionally modified, or any combination thereof. A biological material or source would include an organism that produced or possessed any polypeptide or molecule set forth herein. A biological material or source may further refer to a preparation in which the morphological integrity or physical state has been altered, modified or disrupted, for example, by dissection, dissociation, solubilization, fractionation, homogenization, biochemical or chemical extraction, pulverization, lyophilization, sonication or any other means of manipulating or processing a biological source or material. Polypeptides, such as immunogenic molecules disclosed herein may be modified by substituting, deleting or adding one or more amino acid residues in the amino acid sequence and screening for biological activity, for example eliciting an immune response. A skilled person will understand how to make such derivatives or variants, using standard molecular biology techniques and methods, described for example in Sambrook et al. (2001) Molecular Cloning: a Laboratory Manual, 3^{rd} ed., Cold Spring Harbour Laboratory Press).

Polypeptides and molecules that are provided herein can be employed in various methods and uses. Such methods and uses include, for example, administration in vitro and in vivo of one or more polypeptides or molecules thereof. The methods and uses provided include methods and uses of modulating an immune response (e.g. an allergic immune response), including, among others, methods and uses of relieving an immune response (e.g. allergic immune response), protecting and treating subjects against a disorder, disease (e.g. allergic disease); and methods and uses of providing immunotherapy, such as specific immunotherapy against an allergic immune response, e.g. allergy.

In particular embodiments, methods and uses include administration or delivery of an immunogen provided herein to modulate an immune response in a subject, including, for example, modulating an immune response to a pollen allergen or the immunogen.

As used herein, the term “modulate,” means an alteration or effect on the term modified. In certain embodiments, modulating involves decreasing, reducing, inhibiting, suppressing, relieving an immune response in a subject to an allergen or an immunogen provided herein. In other embodiments, modulating involves eliciting, stimulating, inducing, promoting, increasing or enhancing an immune response in a subject to an antigen or allergen. Thus, where the term “modulate” is used to modify the term “immune response against an allergen in a subject” this means that the immune response in the subject to the allergen or immunogen is altered or affected (e.g., decreased, reduced, inhibited, suppressed, limited, controlled, prevented, elicited, promoted, stimulated, increased, induced, enhanced, etc.

Methods and uses of modulating an immune response against an allergen or immunogen as described herein may be used to provide a subject with protection against an allergic immune response or immune reaction to the allergen or immunogen, or symptoms or complications caused by or associated with the allergen or immunogen. Accordingly, in other embodiments, methods and uses include administering an immunogen of the invention to protect or treat a subject against an allergic immune response, or one or more symptoms caused by or associated with an allergen. In still other embodiments, methods and uses include administering or delivering an immunogen of the invention to elicit, stimulate, induce, promote, increase or enhance immunological tolerance of a subject to an allergen or immunogen disclosed herein.

In various embodiments, there are provided methods and uses of providing a subject with protection against an allergic immune response, or one or more symptoms caused by or associated with an allergen or immunogen disclosed herein. In various aspects, a method or use includes administering to the subject an amount of an immunogen of the invention sufficient to provide the subject with protection against the allergic immune response, or symptoms caused by or associated with the allergen or immunogen.

Methods and uses of the invention include providing a subject with protection against an allergen or an immunogen, or symptoms caused by or associated with the subject's exposure to the allergen or immunogen, for example, vaccinating the subject to protect against an allergic immune response to the allergen or immunogen, for example with an immunogen provided herein. In certain embodiments, methods and uses include protecting the subject against an allergic immune response by inducing tolerance of the subject (desensitizing) to the allergen, and optionally to the immunogen.

As used herein, the terms “protection,” “protect” and grammatical variations thereof, when used in reference to an allergic immune response or symptoms caused by or associated with the exposure to allergen, means preventing an allergic immune response or symptoms caused by or associated with the exposure to the allergen, or reducing or decreasing susceptibility to an allergic immune response or one or more symptoms caused by or associated with the exposure to the allergen.

An allergic immune response includes but is not limited to an allergic reaction, hypersensitivity, an inflammatory response or inflammation. In certain embodiments allergic immune response may involve one or more of cell infiltration, production of antibodies, production of cytokines, lymphokines, chemokines, interferons and interleukins, cell growth and maturation factors (e.g., differentiation factors), cell proliferation, cell differentiation, cell accumulation or migration (chemotaxis) and cell, tissue or organ damage or remodeling. In particular aspects, an allergic immune response may include allergic rhinitis; atopic dermatitis; allergic conjunctivitis and asthma. Allergic responses can occur systemically, or locally in any region, organ, tissue, or cell. In particular aspects, an allergic immune response occurs in the skin, the upper respiratory tract, the lower respiratory tract, pancreas, thymus, kidney, liver, spleen, muscle, nervous system, skeletal joints, eye, mucosal tissue, gut or bowel.

Methods and uses herein include relieving, including treating, a subject for an allergic immune response, or one or more symptoms caused by or associated with an allergen. Such methods and uses include administering to a subject an amount of an immunogen sufficient to relieve, such as treat, the subject for the allergic immune response, or one or more symptoms caused by or associated with the allergen.

Methods and uses of the invention include treating or administering a subject previously exposed to an allergen or immunogen. Thus, in certain embodiments, methods and uses are for treating or protecting a subject from an allergic immune response, or one or more symptoms caused by or associated with secondary or subsequent exposure to an allergen or an immunogen.

Immunogens described herein may elicit, stimulate, induce, promote, increase or enhance immunological tolerance to an allergen and/or to the immunogen. Methods and uses of the invention therefore further include inducing immunological tolerance of a subject to an allergen or the immunogen itself. Thus, for example, immunogens described herein can be effective in relieving, such as treating an allergic immune response, including but not limited to an allergic immune response following a secondary or subsequent exposure of a subject to an allergen. In one embodiment, a method or use includes administering to the subject an amount of an immunogen sufficient to induce tolerance in the subject to the allergen or immunogen itself. In particular aspects, the immunological tolerance elicited, stimulated, induced, promoted, increased or enhanced may involve modulation of T cell activity, including but not limited to CD4+ T cells, CD8+ T cells, Th1 cells, Th2 cells and regulatory T cells. For example, immunological tolerance elicited, stimulated, induced, promoted, increased or enhanced from administration of the immunogen, may involve modulation of the production or activity of pro-inflammatory or anti-inflammatory cytokines produced by T cells.

In additional embodiments, a method or use of inducing immunological tolerance in a subject to an allergen includes a reduction in occurrence, frequency, severity, progression, or duration of physiological conditions, disorders, illnesses, diseases, symptoms or complications caused by or associated an allergic response to the allergen in the subject. Thus, in certain embodiments, inducing immunological tolerance can protect a subject against or treat a subject for an allergic immune response, or one or more symptoms caused by or associated with an allergen or the immunogen.

Methods and uses of the invention include treating a subject via immunotherapy, including specific immunotherapy. In one embodiment, a method or use includes administering to the subject an amount of an immunogen described herein. In one aspect, an immunogen administered to a subject during specific immunotherapy to treat the subject is the same immunogen to which the subject has been sensitized or is hypersensitive (e.g., allergic). In another non-limiting aspect, an immunogen is administered to a subject to treat the subject to a different immunogen, e.g. a pollen allergen to which the subject has been sensitized or is hypersensitive (e.g., allergic). Thus, the immunotherapeutic mechanism may involve bystander suppression of an allergic immune response caused by a pollen allergen by administering an unrelated immunogen, e.g. an immunogen disclosed herein.

As described herein, immunogens include T cell epitopes, such as Th2 cell epitopes. In methods and uses herein, the subject to be treated has a specific T-cell response to the immunogen before administering the first dose.

Accordingly, methods and uses of the invention include administering an amount of an immunogen (e.g., a T cell epitope-containing immunogen) to a subject sufficient to provide the subject with protection against an allergic immune response, or one or more symptoms caused by or associated with an allergen. In another embodiment, a method includes administering an amount of an immunogen (e.g., a T cell epitope-containing immunogen) to a subject sufficient to relieve, e.g. treat, vaccinate or immunize the subject against an allergic immune response, or one or more symptoms caused by or associated with an allergen.

The specific T-cell response may be monitored by determining by way of contacting a sample of PBMCs obtained from the subject with the immunogens and measuring the IL-5 secretion or IL-5 mRNA gene expression in response to the immunogen.

In accordance with the invention, methods and uses of modulating anti-allergen activity of T cells, including but not limited to CD8⁺ T cells, CD4⁺ T cells, Th1 cells or Th2 cells, in a subject are provided. In one embodiment, a method or use includes administering to a subject an amount of a polypeptide described herein or derivative thereof including an immunogenic molecule described herein, such as a T cell epitope, sufficient to modulate Th2 cell activity in the subject.

In certain embodiments, two or more immunogens may be administered to a subject, e.g. may be administered as a combination composition, or administered separately, such as concurrently or in series or sequentially. For example, methods and uses described herein comprise administration separately or as a combination: at least 2-25 polypeptides defined herein, or separately or as a combination of 3-25, 4-25, 5-25, 6-25, 7-25 polypeptides defined herein, or separately or as a combination of 2-20, 3-20, 4-20, 5-20, 6-20 defined herein, or separately or as a combination of 2-12, 3-12, 4-12, 5-12, 6-12, 7-12 polypeptides defined herein, or separately or as a combination of 2-10, 3-10, 4-10, 5-10, 6-10, 7-10 polypeptides defined herein.

For example, a there may be administered to a subject, e.g. as a combination composition, one or more immunodominant PG+ peptides, like those recognized by at least 3 subjects in a population of 20 subjects, e.g. composition comprising one more polypeptides of option a), wherein each polypeptide of option a) may independently include one or more sequences selected from any one of SEQ ID NOs: 23, 24, 32, 57, 59, 60, 64, 65, 67, 68, 74, 75, 76, 78, 83, 143, 148, 244, 246, 258, 387, 391, 393 and 397, or a sequence with 0, 1 or 2 mismatches compared to the SEQ ID NOs: 23, 24, 32, 57, 59, 60, 64, 65, 67, 68, 74, 75, 76, 78, 83, 143, 148, 244, 246, 258, 387, 391, 393 and 397.

Compositions may comprise one or more polypeptides, comprising a conserved region of Table 3 from different NTGA's or a subsequence thereof. For example, a composition may comprise 2-25 polypeptides of option d), wherein each option d) polypeptide independently comprises one or more amino acid sequences having at least 65% sequence similarity or identity to a subsequence of at least 15 contiguous amino acid residues of a sequence selected from any of SEQ ID NOs: 444-449, 460-465, 466-473, 474-479, 480-485, 532-537, 538-545, 554-561, 532-574, 585-592, 594-598, 602-605, 649-658 and 664, in particularly, wherein a polypeptide of option d) comprises one or more amino acid sequences having at least 65% sequence similarity or identity to a subsequence of at least 15 contiguous amino acid residues of a sequence selected from any of SEQ ID NOs: 532-537, 554-561, 614-620 and 664.

Compositions may comprise one or more polypeptides of option a), wherein each polypeptide of option a) may include one or more sequences selected from any one of SEQ ID NOs: 1-7, 34-45, 46-51, 52-74, 75-83, 143-153, 154-161, 168-175, 176-193, 223-229, 270-277, 240-242, 357-370,249-251 and 397, or a sequence with 0, 1 or 2 mismatches compared to the SEQ ID NOs: 1-7, 34-45, 46-51, 52-74, 75-83, 143-153, 154-161, 168-175, 176-193, 223-229, 270-277, 240-242, 357-370,249-251 and 397, in particular a polypeptide of option a) may include one or more sequences selected from any one of SEQ ID NOs: 143-153, 168-175, 262-265 and 39, or a sequence with 0, 1 or 2 mismatches compared to the SEQ ID NOs: 143-153, 168-175, 262-265 and 397.

Methods and uses of the invention therefore include any therapeutic or beneficial effect. In various methods embodiments, an allergic immune response, or one or more symptoms caused by or associated with an allergen is reduced, decreased, inhibited, limited, delayed or prevented. Methods and uses of the invention moreover include reducing, decreasing, inhibiting, delaying or preventing onset, progression, frequency, duration, severity, probability or susceptibility of one or more adverse symptoms, disorders, illnesses, diseases or complications caused by or associated with an antigen/allergen. In further various particular embodiments, methods and uses include improving, accelerating, facilitating, enhancing, augmenting, or hastening recovery of a subject from an allergic immune response, or one or more physiological conditions, symptoms or complications caused by or associated with an antigen/allergen. In yet additional various embodiments, methods and uses include stabilizing an allergic immune response, or one or more physiological conditions, symptoms or complications caused by or associated with an antigen/allergen.

A therapeutic or beneficial effect is therefore any objective or subjective measurable or detectable improvement or benefit provided to a particular subject. A therapeutic or beneficial effect can but need not be complete ablation of all or any allergic immune response, or one or more symptoms caused by or associated with an allergen. Thus, a satisfactory clinical endpoint is achieved when there is an incremental improvement or a partial reduction in an allergic immune response, or one or more symptoms caused by or associated with an allergen, or an inhibition, decrease, reduction, suppression, prevention, limit or control of worsening or progression of an allergic immune response, or one or more symptoms caused by or associated with an allergen, over a short or long duration (hours, days, weeks, months, etc.).

A therapeutic or beneficial effect also includes reducing or eliminating the need, dosage frequency or amount of a second therapeutic protocol or active such as another drug or other agent (e.g., anti-inflammatory) used for treating a subject having or at risk of having an allergic immune response, or one or more symptoms caused by or associated with an allergen. For example, reducing an amount of an adjunct therapy, such as a reduction or decrease of a treatment for an allergic immune response, or one or more symptoms caused by or associated with an allergen, or a specific immunotherapy, vaccination or immunization protocol is considered a beneficial effect. In addition, reducing or decreasing an amount of the immunogen used for specific immunotherapy, vaccination or immunization of a subject to provide protection to the subject is considered a beneficial effect.

Methods and uses described herein may relieve one or more symptoms of an allergic immune response or delays the onset of symptoms, slow the progression of symptoms, or induce disease modification. For example, the following symptoms may be decreased or eliminated; nasal symptoms in the form of itchy nose, sneezing, runny nose, blocked nose; conjunctival symptoms in the form of itchy eyes, red eyes, watery eyes; and respiratory symptoms in the form of decreased lung function. Furthermore, the beneficial effect of methods and uses described herein may be observed by the patient's need for less concomitant treatment with corticosteroids or H1 antihistamines to suppress the symptoms.

When an immunogen is administered to induce tolerance, an amount or dose of the immunogen to be administered, and the period of time required to achieve a desired outcome or result (e.g., to desensitize or develop tolerance to the allergen or immunogen) can be determined by one skilled in the art. The immunogen may be administered to the patient through any route known in the art, including, but not limited to oral, inhalation, sublingual, epicutaneous, intranasal, and/or parenteral routes (intravenous, intramuscular, subcutaneously, intradermal, and intraperitoneal).

Methods and uses of the invention include administration of an immunogen to a subject prior to contact by or exposure to an allergen; administration prior to, substantially contemporaneously with or after a subject has been contacted by or exposed to an allergen; and administration prior to, substantially contemporaneously with or after an allergic immune response, or one or more symptoms caused by or associated with an allergen.

As used herein, a “sufficient amount” or “effective amount” or an “amount sufficient” or an “amount effective” refers to an amount that provides, in single (e.g., primary) or multiple (e.g., booster) doses, a long term or a short term detectable or measurable improvement in a given subject or any objective or subjective benefit to a given subject of any degree or for any time period or duration (e.g., for minutes, hours, days, months, years, or cured).

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to a method of the invention, such as immunization, vaccination, specific immunotherapy and therapeutic treatments.

The term “subject” includes but is not limited to a subject at risk of allergen contact or exposure as well as a subject that has been contacted by or exposed to an allergen. A subject also includes those having or at risk of having or developing an immune response to an antigen or an allergen. Such subjects include mammalian animals (mammals), such domestic animal (dogs and cats), a farm animal (poultry such as chickens and ducks, horses, cows, goats, sheep, pigs), experimental animal (mouse, rat, rabbit, guinea pig) and humans.

Target subjects and subjects in need of treatment also include those at risk of allergen exposure or contact or at risk of having exposure or contact to an allergen. Accordingly, subjects include those at increased or elevated (high) risk of an allergic reaction; has, or has previously had or is at risk of developing hypersensitivity to an allergen; and those that have or have previously had or is at risk of developing asthma.

As mentioned, methods and uses described herein, relates to relieving an allergic immune response, e.g. preventing or treating an allergic immune response against a pollen allergen, which is not a grass pollen allergen by administering an immunogen described herein.

Non-grass pollen allergens are but not limited to pollen allergens of the plant families Asteraceae, Betulaceae, Fagaceae, Oleaceae, and/or Plantaginaceae, for example from pollen of a plant genus selected from any of Ambrosia, Artemisia, Helianthus, Alnus, Betula, Carpinus, Castanea, Corylus, Ostrya, Ostryopsis, Fagus, Quercus, Fraxinus, Ligustrum, Lilac or Plantago. Immunogens disclosed herein are conserved across a grass and at least a weed pollen and in particular embodiments, a non-grass pollen allergen is of the genus Ambrosia (e.g. Amb a and/ or Amb p). Immunogens disclosed herein are conserved across a grass and at least a Oak pollen and in particular embodiments, a non-grass pollen allergen is of the genus Quercus (e.g. Que a). Immunogens disclosed herein are conserved across a grass and at least a birch pollen and in particular embodiments, a non-grass pollen allergen is of the genus Betula (E.g. Bet v). Some immunogens are conserved across a grass, a weed and a tree pollen and in particular embodiments, a non-grass pollen allergen is of the genus Ambrosia, Betula and/or Oak. Where immunogens are conserved across several other pollen species, a non-grass pollen allergen may be e.g. Fraxinus, Alternaria or Plantago.

A grass pollen allergen includes for example a grass pollen allergen of the plant family Poales. The plant family Poales typically encompasses plant genera from any of Anthoxanthum, Conydon, Dactylis, Lollium, Phleum or Poa. In a particular embodiment, the allergic immune response is not against a grass pollen allergen of the plant genus Phleum, e.g. Phleum Pratense.

As immunogens of the invention are conserved across grass a pollen (e.g. Timothy grass pollen), the methods and uses described herein, comprises relieving an allergic immune response against grass pollen allergens as well as a non-grass pollen allergen.

Examples on well known non-grass pollen allergens are, but not limited to: Aln g 1, Aln g 4, Amb a 1, Amb a 2, Amb a 3, Amb a 4, Amb a 5, Amb a 6, Amb a 7, Amb a 8, Amb a 9, Amb a 10, Amb p 5, Amb t 5, Art v 1, Art v 2, Art v 3, Art v 4, Art v 5, Art v 6, Bet v 1, Bet v 2, Bet v 3, Bet v 4, Bet v 6, Bet v 7,Car b 1, Cas s 1, Cor a 6, Cor a 10, Fag s 1, Fra e 1, Hel a 1, Hel a, Lig v 1, Ole e 1, Ole e 2, Ole e 3, Ole e 4, Ole e 5, Ole e 6, Ole e 7, Ole e 8, Ole e 9, Ole e 10, Ole e 11, Ost c 1, Pla I, Que a 1, Syr v 1, Syr v 3.

Many of the well known pollen allergens are major allergens and thought to be the most important allergens in eliciting an allergic immune in a subject. Thus, in some embodiments, the non-grass pollen allergen at least is Amb a 1, Que a 1, Bet v 1, Bet v 2 and/ or Ole e 1.

Examples on grass pollen allergens are but not limited to; Ant o 1, Cyn d 1, Cyn d 7, Cyn d 12, Cyn d 15, Cyn d 22w, Cyn d 23, Cyn d 24, Dac g 1, Dac g 2, Dac g 3, Dac g 4, Dac g 5, Fes p 4, Hol I 1, Hol I 5, Hor v 1, Hor v 5, Lol p 1, Lol p 2, Lol p 3, Lol p 4, Lol p 5, Lol p 11, Ory s 1, Pas n 1, Pha a 1, Pha a 5, Phl p 1, Phl p 2, Phl p 4, Phl p 5, Phl p , Phl p 7, Phl p 11, Phl p 12, Phl p 13, Poa p 1, Poa p 5, Sec c 1, Sec c 5, Sec c 38 and/or Sor h 1, of which group 1 (e.g. Ant o 1, Cyn d 1, Dac g 1, Hol 1, Lol p 1, Pha a 1, Phl p 1 and Poa p) or group 5 allergens (Dac g 5, Lol p 5, Pha a 5, Phl p 5, Poa p 5) are considered major allergens important for the allergic immune response triggered by a grass pollen in a subject,

“Prophylaxis” and grammatical variations thereof mean a method or use in which contact, administration or in vivo delivery to a subject is prior to contact with or exposure to an allergen. In certain situations it may not be known that a subject has been contacted with or exposed to an allergen, but administration or in vivo delivery to a subject can be performed prior to manifestation of an allergic immune response, or one or more symptoms caused by or associated with an allergen. For example, a subject can be provided protection against an allergic immune response, or one or more symptoms caused by or associated with an allergen or provided immunotherapy with an immunogen of the present invention. In such case, a method or use can eliminate, prevent, inhibit, suppress, limit, decrease or reduce the probability of or susceptibility towards an allergic immune response, or one or more physiological conditions, symptoms or complications caused by or associated with an antigen/allergen.

“Prophylaxis” can also refer to a method or use in which contact, administration or in vivo delivery to a subject is prior to a secondary or subsequent exposure to an antigen/ allergen. In such a situation, a subject may have had a prior contact or exposure to an allergen. In such subjects, an acute allergic reaction may but need not be resolved. Such a subject typically may have developed anti-allergen antibodies due to the prior exposure. Immunization or vaccination, by administration or in vivo delivery to such a subject, can be performed prior to a secondary or subsequent allergen exposure. Such a method or use can eliminate, prevent, inhibit, suppress, limit, decrease or reduce the probability of or susceptibility towards a secondary or subsequent allergic immune response, or one or more symptoms caused by or associated with an allergen. In certain embodiments, such a method or use includes providing specific immunotherapy to the subject to eliminate, prevent, inhibit, suppress, limit, decrease or reduce the probability of or susceptibility towards a secondary or subsequent allergic immune response, or one or more physiological conditions, symptoms or complications caused by or associated with an antigen/allergen.

Treatment of an allergic reaction or response can be at any time during the reaction or response. An immunogen can be administered as a single or multiple dose e.g., one or more times hourly, daily, weekly, monthly or annually or between about 1 to 10 weeks, or for as long as appropriate (e.g. 3 months, 6 months or more, for example, to achieve a reduction in the onset, progression, severity, frequency, duration of one or more symptoms or complications associated with or caused by an allergic immune response, or one or more physiological conditions, symptoms or complications caused by or associated with an antigen/allergen.

Accordingly, methods and uses of the invention can be practiced one or more times (e.g., 1-10, 1-5 or 1-3 times) an hour, day, week, month, or year. The skilled artisan will know when it is appropriate to delay or discontinue administration. Doses can be based upon current existing protocols, empirically determined, using animal disease models or optionally in human clinical trials. Initial study doses can be based upon animal studies, e.g. a mouse, and the sufficient amount of immunogen to be administered for being effective can be determined. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, whether a subject has been previously exposed to the antigen/allergen, the onset, progression, severity, frequency, duration, probability of or susceptibility of the symptom, condition, pathology or complication, or vaccination or specific immunotherapy to which treatment is directed, the clinical endpoint desired, previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

Immunogens of the invention can be provided in compositions, and in turn such compositions can be used in accordance with the invention methods and uses. Such compositions, methods and uses include pharmaceutical compositions and formulations. In certain embodiments, a pharmaceutical composition includes one or more immunogens. In particular, aspects, such compositions and formulations may be a vaccine, including but not limited to a vaccine to protect against an allergic immune response, or one or more symptoms caused by or associated with an allergen.

A pharmaceutical comprises an immunogen of the invention and a pharmaceutically acceptable ingredient or carrier.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals.

Supplementary active compounds (e.g., preservatives, antibacterial, antiviral and antifungal agents) can also be incorporated into the compositions.

A composition may be lyophilized so as to enhance stability and ease of transportation. For the purpose of being used as a vaccine, the composition may be sterile.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery for which a composition can optionally be formulated include inhalation, intranasal, oral, buccal, sublingual, subcutaneous, intradermal, epicutaneous, rectal, transdermal, or intralymphatic.

In some embodiments, the pharmaceutical composition is aqueous and, in other embodiments, the composition is non-aqueous solutions, suspensions or emulsions of the peptide/protein, which compositions are typically sterile and can be isotonic with the biological fluid or organ of the intended recipient. Non-limiting illustrative examples include water, saline, dextrose, fructose, ethanol, vegetable or synthetic oils.

For oral, buccal or sublingual administration, a composition can take the form of for example a solid dosage form, e.g. tablets or capsules, optionally formulated as fast-integrating tablets/capsules or slow-release tablets/capsules. In some embodiments, the tablet is a freeze-dried, optionally fast-disintegrating tablet suitable for being administered under the tongue. A solid dosage form optionally is sterile, optionally anhydrous.

The pharmaceutical composition may also be formulated into a “unit dosage form”. As used herein a unit dosage form refers to physically discrete units suited as unitary dosages for the subject to be treated; each unit containing a predetermined quantity of a peptide/protein optionally in association with a pharmaceutical carrier (excipient, diluent, vehicle or filling agent) which, when administered in one or more doses, is calculated to produce a desired effect. Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

To increase an immune response, immunological tolerance or protection against an allergic immune response, or one or more symptoms caused by or associated with an allergen, immunogens can be mixed with adjuvants.

Adjuvants include, for example: oil (mineral or organic) emulsion adjuvants such as Freund's complete (CFA) and incomplete adjuvant (IFA) (WO 95/17210; WO 98/56414; WO 99/12565; WO 99/11241; and U.S. Pat. No. 5,422,109); metal and metallic salts, such as aluminum and aluminum salts, such as aluminum phosphate or aluminum hydroxide, alum (hydrated potassium aluminum sulfate); bacterially derived compounds, such as Monophosphoryl lipid A and derivatives thereof (e.g., 3 De-O-acylated monophosphoryl lipid A, aka 3D-MPL or d3-MPL, to indicate that position 3 of the reducing end glucosamine is de-O-acylated, 3D-MPL consisting of the tri and tetra acyl congeners), and enterobacterial lipopolysaccharides (LPS); plant derived saponins and derivatives thereof, for example Quil A (isolated from the Quilaja Saponaria Molina tree, see, e.g., “Saponin adjuvants”, Archiv. fur die gesamte Virusforschung, Vol. 44, Springer Verlag, Berlin, p243-254; U.S. Pat. No. 5,057,540), and fragments of Quil A which retain adjuvant activity without associated toxicity, for example QS7 and QS21 (also known as QA7 and QA21), as described in WO96/33739, for example; surfactants such as, soya lecithin and oleic acid; sorbitan esters such as sorbitan trioleate; and polyvinylpyrrolidone; oligonucleotides such as CpG (WO 96/02555, and WO 98/16247), polyriboA and polyriboU; block copolymers; and immunostimulatory cytokines such as GM-CSF and IL-1, and Muramyl tripeptide (MTP). Additional examples of adjuvants are described, for example, in “Vaccine Design—the subunit and adjuvant approach” (Edited by Powell, M. F. and Newman, M. 3.; 1995, Pharmaceutical Biotechnology (Plenum Press, New York and London, ISBN 0-306-44867-X) entitled “Compendium of vaccine adjuvants and excipients” by Powell, M. F. and Newman M.

Cosolvents may be added to the composition. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non- pathogenic microbial organism. Classes of antimicrobials include antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Pharmaceutical formulations and delivery systems appropriate for the compositions, methods and uses of the invention are known in the art (see, e.g. Remington: The Science and Practice of Pharmacy (David B. Troy, Paul Beringer Lippincott Williams & Wilkins) 2006).

Pharmaceutical compositions can be formulated to be compatible with a particular route of administration. Thus, pharmaceutical compositions include carriers, diluents, or excipients suitable for administration by various routes (For example excipients recorded in a Pharmacopiea). Exemplary routes of administration for contact or in vivo delivery, which a composition can optionally be formulated, include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, or intralymphatic.

Formulations suitable for parenteral administration include aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

Methods and uses of the invention may be practiced by any mode of administration or delivery, or by any route, systemic, regional and local administration or delivery.

Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic.

For oral administration, a composition can take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the invention is not entitled to antedate such publication by virtue of prior invention.

As used in this specification and the appended claims, the use of an indefinite article or the singular forms “a,” “an” and “the” include plural reference unless the context clearly dictates otherwise. In addition, it should be understood that the individual peptides, proteins, antigens, allergens (referred to collectively as compositions), or groups of compositions, modeled or derived from the various components or combinations of the compositions, and substituents described herein, are disclosed by the application to the same extent as if each composition or group of compositions was set forth individually. Thus, selection of particular peptides, proteins, antigens, allergens, etc. is clearly within the scope of the invention.

As used in this specification and the appended claims, the terms “comprise”, “comprising”, “comprises” and other forms of these terms are intended in the non-limiting inclusive sense, that is, to include particular recited elements or components without excluding any other element or component. Unless defined otherwise all technical and scientific terms used herein have the same meaning as commonly understood to one of ordinary skill in the art to which this invention belongs. As used herein, “about” means + or −5%. The use of the wild type (e.g., “or”) should be understood to mean one, both, or any combination thereof of the wild types, i.e., “or” can also refer to “and.”

As used in this specification and the appended claims, any concentration range, percentage range, ratio range or other integer range is to be understood to include the value of any integer within the recited range and, when appropriate, fractions thereof (such as one tenth and one hundredth of an integer), unless otherwise indicated. For example, although numerical values are often presented in a range format throughout this document, a range format is merely for convenience and brevity and should not be construed as an inflexible limitation on the scope of the invention. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 5-10, 10-20, 20-30, 30-40, 40-50, 50-75, 75-100, 100-150, and 150-175, includes ranges such as 5-20, 5-30, 5-40, 5-50, 5-75, 5-100, 5-150, 5-171, and 10-30, 10-40, 10-50, 10-75, 10-100, 10-150, 10-175, and 20-40, 20-50, 20-75, 20-100, 20-150, 20-175, and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. The invention is further exemplified by way of the following non-limited examples.

### REFERENCES

Oseroff C, Sidney 3, Kotturi MF, Kolla R, Alam R, Broide DH, et al. Molecular determinants of T cell epitope recognition to the common Timothy grass allergen. Journal of immunology 2010; 185:943-55.

P. Wang, 3. Sidney, C. Dow, B. Mothe, A. Sette, B. Peters. A systematic assessment of MHC class II peptide binding predictions and evaluation of a consensus approach. PLoS Comput Biol, 4 (2008), p. e1000048

P. Wang, 3. Sidney, Y. Kim, A. Sette, 0. Lund, M. Nielsen, et al. Peptide binding predictions for HLA DR, DP and DQ molecules. BMC Bioinform, 11 (2010), p. 568

Karosiene, Edita, Michael Rasmussen, Thomas Blicher, Ole Lund, Soren Buus, and Morten Nielsen. “NetMHClIpan-3.0, a Common Pan-specific MHC Class II Prediction Method Including All Three Human MHC Class II Isotypes, HLA-DR, HLA-DP and HLA-DQ.” Immunogenetics

### Tables

Table 1 indicates for each of the 397 PG+ peptides in which non-grass pollen species a matching peptide with either less than 3, less than 2 or zero mismatches are found. The number of TG grass allergic donors (n=20) with an in vitro T cell response to the TG peptide sequence is also shown.

TABLE 1
Table 1 Identification of conserved sequences across grass pollen and non-grass pollen.

| | | | | | | | | | | | | | | | | | | | | | # T cell | | |
|---|---|---|---|---|---|---|---|---|---|---|---|---|---|---|---|---|---|---|---|---|---|---|---|
| | | | | | | | | | | | | | | | | | | | | | responders | | |
| SEQ | | | Non-grass pollen species | | | | | | | | | | | | | | | | | | to TG | | |
| ID | NTGA | Phl p Sequence | Amb p | | | Pla l | | | Ole e | | | Fra e | | | Que a | | | Bet v | | | sequence | | |
| No | ID | (PG + peptide) | <3 | <2 | 0 | <3 | <2 | 0 | <3 | <2 | 0 | <3 | <2 | 0 | <3 | <2 | 0 | <3 | <2 | 0 | >0 | >1 | >2 |
| 1 | 1 | SDGTFARAAVPSGAS | x | x | | | | | | | | | | | x | | | | | | | | |
| 2 | 1 | KLGANAILAVSLAVC | x | x | x | x | x | | x | x | x | x | x | x | x | x | x | x | x | x | | | |
| 3 | 1 | KKIPLYQHIANLAGN | x | x | | | | | | | | | | | x | x | x | x | x | x | | | |
| 4 | 1 | GNKQLVLPVPAFNVI | x | x | | x | x | | x | x | | x | x | | x | x | | x | x | | x | | |
| 5 | 1 | KLAMQEFMILPTGAS | x | x | | x | x | x | x | x | | x | x | | x | x | | x | x | | | | |
| 6 | 1 | KMGVEVYHNLKSVIK | x | x | x | x | | | x | x | | x | x | | x | x | x | x | | | | | |
| 7 | 1 | GKVVIGMDVAASEFY | x | x | | x | x | x | x | x | x | x | x | x | x | x | x | x | x | x | | | |
| 8 | 1 | VYKSFVSEYPIVSIE | x | x | | x | | | x | x | | x | x | | x | x | | x | | | x | | |
| 9 | 1 | IVGDDLLVTNPTRVA | x | x | | x | x | | x | | | x | | | x | | | x | x | | x | | |
| 10 | 1 | NALLLKVNQIGSVTE | x | x | x | x | x | x | x | x | x | x | x | x | x | x | x | x | x | x | x | | |
| 11 | 1 | ETEDTFIADLAVGLS | x | x | | x | | | x | | | x | | | x | | | x | | | | | |
| 12 | 1 | RAAVPSGASTGVYEA | x | x | | x | x | | x | x | | x | x | x | x | x | | x | x | x | | | |
| 13 | 1 | ERLAKYNQLLRIEEE | x | x | x | x | x | x | x | x | x | x | x | x | x | x | x | x | x | x | | | |
| 14 | 1 | LGAAAVYAGLKFRAP | x | x | | | | | | | | | | | x | | | | | | x | | |
| 15 | 1 | GASTGVYEALELRDG | x | x | | x | x | | x | x | | x | x | x | x | x | | x | x | x | | | |
| 16 | 1 | QTELDNFMVHQLDGT | | | | x | | | | | | | | | | | | | | | | | |
| 17 | 1 | VDNVNSIIGPALIGK | | | | x | | | x | x | | x | x | | | | | x | | | | | |
| 18 | 2 | ENRSVLHVALRAPRD | | | | x | x | x | x | x | | x | x | | x | x | | x | | | x | x | |
| 19 | 2 | FLGPLFVHTALQTDP | | | | x | x | x | x | x | | x | x | | x | x | x | x | x | x | x | | |
| 20 | 2 | RQLRFLANVDPVDVA | x | x | x | x | x | | x | x | | x | x | | x | x | | x | x | | x | | |
| 21 | 2 | VVSKTFTTAETMLNA | x | x | x | x | x | x | x | x | x | x | x | x | x | x | x | x | x | x | x | | |
| 22 | 2 | VSKHMIAVSTNLKLV | x | x | | x | | | | | | | | | | | | | | | x | x | |
| 23 | 2 | RYSVCSAVGVLPLSL | x | x | x | x | x | | x | x | x | x | x | x | x | x | x | x | x | x | x | x | x |
| 24 | 2 | AVGVLPLSLQYGFPI | x | | | | | | x | | | x | | | x | | | x | | | x | x | x |
| 25 | 2 | VLLGLLSVWNVSFLG | x | x | x | x | x | x | x | x | | x | x | x | x | x | | x | x | | x | x | |
| 26 | 2 | SVWNVSFLGYPARAI | x | x | x | x | x | x | x | x | | x | x | x | x | x | | x | x | | x | x | |
| 27 | 2 | ARAILPYSQALEKLA | x | x | | x | x | x | x | x | | x | x | | x | x | | x | x | | x | | |
| 28 | 2 | NGQHSFYQLIHQGRV | x | x | x | x | x | x | x | x | x | x | x | x | x | x | x | x | x | x | x | x | |
| 29 | 2 | AYEIGQLLAIYEHRI | x | x | | | | | x | x | | x | x | | x | x | | x | x | | x | | |
| 30 | 2 | LLAIYEHRIAVQGFI | x | x | | | | | x | | | x | | | x | | | x | | | x | x | |
| 31 | 2 | QGFIWGINSFDQWGV | x | x | | x | | | x | | | x | | | x | | | x | | | x | | |
| 32 | 2 | ELMSNFFAQPDALAY | x | x | x | x | x | x | x | x | x | x | x | x | x | x | x | x | x | x | x | x | x |
| 33 | 3 | DDGKVYLEMSYYFEI | x | x | x | x | x | x | | | | x | x | x | x | x | x | | | | | | |
| 34 | 4 | PNLTYAKELVERMGL | x | x | | x | x | | x | x | | x | x | | x | x | | x | x | | x | | |
| 35 | 4 | RNMVLGKRFFVTPSD | x | x | | x | x | x | x | x | | x | x | | x | x | | x | x | | x | | |
| 36 | 4 | KRFFVTPSDSVAIIA | x | x | x | x | x | x | x | x | x | x | x | x | x | x | x | x | x | x | | | |
| 37 | 4 | SDSVAIIAANAVQSI | x | x | | x | x | x | x | x | | x | x | | x | x | | x | x | | | | |
| 38 | 4 | AVQSIPYFASGLKGV | x | | | x | x | | | | | | | | | | | | | | x | | |
| 39 | 4 | KNLNLKFFEVPTGWK | x | x | | x | x | x | x | x | | x | | | x | x | | x | x | | | | |
| 40 | 4 | GIWAVLAWLSIIAYK | x | x | | x | x | | x | x | | x | x | | x | x | | x | x | | x | | |
| 41 | 4 | LVSVEDIVLQHWATY | | | | | | | x | x | | | | | x | | | x | | | | | |
| 42 | 4 | HQGIRYLFGDGSRLV | x | | | x | | | x | x | | x | x | x | x | x | | x | x | | | | |
| 43 | 4 | SRLVFRLSGTGSVGA | x | x | | x | x | | x | x | | x | x | | x | x | | x | x | | | | |
| 44 | 4 | GATIRIYIEQYEKDS | x | x | | x | | | x | | | x | x | | x | | | | | | | | |
| 45 | 4 | DALSPLVDVALKLSK | x | | | x | x | | | | | x | | | x | | | | | | | | |
| 46 | 5 | LDIAVRLLEPIKEQV | x | x | | | | | | | | | | | x | x | | | | | | | |
| 47 | 5 | IKEQVPILSYADFYQ | | | | x | x | | x | | | | | | x | | | x | x | | | | |
| 48 | 5 | ILSYADFYQLAGVVA | x | | | x | x | | x | x | x | | | | x | x | | x | x | x | | | |
| 49 | 5 | FYQLAGVVAVEITGG | x | x | | x | x | x | x | x | | x | | | x | x | x | x | x | | | | |
| 50 | 5 | NPLIFDNSYFTELLT | x | x | | x | x | | x | | | x | | | x | | | x | | | | | |
| 51 | 5 | EDAFFADYAEAHLKL | x | x | x | x | x | x | x | x | x | x | x | x | x | x | x | x | x | x | | | |
| 52 | 6 | DNEKSGFISLVSRYL | | | | x | | | | | | | | | | | | | | | x | x | |
| 53 | 6 | IEVRNGFTFLDLIVL | x | | | x | | | x | | | x | | | x | | | x | | | x | x | |
| 54 | 6 | FLDLIVLQIESLNKK | x | x | | x | | | x | | | x | | | | | | x | | | x | | |
| 55 | 6 | LNKKYGSNVPLLLMN | x | | | | | | x | | | x | | | x | | | x | | | x | | |
| 56 | 6 | NVPLLLMNSFNTHED | x | x | | x | | | x | | | x | | | x | x | | x | x | | x | | |
| 57 | 6 | LKIVEKYANSSIDIH | | | | | | | x | | | | | | | | | | | | x | x | x |
| 58 | 6 | GKLDLLLSQGKEYVF | x | x | | | | | x | | | x | | | x | x | x | x | x | | x | x | |
| 59 | 6 | GKEYVFIANSDNLGA | x | x | | x | x | | x | x | | x | x | | x | x | x | x | x | x | x | x | x |
| 60 | 6 | SDNLGAIVDMKILNH | x | | | x | | | x | x | | x | | | x | | | x | | | x | x | x |
| 61 | 6 | ISYEGRVQLLEIAQV | x | | | x | x | | x | x | | x | x | | x | x | x | x | x | x | | | |
| 62 | 6 | VQLLEIAQVPDAHVD | x | x | | x | | | x | | | | | | x | | | x | | | x | | |
| 63 | 6 | FKSIEKFKIFNTNNL | x | x | x | x | x | x | x | x | x | x | x | x | x | x | x | x | x | x | x | | |
| 64 | 6 | FKIFNTNNLWVNLKA | x | x | | x | | | x | x | | x | x | | x | x | | x | x | x | x | x | x |
| 65 | 6 | NNLWVNLKAIKRLVE | x | | | | | | x | | | x | | | x | x | | x | x | x | x | x | x |
| 66 | 6 | IKRLVEADALKMEII | x | x | | | | | x | x | | x | x | | x | x | x | x | x | | x | x | |
| 67 | 6 | VKVLQLETAAGAAIR | x | x | | x | | | x | | | x | x | | x | x | | x | | | x | x | x |
| 68 | 6 | AAIRFFDHAIGINVP | x | | | x | x | | x | | | x | x | | x | | | x | | | x | x | x |
| 69 | 6 | GINVPRSRFLPVKAT | x | x | | x | | | x | x | x | x | x | x | x | x | | x | x | x | x | | |
| 70 | 6 | RFLPVKATSDLQLVQ | x | | | x | x | | x | x | | x | x | | x | x | | x | x | | x | x | |
| 71 | 6 | TSDLQLVQSDLYTLV | | | | x | | | x | | | x | | | x | | | | | | x | | |
| 72 | 6 | VQSDLYTLVDGFVTR | | | | x | | | x | x | | x | x | | x | | | x | | | x | x | |
| 73 | 6 | GPEFKKVGSFLGRFK | x | | | x | | | | | | | | | x | | | x | | | x | x | |
| 74 | 6 | GRFKSIPSIVELDSL | | | | x | | | | | | x | | | x | x | | | | | x | x | x |
| 75 | 7 | GTIRNIINGTVFREP | x | x | | x | x | | x | x | | x | x | | x | x | | x | x | | x | x | x |
| 76 | 7 | VFNFTGAGGVALAMY | x | | | x | | | x | | | x | | | x | x | | x | x | | x | x | x |
| 77 | 7 | EKKWPLYLSTKNTIL | x | x | | x | x | x | x | x | | x | x | | x | x | | x | x | | x | | |
| 78 | 7 | GRFKDIFQAVYEADW | x | | | x | | | x | | | | | | x | | | | | | x | x | x |
| 79 | 7 | WYEHRLIDDMVAYAL | x | x | x | x | x | x | x | x | x | x | x | x | x | x | | x | x | x | x | | |
| 80 | 7 | VQSDFLAQGFGSLGL | x | x | x | x | x | x | x | x | x | x | x | x | x | x | x | x | x | x | x | x | |
| 81 | 7 | NSIASIFAWTRGLAH | x | x | x | x | x | x | x | x | x | x | x | x | x | x | x | x | x | x | x | | |
| 82 | 7 | DNARLLDFTQKLEDA | | | | | | | | | | | | | x | | | | | | x | x | |
| 83 | 7 | LNTEEFIDAVAAELQ | x | | | x | | | x | | | | | | x | | | x | x | | x | x | x |
| 84 | 9 | KSLVRAFMWDSGSTV | x | | | x | | | x | | | x | | | x | | | x | | | | | |
| 85 | 9 | RVLSCDFKPTRPFRI | x | x | | x | x | | x | x | | x | x | | x | x | | x | x | | x | x | |
| 86 | 9 | HTGSIYAVSWSADSK | x | | | x | x | | x | | | x | | | | | | | | | | | |
| 87 | 9 | IHYSPDVSMFASADA | x | x | x | | | | x | x | | | | | | | | | | | x | x | |
| 88 | 9 | IKLKNMLFHTARINC | | | | x | | | | | | | | | x | | | x | | | x | | |
| 89 | 10 | GRYFSKDAVQIITKM | | | | x | | | | | | | | | x | | | | | | x | | |
| 90 | 10 | DAVQIITKMAAANGV | | | | x | | | x | | | x | | | x | | | x | | | x | | |
| 91 | 10 | GVRRVWVGQDSLLST | | | | | | | | | | | | | x | | | x | | | x | x | |
| 92 | 11 | SVGFVETLENDLAQL | | | | | | | x | x | | x | | | x | | | x | x | | x | | |
| 93 | 11 | LGEAPYKFKSALEAV | | | | x | x | | x | | | x | | | | | | x | x | | x | x | |
| 94 | 11 | KFKSALEAVKTLRAE | | | | x | x | | | | | | | | | | | | | | x | | |
| 95 | 11 | VVTFNFRADRMVMLA | x | x | | x | x | | x | x | | x | x | | x | | | x | | | x | x | |
| 96 | 11 | ADRMVMLAKALEFAD | | | | | | | x | | | x | | | | | | | | | x | | |
| 97 | 11 | FDKFDRVRVPKIKYA | x | x | | x | x | | x | | | x | | | x | x | | | | | | | |
| 98 | 11 | PKIKYAGMLQYDGEL | x | x | | x | x | | x | x | | x | x | | x | x | | x | x | | | | |
| 99 | 13 | ECILSGLLSVDGLKV | x | x | x | x | x | x | x | x | x | x | x | x | x | x | x | x | x | x | | | |
| 100 | 13 | LLSVDGLKVLHMDRN | x | x | x | x | x | | x | x | x | x | x | x | x | x | x | x | x | x | | | |
| 101 | 13 | VPKFMMANGALVRVL | x | | | x | x | | x | | | x | | | x | | | x | | | | | |
| 102 | 13 | VRVLIRTSVTKYLNF | x | x | | x | | | | | | | | | | | | | | | | | |
| 103 | 13 | TKYLNFKAVDGSFVY | x | x | x | x | x | | x | x | x | x | x | x | x | x | | x | x | | | | |
| 104 | 13 | TDVEALKSNLMGLFE | x | x | | x | x | | x | | | x | | | x | x | | | | | | | |
| 105 | 13 | EKRRARKFFIYVQDY | x | x | | x | x | x | x | x | | x | x | | x | x | x | x | x | x | | | |
| 106 | 13 | KFFIYVQDYEEEDPK | x | | | x | | | | | | x | | | x | | | x | | | | | |
| 107 | 13 | TVDFIGHALALHRDD | x | x | x | x | x | | x | x | | x | x | x | x | x | x | x | x | x | | | |
| 108 | 13 | VKRM KLYAESLARFQ | | | | x | x | x | x | x | x | x | x | x | x | x | x | x | x | x | | | |
| 109 | 13 | GELPQAFARLSAVYG | x | x | x | x | x | x | x | x | x | x | x | x | x | x | x | x | x | x | | | |
| 110 | 13 | FARLSAVYGGTYMLN | x | x | x | x | x | x | x | x | x | x | x | x | x | x | x | x | x | x | | | |
| 111 | 13 | KGKFIAFVSTEAETD | x | x | x | x | x | x | x | x | x | x | x | x | x | x | x | x | x | x | | | |
| 112 | 13 | ETTVKDVLALYSKIT | | | | | | | | | | x | x | x | x | x | | | | | | | |
| 113 | 13 | LDLSVDLNAASAGES | | | | | | | | | | x | x | | x | x | | | | | | | |
| 114 | 16 | DEKLLSVFREGVVYG | x | x | | x | x | x | x | x | | x | x | | x | x | | x | x | | x | | |
| 115 | 16 | GPGVYDIHSPRIPSK | x | x | | x | x | | x | x | | x | x | | x | x | | x | x | | x | x | |
| 116 | 18 | GAMEKLYDAGKARAI | x | | | | | | | | | | | | | | | | | | | | |
| 117 | 18 | KARAIGVSNLASKKL | | | | | | | | | | | | | x | | | | | | | | |
| 118 | 18 | KKLGDLLAVARIPPA | x | | | x | x | | | | | x | x | | | | | x | | | | | |
| 119 | 19 | LNGPFIATVQQRGAA | | | | | | | | | | | | | x | | | | | | | | |
| 120 | 19 | QQRGAAIIKARKLSS | x | x | x | x | x | | x | x | x | x | x | x | x | x | x | x | x | x | | | |
| 121 | 19 | IIKARKLSSALSAAS | x | x | x | x | x | | x | x | x | x | x | x | x | x | x | x | x | x | | | |
| 122 | 19 | LSSALSAASSACDHI | x | x | x | x | x | | x | x | x | x | x | x | x | x | x | x | x | x | | | |
| 123 | 19 | GTPEGTFVSMGVYSD | x | | | x | x | | x | x | | x | x | | x | x | | x | x | | | | |
| 124 | 20 | LGLPVFNSVAEAKAE | | | | x | x | x | x | x | x | x | x | x | x | x | | x | x | x | | | |
| 125 | 20 | TKANASVIYVPPPFA | x | x | x | x | x | x | x | x | x | x | x | x | x | x | x | x | x | x | | | |
| 126 | 20 | VIYVPPPFAAAAIME | x | x | x | x | | | x | x | | x | x | | x | | | x | x | x | | | |
| 127 | 20 | PFAAAAIMEALEAEL | x | x | x | | | | x | | | x | | | | | | x | x | x | | | |
| 128 | 20 | QHDMVKVKAALNRQS | x | | | x | | | x | | | x | x | | x | | | | | | | | |
| 129 | 20 | TLTYEAVFQTTAVGL | x | | | x | x | x | x | x | x | x | x | x | x | x | x | x | x | x | | | |
| 130 | 20 | DKPVVAFIAGLTAPP | | | | x | x | x | x | | | x | | | x | | | | | | x | | |
| 131 | 20 | KIKALREAGVTVVES | x | | | x | | | x | x | | x | x | | x | x | | x | x | | x | | |
| 132 | 21 | GSGDFKTIKEALAKV | x | x | | x | x | | x | x | | x | x | | x | x | | | | | | | |
| 133 | 21 | MYVMYIKEGTYKEYV | x | x | | x | x | | x | x | | x | x | | x | x | | | | | | | |
| 134 | 21 | VTNLVMIGDGAAKTI | x | x | | x | | | x | x | | x | x | | x | x | | | | | | | |
| 135 | 21 | YQDTLYTHAQRQFFR | x | x | x | x | | | x | x | x | x | x | x | x | x | x | x | | | | | |
| 136 | 21 | GTIDFIFGNSQVVIQ | x | | | x | | | x | | | x | | | x | | | | | | | | |
| 137 | 22 | DGYYIHGQCAIIMFD | x | x | x | x | x | x | x | x | x | x | x | x | x | x | x | x | x | x | x | | |
| 138 | 22 | QCAIIMFDVTSRLTY | x | x | | x | x | | x | x | | x | x | | x | x | | x | x | | x | | |
| 139 | 22 | RKKNLQYYEISAKSN | x | x | x | x | x | x | x | x | x | x | x | x | x | x | x | x | x | x | | | |
| 140 | 22 | SAKSNYNFEKPFLYL | x | x | x | x | x | x | x | x | x | x | x | x | x | x | x | x | x | x | | | |
| 141 | 22 | KPFLYLARKLAGDAN | x | x | | x | x | x | x | x | x | x | x | | x | x | x | | | | x | x | |
| 142 | 22 | EAELAAAAAQPLPDD | x | | | x | x | | x | x | | x | x | | | | | x | | | x | | |
| 143 | 24 | KGKKVFLRADLNVPL | | | | x | x | | x | | | | | | x | x | | x | x | | x | x | x |
| 144 | 24 | EKGAKVILASHLGRP | | | | | | | x | | | x | | | | | | | | | x | | |
| 145 | 24 | VPRLSELLGVEVVMA | x | x | | x | x | | x | | | x | | | x | x | | x | | | x | x | |
| 146 | 24 | GGVLLLENVRFYKEE | x | x | x | x | x | x | x | x | x | x | x | x | x | x | | x | x | x | x | x | |
| 147 | 24 | PEFAKKLASVADLYV | x | x | | x | x | | x | x | | x | x | | x | x | | x | x | | x | x | |
| 148 | 24 | KFLRPSVAGFLMQKE | | | | x | x | | x | | | x | | | x | x | | x | | | x | x | x |
| 149 | 24 | VAGFLMQKELDYLVG | x | x | x | x | x | x | x | x | x | x | x | x | x | x | x | x | x | x | x | | |
| 150 | 24 | KELDYLVGAVANPKK | x | x | | x | x | x | x | x | | x | x | x | x | | | x | | | x | | |
| 151 | 24 | KIGVIESLLAKVDIL | x | | | x | x | | x | | | x | x | | x | x | | x | x | | x | | |
| 152 | 24 | GMIFTFYKAQGKAVG | x | x | | x | | | x | x | | x | | | x | | | x | | | x | x | |
| 153 | 24 | GVSLLLPTDVVVADK | x | | | x | x | x | x | | | x | x | | x | x | | x | | | x | | |
| 154 | 26 | VELVAVNDPFITTDY | x | x | | x | x | | x | x | | x | x | x | x | x | x | x | x | x | | | |
| 155 | 26 | DYMTYMFKYDTVHGQ | x | x | x | x | x | | x | x | | x | x | | x | x | x | x | x | x | | | |
| 156 | 26 | GGAKKVIISAPSKDA | x | x | x | x | x | | x | x | x | x | x | x | x | x | x | x | x | x | | | |
| 157 | 26 | YTSDITIVSNASCTT | x | x | | | | | | | | | | | | | | | | | | | |
| 158 | 26 | KVINDRFGIVEGLMT | x | x | x | x | x | x | x | x | x | x | x | x | x | x | x | x | x | x | x | | |
| 159 | 26 | FGIVEGLMTTVHAMT | x | x | x | x | x | x | x | | | x | | | x | x | x | x | x | | | | |
| 160 | 26 | GGRAASFNIIPSSTG | x | x | x | x | x | x | x | x | x | x | x | x | x | x | x | x | x | x | | | |
| 161 | 26 | ALNDNFVKLVSWYDN | x | x | x | x | x | x | x | x | x | x | x | x | x | x | x | x | x | | | | |
| 162 | 27 | LQHISGVILFEETLY | x | | | | | | x | | | x | x | | x | | | x | x | | x | | |
| 163 | 27 | YEAGARFAKWRAVLK | x | x | x | x | x | | x | x | | x | x | | x | x | x | x | x | | x | | |
| 164 | 27 | GLARYAIICQENGLV | x | x | x | x | x | x | x | x | x | x | x | x | x | x | x | x | x | x | x | x | |
| 165 | 27 | RCAYVTEVVLAACYK | | | | | | | x | x | | x | x | | x | x | | | | | x | | |
| 166 | 27 | WFLSFSFGRALQQST | x | x | | x | x | | x | x | | x | x | | x | x | | x | x | | x | | |
| 167 | 28 | VVDTNLESPNDIVPE | x | x | | | | | x | | | x | | | | | | | | | | | |
| 168 | 29 | EKHFKYVILGGGVAA | | | | x | | | x | x | | x | | | | | | | | | | | |
| 169 | 29 | TEKGIELILSTEIVK | x | x | | x | x | | x | | | x | x | | x | x | | | | | x | | |
| 170 | 29 | GGGYIGLELSAALKL | x | | | x | | | x | x | | x | x | | | | | | | | | | |
| 171 | 29 | LKLNNFDVTMVYPEP | x | | | | | | | | x | x | | | | | | | | | | | |
| 172 | 29 | MPRLFTAGIAHFYEG | x | | | x | | | x | x | | x | x | x | x | | | x | | | | | |
| 173 | 29 | HFYEGYYASKGINIV | | | | | | | | | | x | x | | | | | | | | | | |
| 174 | 29 | VYAIGDVASFPMKLY | | | | x | | | x | | | x | | | | | | | | | | | |
| 175 | 29 | DYLPYFYSRSFDIAW | x | | | | | | | | | | | | | | | x | | | | | |
| 176 | 30 | RDAHYLRGLLPPAIV | | | | x | | | x | | | | | | x | | | | | | | | |
| 177 | 30 | MHNLRQYTVPLQRYI | | | | | | | x | | | x | | | | | | | | | | | |
| 178 | 30 | VPLQRYIAMMDLQER | | | | x | x | | x | x | | x | x | | x | | | x | | | | | |
| 179 | 30 | ERLFYKLLIDNVEEL | x | | | x | x | x | x | x | x | x | x | x | x | x | x | x | x | | | | |
| 180 | 30 | EELLPVVYTPVVGEA | x | x | | x | x | | x | x | | x | x | | x | x | | x | x | | | | |
| 181 | 30 | RSIQVIVVTDGERIL | x | | | x | x | | x | | | x | | | x | x | | x | x | | | | |
| 182 | 30 | GEKVLVQFEDFANHN | x | | | x | x | x | x | x | x | x | x | x | x | x | x | x | x | x | | | |
| 183 | 30 | FDLLAKYSKSHLVFN | | | | x | | | x | | | x | | | | | | | | | | | |
| 184 | 30 | VFNDDIQGTASVVLA | x | x | x | x | x | x | x | x | x | x | x | x | x | x | x | x | x | x | x | | |
| 185 | 30 | SVVLAGLLAALKVIG | | | | | | | x | | | x | | | | | | | | | | | |
| 186 | 30 | TGIAELIALEMSKHT | x | | | x | x | | x | x | | x | x | | x | x | | x | x | | | | |
| 187 | 30 | CRKKIWLVDSKGLLV | x | | | x | | | x | | | x | | | x | | | | | | | | |
| 188 | 30 | EEAYTWTKGTAVFAS | | | | | | | | | | | | | | | | x | | | | | |
| 189 | 30 | GFGLGVVISGAIRVH | x | | | x | | | x | | | x | | | x | | | x | x | | | | |
| 190 | 30 | VISGAIRVHDDMLLA | x | x | | x | | | x | x | x | x | x | | x | x | | x | x | | | | |
| 191 | 30 | HDDMLLAASEALAEQ | x | x | x | x | x | | x | x | | x | x | x | x | x | | x | x | | | | |
| 192 | 30 | FPPFTNIRKISANIA | x | | | x | | | x | | | x | | | x | | | x | x | | | | |
| 193 | 30 | IRKISANIAAKVAAK | x | x | | x | x | x | x | x | | x | x | | x | x | | x | x | | | | |
| 194 | 31 | VEHKGQVDLVTETDK | | | | x | x | | x | x | | x | x | | x | x | | x | x | | | | |
| 195 | 31 | TDKACEDLIFNHLRK | | | | | | | | | | | | | x | | | | | | | | |
| 196 | 32 | IEIDSLFEGIDFYST | x | x | | x | x | | x | x | | x | x | | x | x | | x | x | | x | | |
| 197 | 32 | IDFYSTITRARFEEL | x | x | x | x | x | x | x | x | x | x | x | x | x | x | x | x | x | x | x | | |
| 198 | 32 | IPKVQQLLQDFFNGK | x | x | x | x | x | x | x | x | x | x | x | x | x | x | x | x | x | x | | | |
| 199 | 32 | EAVAYGAAVQAAILS | x | x | x | x | x | x | x | x | x | x | x | x | x | x | x | x | x | x | x | | |
| 200 | 32 | VQDLLLLDVTPLSLG | x | x | x | x | x | x | x | x | x | x | x | x | x | x | x | x | x | x | x | | |
| 201 | 33 | LAWNCERCRKGESKK | x | | | x | | | x | | | x | | | x | | | | | | | | |
| 202 | 34 | VRVKILFTALCHTDV | x | | | x | | | x | | | x | | | x | x | | x | x | | | | |
| 203 | 34 | MCDLLRINTDRGVMI | x | x | x | x | x | x | x | x | | x | x | x | x | x | | x | x | | | | |
| 204 | 34 | KPIFHFVGTSTFSEY | x | x | x | x | | | x | x | | x | x | | x | | | x | | | x | | |
| 205 | 34 | VGTSTFSEYTVMHVG | x | x | | x | x | | x | | | x | x | | x | x | | x | x | | | | |
| 206 | 34 | VAIFGLGAVGLAAAE | x | x | x | x | x | x | x | x | x | x | x | x | x | x | | x | x | | | | |
| 207 | 34 | GAVGLAAAEGARIAG | x | x | x | x | | | x | x | x | x | x | x | x | x | x | x | x | x | | | |
| 208 | 34 | GNINAMIQAFECVHD | | | | x | x | | x | | | x | | | x | | | | | | | | |
| 209 | 34 | LKGTFFGNFKPRTDL | x | | | x | | | x | x | | x | x | | x | x | | x | | | | | |
| 210 | 34 | KFITHSVTFSEINKA | x | | | x | | | | | | x | | | x | x | | x | | | x | | |
| 211 | 34 | VTFSEINKAFDLMAK | x | | | | | | | | | | | | | | | | | | x | | |
| 212 | 35 | ALRWNLQMGHSVLPK | x | | | x | | | x | x | | x | x | | x | x | | x | x | | x | | |
| 213 | 35 | DDLLAKFSEIKQTRL | | | | x | | | | | | | | | | | | | | | x | | |
| 214 | 36 | QDFKKVNEIYAKYFP | x | x | | x | x | | x | | | x | | | x | x | | x | x | | | | |
| 215 | 36 | NEIYAKYFPSPAPAR | x | x | | x | x | | x | x | x | x | x | x | x | x | x | x | x | x | | | |
| 216 | 36 | YFPSPAPARSTYQVA | x | x | | x | x | | x | x | x | x | x | x | x | x | x | x | x | | | | |
| 217 | 36 | ARSTYQVAALPLDAR | x | x | x | x | | | x | x | x | x | x | | x | x | | x | | | | | |
| 218 | 36 | LPLDARIEIECIAAL | x | x | x | | | | x | x | | x | | | x | | | x | x | | | | |
| 219 | 38 | GWYHLFYQYNPEGAV | | | | x | x | | x | | | x | | | x | | | x | | | | | |
| 220 | 38 | SRDLIHWRHLPLAMV | | | | x | | | x | x | | x | x | | x | | | | | | | | |
| 221 | 38 | LNMLYTGSTNASVQV | | | | x | | | x | | | x | | | | | | | | | | | |
| 222 | 38 | EAFSVRVLVDHSIVE | | | | | | | | | | | | | x | | | | | | | | |
| 223 | 39 | GAFTGEVSAEMLANL | x | x | x | x | | | x | x | | x | x | | x | x | | x | | | | | |
| 224 | 39 | VSAEMLANLGIPWVI | x | x | | | | | x | | | x | x | | x | x | | x | x | | | | |
| 225 | 39 | GESSEFVGDKVAYAL | x | | | | | | x | x | | x | | | | | | | | | x | | |
| 226 | 39 | GDKVAYALAQGLKVI | x | x | | x | | | x | | | x | | | x | | | x | | | x | | |
| 227 | 39 | DWTNVVIAYEPVWAI | x | | | x | x | | x | x | | x | x | | | | | | | | | | |
| 228 | 39 | IAYEPVWAIGTGKVA | x | x | x | x | x | x | x | x | | x | x | x | x | x | x | x | x | x | | | |
| 229 | 39 | LKPEFIDIINAATVK | x | | | x | | | | | | | | | x | | | x | | | | | |
| 230 | 40 | VWQHDRVEIIANDQG | x | x | x | x | x | x | x | x | x | x | x | x | x | x | x | x | x | x | x | | |
| 231 | 40 | VEIIANDQGNRTTPS | x | x | x | x | x | x | x | x | x | x | x | x | x | x | x | x | x | x | x | | |
| 232 | 40 | TTPSYVAFTDSERLI | x | x | | x | x | | x | x | x | x | x | x | x | x | | x | x | x | | | |
| 233 | 42 | EEKQFAAEEISSMVL | x | x | x | x | x | | x | x | x | x | x | x | x | x | x | x | x | | | | |
| 234 | 42 | SSMVLIKMREIAEAF | x | x | | x | x | x | x | x | | x | x | | x | x | | x | x | | | | |
| 235 | 42 | SIKNAVVTVPAYFND | x | x | x | x | x | | x | x | x | x | x | | x | x | | x | x | | x | | |
| 236 | 42 | GVIAGLNVLRIINEP | x | x | | x | x | | x | x | | x | x | | x | x | | x | x | x | | | |
| 237 | 42 | VLRIINEPTAAAIAY | x | x | x | x | x | x | x | x | | x | x | | x | x | | x | x | x | | | |
| 238 | 43 | FAWSLLDNFEWRMGF | | | | x | | | | | | | | | x | | | x | | | | | |
| 239 | 44 | IELWQVKSGTLFDNI | x | x | | x | x | | x | x | x | x | x | x | x | | | x | | | | | |
| 240 | 47 | EDVAVSLAKYTAELS | | | | | | | x | | | x | | | | | | x | | | | | |
| 241 | 47 | DSNYKLAVDGLLSKV | | | | | | | | | | x | | | x | x | | x | | | x | | |
| 242 | 47 | PPPQRITFTFPVIKS | x | | | | | | x | | | x | | | x | | | | | | | | |
| 243 | 48 | APWLLTVGASTSDRR | | | | x | | | x | | | x | | | | | | | | | | | |
| 244 | 49 | ELRKTYNLLDAVSRH | x | x | | x | x | x | x | x | | x | x | | x | x | x | | | | x | | x |
| 245 | 49 | QVYPRSWSAVMLTFD | x | x | | x | x | x | x | x | x | x | x | | x | x | | | | | x | x | |
| 246 | 49 | AVMLTFDNAGMWNVR | x | x | x | x | x | x | x | x | x | x | x | x | x | x | x | | | | x | x | x |
| 247 | 49 | GEQLYISVISPARSL | x | x | | x | | | x | x | | x | x | | x | | | | | | x | | |
| 248 | 50 | LKSIKAFASGILVPK | | | | x | | | | | | x | | | x | | | | | | | | |
| 249 | 51 | PESKVFYLKMKGDYH | x | | | x | x | | x | x | | x | x | | x | x | | | | | x | | |
| 250 | 51 | MNSYKAAQDIALADL | | | | x | | | x | x | | | | | x | | | x | | | x | | |
| 251 | 51 | APTHPIRLGLALKIS | x | | | x | | | x | | | x | | | x | | | x | | | | | |
| 252 | 53 | WSEIQTLKPNLIGPF | | | | | | | x | | | x | | | | | | | | | x | | |
| 253 | 53 | KFMTLAGFLDYAKAS | | | | | | | x | | | x | | | x | | | | | | | | |
| 254 | 53 | NISGILIGIEHAAYL | | | | | | | x | | | x | | | x | | | | | | | | |
| 255 | 53 | AAYLATRGLDVVDAV | | | | | | | x | | | x | | | x | | | | | | x | | |
| 256 | 53 | GLVTEFPSTAAAYFR | x | x | | x | | | x | x | | x | x | | x | | | | | | x | | |
| 257 | 54 | NIVVNVFNQLDQPLL | x | x | x | x | | | x | x | x | x | | | x | | | | | | x | | |
| 258 | 54 | IGSFFYFPSIGMQRT | x | | | x | x | | x | x | | x | x | | x | | | | | | x | x | x |
| 259 | 54 | GYGLISVVSRLLIPV | x | x | | x | x | x | x | x | x | | | | x | x | | | | | | | |
| 260 | 54 | VVSRLLIPVPFDPPA | x | x | x | x | x | x | x | x | x | x | x | x | x | x | x | | | | x | | |
| 261 | 55 | SVFKKFPKFRRVLVI | | | | x | | | x | | | x | | | x | | | | | | | | |
| 262 | 56 | KELGGKILRQPGPLP | | | | | | | x | | | x | | | | | | | | | | | |
| 263 | 56 | ILRQPGPLPGLNTKI | | | | x | | | x | | | x | | | x | | | x | | | | | |
| 264 | 56 | KIASFLDPDGWKVVL | x | | | x | | | x | | | x | | | x | | | x | | | x | | |
| 265 | 56 | GWKVVLVDHADFLKE | x | | | | | | x | x | | x | x | | | | | | | | | | |
| 266 | 57 | RLVCLRVHPTFTLLH | x | | | x | | | x | | | x | | | x | x | | x | | | | | |
| 267 | 58 | YFVEAYLNNPLVQKA | x | x | | x | x | | x | x | x | x | x | | x | x | | | | | | | |
| 268 | 58 | VQKAIHANTALNYPW | x | x | | x | x | | x | x | | x | x | | x | x | | | | | | | |
| 269 | 58 | LYSGDLDAMVPVTAS | x | x | x | x | x | x | x | x | | x | x | x | x | x | x | | | | | | |
| 270 | 59 | VKKIVTVLNEAEVPS | | | | | | | | | | x | x | | x | x | | | | | | | |
| 271 | 59 | EDAVEVVVSPPFVFL | x | | | | | | x | | | x | x | | x | x | | x | | | | | |
| 272 | 59 | ALLRPDFAVAAQNCW | | | | | | | | | | | | | x | | | | | | x | | |
| 273 | 59 | GAFTGEISAEMLVNL | x | | | x | x | | x | x | | x | x | | x | x | x | x | x | x | | | |
| 274 | 59 | ISAEMLVNLQVPWVI | | | | x | | | x | | | | | | x | x | | x | | | x | | |
| 275 | 59 | ADKVAYALAQGLKVI | x | | | | | | | | | | | | | | | | | | x | x | |
| 276 | 59 | TTMEVVAAQTKAIAE | x | | | | | | | | | | | | x | x | | | | | x | | |
| 277 | 59 | WTNVVLAYEPVWAIG | x | x | | x | x | x | x | x | x | x | x | x | x | | | x | x | | x | | |
| 278 | 60 | EDSHFVVELTYNYGV | x | x | | x | x | | x | x | x | x | x | x | x | x | | x | x | | | | |
| 279 | 60 | RAIKFYEKAFGMELL | | | | | | | | | | | | | | | | x | | | | | |
| 280 | 60 | NPQYKYTIAMMGYGP | x | x | | x | | | | | | x | x | | x | x | | x | x | | | | |
| 281 | 60 | KNAVLELTYNYGVKE | x | x | | | | | | | | x | | | x | x | | x | | | | | |
| 282 | 60 | DGWKSVFVDNLDFLK | x | | | x | | | | | | x | | | | | | x | | | | | |
| 283 | 62 | FTVQEMVALSGAHTL | x | | | x | | | | | | | | | x | | | x | | | x | | |
| 284 | 64 | YSDLYQLAGWAVEV | x | | | x | | | x | x | | x | x | | x | x | | x | x | | x | | |
| 285 | 64 | DHLRQVFGKQMGLSD | | | | x | x | | x | | | x | | | x | | | x | | | | | |
| 286 | 65 | FSCDSAYQVTYIVRG | | | | x | x | | x | x | | x | x | | x | x | | x | | | x | | |
| 287 | 65 | YQVTYIVRGSGRVQV | | | | x | | | x | | | x | | | x | x | | | | | x | | |
| 288 | 65 | GMEWFSIITTPNPIF | | | | x | x | | x | x | | x | x | | x | x | x | x | x | x | | | |
| 289 | 65 | GKTSVWKAISPEVLE | | | | | | | | | | | | | | | | x | | | | | |
| 290 | 69 | ARSALTISVLRISSM | | | | | | | | | | | | | x | | | | | | | | |
| 291 | 69 | ISVLRISSMPFSVYH | x | | | | | | | | | | | | | | | | | | | | |
| 292 | 72 | KHLIYVTGWSVYTEI | x | | | x | | | x | x | | x | x | | x | x | | x | | | | | |
| 293 | 72 | TGWSVYTEITLLRDA | | | | x | | | x | | | x | | | | | | | | | | | |
| 294 | 72 | SEGVRVLMLVWDDRT | x | | | x | x | | x | x | | x | x | | x | x | x | x | x | x | | | |
| 295 | 72 | DDSGSIVQDLQISTM | | | | x | | | | | | x | | | x | | | x | | | | | |
| 296 | 72 | LQISTMFTHHQKIVV | x | | | x | x | x | | | | x | x | x | x | x | | x | x | x | | | |
| 297 | 72 | PVAWDVLYNFEQRWR | | | | x | x | x | x | x | x | x | x | | x | | | x | | | | | |
| 298 | 72 | AWNVQLFRSIDGGAA | x | x | | x | x | | x | | | x | x | | x | x | | | | | | | |
| 299 | 72 | DAYICAIRRAKSFIY | x | | | x | | | x | | | x | | | x | | | x | | | x | | |
| 300 | 72 | IRRAKSFIYIENQYF | x | x | | x | x | | x | x | | x | x | | x | x | | x | x | | | | |
| 301 | 72 | FIYIENQYFLGSSYC | x | x | | x | x | | x | x | | x | x | | x | x | | | | | | | |
| 302 | 72 | RFTVYVVVPMWPEGI | x | | | x | x | x | x | x | | x | x | | x | x | | | | | | | |
| 303 | 72 | DYLKAQQNRRFMIYV | | | | x | x | | x | | | x | | | | | | | | | x | | |
| 304 | 72 | FMIYVHTKMMIVDDE | x | x | | x | x | | x | x | | x | x | | x | x | | x | x | | | | |
| 305 | 72 | IVDDEYIIVGSANIN | x | x | x | x | x | x | x | x | x | x | x | x | x | x | x | x | x | | | | |
| 306 | 72 | GQVHGFRMALWYEHL | x | | | x | x | | x | x | x | x | x | x | x | | | x | | | | | |
| 307 | 72 | LPGVEFFPDTQARIL | | | | | | | x | | | x | | | | | | x | | | | | |
| 308 | 73 | EPPQFIALFQPMVIL | x | | | | | | x | | | x | | | x | | | | | | | | |
| 309 | 73 | QQQWAAKVAEFLKPG | x | | | | | | x | | | x | | | | | | x | x | | | | |
| 310 | 73 | RASALAALSSAFNPS | | | | | | | x | | | x | | | x | x | | | | | | | |
| 311 | 73 | SQRAAAVAALSNVLT | x | x | x | x | x | | x | x | x | x | x | x | x | x | | | | | | | |
| 312 | 76 | NIWADDLAASLSTLE | | | | | | | | | | | | | x | | | | | | x | | |
| 313 | 76 | MVEYFGEQLSGFAFT | | | | x | x | x | x | x | x | x | x | x | x | x | x | x | x | x | | | |
| 314 | 76 | LSGFAFTANGWVQSY | | | | x | x | x | x | x | x | x | x | x | x | x | | x | x | | x | | |
| 315 | 76 | NPMTVFWSKMAQSMT | | | | x | | | x | | | x | | | x | | | | | | x | x | |
| 316 | 76 | KDKLVVSTSCSLMHT | | | | x | | | | | | x | x | | x | x | | x | x | | | | |
| 317 | 76 | TSCSLMHTAVDLVNE | | | | x | | | x | x | | x | x | | x | x | | x | x | | x | | |
| 318 | 76 | TKLDSEIKSWLAFAA | | | | x | | | x | x | | x | x | | x | x | | x | x | | x | | |
| 319 | 76 | IKSWLAFAAQKVVEV | | | | x | x | | x | x | x | x | x | x | x | x | x | x | x | x | | | |
| 320 | 77 | EGPLMLYVSKMIPAS | | | | x | x | | x | x | | x | x | | x | x | x | x | x | x | | | |
| 321 | 77 | KGRFFAFGRVFAGRV | | | | x | | | x | | | x | | | x | | | x | | | | | |
| 322 | 77 | GNTVALVGLDQFITK | | | | x | x | | x | | | x | x | | x | x | | x | x | | | | |
| 323 | 77 | VGLDQFITKNATLTG | | | | x | x | | x | | | x | | | x | | | | | | | | |
| 324 | 77 | PIRAMKFSVSPVVRV | | | | x | x | x | x | x | x | x | x | x | x | x | x | x | x | x | | | |
| 325 | 77 | FMGGAEIIVSPPVVS | x | | | x | x | | x | | | x | | | x | | | x | | | | | |
| 326 | 77 | SPPVVSFRETVLDKS | x | | | | | | | | | | | | | | | x | | | x | | |
| 327 | 77 | NKHNRLYMEARPLEE | x | | | x | x | | x | x | x | x | x | x | x | x | | x | x | x | x | | |
| 328 | 77 | PTARRVIFASQLTAK | x | x | | x | | | x | | | | | | x | x | | x | | | | | |
| 329 | 77 | AKPRLLEPVYLVEIQ | x | x | x | x | x | | x | x | x | x | x | | x | x | | x | x | x | | | |
| 330 | 77 | EPVYLVEIQAPEGAL | x | x | | x | | | x | x | | x | | | x | | | x | x | | | | |
| 331 | 77 | PLYNIKAYLPVIESF | x | x | | x | x | | x | x | x | x | x | | x | x | | x | x | | | | |
| 332 | 77 | LPVIESFGFSATLRA | | | | | | | x | x | | x | | | | | | x | | | x | | |
| 333 | 77 | FGFSATLRAATSGQA | x | | | x | | | x | x | | x | x | | x | | | x | x | | x | | |
| 334 | 79 | EVYEARLTKFKYLAG | | | | x | | | | | | | | | | | | | | | x | | |
| 335 | 83 | GMTGMLWETSLLDPE | | | | x | x | | x | | | x | x | | | | | x | | | x | | |
| 336 | 83 | PEGLLWLLLTGKVPT | x | x | | x | x | x | x | x | x | x | x | x | x | x | | x | | | x | | |
| 337 | 83 | QFTTGVMALQVESEF | x | x | | x | | | x | | | x | | | x | | | | | | | | |
| 338 | 83 | DPKMLELMRLYITIH | | | | | | | | | | | | | x | | | | | | x | | |
| 339 | 83 | ALSDPYLSFAAALNG | x | x | x | x | x | x | x | x | x | x | x | x | x | x | x | x | x | x | x | | |
| 340 | 83 | LSFAAALNGLAGPLH | x | x | x | x | x | x | x | x | x | x | x | x | x | x | x | x | x | x | x | | |
| 341 | 83 | PLHGLANQEVLLWIK | x | x | x | x | x | x | x | x | x | x | x | x | x | x | | x | x | x | | | |
| 342 | 83 | QEVLLWIKSVMEETG | | | | | | | x | | | x | | | | | | | | | | | |
| 343 | 83 | QLKEYVWKTLKSGKV | x | | | | | | | | | | | | x | x | | x | x | | x | | |
| 344 | 83 | EDPLFQLVSKLYEVV | x | x | | x | | | x | | | | | | x | x | | x | x | | x | | |
| 345 | 83 | LVSKLYEVVPGILTE | x | x | | | | | | | | | | | x | x | | x | x | | x | | |
| 346 | 83 | SGVLLNHFGLVEARY | | | | | | | x | | | x | | | x | x | | x | | | x | | |
| 347 | 83 | TVLFGVSRSMGIGSQ | | | | x | | | | | | | | | x | | | x | x | | x | | |
| 348 | 83 | GSQLIWDRALGLPLE | x | x | | x | x | | x | x | | x | x | | x | x | | x | x | x | x | | |
| 349 | 84 | GPVTILNWSFVRNDQ | | | | x | x | x | x | x | x | x | x | x | x | x | x | x | x | x | x | | |
| 350 | 84 | PRFETCYQIALAIKK | | | | x | x | | x | x | | x | x | | x | x | | | | | | | |
| 351 | 84 | GIQVIQIDEAALREG | x | x | | x | x | | x | x | | x | x | | x | x | | x | x | | | | |
| 352 | 84 | EHAFYLDWAVHSFRI | x | x | x | x | x | x | x | x | x | x | x | x | x | x | | x | x | | x | | |
| 353 | 84 | FNDIIHSIINMDADV | x | x | x | x | x | x | x | x | | x | x | | x | x | | x | x | | x | | |
| 354 | 84 | SDEKLLSVFREGVTY | x | x | | x | x | | x | x | | x | x | | x | x | | x | x | | | | |
| 355 | 84 | VNKMLAVLDTNILWV | x | | | x | | | x | | | x | | | x | | | x | | | x | | |
| 356 | 84 | TRKYAEVMPALTNMV | | | | | | | x | | | x | | | | | | | | | | | |
| 357 | 86 | TREENVYMAKLAEQA | x | | | x | x | | x | x | | x | x | | x | x | | x | x | | | | |
| 358 | 86 | YEEMVEFMEKVAKTA | | | | x | x | | x | x | | x | x | | x | x | | | | | | | |
| 359 | 86 | EERNLLSVAYKNVIG | x | x | x | x | x | x | x | x | x | x | x | x | x | x | x | x | x | x | | | |
| 360 | 86 | AYKNVIGARRASWRI | x | x | x | x | x | x | x | x | x | x | x | x | x | x | x | x | x | x | | | |
| 361 | 86 | RRASWRIISSIEQKE | x | x | x | x | x | x | x | x | x | x | x | x | x | x | x | x | x | x | | | |
| 362 | 86 | SKICDGILKLLDSHL | x | | | x | | | x | | | x | | | | | | x | | | | | |
| 363 | 86 | AESKVFYLKMKGDYH | x | x | | x | x | x | x | x | x | x | x | x | x | x | x | x | x | x | | | |
| 364 | 86 | GDYHRYLAEFKAGAE | x | x | | x | x | | x | x | | x | x | | x | x | | x | | | | | |
| 365 | 86 | NTLVAYKSAQDIALA | | | | x | x | | x | | | x | x | | x | | | x | | | | | |
| 366 | 86 | LPTTHPIRLGLALNF | x | x | | x | x | | x | x | | x | x | | x | x | | x | x | | | | |
| 367 | 86 | IRLGLALNFSVFYYE | x | x | x | x | x | x | x | x | x | x | x | x | x | x | x | x | x | x | | | |
| 368 | 86 | LNFSVFYYEILNSPD | x | x | x | x | x | x | x | x | x | x | x | x | x | x | x | x | x | x | | | |
| 369 | 86 | YKDSTLIMQLLRDNL | x | x | x | x | x | x | x | x | x | x | x | x | x | x | x | x | x | x | | | |
| 370 | 86 | IMQLLRDNLTLWTSD | x | x | x | x | x | x | x | x | x | x | x | x | x | x | x | x | x | x | x | | |
| 371 | 87 | ADGILFGFPTRFGMM | x | | | x | | | x | x | | x | x | | x | x | | x | | | | | |
| 372 | 89 | QTYYLSMEYLQGRAL | | | | x | x | x | x | | | x | | | x | | | x | x | x | x | | |
| 373 | 89 | RLAACFLDSMATLNL | | | | x | x | | | | | | | | x | | | x | x | | | | |
| 374 | 89 | LRYRYGLFKQRIAKE | | | | x | x | x | x | | | | | | x | | | | | | x | | |
| 375 | 89 | FSPWEIVRHDVVYPV | | | | x | | | | | | | | | | | | x | | | x | | |
| 376 | 89 | GEVLNALAYDVPIPG | | | | x | | | | | | | | | x | | | x | | | x | x | |
| 377 | 89 | IPGYKTKNAISLRLW | | | | x | | | | | | | | | x | x | | x | | | | | |
| 378 | 89 | AEDFNLFQFNDGQYE | | | | x | | | | | | | | | x | | | x | x | x | | | |
| 379 | 89 | EGKLLRLKQQFFLCS | | | | x | x | | | | | | | | x | x | | x | x | | | | |
| 380 | 89 | LKQQFFLCSASLQDI | | | | x | x | x | | | | x | | | x | x | x | x | x | x | | | |
| 381 | 89 | PTLAIPELMRLLMDE | x | x | x | x | x | x | | | | | | | x | x | x | x | x | | | | |
| 382 | 89 | PQKPVVRMANLCVVS | | | | x | x | | | | | | | | x | x | x | x | x | x | | | |
| 383 | 89 | ILKEELFADYVSIWP | | | | x | | | | | | | | | x | | | x | x | | | | |
| 384 | 89 | PRRWLRFCNPELSEI | | | | x | | | | | | | | | x | | | x | | | x | | |
| 385 | 89 | IKRIHEYKRQLMNIL | | | | x | | | x | | | x | x | | x | | | x | x | | x | x | |
| 386 | 89 | YKRQLMNILGAVYRY | | | | x | x | | x | | | | | | x | | | x | x | | x | x | |
| 387 | 89 | LGAVYRYKKLKEMSA | | | | x | | | | | | | | | | | | | | | x | x | x |
| 388 | 89 | GKAFATYTNAKRIVK | | | | x | x | x | x | | | | | | x | x | x | x | x | x | x | x | |
| 389 | 89 | KRIVKLVNDVGAVVN | | | | x | x | x | | | | | | | x | x | x | x | x | x | x | x | |
| 390 | 89 | VNKYLKVVFIPNYNV | | | | | | | | | | | | | x | | | x | | | x | | |
| 391 | 89 | VFIPNYNVSVAEVLI | | | | x | x | | | | | | | | x | | | x | | | x | x | x |
| 392 | 89 | FLVGYDFPSYIDAQA | | | | | | | | | | | | | x | | | x | | | | | |
| 393 | 89 | KRWIKMSILNTAGSG | | | | x | | | | | | x | x | | x | | | x | | | x | x | x |
| 394 | 90 | PDLPYDYGALEPAIS | x | x | x | x | x | x | x | x | x | x | x | x | x | x | | x | x | | x | x | |
| 395 | 90 | HAYYLQYKNVRPDYL | x | x | x | x | x | x | x | x | x | x | x | x | x | x | x | x | x | x | x | x | |
| 396 | 90 | PDYLTNIWKVVNWKY | x | | | x | | | x | | | x | | | x | | | x | | | x | x | |
| 397 | 91 | HYKGSSFHRVIPGFM | x | x | | x | x | x | x | x | | x | x | | x | x | x | x | x | | x | x | x |

“x” indicates that a matching sequence with zero, less than 2 or less than 3 mismatches to the (Phl p) sequence is found in the non-grass pollen species

Table 2 shows wild type full length sequences of NTGA's detected by combined transcriptomic analysis and Mass spectrometry analysis of grass pollen extracts.

TABLE 2

| SEQ | | |
|---|---|---|
| ID | NTGA | |
| No | No | Phl p wild type sequence (SEQ ID Nos: 398-443) |
| 398 | 1 | MAATIQSVKARQIFDSRGNPTVEVDVCCSDGTFARAAVPSGASTGVYEALELRDGGSDYLGK |
| | | GVLKAVDNVNSIIGPALIGKDPTEQTELDNFMVHQLDGTKNEWGWCKQKLGANAILAVSLAV |
| | | CKAGALVKKIPLYQHIANLAGNKQLVLPVPAFNVINGGSHAGNKLAMQEFMILPTGASSFKEA |
| | | MKMGVEVYHNLKSVIKKKYGQDATNVGDEGGFAPNIQENKEGLELLKTAIEKAGYTGKVVIG |
| | | MDVAASEFYGEKDQTYDLNFKEENNDGSQKISGDSLKNVYKSFVSEYPIVSIEDPFDQDDWV |
| | | HYAKMTEEIGEQVQIVGDDLLVTNPTRVAKAIAEKSCNALLLKVNQIGSVTESIEAVKMSKRA |
| | | GWGVMTSHRSGETEDTFIADLAVGLSTGQIKTGAPCRSERLAKYNQLLRIEEELGAAAVYAGL |
| | | KFRAPVEPY |
| 399 | 2 | MASPALISDTDQWKALQAHVGAIHKTHLRDLMADADRCKALTAEFEGVFLDYSRQQATTETV |
| | | DKLFKLAEAAKLKEKIAKMFNGDKINSTENRSVLHVALRAPRDAVINSDGVNVVPEVWAVIDK |
| | | IKQFSETFRSGSWVGATGKPLTNVVSVGIGGSFLGPLFVHTALQTDPEAAESAKGRQLRFLAN |
| | | VDPVDVARSIKDLDPETTLVVVVSKTFTTAETMLNARTIKEWIVSSLGPQAVSKHMIAVSTNLK |
| | | LVKEFGIDPNNAFAFWDWVGGRYSVCSAVGVLPLSLQYGFPIVQRFLEGASSIDNHFRTASFE |
| | | KNIPVLLGLLSVWNVSFLGYPARAILPYSQALEKLAPHIQQLSMESNGKGVSIDGVPLPYEAGEI |
| | | DFGEPGTNGQHSFYQLIHQGRVIPCDFIGVIKSQQPVYLKGETSNHDELMSNFFAQPDALASR |
| | | KTPAPLRSENVSENLIPHKTFKGNRPSLSFLLSSLSAYEIGQLLAIYEHRIAVQGFIWGINSFDQ |
| | | WGVELGKSLASQVRKQLHASRMEGKPVEGFNPSSASLLARYLAVEPSTPYDTTVLPKV |
| 400 | 3 | MDDHKEHKEKEHTGGNPEVNEEEEEDEEAKRAVLLGPQVPLKEQLELDKDDESLRRWKEQLL |
| | | GQVDTEQLGETAEPEVKVVDLTILSPDRPDLVLPIPFVADEKGYAFALKDGSTYSFRFSFIVSNN |
| | | IVSGLKYTNTVWKTGVRVENQKMMLGTFSPQPEPYIYVGEEETTPAGIFARGSYSAKLKFVDD |
| | | DGKVYLEMSYYFEIRKDWPTGQ |
| 401 | 4 | YIKLMKTIFDFESIKKLLASPKFSFCFDGLHGVAGAYAKRMFVDELGASESSLLNCVPKEDFGG |
| | | GHPDPNLTYAKELVERMGLGKSSSNVEPPEFGAAADGDADRNMVLGKRFFVTPSDSVAIIAAN |
| | | AVQSIPYFASGLKGVARSMPTSAALDVVAKNLNLKFFEVPTGWKFFGNLMDAGMCSVCGEES |
| | | FGTGSDHIREKDGIWAVLAWLSIIAYKNKDNLGGDKLVSVEDIVLQHWATYGRHYYTRYDYE |
| | | NVDAEAAKELMANLVKMQSALSDVNKLIKEIQPDVAEVVSADEFEYKDPVDGSVSKHQGIRY |
| | | LFGDGSRLVFRLSGTGSVGATIRIYIEQYEKDSSKTGRESSDALSPLVDVALKLSKIKEYTGRS |
| | | APTVIT |
| 402 | 5/64 | MAAKCYPTVSDEYLAAVAKARRKLRGLIAEKNCAPLMLRIAWHSAGTFDVATKTGGPFGTMRC |
| | | PAELAHGANAGLDIAVRLLEPIKEQVPILSYADFYQLAGVVAVEITGGPEVPFHPGRQDKTEPPP |
| | | EGRLPDATLGSDHLRQVFTAQMGLSDQDIVALSGGHTLGRCHKERSGFEGAWTANPLIFDNS |
| | | YFTELLTGEKEGLLQLPTDKTLLTDPAFRPLVEKYAADEDAFFADYAEAHLKLSELGFGE |
| 403 | 6 | MADEKLAKLREAVAGLPQISDNEKSGFISLVSRYLSGEEEHIEWPKIHTPTDEVVVPYDTVDAP |
| | | PEDLEATKALLDKLAVLKLNGGLGTTMGCTGPKSVIEVRNGFTFLDLIVLQIESLNKKYGSNVPL |
| | | LLMNSFNTHEDTLKIVEKYANSSIDIHTFNQSQYPRVVADEFLPWPSKGKTDKDGWYPPGHG |
| | | DIFPSLMNSGKLDLLLSQGKEYVFIANSDNLGAIVDMKILNHLIHKQNEYCMEVTPKTLADVKG |
| | | GTLISYEGRVQLLEIAQVPDAHVDEFKSIEKFKIFNTNNLWVNLKAIKRLVEADALKMEIIPNPK |
| | | EVDGVKVLQLETAAGAAIRFFDHAIGINVPRSRFLPVKATSDLQLVQSDLYTLVDGFVTRNSAR |
| | | TDPSNPSIELGPEFKKVGSFLGRFKSIPSIVELDSLKVSGDVWFGSGIVLKGKVTITAKPGVKLE |
| | | IPDGAVLENKDINGAEDL |
| 404 | 7 | MAFEKIKVANPIVEMGDEMTRVFWQSIKEKLIFPFLDLDIKYYDLGVLHRDATDDKVTVEAAEA |
| | | TLKYNVAIKCATITPDEDRVKEFNLKQMWRSPNGTIRNIINGTVFREPIICKNVPKLVPGWTKPI |
| | | CIGRHAFGDQYRATDAVLKGPGKLRLVFEGKDETVDLEVFNFTGAGGVALAMYNTDESIQGFA |
| | | EASMAIAYEKKWPLYLSTKNTILKKYDGRFKDIFQAVYEADWKSKYEAAGIWYEHRLIDDMVA |
| | | YALKSEGGYVWACKNYDGDVQSDFLAQGFGSLGLMTSVLMCPDGKTIEAEAAHGTVTRHFR |
| | | VHQKGGETSTNSIASIFAWTRGLAHRAKLDDNARLLDFTQKLEDACVGTVESGKMTKDLALL |
| | | VHGSSKVTRGDYLNTEEFIDAVAAELQSRLAAN |
| 405 | 8 | FEGCLAKSYKSEKSDKSATYDYSANIECEKEPPKPLYGGGILTGAEAPAPVSAGGKKLLMAKSK |
| | | SAPAKGSTLKVELEKDTHYTLSAWLQLSKSTGDVKAILVTPDGNFNTAGMLVVQSGCWTMLK |
| | | GGATSFAAGKGELFFETNVTAELMVDSMSLQPFSFEEWKSHRHESIAKERKKKVKITVHGSD |
| | | GKVLPDAELSLERVAKGFPLGNAMTKEILDIPEYEKWFTSRFTVATMENEMKWYSTEYDQNQE |
| | | LYEIPDKMLALAEKYNISVRGHNVFWDDQSKQMDWVSKLSAPQLKKAMEKRMKNVVSRYAG |
| | | KLIHWDVLNENLHYSFFEDKLGKDASAEVFKEVAKLDDKPILFMNEYNTIEEPNDAAPLPTKYL |
| | | AKLKQIQSYPGNSKLKYGIGLESHFDTPNIPYVRGSLDTLAQAKVPIWLTEIDVKKGPKQVEYL |
| | | EEVMREGFAHPGVKGIVLWGAWHAKECYVMCLTDKNFKNLPVGDVVDKLITEWKAVPEDAK |
| | | TDDKGVFEAELFHGEYNVTVKHKS |
| 406 | 9 | MAQLQETYACSPATERGRGILLGGDAKTDTIVYCAGRTVFFRRLDAPLDAWTYTEHAYPTTVA |
| | | RISPNGEWVASADVSGCVRVWGRNGDRALKAEFRPISGRVDDLRWSPDGLRIVVSGDGKG |
| | | KSLVRAFMWDSGSTVGDFDGHSKRVLSCDFKPTRPFRIVTCGEDFLANYYEGPPFKFKHSIRD |
| | | HSNFVNCIRYSPDGSKFITVSSDKRGLIYDGKTGDKIGELSSEDSHTGSIYAVSWSADSKQVL |
| | | TVSADKTAKVWDIMEDASGKVNRTLVCTGIGGVDDMLVGCLWQNDHLVTVSLGGTFNVFSA |
| | | SNPDKEPVSFAGHLKTVSSLTYFPQSNPRTMLSTSYDGVIIRWIQGVGYGGRLIRKNNTQIKC |
| | | FVAAEEELITSGYDNMVFRIPLNGDQCGDAESVDVGGQPNALNIAVQQPEFALITTDSAIVLLH |
| | | KSTVTSTTKVSYTITSSAVSPDGTEAIVGAQDGKLRIYSISGDTLTEEAVLERHRGAITSIHYSP |
| | | DVSMFASADANREAVAWDRATREIKLKNMLFHTARINCLAWSPDSRLVATGSIDTCAIIYDVD |
| | | KPASSRITIKGAHLGGVHGLTFADNDTLVTAGEDACVRVWKLV |
| 407 | 10 | MVFSVTKKDTKPFDGQKPGTSGLRKKVTVFQQPHYLANFVQSTFNALPADQVKGATIVVSGD |
| | | GRYFSKDAVQIITKMAAANGVRRVWVGQDSLLSTPAVSAIIRERIAADGSKATGAFILTASHN |
| | | PGGPTEDFGIKYNMGNGGPAPESVTDKIFSNTTTITEYLIAEDLPDVDISALGVTTFTGPEGPFD |
| | | VDVFDSATDYIKLMKTIFDFESIKKLLASPKFSFCFDGLHGVAGAYAKRMFVDELGASESSLLN |
| | | CVPKEDFGGGHPDPNLTYAKELVERMGLGKSSSNVEPPEFGAAADGDADRNMVLGKRFFVTP |
| | | SDSVAIIAANAVQSIPYFASGLKGVARSMPTSAALDVVAKNLNLKFFEVPTGWKFFGNLMDAG |
| | | MCSVCGEESFGTGSDHIREKDGIWAVLAWLSIIAYKNKDNLGGDKLVSVEDIVLQHWATYGR |
| | | HYYTRYDYENVDAEAAKELMANLVKMQSALSDVNKLIKEIQPDVAEVVSADEFEYKDPVDGSV |
| | | SKHQGIRYLFGDGSRLVFRLSGTGSVGATIRIYIEQYEKDSSKTGRESSDALSPLVDVALKLSK |
| | | IKEYTGRSAPTVIT |
| 408 | 11 | MATSWTLPDHPTLPKGKTVAVIVLDGWGEASADQYNCIHRAETPVMDSLKNGAPEKWTLVKA |
| | | HGTAVGLPSDDDMGNSEVGHNALGAGRIFAQGAKLVDAALASGKIWEAEGFNYIKESFAEGT |
| | | LHLIGLLSDGGVHSRLDQVQLLVKGASERGAKRIRLHILTDGRDVLDGSSVGFVETLENDLAQ |
| | | LREKGVDAQVASGGGRMYVTMDRYENDWDVVKRGWDAQVLGEAPYKFKSALEAVKTLRAE |
| | | PKANDQYLPAFVIVDESGKSVGPIVDGDAVVTFNFRADRMVMLAKALEFADFDKFDRVRVPKI |
| | | KYAGMLQYDGELKLPNKFLVSPPLIERTSGEYLVKNGVRTFACSETVKFGHVTFFWNGNRSGY |
| | | FDETKEEYIEIPSDSGITFNEQPKMKALEIAEKTRDAILSGKFDQVRINLPNGDMVGHTGDIEA |
| | | TVVACKAADEAVKIVLDAVEQVGGIYLVTADHGNAEDMVKRNKSGQPALDKSGSIQILTSHTL |
| | | QPVPVAIGGPGLHPGVKFRSDINTPGLANVAATVMNLHGFQAPDDYETTLIEVAD |
| 409 | 13 | MDEEYDVIVLGTGLKECILSGLLSVDGLKVLHMDRNDYYGGESTSLNLTKIWKRFKGSEATPD |
| | | HLGVSKEYNVDMVPKFMMANGALVRVLIRTSVTKYLNFKAVDGSFVYNNGKIHKVPATDVEAL |
| | | KSNLMGLFEKRRARKFFIYVQDYEEEDPKSHEGLDLHKVTTREVISKYGLEDDTVDFIGHALAL |
| | | HRDDNYLDEPAIDTVKRMKLYAESLARFQGGSPYIYPLYGLGELPQAFARLSAVYGGTYMLNKP |
| | | ECKVEFDESGKAFGVTSEGETAKCKKVVCDPSYLPDKVTKVGRVARAICIMKHPIPDTKDSHS |
| | | VQIILPKKQLKRKSDMYVFCCSYAHNVAPKGKFIAFVSTEAETDKPEIELKPGIDLLGPVEETFF |
| | | DIYDRYEPANAPEEDNCFVTNSYDATTHFETTVKDVLALYSKITGKELDLSVDLNAASAGESE |
| 410 | 19 | GVVATTDAVEACTGVNVAVMVGGFPRKEGMERKDVMSKNVSTYKSQASALEAHAAPNCKVL |
| | | VVANPANTNALILKEFAPSIPEKNISCLTRLDHNRALGQVSERLNVQVSDVKNVLIWGNHSSS |
| | | QYPDVNHATVKTSSGEKPVRELVQDDEWLNGPFIATVQQRGAAIIKARKLSSALSAASSACD |
| | | HIRDWVLGTPEGTFVSMGVYSDGSYGVPAGLIYSFPVTCSGGEWTIVQGLPIDEFSRKKMDA |
| | | TAQELSEEKALAYSCL |
| 411 | 20 | MAASSRRASQLLGSAASRFLHSRGYAAAAAAPSPAVFVDKSTRVICQGITGKNGTFHTEQATE |
| | | YGTNMVGGVTPKKGGTEHLGLPVFNSVAEAKAETKANASVIYVPPPFAAAAIMEALEAELDLVV |
| | | CITEGIPQHDMVKVKAALNRQSKTRLIGPNCPGIIKPGECKIGIMPGYIHKPGRIGIVSRSGTLT |
| | | YEAVFQTTAVGLGQSTCVGMGGDPFNGTNFVDCLEKFVADPQTEGIVLIGEIGGTAEEDAAAF |
| | | TQASKTDKPVVAFIAGLTAPPGRRMGHAGAIVSGGKGTAQDKIKALREAGVTVVESPAKIGST |
| | | MFEIFKQRGMVE |
| 412 | 22 | MALPNQGTVDYPSFKLVIVGDGGTGKTTFVKRHLTGEFEKKYEPTIGVEVHPLDFTTNCGKIRF |
| | | YCWDTAGQEKFGGLRDGYYTHGQCAIIMFDVTSRLTYKNVPTWHRDLCRVCENIPIVLCGNKV |
| | | DVKNRQVKAKQVTFHRKKNLQYYEISAKSNYNFEKPFLYLARKLAGDANIHFVEAVALKPPEVT |
| | | FDLAMQQQH |
| 413 | 24 | MATKRSVGTLGEADLKGKKVFLRADLNVPLDDAQKITDDTRIRASIPTIKFLLEKGAKVILASHL |
| | | GRPKGVTPKFSLKPLVPRLSELLGVEVVMANDCIGEEVEKLAAALPEGGVLLLENVRFYKEEEK |
| | | NDPEFAKKLASVADLYVNDAFGTAHRAHASTEGVTKFLRPSVAGFLMQKELDYLVGAVANPKK |
| | | PFAAIVGGSKVSSKIGVIESLLAKVDILILGGGMIFTFYKAQGKAVGKSLVEEDKLELATSLIETA |
| | | KAKGVSLLLPTDVVVADKFAPDAESKTVSADAIPDGWMGLDVGPDSIKTFSEALDTTKTVIWN |
| | | GPMGVFEFEKFAAGTDAIAKQLADLTGKGVTTIIGGGDSVAAVEKAGLADKMSHISTGGGAS |
| | | LELLEGKPLPGVLALDEA |
| 414 | 26 | GVFTDKDKAAAHMKGGAKKVVISAPSKDAPMFVVGVNEDKYTSDVNIVSNASCTTNCLAPLA |
| | | KIINDNFGIVEGLMTTVHSITATQKTVDGPSSKDWRGGRAASFNIIPSSTGAAKAVGKVLPEL |
| | | NGKLTGMSFRVPTVDVSVVDLTVRIEKAASYEDIKKAIKAASEGNLKGIMGYVEEDLVSTDFIG |
| | | DSRSSIFDAKAGIALNDNFVKLVSWYDNEWGY |
| 415 | 27 | MSAYCGKYKDELIKNAAYIGTPGKGILAADESTGTIGKRFASINVENVEDNRRALRELLFTTPG |
| | | ALQHISGVILFEETLYQSSKAGKPFVDILKENNVLPGIKVDKGTVELAGTDKETTTQGHDDLGK |
| | | RCAKYYEAGARFAKWRAVLKIGPNEPSQLSIDQNAQGLARYAIICQENGLVPIVEPEILVDGPH |
| | | DIERCAYVTEVVLAACYKALNDQHVLLEGSLLKPNMVTPGSDAKKVAPEVIAEYTVRTLQRTVP |
| | | PAVPAIVFLSGGQSEEEATVNLNAMNKLQTKKPWFLSFSFGRALQQSTLKAWSGKEENVEKA |
| | | QKAFLVRCKANSEATLGTYKGDATLGEGASESLHVKDYKY |
| 416 | 29 | MASEKHFKYVILGGGVAAGYAAREFAKQGVQPGELAIISKESVAPYERPALSKGYLFPQNAARL |
| | | PGFHTCVGSGGEKLLPEWYTEKGIELILSTEIVKADLASKTLTSAAGATFTYETLLIATGSSTIKL |
| | | TDFGVQGAEANNILYLRDINDADKLVAAMQAKKDGKAVVVGGGYIGLELSAALKLNNFDVTM |
| | | VYPEPWCMPRLFTAGIAHFYEGYYASKGINIVKGTVASGFDADANGDVAVVKLKDGRVLDANI |
| | | VIVGVGGRPLTGLFKGQVDEEKGGLKTDTFFETSVAGVYAIGDVASFPMKLYNEPRRVEHVDH |
| | | ARKSAEQAVKAIKAKESGETVAEYDYLPYFYSRSFDIAWQFYGDNVGESVLFGDNDPAAAKAK |
| | | FGTYWVKDGKVVGVFLEGGSADENQATAKVARAQPLVAANLGELGKEGLDFAAKI |
| 417 | 30 | MAGGGVEDVYGEDRATEEQFVTPWSFSVASGHSLLRDPRHNKGLAFSEAERDAHYLRGLLPP |
| | | AIVSQEHQEKKIMHNLRQYTVPLQRYIAMMDLQERNERLFYKLLIDNVEELLPVVYTPVVGEAC |
| | | QKYGSTYRRPQGLYISLKDKGKVLEVLKNWPERSIQVIVVTDGERILGLGDLGCQGMGIPVGK |
| | | LSLYTALGGVRPSACLPITIDVGTNNQTLLDDEYYIGLKQRRATGEEYHELLQEFMNAVKQNYG |
| | | EKVLVQFEDFANHNAFDLLAKYSKSHLVFNDDIQGTASVVLAGLLAALKVIGGGLADQTYLFLG |
| | | AGEAGTGIAELIALEMSKHTDLPLDDCRKKIWLVDSKGLLVESRKESLQHFKKPFAHEHEPLTT |
| | | LLEAVQSLKPTVLIGTSGVGKTFTQEVVEAMASFNEKPVIFSLSNPTSHSECTAEEAYTWTKGT |
| | | AVFASGSPFDPVEYEGKTYVPGQSNNAYVFPGFGLGVVISGAIRVHDDMLLAASEALAEQVSQ |
| | | ENFDKGLIFPPFTNIRKISANIAAKVAAKAYDLGLASRLPRPDDLVKYAESCMYTPLYRSYR |
| 418 | 32 | MAPIKIGINGFGRIGRLVARVALQCPDVELVAVNDPFITTDYMTYMFKYDTVHGQWKHHDVKV |
| | | KDAKTLLFGEKEVAVFGCRNPEEIPWGAAGADYVVESTGVFTDKDKAAAHIKGGAKKVIISAP |
| | | SKDAPMFVCGVNEKEYTSDITIVSNASCTTNCLAPLAKVINDRFGIVEGLMTTVHAMTATQKT |
| | | VDGPSSKDWRGGRAASFNIIPSSTGAAKAVGKVLPVLNGKLTGMAFRVPTVDVSVVDLTVRL |
| | | EKAATYEQIKAAIKEESEGNLKGILGYVDEDLVSTDFQGDSRSSIFDAKAGIALNDNFVKLVS |
| | | WYDNEWGYSTRVVDLIRHIHATK |
| 419 | 34 | MSFSWICACVRAAAVAWEAGKPLSIEEVEVAPPQAMEVRVKILFTALCHTDVYFWEAKGQTPV |
| | | FPRIFGHEAGGIVESVGEGVTDVAPGDHVLPVFTGECKECRHCKSAESNMCDLLRINTDRGV |
| | | MISDGKSRFSIDGKPIFHFVGTSTFSEYTVMHVGCVAKINPEAPLDKVCVLSCGISTGLGASIN |
| | | VAKPPKGSTVAIFGLGAVGLAAAEGARIAGASRIIGIDLNANRFEEARKFGCTEFVNPKDHTKP |
| | | VQEVLAEMTDGGVDRSVECTGNINAMIQAFECVHDGWGVAVLVGVPHKDAEFKTHPMNFLN |
| | | ERTLKGTFFGNFKPRTDLPNVVEMYMKKELEVEKFITHSVTFSEINKAFDLMAKGEGIRCIIRME |
| | | H |
| 420 | 39_ | MAPRKFFVGGNWKCNGASDDVKKIVTVLNEAEVPSEDAVEVVVSPPFVFLQQAKALLRPDFA |
| | 59 | VAAQNCWVRKGGAFTGEISAEMLVNLQVPWVILGHSERRALLSESNDFVADKVAYALAQGLK |
| | | VIACIGETLEQREAGTTMEVVAAQTKAIAEKISDWTNVVLAYEPVWAIGTGKVASRAQAQEVH |
| | | DGLRKWLHANVGPAVAESTRIIYGGSVNGANCKELAAQPDLDGFLVGGASLKPEFVDIIKSAT |
| | | VKSSS |
| 421 | 43 | GCRAGGNSATEPYIAGHHLLLAHAAAVKIYRDKYQPAQQGKIGILLDFVWYEPLTYNTEDEFAA |
| | | HRAREFTLGWFMHPITYGHYPETMQRLVADRLPNFTDEQTRLLQGSADIVGVNHYTTYYAKNH |
| | | ENLTHMSYANDWQVQLVYERNGIPIGKQGYSKWLYVVPWGFYKAVMHVKDKYRNPLMIIGE |
| | | NGIDQSGSDTLPHALYDKFRIDYFDQYLHELKRATDDGARVTGYFAWSLLDNFEWRMGFTSK |
| | | FGIVYVDRKTFTRYPKDSTRWFRKV |
| 422 | 43 | KTNKDGVDYYHRLINYMLANKITPYVVLYHYDLPEVLNNQYNGWLSPRVVPDFAYFADFCFK |
| 423 | 43 | LTRHSFPKGFVFGTASSAYQVEGNALQYGRGPCIWDTFLKFPGATPDNATANVTVDEYHRYM |
| 424 | 47 | MATDAAAPAAASKWNLLTFDTEEDVAVSLAKYTAELSGKFAAERGAFTVVLSGGTLIDTLRKL |
| | | AEPPYLETVQWSKWHVFWVDERVVPKDHVDSNYKLAVDGLLSKVPIPTDQVYAINDTLSAEG |
| | | AAADYETVLKQLVKNGVLAMSTATGFPRFDLMLLGMGPDGHLASLFPGHPLLNENQKWVTHI |
| | | MDSPKPPPQRITFTFPVIKSSAYVAMVVTGPGEASAVKKVLSDDKTLP |
| 425 | 47 | DGHLASLFPGHPLLNENQKWVTHIMDSPKPPPQRITFTFPVIKSSAYVAMVVTGPGEASAVKK |
| | | VLSDDKTLPLLPTEMAILQDGEFTWFTDKQAVSMLQNK |
| 426 | 49/54 | STNVARAEDPYVFFEWHVTYGTKTVLGVPQKVILINGEFPGPRINCSSNNNIVVNVFNQLDQP |
| | | LLFTWNGIQHRKNSWQDGLPGTNCPVAPGTNFTYKWQPKDQIGSFFYFPSIGMQRTVGGYGL |
| | | ISVVSRLLIPVPFDPPADDLQVIIGDWYTKDHAVMASLLDAGKSFGRPAGVLINGRGGKDATN |
| | | PPMFTFEAGKTYRLRVCNVGIKSSLNFRIQGHDMRLVEMDGSHTLQDSYDSLDVHVGHCFSV |
| | | LVDADQKPADYLMVASTRFIADGSSASAVIRYAGSNTPPAANVPEPPAGWAWSLNQWRSFR |
| | | WNLTASAARPNPQGSYHYGQINITRTIKLMITRGHLDGKLKYGFNGVSHVDADTPLKLAEYFN |
| | | VSDQVFKYNQMGDAPPGVNGPMHVTPNVITAEFRTFIEVVFENPEKSMDSLHIDGYAFFAVG |
| | | MGPGKWKPELRKTYNLLDAVSRHSIQVYPRSWSAVMLTFDNAGMWNVRSNVWERHYLGEQ |
| | | LYISVISPARSLRDEYNFPENALRCGKVVGLPLPPSYLPA |
| 427 | 49/54 | MATTTTRGTAAAGGVLLLALLLLSTNVARAEDPYVFFEWHVTYGTKTVLGVPQKVILINGEFPG |
| | | PRINCSSNNNIVVNVFNQLDQPLLFTWNGIQHRKNSWQDGLPGTNCPVAPGTNFTYKWQPK |
| | | DQIGSFFYFPSIGMQRTVGGYGLISVVSRLLIPVPFDPPADDLQVIIGDWYTKDHAVMASLLDA |
| | | GKSFGRPAGVLINGRGGKDATNPPMFTFEAGKTYRLRVCNVGIKSSLNFRIQGHDMRLVEMD |
| | | GSHTLQDSYDSLDVHVGHCFSVLVDADQKPADYLMVASTRFIADGSSASAVIRYAGSNTPPA |
| | | ANVPEPPAGWAWSLNQWRSFRWNLTASAARPNPQGSYHYGQINITRTIKLMITRGHLDGKLK |
| | | YGFNGVSHVDADTPLKLAEYFNVSDQVFKYNQMGDAPPGVNGPMHVTPNVITAEFRTFIEVVF |
| | | ENPEKSMDSLHIDGYAFFAVGMGPGKWKPELRKTYNLLDAVSRHSIQVYPRSWSAVMLTFDN |
| | | AGMWNVRSNVWERHYLGEQLYISVISPARSLRDEYNFPENALRCGKVVGLPLPPSYLPA |
| 428 | 49/54 | TKDHAVMASLLDAGKSFGRPAGVLINGRGGKDATNPPMFTFEAGKTYRLRVCNVGIKSSLNF |
| | Fragment | RIQGHDMRLVEMDGSHTLQDSYDSLDVHVGHCFSVLVDADQKPADYLMVASTRFIADGSSA |
| | | SAVIRYAGSNTPPAANVPEPPAGWAWSLNQWRSFRWNLTASAARPNPQGSYHYGQINITRTI |
| | | KLMITRGHLDGKLKYGFNGVSHVDADTPLKLAEYFNVSDQVFKYNQMGDAPPGVNGPMHVTP |
| | | NVITAEFRTFIEVVFENPEKSMDSLHIDGYAFFAVGMGPGKWKPELRK |
| 429 | 51 | MSPAEPTREESVYMAKLAEQAERYEEMVEFMERVAKATGGAGPGEELSVEERNLLSVAYKNVI |
| | | GARRASWRIISSIEQKEEGRGNDAHATTIRSYRSKIEAELAKICDGILALLDSHLVPSAGAAES |
| | | KVFYLKMKGDYHRYLAEFKSGAERKEAAESTMNSYKAAQDIALADLAPTHPIRLGLALNFSVFY |
| | | YEILNSPDRACNLAKQAFDEAISELDSLGEESYKDSTLIMQLLRDNLTLWTSDTNEDGGDEIKE |
| | | APAPKESGD |
| 430 | 52 | MDACRLLLLLLLGLLGLLAPLASAQLSREFYKASCPDAEKIVAAVIEKKLKEDPGTAAGLLRLLFH |
| | | DCFANGCDASILIDPLSNQSAEKEAGPNISVRGFEVIDDIKKELEAKCPKTVSCADIVALGTRD |
| | | AVRISGGPAYEVPTGRRDSLVSNREEADNNLPGPDIPIPKLTSEFLSRGFTPEEMVVLLAGGHS |
| | | IGKVRCIFIEPDATPMDPGYQASISKLCDGPNRDTGFVNMDEHNPNVIDSSYFANVLAKKMPL |
| | | TVDRLLGLDSKTTPIIKNMLNKPNDFMPTFAKAMEKLSVLKVITGKDG |
| 431 | 53 | MDRNPVAKNAGKFMTLAGVLDYAKASNISGILIGIEHAAYLATRGLDVVDAVSNALIKSGYDK |
| | | ETKQQVFIQSEDPPVLSAFKKFPKFNRVFEIEFDIRDVSKPSVVEIKEFANAVKLRRSSAAQVD |
| | | GFYLTGFNAVVERLRDADIQVHVGVLKNEFMSLAFDYWADPMVEIATDTWSVLADGLVTEFP |
| | | STAAAYFRSPCSDIKRNMSYTIKPGEPGALVDMAAYGALPPAPPPAPVLEPADVHRQPLPLCPT |
| | | EPMFRTFRCRLPPKETGKNAEYTANLAADG |
| 432 | 56 | MARLLFPLPIAAAAVSASSIHLAASRFRLPVVSAARRGTLFGGRVAVRAPARLATRGVSAGAEA |
| | | GGSAARAGTVIGPEEALEWVKNDRRRLLHVVYRVGDLDKTIKFYTECLGMKLLRKRDIPEERY |
| | | TNAFLGYGPEDSHFVVELTYNYGVESYDIGSGFGHFGIAVEDVEKTVELIKAKGGTVTREPGPV |
| | | KGGKSVIAFIEDPDGYKFELIERGPTPEPLCQVMLRVGDLDRAIKFYEKAFGMELLRRKDNPQY |
| | | KYTIAMMGYGPEDKNAVLELTYNYGVKEYDKGNAYAQIAIGTDDVYKTAEVVRQNGGQITREP |
| | | GPLPGISTKITACTDPDGWKSVFVDNLDFLKELEE |
| 433 | 62 | MRRLSLILLAAAALLAAAVSAEPGPAPKLSPDFYSQTCPRAERIIAEVVQSKQMANPTTAAGVL |
| | | RVFFHDCFVSGCDASVLIAPTHYAKSEKDADINHSLPGDAFDAVVRSKLALELECPGVVSCAD |
| | | ILAIASRVLVTMTGGPRYPVPLGRKDSLSSNPAAPDVELPHSNFTVGRIIELFTAKGFTVQEMVA |
| | | LSGAHTLGFSHCQEFASRIYNYRDKGGKPAPFDPSMNPTYAKGLQAACQDYQKDPTIAAFNDI |
| | | MTPGKFDNMYYVNIERGLGLLSTDEDMWSDMRTKPFVQRYAANNTDFFEDFAKAIEKLSMYG |
| | | VKTGADGEIRRRCDAFNSGPNIQ |
| 434 | 65 | AMAVDLTPRQPTKAYGGDGGAYYEWSPAELPMLGVASIGAAKLSLAAGGMSLPSYSDSAKVA |
| | | YVLQGKGTCGIVLPEATKEKVVAIKEGDALALPFGVVTWWHNTPESSTELVVLFLGDTSKGHT |
| | | PGKFTNFQLTGATGIFTGFSTEFVARAWDLDQDAAASLVSTQPGTGIVKLAPGHKMPVARAED |
| | | RKGMALNCLEAKLDVDIPNGGRVVVLNTVNLPLVKEVGLGADLVRIDAHSMCSPGFSCDSAY |
| | | QVTYIVRGSGRVQVVGPDGKRVLETRIEGGSLFIVPRFHVVSKIADASGMEWFSIITTPNPIFS |
| | | HLAGKTSVWKAISPEVLEAAFNTTPEMEKLFRSKRLDSEIFFAPS |
| 435 | 73 | MSSAKQVLEPAFQGAGQKPGTEIWRIENFNPVPLPKSDYGKFYCGDSYIVLQTTCNKGGAYLF |
| | | DIHFWIGKDSSQDEAGTSAIKTVELDTMLGGRAVQHREPQGYESDKFLSYFKPCIIPLEGGFA |
| | | SGFKTPEEEKFETRLYICKGKRAIRVKEVPFARSSLNHDDVFILDTEKKIYQFNGANSNIQERAK |
| | | ALEVIQHLKDKYHEGVCDVAIVDDGKLQAESDSGEFWVVFGGFAPIGKKTVSDDDVILETSPT |
| | | KLYSINNGKLKLEDIVLTKSILENTKCFLLDCGSELFVWVGRVTQVDDRKAASAAVEEFIVKQN |
| | | RPKTTRVTQVIQGYENHTFKSLFESWPVSSTGNASTEEGRGKVAALLKKKGDVKGASKNSTP |
| | | VNEEVPPLLEGSGKLEVWCVDGSAKTALPKEDLGKFHSGDCYIVLYTYHSGEKREEFYLTYWI |
| | | GKDSVLEDQHMALQIATTIWNSMKGRPVLGRIYQGKEPPQFIALFQPMVILKGGISSGYKKSI |
| | | EENGLKDETYSGTGIALVHIHGTSIHNNKTLQVDAVSISLSSTDCFVLQSGNSMFTWIGNTSS |
| | | YEQQQWAAKVAEFLKPGASVKHCKEGTESSAFWSALGGKQNYTSKNATQDVLREPHLYTFSF |
| | | RNGKLEVTEVFNFSQDDLLTEDVMILDTHAEVFVWMGQCVDTKEKQTAFETGQKYVEHAVNF |
| | | EGLSPDVPLYKVSEGNEPCFFRTYFSWDNTRSVIHGNSFQKKLSLLFGMRSESGSKGSGDGG |
| | | PTQRASALAALSSAFNPSSQDKQSNDRPKSSGDGGPTQRASALAALSSSLNPSSKPKSPHSQ |
| | | SRSGQGSQRAAAVAALSNVLTAEGSTLSPRNDAEKTELAPSEFHTDQDAPGDEVPSEGERTE |
| | | PDVSQEETANENGGETTFSYDRLISKSTDPVRGIDYKRRETYLSDSEFETVFGVTKEEFYQQPR |
| | | WKQELQKRKADLF |
| 436 | 76 | MASHIVGYPRMGPKRELKFALESFWDGKSSAEDLEKVATDLRASIWKQMSEAGIKYIPSNTFS |
| | | YYDQVLDTTAMLGAVPDRYSWTGGEIGHSTYFSMARGNATVPAMEMTKWFDTNYHFIVPELG |
| | | PETKFSYASHKAVSEYKEAKALGVDTVPVLVGPVSYLLLSKAAKGVEKSFSLLSLLGGILPIYKE |
| | | VVAELKAAGASWIQFDEPTLVKDLAAHELAAFSSAYAELESSLSGLNVLIETYFADVPAESYKTL |
| | | TSLSGVTAYGFDLVRGTKTLDL- |
| | | LKSVGIPSGKYLFAGVVDGRNIWADDLAASLSTLESLEAIVGKDKLVVSTSCSLMHTAVDLVN |
| | | ETKLDSEIKSWLAFAAQKVVEVNALGKALVGLKDEAYFAANAAAQASRRSSPRVNNEEVQKA |
| | | AAALKGSDHRRATTVSARLDAQQKKLNLPVLPTTTIGSFPQTMDLRRVRREYKAKKISEEAYV |
| | | SAIKEEISKVVKIQEELDIDVLVHGEPERNDMVEYFGEQLSGFAFTANGWVQSYGSRCVKPPII |
| | | YGDVSRPNPMTVFWSKMAQSMTPRPMKGMLTGPV |
| 437 | 77 | QEVAGDVRMTDTRADEAERGITIKSTGISLYYEMSEESLASYKGDRDGNDYLINLIDSPGHVD |
| | | FSSEVTAALRITDGALVVVDCIEGVCVQTETVLRQALGERIRPVLTVNKMDRCFLELQVDGEEA |
| | | YQTFSRVIENANVIMATYEDALLGDVQVYPEKGTVAFSAGLHGWAFTLTNFAKMYASKFGVDE |
| | | SKMMERLWGENFFDPATKKWTSKNTGSGTCKRGFVQFCYEPIKQIIEICMNDQKDKLWPMLK |
| | | KLGVTMKNDEKDLMGKALMKRVMQAWLPASRALLEMMVYHLPSPSKAQRYRVENLYEGPLD |
| | | DVYANAIRNCDPEGPLMLYVSKMIPASDKGRFFAFGRVFAGRVATGMKVRIMGPNFVPGQKK |
| | | DLYTKSVQRTVIWMGKKQESVEDVPCGNTVALVGLDQFITKNATLTGEKEVDACPIRAMKFS |
| | | VSPVVRVAVQCKVASDLPKLVEGLKRLAKSDPMVLCSIEESGEHIIAGAGELHLEICLKDLQDD |
| | | FMGGAEIIVSPPVVSFRETVLDKSCRTVMSKSPNKHNRLYMEARPLEEGLPEAIDEGRIGPRDD |
| | | PKVRSKILSEEFGWDKDLAKKIWCFGPETTGPNMVVDMCKGVQYLNEIKDSVVAGFQWASK |
| | | EGALADENMRGICFEVCDVVLHTDAIHRGGGQVIPTARRVIFASQLTAKPRLLEPVYLVEIQAP |
| | | EGALGGIYGVLNQKRGHVFEEMQRPGTPLYNIKAYLPVIESFGFSATLRAATSGQAFPQCVFDH |
| | | WDVMNSDPLEVDSQSFNLVKEIRKRKGLKEQMTPLSDFEDKL |
| 438 | 86 | MSTAEATREENVYMAKLAEQAERYEEMVEFMEKVAKTADVGELTVEERNLLSVAYKNVIGARR |
| | | ASWRIISSIEQKEESRGNEAYVASIKEYRTRIETELSKICDGILKLLDSHLVPSATAAESKVFYLK |
| | | MKGDYHRYLAEFKAGAERKEAAENTLVAYKSAQDIALADLPTTHPIRLGLALNFSVFYYEILNSP |
| | | DRACNLAKQAFDEAIAELDSLGEESYKDSTLIMQLLRDNLTLWTSDNADEGGDEIKEASKPEG |
| | | EGH |
| 439 | 86/51 | MSPAEPTREESVYMAKLAEQAERYEEMVEFMERVAKATGGAGPGEELSVEERNLLSVAYKNVI |
| | | GARRASWRIISSIEQKEEGRGNDAHATTIRSYRSKIEAELAKICDGILALLDSHLVPSAGAAES |
| | | KVFYLKMKGDYHRYLAEFKSGAERKEAAESTMNSYKAAQDIALADLAPTHPIRLGLALNFSVFY |
| | | YEILNSPDRACNLAKQAFDEAISELDSLGEESYKDSTLIMQLLRDNLTLWTSDTNEDGGDEIKE |
| | | APAPKESGD |
| 440 | 87 | MAVKVYVVYYSMYGHVGKLAEEIKKGASSVEGVEVKVWQVPEILSEEVLGKMGAPPKTDVPII |
| | | SPQELAEADGILFGFPTRFGMMASQMKAFFDATGGLWREQSLAGKPAGVFFSTGTQGGGQE |
| | | TTPLTAVTQLTHHGMVFVPVGYTFGAKMFDMEKVQGGSPYGAGTFAGDGSRWPSEMELEHA |
| | | FHQGKYFAGIAKKLKGS |
| 441 | 89 | MSAADKVKPAASPAAEDPAAIAGNISYHAHYSPHFSPLAFGPEPAYFATAESVRDHLLQRWND |
| | | TYLHFHKTDPKQTYYLSMEYLQGRALTNAVGNLGITGAYAEAVKKFGYELEALAGQERDMALG |
| | | NGGLGRLAACFLDSMATLNLPAWGYGLRYRYGLFKQRIAKEGQEEIAEDWLEKFSPWEIVRH |
| | | DVVYPVRFFGHVEILPDGRRKSAGGEVLNALAYDVPIPGYKTKNAISLRLWDAKASAEDFNLF |
| | | QFNDGQYESAAQLHSRAQQICAVLYPGDATEEGKLLRLKQQFFLCSASLQDIIFRFKERKSDR |
| | | VSGKWSEFPSKVAVQMNDTHPTLAIPELMRLLMDEEGLGWDEAWDVTNKTVAYTNHTVLPE |
| | | ALEKWSQSVMRKLLPRQMEIIEEIDKRFREMVISTRKDMEGKLDSMSVLDNSPQKPVVRMAN |
| | | LCVVSAHTVNGVAELHSNILKEELFADYVSIWPKKFQNKTNGITPRRWLRFCNPELSEIVTKWL |
| | | KTDQWTSNLDLLTGLRKFADDEKLHAEWAAAKLASKKRLAKHVLDATGVTIDPTSLFDIQIKR |
| | | IHEYKRQLMNILGAVYRYKKLKEMSAEEKQKVTPRTVMVGGKAFATYTNAKRIVKLVNDVGAV |
| | | VNNDPDVNKYLKVVFIPNYNVSVAEVLIPGSELSQHISTAGMEASGTSNMKFSLNGCVIIGTLD |
| | | GANVEIREEVGEDNFFLFGAKADQVAGLRKDRENGLFKPDPRFEEAKQYIRSGTFGTYDYTPLL |
| | | DSLEGNSGFGRGDYFLVGYDFPSYIDAQARVDEAYKDKKRWIKMSILNTAGSGKFSSDRTID |
| | | QYAKEIWGITANPVP |
| 442 | 91 | MAANPRVFFDVTIGGAPAGRIVMELYADVVPKTAENFRALCTGEKGVGKMGKPLHYKGSSFH |
| | | RVIPGFMCQGGDFTAGNGTGGESIYGAKFADENFVKKHTGPGVLSMANAGPGTNGSQFFLCT |
| | | AKTAWLDGKHVVFGQVVEG |
| 443 | 91 | AANPRVFFDVTIGGAPAGRIVMELYADVVPKTAENFRALCTGEKGVGKMGKPLHYKGSSFHRV |
| | | IPGFMCQGGDFTAGNGTGGESIYGAKFADENFVKKHTGPGVLSMANAGPGTNGSQFFLCTAK |
| | | TAWLDGKHVVFGQVVEGMDVVKAVEKVGSQSGRCSKPVVIADCGQL |

Table 3 shows conserved regions of NTGA's shown in Table 2 that are conserved across a grass pollen (Phl p), a weed pollen (Amb a and/or Amb p) and a tree pollen (Que a and/or Bet v). The conserved regions are denoted GWT.

TABLE 3
Table 3 Conserved regions (GWT) (SEQ ID Nos: 444-664)

| SEQ | | | |
|---|---|---|---|
| ID | NTGA | | |
| NO | ID | The conserved Phl p sequence is shown | |
| 444 | 1 | a | TIQSVKARQIFDSRGNPTVEVDVC |
| 445 | 1 | b | SDGTFARAAVPSGASTGVYEALELRDGGSDYLGKGVLKAVDNVNSIIGPALIGKDPTEQT |
| 446 | 1 | c | DNFMVHQLDGTKNEWGWCKQKLGANAILAVSLAVCKAGALVKKIPLYQHIANLAGNKQLV |
| | | | LPVPAFNVINGGSHAGNKLAMQEFMILPTGASSFKEAMKMGVEVYHNLKSVIKKKYGQDAT |
| | | | NVGDEGGFAPNIQENKEGLELLKTAIEKAGYTGKVVIGMDVAASEFYGE |
| 447 | 1 | d | DQTYDLNFKEENNDGSQKISG |
| 448 | 1 | e | LKNVYKSFVSEYPIVSIEDPFDQDDWVHY |
| 449 | 1 | f | FVSEYPIVSIEDPFDQDDWVHYAKMTEEIGEQVQIVGDDLLVTNPTRVAKAIAEKSCNALLL |
| | | | KVNQIGSVTESIEAVKMSKRAGWGVMTSHRSGETEDTFIADLAVGLSTGQIKTGAPCRSER |
| | | | LAKYNQLLRIEEELGAAAVYAG |
| 450 | 2 | a | MFNGEKINSTENRSVLHVALRAPRD |
| 451 | 2 | b | SVGIGGSFLGPLFVHTALQTDPEAAESAKGRQLRFLANVDPVDVARSI |
| 452 | 2 | c | DLDPETTLVVVVSKTFTTAETMLNARTIKEWI |
| 453 | 2 | d | LVKEFGIDPNNAFAFWDWVGGRYSVCSAVGVLPLSLQYGFPIV |
| 454 | 2 | e | ASFEKNIPVLLGLLSVWNVSFLGYPARAILPYSQALEKLAPHIQQLSMESNGKGVSIDGVPLP |
| | | | YEAGEIDFGEPGTNGQHSFYQLIHQGRVIPCDFIGVIKSQQPVYLKGETVSNHDELMSNFFA |
| | | | QPDALAYGKTPEQLRSENVS |
| 455 | 2 | f | LIPHKTFKGNRPSLSFLL |
| 456 | 2 | g | SLSAYEIGQLLAIYEHRIAVQGFIWGINSFDQWGVELGKSLASQVRKQLHASR |
| 457 | 3 | a | LKEQLELDKDDESLRRWKEQLLGQVDT |
| 458 | 3 | b | NIVSGLKYTNTVWKTGVRV |
| 459 | 3 | c | EETTPAGIFARGSYSAKLKFVDDD |
| 460 | 4 | a | KLMKTIFDFESIKKL |
| 461 | 4 | b | FCFDGLHGVAGAYAKRMFVDELGASESSLLNCVPKEDFGGGHPDPNLTYAKELVERMGLG |
| 462 | 4 | c | VEPPEFGAAADGDADRNMVLGKRFFVTPSDSVAIIAANAVQSIPYFASGLKGVARSMPTSA |
| | | | ALDVVAKNLNLKFFEVPTGWKFFGNLMDAGMCSVCGEESFGTGSDHIREKDGIWAVLAWL |
| | | | SIIAYKNK |
| 463 | 4 | d | KLVSVEDIVLQHWATYGRHYYTRYDYENVDAEAAKELMA |
| 464 | 4 | e | DVAEVVSADEFEYKDPVDGSVSKHQGIRYLFGDGSRLVFRLSGTGSVGATIRIYIEQYEKDS |
| | | | SKTGRES |
| 465 | 4 | f | DALSPLVDVALKLSK |
| 466 | 5/64 | a | KLRGLIAEKNCAPLMLRIAWHSAGTFDVATKTGGPFGTMR |
| 467 | 5/64 | b | AELAHGANAGLDIAVRLLEPIKEQVPILSYADFYQLAGVVAVEITGGPEVPFHPGRQDKTEPP |
| | | | PEGRLPDATLGSDHLR |
| 468 | 5/64 | c | AQMGLSDQDIVALSGGHTLGRCHKERSGFEGAWTANPLIFDNSYFTELLTGEKEGLLQLPT |
| | | | DKTLLTDPAFRPLVEKYAADEDAFFADYAEAHLKLSELGFGE |
| 469 | 5/64 | d | KLRGLIAEKNCAPLMLRIAWHSAGTFDVATKTGGPFGTMR |
| 470 | 5/64 | e | AELAHGANAGLDIAVRLLEPIKEQVPILSYADFYQLAGVVAVEITGGPEVPFHPGRQDKTEPP |
| | | | PEGRLPDATLGSDHLR |
| 471 | 5/64 | f | AQMGLSDQDIVALSGGHTLGRCHKERSGFEGAWTANPLIFDNSYFTELLTGEKEGLLQLPT |
| | | | DKTLLTDPAFRPLVEKYAADEDAFFADYAEAHLKLSELGFGEA |
| 472 | 5/64 | g | PFHPGREDKPQPPPEGRLPDATKGSDHLRQVFGKQMGLSDQDIVALSGGHTLGRCHKERS |
| | | | GFEGPWTKNPLKFDN |
| 473 | 5/64 | h | DKTLLTDPVFRPLVEKYAADEKAFFEDY |
| 474 | 6 | a | TKALLDKLAVLKLNGGLGTTMGCTGPKSVIEVRNGFTFLDLIVLQIESLNKKYGSNVPLLLMN |
| | | | SFNTHEDTLKIVEKY |
| 475 | 6 | b | IHTFNQSQYPRVVAD |
| 476 | 6 | c | PSKGKTDKDGWYPPGHGDIFPSLMNSGKLDLLLSQGKEYVFIANSDNLGAIVDMKILNHL |
| 477 | 6 | d | KQNEYCMEVTPKTLADVKGGTLISYEGRVQLLEIAQVPDAHVDEFKSIEKFKIFNTNNLWVN |
| | | | LKAIKRLVEADALKMEIIPNPKEVDGVKVLQLETAAGAAIRFFDHAIGINVPRSRFLPVKATS |
| | | | DLQLVQSDLYT |
| 478 | 6 | e | ARTDPSNPSIELGPEFKKVGSFLGRFKSIPSIVELDSLKVSGDVWFGSG |
| 479 | 6 | f | PGVKLEIPDGAVLENKDI |
| 480 | 7 | a | GDEMTRVFWQSIKEKLIFPFLDLDIKYYDLGVLHRDATDDKVTVEAAEATLKYNVAIKCATIT |
| | | | PDEDRVKEF |
| 481 | 7 | b | LKQMWRSPNGTIRNIINGTVFREPIICKNVPKLVPPhl pKPICIGRHAFGDQYRATDAVLKG |
| 482 | 7 | c | DLEVFNFTGAGGVALAMYNTDESIQGFAEASM |
| 483 | 7 | d | IAYEKKWPLYLSTKNTILKKYDGRFKDIFQAVYEADWKSKYEAAGIWYEHRLIDDMVAYALK |
| | | | SEGGYVWACKNYDGDVQSDFLAQGFGSLGLMTSVLMCPDGKTIEAEAAHGTVTRHFRVH |
| | | | QKGGETSTNSIASIFAWTRGLAHRAKLDDNARLLDFTQKLE |
| 484 | 7 | e | ACVGTVESGKMTKDLALLVHG |
| 485 | 7 | f | RGDYLNTEEFIDAVAAELQ |
| 486 | 9 | a | ETYACSPATERGRGIL |
| 487 | 9 | b | EHAYPTTVARISPNGEWVASADVSGCVR |
| 488 | 9 | c | RIVVSGDGKGKSLVRAFMWDSGSTVG |
| 489 | 9 | d | FDGHSKRVLSCDFKPTRPFRIVTCGEDFLANYYEGPPFKFKHSIRDHSNFVNCIRYSPDGSK |
| | | | FITVSSDKRGLIYD |
| 490 | 9 | e | GELSSEDSHTGSIYAVSWSADSKQVLTVSADKTAKVW |
| 491 | 9 | f | GIGGVDDMLVGCLWQNDHLVTVSLGGT |
| 492 | 9 | g | SPDGTEAIVGAQDGKLRIYS |
| 493 | 9 | h | GDTLTEEAVLERHRGAI |
| 494 | 9 | i | YSPDVSMFASADANREAV |
| 495 | 9 | j | REIKLKNMLFHTARINCLAWSPD |
| 496 | 9 | k | DKPASSRITIKGAHLGGVH |
| 497 | 10 | a | PFDGQKPGTSGLRKKVTVFQQPHYLANFVQSTFNALP |
| 498 | 10 | b | TIVVSGDGRYFSKDAVQIITKMAAANGVRRVWVGQDSLLSTPAVSA |
| 499 | 10 | c | DGSKATGAFILTASHNPGGPTEDFGIKYNMGNGGPAPES |
| 500 | 10 | d | EYLIAEDLPDVDISALGV |
| 501 | 10 | e | FDVDVFDSATDYIKLMKTIFDFESIKKL |
| 502 | 10 | f | FCFDGLHGVAGAYAKRMFVDELGASESSLLNCVPKEDFGGGHPDPNLTYAKELVERMGLG |
| 503 | 10 | g | VEPPEFGAAADGDADRNMVLGKRFFVTPSDSVAIIAANAVQSIPYFASGLKGVARSMPTSA |
| | | | ALDVVAKNLNLKFFEVPTGWKFFGNLMDAGMCSVCGEESFGTGSDHIREKDGIWAVLAWL |
| | | | SIIAYKNK |
| 504 | 10 | h | KLVSVEDIVLQHWATYGRHYYTRYDYENVDAEAAKELMA |
| 505 | 10 | i | DVAEVVSADEFEYKDPVDGSVSKHQGIRYLFGDGSRLVFRLSGTGSVGATIRIYIEQYEKDS |
| | | | SKTGRES |
| 506 | 10 | j | DALSPLVDVALKLSK |
| 507 | 11 | a | TPVMDSLKNGAPEKWTLVKAHGTAVGLPSDDDMGNSEVGHNALGAGRIFAQGAKLVDAA |
| | | | LASGKIWE |
| 508 | 11 | b | GTLHLIGLLSDGGVHSRLDQVQLLVKGASERGAKRIRLHILTDGRDVLDGSSVGFVETLEN |
| | | | DLA |
| 509 | 11 | c | LREKGVDAQVASGGGRMYVTMDRYENDWDVVKRGWDAQVLGEAPYKFKS |
| 510 | 11 | d | DQYLPAFVIVDESGKSVGPIVDGDAVVTFNFRADRMVMLAKALE |
| 511 | 11 | e | DFDKFDRVRVPKIKYAGMLQYDGELKLPNK |
| 512 | 11 | f | LVSPPLIERTSGEYLVKNGVRTFACSETVKFGHVTFFWNGNRSGYFDE |
| 513 | 11 | g | KEEYIEIPSDSGITFNEQPKMKALEIAEKTRDAILSGKFDQVRINLPNGDMVGHTGDIEATVV |
| | | | ACKAADEAVKIVLDAVEQVGGIYLVTADHGNAEDMVKRNKSGQP |
| 514 | 11 | h | GSIQILTSHTLQPVPVAIGGPGLH |
| 515 | 11 | i | TPGLANVAATVMNLHGFQAPDDYE |
| 516 | 13 | a | MDEEYDVIVLGTGLKECILSGLLSVDGLKVLHMDRNDYYGGESTSLNLTK |
| 517 | 13 | b | SKEYNVDMVPKFMMANGALVRVLI |
| 518 | 13 | c | TSVTKYLNFKAVDGSFVYN |
| 519 | 13 | d | GKIHKVPATDVEALKSNLMGLFEKRRARKFFIYVQDYE |
| 520 | 13 | e | KYGLEDDTVDFIGHALALHRDDNYLD |
| 521 | 13 | f | KRMKLYAESLARFQGGSPYIYPLYGLGELPQAFARLSAVYGGTYMLNKPECKVEF |
| 522 | 13 | g | GKAFGVTSEGETAKCKKVVCDPSYLPDKVTKVGRVARAICIMKHPIPDT |
| 523 | 13 | h | KQLKRKSDMYVFCCSYAHNVAPKGKFIAFVSTEAETDKPEIELKPGIDLLGPVE |
| 524 | 13 | i | SYDATTHFETTVKDV |
| 525 | 13 | j | YSKITGKELDLSVDLNAASA |
| 526 | 19 | a | GVVATTDAVEACTGVNVAVMVGGFPRKEGMERKDVMSKNVSIYKSQASALEAHAAPNCKV |
| | | | LVVANPANTNALILKEFAPSIPEKNISCLTRLDHNRALGQVSERLNVQVSDVKNVLIWGNHS |
| | | | SSQYPDVNHATV |
| 527 | 19 | b | GPFIATVQQRGAAIIKARKLSSALSAASSACDHIRDWVLGTPEGTFVSMGVYSDGSYGVPA |
| | | | GLIYSFPVTCSGGEWTIVQGLPIDEFSRKKMD |
| 528 | 19 | c | TAQELSEEKALAYSCL |
| 529 | 20 | a | PSPAVFVDKSTRVICQGITGKNGTFHTEQAIEYGTNMVGGVTPKKGGTEHLGLPVFNSVAE |
| | | | AKAETKANASVIYVPPPFAAAAIMEALEAELDLVVCITEGIPQHDMVKVKAALNRQSKTRLIG |
| | | | PNCPGIIKPGECKIGIMPGYIHKPGRIGIVSRSGTLTYEAVFQTTAVGLGQSTCVGMGGDPF |
| | | | NGTNFVDCLEKFVADPQTEGIVLIGEIGGTAEEDAAAFIQ |
| 530 | 20 | b | KPVVAFIAGLTAPPGRRMGHAGAIVSGGKGTAQDKIKALREAGVTVVESPAKIGSTMF |
| 531 | 22 | a | MALPNQGTVDYPSFKLVIVGDGGTGKTTFVKRHLTGEFEKKYEPTIGVEVHPLDFTTNCGKI |
| | | | RFYCWDTAGQEKFGGLRDGYYIHGQCAIIMFDVTSRLTYKNVPTWHRDLCRVCENIPIVLC |
| | | | GNKVDVKNRQVKAKQVTFHRKKNLQYYEISAKSNYNFEKPFLYLARKLAGDANIHFVE |
| 532 | 24 | a | ITDDTRIRASIPTIK |
| 533 | 24 | b | GAKVILASHLGRPKGVTPKFSLKPLVPRLSELLGVEVVMA |
| 534 | 24 | c | AALPEGGVLLLENVRFYKEEEKNDPEFAKKLASVADLYVNDAFGTAHRAHASTEGVTKFLRP |
| | | | SVAGFLMQKELDYLVGAVANPKKPFAAIVGGSKVSSKIGVIESLLAKVDILILGGGMIFTFYK |
| | | | AQGKAVGKSLVEEDKLELAT |
| 535 | 24 | d | AKAKGVSLLLPTDVVVADKFA |
| 536 | 24 | e | AIPDGWMGLDVGPDSIKTFSEALDTTKTVIWNGPMGVFEFEKFAAGT |
| 537 | 24 | f | LADLTGKGVTTIIGGGDSVAAVEKAGLADKMSHISTGGGASLELLEGKPLPGVLALDEA |
| 538 | 26 | a | GVFTDKDKAAAHMKGGAKKVVISAPSKDAPMFVVGVNED |
| 539 | 26 | b | DVNIVSNASCTTNCLAPLAKIINDNFGIVEGLMTTVHSITATQKTVDGPSSKDWRGGRAAS |
| | | | FNIIPSSTGAAKAVGKVLPELNGKLTGMSFRVPTVDVSVVDLTVRIEKAASYE |
| 540 | 26 | c | VSTDFIGDSRSSIFDAKAGIALNDNFVKLVSWYDNEWGY |
| 541 | 26 | d | PIKIGINGFGRIGRLVARVALQC |
| 542 | 26 | e | ELVAVNDPFITTDYMTYMFKYDTVHGQWK |
| 543 | 26 | f | AAGADYVVESTGVFTDKDKAAAHIKGGAKKVIISAPSKDAPMFVCGVNEKEYT |
| 544 | 26 | g | ITIVSNASCTTNCLAPLAKVINDRFGIVEGLMTTVHAMTATQKTVDGPSSKDWRGGRAASF |
| | | | NIIPSSTGAAKAVGKVLPVLNGKLTGMAFRVPTVDVSVVDLTVRLEKAATYEQIKAAIKEESE |
| | | | GNLKGILGYV |
| 545 | 26 | h | VSTDFQGDSRSSIFDAKAGIALNDNFVKLVSWYDNEWGYSTRVVDLI |
| 546 | 27 | a | GKYKDELIKNAAYIGTPGKGILAADESTGTIGKRFASINVENVEDNRRALRELLFTTPGALQH |
| | | | ISGVILFEETLYQ |
| 547 | 27 | b | LKENNVLPGIKVDKGTVELAGTD |
| 548 | 27 | c | KRCAKYYEAGARFAKWRAVLKIGPNEPSQLSI |
| 549 | 27 | d | QNAQGLARYAIICQENGLVPIVEPEILVDGPHDIE |
| 550 | 27 | e | CAYVTEVVLAACYKALNDQHVLLEGSLLKPNMVTPGSDAKKVAPEVI |
| 551 | 27 | f | PPAVPAIVFLSGGQSEEEATVNLNAMNK |
| 552 | 27 | g | LSFSFGRALQQSTLKAWSGKEENV |
| 553 | 27 | h | GEGASESLHVKDYKY |
| 554 | 29 | a | FKYVILGGGVAAGYAAREFAKQGVQPGELAIISKESVAPYERPALSKGYLFPQ |
| 555 | 29 | b | AARLPGFHTCVGSGGEKLLPEWYTEKGIELILSTEIVKADLASKTLTSAAG |
| 556 | 29 | c | QAKKDGKAVVVGGGYIGLELSAALK |
| 557 | 29 | d | NNFDVTMVYPEPWCMPRLFTAGIAHFYEGYY |
| 558 | 29 | e | VGVGGRPLTGLFKGQV |
| 559 | 29 | f | PRRVEHVDHARKSAEQAVKAIKAKE |
| 560 | 29 | g | AEYDYLPYFYSRSFDIAWQFYGDNVG |
| 561 | 29 | h | YWVKDGKVVGVFLEGG |
| 562 | 30 | a | SGHSLLRDPRHNKGLAFSE |
| 563 | 30 | b | YIAMMDLQERNERLFYKLLIDNVEELLPVVYTPVVGEACQKYGSI |
| 564 | 30 | c | NWPERSIQVIVVTDGERILGLGDLGCQGMGIPVGKLSLYTALGGVRPSACLPITIDVGTNNQ |
| | | | TLL |
| 565 | 30 | d | NYGEKVLVQFEDFANHNAFDLLA |
| 566 | 30 | e | KSHLVFNDDIQGTASVVLAGLLAAL |
| 567 | 30 | f | DQTYLFLGAGEAGTGIAELIALEMSK |
| 568 | 30 | g | ESLQHFKKPFAHEHEP |
| 569 | 30 | h | VLIGTSGVGKTFTQEV |
| 570 | 30 | i | LSNPTSHSECTAEEAYTW |
| 571 | 30 | j | AVFASGSPFDPVEYE |
| 572 | 30 | k | VPGQSNNAYVFPGFGLG |
| 573 | 30 | I | GAIRVHDDMLLAASEALA |
| 574 | 30 | m | LPRPDDLVKYAESCMY |
| 575 | 34 | a | AAVAWEAGKPLSIEEVEVAPPQAMEVRVKILFTALCHTDVYFWEAKGQTPVFPRIFGHEAGG |
| | | | IVESVGEGVTDVAPGDHVLPVFTGECKECRHCKSAESNMCDLLRINTDRGVMISDGKSRFS |
| 576 | 34 | b | GKPIFHFVGTSTFSEYTVMHVGCVAKINPEAPLDKVCVLSCGISTGLGASINVAKP |
| 577 | 34 | c | GSTVAIFGLGAVGLAAAEGARIAGASRIIGIDLNA |
| 578 | 34 | d | TEFVNPKDHTKPVQEV |
| 579 | 34 | e | AEMTDGGVDRSVECTGNINAMIQAFECVHDGWGVAVLVGVPHKDA |
| 580 | 34 | f | FKTHPMNFLNERTLKGTFFGNFKPRTD |
| 581 | 34 | g | EKFITHSVTFSEINKAFD |
| 582 | 34 | h | CLAKINPEAPLDKVCVLSCGISTGLGAMLNVAKPKKGSTVAIFGLGAVGLAAMEGARMAGA |
| | | | SRIIGVDLNP |
| 583 | 34 | i | EMTNGGVDRAVECTGHIDAMIAAFECVHDGWGVAVLVGVPHKE |
| 584 | 34 | j | VFKTHPMNFLNERTLKGTFFGNYKPRTDLP |
| 585 | 39/59 | a | PSEDAVEVVVSPPFVFLQ |
| 586 | 39/59 | b | AVAAQNCWVRKGGAFTGEISAEMLVNLQVPWVILGHSERRALLSESNDFV |
| 587 | 39/59 | c | DKVAYALAQGLKVIACIGETLEQREAGTTMEVVAAQTKAIAEKISDWTNVVLAYEPVWAIGT |
| | | | GKVASRAQAQEVH |
| 588 | 39/59 | d | TRIIYGGSVNGANCKELAAQPDLDGFLVGGASLKPEFVDIIK |
| 589 | 39/59 | e | AEMLANLGIPWVILGHSERRALLGESSEFVGDKVAYALAQGLKVIACVGETLEQREAGSTM |
| 590 | 39/59 | f | WTNVVIAYEPVWAIGTGKVATPAQAQEVHANLR |
| 591 | 39/59 | g | SPEVAETTRIIYGGSVTG |
| 592 | 39/59 | h | NELAAQPDVDGFLVGGASLKPEFIDIINAA |
| 593 | 43 | a | VTGYFAWSLLDNFEW |
| 594 | 47 | a | VQWSKWHVFWVDERVVPKDHVDSNYKLA |
| 595 | 47 | b | ATGFPRFDLMLLGMGPDGHLASLFPGHPLLNE |
| 596 | 47 | c | DSPKPPPQRITFTFPVIKSSAYVA |
| 597 | 47 | d | DGHLASLFPGHPLLNE |
| 598 | 47 | e | DSPKPPPQRITFTFPVIKSSAYVA |
| 599 | 49 | a | YNLLDTVSRHTIQVYPRSWTAVMLTFDNAGMWNLRSNLWERYY |
| 600 | 49 | b | SCTSPARSLRDEYNMPENGLRCGKIVGLPLPPSY |
| 601 | 49 | c | MGPGKWKPELRKTYNLLDAVSRHSIQVYPRSWSAVMLTFDNAGMWNVRSNVWERHYLGE |
| | | | QLYISVISPARSLRDEYNFPENALRCGKVVGLPLPPSYLPA |
| 602 | 51 | a | SVYMAKLAEQAERYEEMVEFM |
| 603 | 51 | b | ELSVEERNLLSVAYKNVIGARRASWRIISSIEQKEEG |
| 604 | 51 | c | AGAAESKVFYLKMKGDYHRYLAEFKSGAERKEAAESTM |
| 605 | 51 | d | AQDIALADLAPTHPIRLGLALNFSVFYYEILNSPDRACNLAKQAFDEAISELDSLGEESYKDS |
| | | | TLIMQLLRDNLTLWTSDTN |
| 606 | 54 | a | AEDPYVFFEWHVTYGT |
| 607 | 54 | b | FPGPRINCSSNNNIVVNVFNQLDQP |
| 608 | 54 | c | LFTWNGIQHRKNSWQDG |
| 609 | 54 | d | CNVGIKSSLNFRIQGHDMRLVE |
| 610 | 54 | e | GWAWSLNQWRSFRWNLTASAARPNPQGSYHYGQINITRTIKLMI |
| 611 | 54 | f | NGVSHVDADTPLKLAEYF |
| 612 | 54 | g | PELRKTYNLLDAVSRHSIQVYPRSWSA |
| 613 | 54 | h | QLYISVISPARSLRDEYNFPEN |
| 614 | 56 | a | QVAIGTDDVYKSAEA |
| 615 | 56 | b | ELGGKILRQPGPLPGLNTKIASFLDPDGWKVVLVDH |
| 616 | 56 | c | DRRRLLHVVYRVGDLDKTIKFYTECLGMKLLRKRDIPEERY |
| 617 | 56 | d | GPEDSHFVVELTYNYGVESYDIG |
| 618 | 56 | e | IKAKGGTVTREPGPVKGGKSVIAF |
| 619 | 56 | f | FELIERGPTPEPLCQVMLRVGDLDRAIKFYEKAFGMELLRRKDNPQYKYTIAMMGYGPEDKN |
| | | | AVLELTYNYGVKEYDKGNAYAQIAIGTDDVYKTAEVV |
| 620 | 56 | g | NGGQITREPGPLPGISTKITACTDPDGWKSVFVDNLDFLKELE |
| 621 | 62 | a | NPTTAAGVLRVFFHDCFVSGCDASVLI |
| 622 | 62 | b | SEKDADINHSLPGDAFDAVVRSK |
| 623 | 62 | c | ALELECPGVVSCADILA |
| 624 | 62 | d | KGFTVQEMVALSGAHTLGFSHCQEF |
| 625 | 62 | e | AAFNDIMTPGKFDNMYYVN |
| 626 | 73 | a | SQDEAGTSAIKTVELDTMLGGRAVQHREPQGYESDKFLSYFKPCIIPLEGG |
| 627 | 73 | b | VPFARSSLNHDDVFILDTEKKIYQFNGANSNIQERAKALEVIQHLKDKYHEGVCDVAIVDD |
| | | | GKLQAESDSGEFWVVFGGFAPIGKKT |
| 628 | 73 | c | DCGSELFVWVGRVTQVD |
| 629 | 73 | d | GDCYIVLYTYHSGEK |
| 630 | 73 | e | KGRPVLGRIYQGKEPPQFIALFQPMVILKGG |
| 631 | 73 | f | YEQQQWAAKVAEFLKPG |
| 632 | 73 | g | EDVMILDTHAEVFVW |
| 633 | 76 | a | SGLNVLIETYFADVPAESYKTLTSL |
| 634 | 76 | b | IPSGKYLFAGVVDGRNIWADDLAASLS |
| 635 | 76 | c | CSLMHTAVDLVNETKLDSEIKSWLAFAAQKVVEVNALGKALVG |
| 636 | 76 | d | ANAAAQASRRSSPRVNNEEVQKAAAALKGSDHRRATTVSARLDAQQKKLNLPVLPTTTIGS |
| | | | FPQT |
| 637 | 76 | e | KISEEAYVSAIKEEI |
| 638 | 76 | f | KVVKIQEELDIDVLVHGEPERNDMVEYFGEQLSGFAFTANGWVQSYGSRCVKPPIIYGDVS |
| | | | RPNPMTVFWS |
| 639 | 76 | a | KISEEAYVSAIKEEI |
| 640 | 76 | b | KVVKIQEELDIDVLVHGEPERNDMVEYFGEQLSGFAFTANGWVQSYGSRCVKPPIIYGDVS |
| | | | RPNPMTVFWS |
| 641 | 77 | a | QEVAGDVRMTDTRADEAERGITIKSTGISLYYEMSEE |
| 642 | 77 | b | RDGNDYLINLIDSPGHVDFSSEVTAALRITDGALVVVDCIEGVCVQTETVLRQALGERIRPV |
| | | | LTVNKMDRCFLELQVDGEEAYQTFSRVIENANVIMATYEDALLGDVQVYPEKGTVAFSAGL |
| | | | HGWAFTLTNFAKMYASKFGVDESKMMERLWGENFFDPATKKWT |
| 643 | 77 | c | KNTGSGTCKRGFVQFCYEPIKQIIEICMND |
| 644 | 77 | d | KDKLWPMLKKLGVTMK |
| 645 | 77 | e | DEKDLMGKALMKRVMQAWLPAS |
| 646 | 77 | f | HLPSPSKAQRYRVENLYEGPLDDVYANAIRNCDPEGPLMLYVSKMIPASDKGRFFAFGRVFA |
| | | | GRV |
| 647 | 77 | g | TGMKVRIMGPNFVPGQKKDLYTKSVQRTVIWMGKKQESVEDVPCGNTVALVGLDQFITKN |
| | | | ATLTGEKEVDACPIRAMKFSVSPVVRVAVQCKVASDLPKLVEGLKRLAKSDPMVLCSIEESG |
| | | | EHIIAGAGELHLEICLKDLQDDFMGGAEIIVSPPVVSFRETVLDKSCRTVMSKSPNKHNRLY |
| | | | MEARPLEEGLPEAIDEGRIGPRDDPKVRSKILSEEFGWDKDLAKKIWCFGPETTGPNMVVD |
| | | | MCKGVQYLNEIKDSVVAGFQWASKEGALADENMRGICFEVCDVVLHTDAIHRGGGQVIPT |
| | | | ARRVIFASQLTAKPRLLEPVYLVEIQAPEGALGGIYGVLNQKRGHVFEEMQRPGTPLYNIKAY |
| | | | LPVIESFGFSATLRAATSGQAFPQCVFDHWDVM |
| 648 | 77 | h | LVKEIRKRKGLKEQMTPLSDFEDKL |
| 649 | 86/51 | a | REESVYMAKLAEQAERYEEMVEFMERV |
| 650 | 86/51 | b | EELSVEERNLLSVAYKNVIGARRASWRIISSIEQKEEGRGND |
| 651 | 86/51 | c | AESKVFYLKMKGDYHRYLAEFKSGAERKEAAESTM |
| 652 | 86/51 | d | YKAAQDIALADLAPTHPIRLGLALNFSVFYYEILNSPDRACNLAKQAFDEAISELDSLGEESY |
| | | | KDSTLIMQLLRDNLTLWTSDTN |
| 653 | 86/51 | e | REENVYMAKLAEQAERYEEMVEFMEKVA |
| 654 | 86/51 | f | GELTVEERNLLSVAYKNVIGARRASWRIISSIEQKEESRGNEAYV |
| 655 | 86/51 | g | IETELSKICDGILKLLDSHL |
| 656 | 86/51 | h | AESKVFYLKMKGDYHRYLAEF |
| 657 | 86/51 | i | DYHRYLAEFKAGAERKEAAENTLVAYKSAQDIA |
| 658 | 86/51 | j | LPTTHPIRLGLALNFSVFYYEILNSPDRACNLAKQAFDEAIAELDSLGEESYKDSTLIMQLLRD |
| | | | NLTLWTSDNAD |
| 659 | 87 | a | VYYSMYGHVGKLAEEIKKGASSVEGVEVK |
| 660 | 87 | b | ELAEADGILFGFPTRFGMMASQMKAF |
| 661 | 87 | c | DATGGLWREQSLAGKPAG |
| 662 | 87 | d | FFSTGTQGGGQETTPLTAVTQLTHHGMVFVPVGYTFGA |
| 663 | 87 | e | MFDMEKVQGGSPYGAGTFAGDGSRWPSE |
| 664 | 91 | a | VFFDVTIGGAPAGRIVMELYADVVPKTAENFRALCTGEKGVGKMGKPLHYKGSSFHRVIPGF |
| | | | MCQGGDFTAGNGTGGESIYGAKFADENFVKKHTGPGVLSMANAGPGTNGSQFFLCTAKTA |
| | | | WLDGKHVVFGQVVEGMDVVKAVEKVGSQSGRCSKPVVIADCGQL |

Table 4 shows wild type sequences of proteins found in non-Timothy grass pollen, which sequences contains PG+ peptides of a peptide thereof with less than 3 mismatches compared to the PG+ peptide and/or contain a GWT sequence of Table 3.

TABLE 4
Table 4 (SEQ ID Nos: 665-1109

| SEQ | | | |
|---|---|---|---|
| ID | NTGA | | |
| NO | No | Species | Sequence |
| 665 | 1 | Amb_a | LMATIKAVKARQIFDSRGNPTVEVDITLSDGTLARAAVPSGASTGIYEALELRDGG |
| | | | SDYLGKGVSKAVANVNTIIGPALVGKDPTDQTGIDNFMVQQLDGTQNEWGWCK |
| | | | QKLGANAILAVSLAVCKAGASVLKTPLYKHIANLAGNKNLVLPVPAFNVINGGSHA |
| | | | GNKLAMQEFMILPIGASSFKEAMKMGVEVYHNLKSVIKKKYGQDATNVGDEGGF |
| | | | APNIQENKEGLELLKTAIAKAGYTDKVVIGMDVAASEFYGEKDKTYDLNFKEENND |
| | | | GKEKISGEQLKDLYKSFVSEYPIVSIEDPFDQDDWEHY |
| 666 | 1 | Amb_p | ARQIFDSRGNPTVEVDITLSDGTLARAAVPSGASTGIYEALELRDGGSDYLGKGVS |
| | | | KAVANVNTIIGPALVGKDPTDQTGIDNFMVQQLDGTQNEWGWCKQKLGANAILA |
| | | | VSLAVCKAGASVLKTPLYKHIANLAGNKNLVLPVPAFNVINGGSHAGNKLAMQEF |
| | | | MILPIGASSFKEAMKMGVEVYHNLKSVIKKKYGQDATNVGDEGGFAPNIQENKEG |
| | | | LELLKTAIAKAGYTDKVVIGMDVAASEFYGEKDKTYDLNFKEENNDGKEKISGEQL |
| | | | KDLYKSFVSEYPIVSIEDPFDQDDWEHYAKMTAECGEQVQIVGDDLLVTNPTRVK |
| | | | KAIDEKTCNALLLKVNQIGSVTESIEAVRMSKHAGWGVMASHRSGETEDTFIADL |
| | | | SVGLATGQIKTGAPCRSERLAKYNQLLRIEEELGSEAVYAGANFRKPVEPY |
| 667 | 1 | Bet_v | AEITHVKARQIFDSRGNPTVEAEVTTANGVVSRAAVPSGASTGVYEALELRDGGS |
| | | | DYLGKGVLKAVENVNAIIGPALIGKDATEQAAIDNFIVQQLDGTVNEWGWCKQKL |
| | | | GANAILAVSLAVCKAGASAKKIPLYKHIANLAGNPKLVLPVPAFNVINGGSHAGNK |
| | | | LAMQEFMILPVGASSFKEAMKMGVEVYHHLKAVIKKKYGQDATNVGDEGGFAPNI |
| | | | QENKEGLELLKTAIAKAGYTGKVVIGMDVAASEFYGEDKRYDLNFKEENNDGSQK |
| | | | IPGDALKDLYKSFVAEYPIVSIEDPFDQDDWEHYSKVTAEIGEKVQIVGDDLLVTN |
| | | | PKRVEKAIKEKSCNALLLKVNQIGSVTESIEAVKMSKRAGWGVMASHRSGETEDT |
| | | | FIADLSVGLATGQIKTGAPCRSERLAKYNQLLRIEEELGSEAVYAGANFRTPVEPY |
| 668 | 1 | Cyn_d | MAATIQSVKARQIFDSRGNPTVEVDVCCSDGTFARAAVPSGASTGVYEALELRDG |
| | | | GSDYLGKGVSKAVNNVNSIIGPALIGKDPTAQTEIDNFMVQQLDGTKNEWGWCK |
| | | | QKLGANAILAVSLAVCKAGASIKKIPLYQHIANLAGNKQLVLPVPAFNVINGGSHA |
| | | | GNKLAMQEFMILPTGASSFKEAMKMGVEVYHNLKSVIKKKYGQDATNVGDEGGF |
| | | | APNIQENKEGLELLKTAIEKAGYTGKVVIGMDVAASEFYNDKDKTYDLNFKEENND |
| | | | GSQKISGDSLKNVYKSFVSEYPIVSIEDPFDQDDWVHYAKMTEEIGEQVQIVGDD |
| | | | LLVTNPTRVSKAIKEKSCNALLLKVNQIGSVTESIEAVKMSKHAGWGVMTSHRSG |
| | | | ETEDTFIADLAVGLATGQIKTGAPCRSERLAKYNQLLRIEEELGAAAVYAGAKFRAP |
| | | | VEPY |
| 669 | 1 | Que_a | MAITIQAIKARQIFDSRGNPTVEVDVTTSDGAFYRAAVPSGASTGIYEALELRDGG |
| | | | SDYLGKGVSKAVENVNAIIAPALIGKDPTDQVAIDNFMVQQLDGTVNEWGWCKQ |
| | | | KLGANAILAVSLAVCKAGAGVNKIPLYKHIANLAGNKKLVLPVPAFNVINGGSHAG |
| | | | NKLAMQEFMILPVGASSFKEAMKMGVEVYHNLKSVIKKKYGQDATNVGDEGGFA |
| | | | PNIQENKEGLELLKTAIAKAGYTSQVVIGMDVAASEFYGEDKRYDLNFKEEKNDGS |
| | | | QKIPGDALKDLYKSFVSEYPIVSIEDPFDQDDWEHYGKMTSEVGEKVQIVGDDLL |
| | | | VTNPKRVEKAIKEKTCNALLLKVNQIGSVTESIEAVKMSKRAGWGVMASHRSGET |
| | | | EDTFIADLSVGLATGQIKTGAPCRSERLAKYNQLLRIEEELGSEAVYAGASFRRPVE |
| | | | PY |
| 670 | 2 | Amb_a | AALISDTAPWKDLKAHVGEIDKTHLRDLMSDTERCSSMMLEFDGIFLDYSRQRAT |
| | | | VDTVSKLFTLAEEAHLKQKINSMFNGEHINSTENRSVLHVALRAAKDTTINSDGKN |
| | | | VVPDVWQVLDKIKEFSDKVRNGSWVGATGKALTNVIAIGIGGSFLGPLFVHTALQ |
| | | | TDPEASKLAGGRQLRFLANVDPVDVARNISGLDPETTLVVVVSKTFTTAETMLNAR |
| | | | TLREWISSALGPQAVSKHMVAVSTNLKLVEKFGIDPNNAFAFWDWVGGRYSVCS |
| | | | AVGVLPLSLQYGFSVVEKFLKGARSIDQHFHSAPFESNIPVLLGLLSVWNVSFLGYP |
| | | | ARAILPYTQALEKLAPHIQQVSMESNGKGVSIDGVRLPFEAGEIDFGEPGTNGQHS |
| | | | FYQLIHQGRVIPCDFIGIVKSQQPVYLKGSVLLVTDSGWKNQLLILDGRISLQLQGL |
| | | | VIPQPL |
| 671 | 2 | Amb_p | GRQLRFLANVDPVDVARNISGLDPETTLVVVVSKTFTTAETMLNARTLREWISSAL |
| | | | GPQAVSKHMVAVSTNLKLVEKFGIDPNNAFAFWDWVGGRYSVCSAVGVLPLSLQ |
| | | | YGFSVIEKFLEGARSIDQHFHSAPFENNIPVLLGLLSVWNVSFLGYPARAILPYTQA |
| | | | LEKLAPHIQQVSMESNGKGVSIDGVRLPFEAGEIDFGEPGTNGQHSFYQLIHQGR |
| | | | VIPCDFIGIVKSQQPVYLKDEVVNNHDELMSNFFAQPDALAYGKTPEQLQSENVAS |
| | | | HLVPHKTFTGNRPSLSLLLPSLDAYRIGQLLAIYEHRIAVEGFIWGINSFDQWGVEL |
| | | | GKSLASQVRKQLHASRKKGESVEGFNFSTTKLLTRYLEASADVPSEPTTLLPKI |
| 672 | 2 | Ant_o | TKSGDGDQTISGPQKRSSRAVRAPSSFLPVCLLRPLPPRDGRPPSSGSLPPKLPRG |
| | | | AGPGTKSSAPMASPALISDTDQWKALQAHVGAIHKTHLRDLMADADRCKALTAEF |
| | | | EGVFLDYSRQQATTETVDKLFKLAEAAKLKEKIAKMFNGDKINSTENRSVLHVALR |
| | | | APRDAVINSDGVNVVPEVWAVIDKIKQFSETFRSGSWVGATGKPLTNVVSVGIGG |
| | | | SFLGPLFVHTALQTDPVAAESAKGRQLRFLANVDPVDVARSIKDLDPETTLVVVVS |
| | | | KTFTTAETMLNARTIKEWIVSSLGPQAVSKHMIAVSTNLKLVKEFGIDPNNAFAFW |
| | | | DWVGGRYSVCSAVGVLPLSLQYGFPVVQKFLEGASSIDNHFRTSSFEKNIPVLLGL |
| | | | LSVWNVSFLGYPARAILPYSQALEKLAPHIQQLSMESNGKGVSIDGVRLPYEAGEI |
| | | | DFGEPGTNGQHSFYQLIHQGRVIPCDFIGVIKSQQPVYLKGETVSNHDELMSNFFA |
| | | | QPDALAYGKTPEQLRSENVSENLIPHKTFQGNRPSLSFLLSSLSAYEIGQLLAIYEH |
| | | | RIAVQGFIWGINSFDQWGVELGKSLASQVRKQLHASRMEGKPIEGFNPSSASLLA |
| | | | RYLSVEPSTPFDTTVLPKV |
| 673 | 2 | Bet_v | MASRTLISDTEAWKNLKAHVEEIKKTHLRDLMSDAERCKSMMVESEGVLLDHSR |
| | | | QRATPETMDKLFKLAEAAHLKEKINRMYSGVHINSTENRPVLHVALRASRDGVIQS |
| | | | DGKNVVPEVWKVLDKIQEFSERVRNGSWVGATGKALKDVVAVGIGGSFLGPLFV |
| | | | HTALQTDPEAIESARGRQLRFLANVDPIDVARNITGLNPETTLVVVVSKTFTTAETM |
| | | | LNARTLREWISAALGPSAVAKHMVAVSTNLTLVEKFGIDPNNAFAFWDWVGGRYS |
| | | | VCSAVGVLPLSLQYGFSVVEKFLKGASSIDQHFYSAPYEKNIPVLLGLLSIWNVSFL |
| | | | GYPARAILPYSQALEKFAPHIQQVSMESNGKGVSIDGVLLPFEAGEIDFGEPGTNG |
| | | | QHSFYQLIHQGRVIPCDFIGIVRSQQPVYLKGEVVSNHDELMSNFFAQPDALAYGK |
| | | | TPEQLHKENVSPHLIPHKTFSGNRPSLSLLLPSLNAYNIGQLLAIYEHRIAVEGFVW |
| | | | GINSFDQWGVELGKSLATQVRKQLNASRTKGEPVEGFNFSTTTLLTRYLEATADIP |
| | | | SDPPTLLPRI |
| 674 | 2 | Bet_v | SFQMASRTLISDTEAWKNLKAHVEEIKKTHLRDLMSDAERCKSMMVESEGVLLDH |
| | | | SRQRATPETMDKLFKLAEAAHLKEKINRMYSGVHINSTENRPVLHVALRASRDGVI |
| | | | QSDGKNVVPEVWKVLDKIQEFSERVRNGSWVGATGKALKDVVAVGIGGSFLGPL |
| | | | FVHTALQTDPEAIESARGRQLRFLANVDPIDVARNITGLNPETTLVVVVSKTFTTAE |
| | | | TMLNARTLREWISAALGPSAVAKHMVAVSTNLTLVEKFGIDPNNAFAFWDWVGG |
| | | | RYSVCSAVGVLPLSLQYGFSVVEKFLKGASSIDQHFYSAPYEKNIPVLLGLLSIWNV |
| | | | SFLGYPARAILPYSQALEKFAPHIQQVSMESNGKGVSIDGVLLPFEAGEIDFGEPGT |
| | | | NGQHSFYQLIHQGRVIPCDFIGIVRSQQPVYLKGEVVSNHDELMSNFFAQPDALA |
| | | | YGKTPEQLHKENVSPHLIPHKTFSGNRPSLSLLLPSLNAYNIGQLLAIYEHRIAVEGF |
| | | | VWGINSFDQWGVELGKSLATQVRKQLNASRTKGEPVEGFNFSTTTLLTRYLEATA |
| | | | DIPSDPPTLLPRI |
| 675 | 2 | Cyn_d | AGVRTHFYRAAVRSAYAGRGCPHRPHQPNIQFKGRGVYVYHHHHYRRLPTGTRRK |
| | | | EAIQNPRKLAGGEEQIRFLFQRSTLHPRRPADEAMASPALICDTEQWKALQAHVSA |
| | | | IQKTHLRDLMADADRCKAMTAEFEGIFLDYSRQQATGETMEKLLKLAEAAKLKEKI |
| | | | EKMFKGDKINSTENRSVLHVALRAPRDAVINSDGVNVVPEVWGVKDKIKQFSETF |
| | | | RSGSWVGATGKALTNVVSVGIGGSFLGPLFVHTALQTDPEAAECAKGRQLRFLAN |
| | | | VDPVDVARSIKDLDPETTLVVVVSKTFTTAETMLNARTLKEWIVSSLGPQAVSKHM |
| | | | IAVSTNLKLVKEFGIDPNNAFAFWDWVGGRYSVCSAVGVLPLSLQYGFPIVQKFLE |
| | | | GASSIDNHFYSCSFEKNIPVLLGLLSVWNVSFLGYPARAILPYAQALEKFAPHIQQL |
| | | | SMESNGKGVSIDGVKLSFETGEIDFGEPGTNGQHSFYQLIHQGRVIPCDFIGVVQ |
| | | | SQRPVYLKGETVSNHDELMSNFFAQPDALAYGKTPEQLHSEKVPENLIPHKTFQG |
| | | | NRPSLSLLLPTLSAYEIGQLLAIYEHRIAVQGFVWGINSFDQWGVELGKSLASQVR |
| | | | KQLHGSRMEGKPVEGFNPSTSSLLARYLAVKPSTPYDSTVLPKV |
| 676 | 2 | Cyn_d | MASPALICDTEQWKALQAHVSAIQKTHLRDLMADADRCKAMTAEFEGIFLDYSRQ |
| | | | QATGETMEKLLKLAEAAKLKEKIEKMFKGDKINSTENRSVLHVALRAPRDAVINSD |
| | | | GVNVVPEVWGVKDKIKQFSETFRSGSWVGATGKALTNVVSVGIGGSFLGPLFVH |
| | | | TALQTDPEAAECAKGRQLRFLANVDPVDVARSIKDLDPETTLVVVVSKTFTTAETM |
| | | | LNARTLKEWIVSSLGPQAVSKHMIAVSTNLKLVKEFGIDPNNAFAFWDWVGGRYS |
| | | | VCSAVGVLPLSLQYGFPIVQKFLEGASSIDNHFYSCSFEKNIPVLLGLLSVWNVSFL |
| | | | GYPARAILPYAQALEKFAPHIQQLSMESNGKGVSIDGVKLSFETGEIDFGEPGTNG |
| | | | QHSFYQLIHQGRVIPCDFIGVVQSQRPVYLKGETVSNHDELMSNFFAQPDALAYG |
| | | | KTPEQLHSEKVPENLIPHKTFQGNRPSLSLLLPTLSAYEIGQLLAIYEHRIAVQGFV |
| | | | WGINSFDQWGVELGKSLASQVRKQLHGSRMEGKPVEGFNPSTSSLLARYLAVKP |
| | | | STPYDSTVLPKV |
| 677 | 2 | Fra_e | MASSSLICETDPWKDLRAHVEDIKKTHLRDLMSDTERCKSMMVEFDGILLDYSRQ |
| | | | RTNLDTLNKLHSLAEAAHLKEKIYRMFNGERINITENRSVLHIALRAPRDSVINGDG |
| | | | KNVVPDVWQVLDKIRDFSESVRSGAWVGATGKVLKDVIAVGIGGSFLGPLFVHTA |
| | | | LQSDPEASEFAHGRQLRFLANVDPIDVARNIAGLNPETTLVVVVSKTFTTAETMLN |
| | | | ARTLREWISAALGPQAVAKHMVAVSTNLTLVEKFGIDPNNAFAFWDWVGGRYSV |
| | | | CSAVGVLPLSLQYGFSVVEKFLKGASSIDQHFYSAPLEKNLPVLLGLLSVWNVSFL |
| | | | GYPARAILPYSQALEKFAPHIQQVSMESNGKGVSIDGVPLPYETGEIDFGEPGTNG |
| | | | QHSFYQLIHQGRVIPCDFIGVVKSQQPVYLKGEMVSNHDELMSNFFAQPDALAYG |
| | | | KTAEQLLKENVPQPLIPHKTFSGNRPSLSLLLPTLNAYNIGQLLAIYEHRIAVEGFLW |
| | | | GINSFDQWGVELGKSLATQVRKQLHASRKKGEPFEGFNFSTTTMLKRYLEESADV |
| | | | PKEDCTILPKI |
| 678 | 2 | Lol_p | LLRRSSPFHRHRSPAARRRHPPLARPTSPRRSAMASPALISDTDQWKALQAHVGA |
| | | | IHKTHLRDLMADADRCKAMTAEFEGIHLDYSRQQATTETVDKLFKLAEAAKLKEKI |
| | | | EKMFSGDKINTTENRSVLHVALRAPRDAVINSDGVNVVPEVWAVIDKIKQFSETF |
| | | | RSGSWVGATGKPLTNVVSVGIGGSFLGPLFVHTALQTDPAAAESAKGRQLRFLAN |
| | | | VDPVDVARSIKDLDPATTLVVVVSKTFTTAETMLNARTIKEWIVSSLGPQAVSKHM |
| | | | IAVSTNLKLVKEFGIDPNNAFAFWDWVGGRYSVCSAVGVLPLSLQYGFPIVQKFLE |
| | | | GASSIDNHFRTSSFEKNIPVLLGLLSVWNVSFLGYPARAILPYTQALEKLAPHIQQL |
| | | | SMESNGKGVSIDGVRLPYEAGEIDFGEPGTNGQHSFYQLIHQGRVIPCDFIGVIKS |
| | | | QQPVYLKGETVSNHDELMSNFFAQPDALAYGKTPEQLRSENVSENLIPHKTFQGN |
| | | | RPSLSFLLSSLSAYEIGQLLSIYEHRIAVQGFIWGINSFDQWGVELGKSLASQVRK |
| | | | QLHASRMEGKPVEGFNPSSASLLARYLAVEPSIPYDTTVLPKV |
| 679 | 2 | Ole_e | MASSSLIYETGAWKDLKAHVEDIEKIHLRDLMSDTVRCKSMIIDFDGVLLDYSRQR |
| | | | ANFDTLNKLHNLAKAAHLKEKINGMFNGERINSTENRSVLHIALRAPRDSVINSDG |
| | | | KNVVPDVWQVLDKIRDFSERVRSGAWVGATGKVLKDVIAIGIGGSFLGPLFVHTA |
| | | | LQKDPEAIEFARGRQLRFLANVDPIDVARNIAGLNPETTLVVVVSKTFTTAETMLNA |
| | | | RTLREWISAALGPQAVAKHMVAVSTNLTLVEKFGIDPNNAFAFWDWVGGRYSVC |
| | | | SAVGVLPLSLQYGFSVVEKFLKGASSIDQHFYSAPFEKNLPVLLGLLSIWNVSFLGY |
| | | | PARAILPYSQALEKFAPHIQQVSMESNGKGVSIDGVPLPYETGEIDFGEPGTNGQH |
| | | | SFYQLIHQGRVIPCDFIGVVKSQQPVYLKGEMVSNHDELMSNFFAQPDALAYGKT |
| | | | AEQLLKENVPQPLIPHKTFSGNRPSLSLLLPTLNAYNIGQLLAIYEHRIAVEGFLWGI |
| | | | NSFDQWGVELGKSLATQVRKQLHASRKKGEPIEGFNFSTTTMLTRYLEESADVPK |
| | | | EDCTILPKI |
| 680 | 2 | Pla_l | KTITSKQTANQPSSQSFFNTFRNMASSPLICETEPWKDLKVHVDDIKKTHLRELMT |
| | | | DTGRCQSMMVEFDELLLDYSRQCATLDTMKKLYALAEAAHLKEKISRMFNGERIN |
| | | | STENRSVLHVALRAPRDSVINSDGKNVVPDVWNVLDKIKDFSERVRSGAWVGAT |
| | | | GKALTEVVAIGIGGSFLGPLFVHTALQTDPEAAQFATGRQLRFLANVDPIDVARNIA |
| | | | GLNPETTLVVVVSKTFTTAETMLNARTLREWISAALGPEAVSKHMVAVSTNLTLVE |
| | | | KFGIDPKNAFAFWDWVGGRYSVCSAVGVLPLALQYGFEVVEKFLKGASSVDQHF |
| | | | SSAPFEKNLPVLLGLLSVWNVSFLGYPARAILPYSQALEKLAPHIQQVSMESNGKG |
| | | | VSIDGVPLPYEAGEIDFGEPGTNGQHSFYQLIHQGRVIPCDFIGVVKSQQPVYLKG |
| | | | EVVSNHDELMSNFFAQPDALAYGKTPEQLLKESVPNHLVTHKTFSGNRPSLSLLLP |
| | | | SLHAYNVGQLLAIYEHRVAVEGFVWGINSFDQWGVELGKSLASQVRKQLHASRK |
| | | | KGEPVEGFNFSTTTVLSRYLKESEADVPKEECTILPKM |
| 681 | 2 | Poa_p | QIRHGHSPVRSSPIHIPPPPPVSFSASSLLLSPSAPINPLPPPPIRRQPAPRHPRRHIL |
| | | | AGPLRGSMASPALISDTDQWKALQAHVGAIHKTHLRDLMADADRCKAMTVEFEG |
| | | | VFLDYARQQATTETVDKLFKLAEAAKLKEKIEKMFSGEKINSTENRSVLHVALRAPR |
| | | | DAVINSDGVNVVPEVWSVKDKIKQFSETFRSGSWVGATGKPLTNVVSVGIGGSF |
| | | | LGPLFVHTALQTDPEAAESAKGRQLRFLANVDPVDVARSIKDLDPETTLVVVVSKT |
| | | | FTTAETMLNARTIKEWIVSSLGPQAVSKHMIAVSTNLKLVKEFGIDPNNAFAFWD |
| | | | WVGGRYSVCSAVGVLPLSLQYGFPIVQKFLEGASSIDNHFRTASFEKNIPVLLGLLS |
| | | | VWNVSFLGYPARAILPYSQALEKLAPHIQQVSMESNGKGVSIDGVPLPYEAGEIDF |
| | | | GEPGTNGQHSFYQLIHQGRVIPCDFIGVIKSQQPVYLKGETVSNHDELMSNFFAQ |
| | | | PDALAYGKTPEQLRSENVSENLIPHKTFKGNRPSLSFLLSSLSAYEIGQLLAIYENRI |
| | | | AVQGFIWGINSFDQWGVELGKSLASQVRKQLHASRMEGKPIEGFNPSSASLLARY |
| | | | LAVEPSTPYDTTVLPKV |
| 682 | 2 | Que_a | QFQMASPTLISDTGAWKDLKGHVEEINKTHLRDLMADAERCKSMMVEFDGVLLD |
| | | | YSRQRATNETVDKLFKLAEEAKLKEKINRMYNGEHINSTENRSVLHVALRASRDAV |
| | | | IKSDGKNVVPEVWSVLDKIKDFSERVRSGSWVGATGKVLKDVVAVGIGGSFLGP |
| | | | LFVHTALQTDPEAIKSARGRQLRFLANVDPIDVARNITGLNPETTLVVVVSKTFTTA |
| | | | ETMLNARTLREWISAALGPSAVAKHMVAVSTNLTLVEKFGIDPNNAFAFWDWVG |
| | | | GRYSVCSAVGVLPLSLQYGFSVVEQFLKGASSIDQHFYSAPHEKNIPVLLGLLSVW |
| | | | NVSFFGYPARAILPYSQALEKFAPHIQQVSMESNGKGVSIDGVPLPFEAGEIDFGEP |
| | | | GTNGQHSFYQLIHQGRVIPCDFIGVVKSQQPVFLKGEVVSNHDELMSNFFAQPDA |
| | | | LAYGKTPEQLHKENVAPHLIPHKTFSGNRPSLSLLLPSLNAYNIGQLLAIYEHRIAVE |
| | | | GFVWGINSFDQWGVELGKSLATQVRKQLHVSRTKGEPVEGFNFSTATLLTRYLEA |
| | | | TADIPADPPTLLPRI |
| 683 | 2 | Que_a | MASPTLISDTGAWKDLKGHVEEINKTHLRDLMADAERCKSMMVEFDGVLLDYSR |
| | | | QRATNETVDKLFKLAEEAKLKEKINRMYNGEHINSTENRSVLHVALRASRDAVIKS |
| | | | DGKNVVPEVWSVLDKIKDFSERVRSGSWVGATGKVLKDVVAVGIGGSFLGPLFV |
| | | | HTALQTDPEAIKSARGRQLRFLANVDPIDVARNITGLNPETTLVVVVSKTFTTAETM |
| | | | LNARTLREWISAALGPSAVAKHMVAVSTNLTLVEKFGIDPNNAFAFWDWVGGRYS |
| | | | VCSAVGVLPLSLQYGFSVVEQFLKGASSIDQHFYSAPHEKNIPVLLGLLSVWNVSF |
| | | | FGYPARAILPYSQALEKFAPHIQQVSMESNGKGVSIDGVPLPFEAGEIDFGEPGTN |
| | | | GQHSFYQLIHQGRVIPCDFIGVVKSQQPVFLKGEVVSNHDELMSNFFAQPDALAY |
| | | | GKTPEQLHKENVAPHLIPHKTFSGNRPSLSLLLPSLNAYNIGQLLAIYEHRIAVEGFV |
| | | | WGINSFDQWGVELGKSLATQVRKQLHVSRTKGEPVEGFNFSTATLLTRYLEATAD |
| | | | IPADPPTLLPRI |
| 684 | 3 | Amb_a | DERENHGNMKRVESDSSLYETEDDGEDGEGNKIVLGPQCTLKEQFEKDKDDESL |
| | | | RKWKEQLLGNVDINNVGESLEPDVKILSLSIVSPGRSDIILPIPESGKPEGRWFTLK |
| | | | EGCHYNLKFSFQVSHNIVAGLKYTNHVWKTGVRVYNIKEMLGTFSPQLEPYTFVTP |
| | | | EETTPSGYFARGSYSAKSRFVDDDNKCYLEINYSFDIRKDWANA |
| 685 | 3 | Amb_p | DEEDTQIQLGPKISIREHLEKDKDDESLRRWKEQLLGSVDVSQVEEVQEPDVKILS |
| | | | LTIISADRPDIVLEIPNPGNPKAPWFTLKEGSKYNLKFSIKVSNDIVCGLRYTNHVW |
| | | | KTGLKVDNSKEMLGTFSPQPEPYTHIMPEEVTPSGFLARGNYSAKTKFFDDDNKCY |
| | | | LELNYTFDIQKDW |
| 686 | 3 | Amb_p | DERENHGNMKRVESDSSLYETEDDGEDGEGNKIVLGPQCTLKEQFEKDKDDESL |
| | | | RKWKEQLLGNVDINNVGESLEPDVKILSLSIVSPGRSDIILPIPESGKPEGRWFTLK |
| | | | EGCHYNLKFSFQVSHNIVAGLKYTNHVWKTGVRVYNIKEMLGTFSPQLEPYTFVTP |
| | | | EETTPSGYFARGSYSAKSKFVDDDNKCYLEINYSFDIRKDWANA |
| 687 | 3 | Amb_p | EPYTYAGEEETTPAGMFARGSYSAKLKFVDDDGKVYLEMSYYFEIRKDWPATQ |
| 688 | 3 | Bet_v | DQEEEDDEGNKLELGPQYTLKQQLEKDKDDESLRRWKEQLLGSVDLNNVGETLD |
| | | | PDVKILSLSIVSPGRSDIVVPIPEDGNPKGLWFTLKEGSKYCLKFSFQVSNNIVSGL |
| | | | KYTNTVWKSGIRVDSSKEMLGTFSPQLEPYVHVMPEESTPSGIFARGSYSAKSKFL |
| | | | DDDNKCYLEINYTFGIRKEW |
| 689 | 3 | Cyn_d | KRTVVLGPQVPLKEQLELDKDDESLRRWKEQLLGQVDTEQLGETAEPEVKVLNLTI |
| | | | LSPGRPDLVLPIPFQPDEKGYAFALKDGSPYSFRFSFIVSNNIVSGLKYTNTVWKTG |
| | | | VRVENQKMMLGTFSPQLEPYVYEGEEETTPAGMFARGSYSAKLKFVDDDGKVYLE |
| | | | MSYYFEIRKEWPAA |
| 690 | 3 | Que_a | TDQEEEDDERSKLQLGPQYTLKEQLEKDKDDESLRRWKEQLLGSVDLNNVGETLE |
| | | | PDVKIFCLSIISPGRSDIVLPIPEDGKPKGIWFTLKEGSKYKLKFSFQVSNNIVSGLK |
| | | | YTNTVWKTGIKVDSSKEMIGTFSPQIEPYTHIMQEETTPSGMFSRGSYSARSKFLD |
| | | | DDNKCYLEINYGFDIRKEWAS |
| 691 | 4 | Amb_a | MANFTVNRVVTSPIEGQKPGTSGLRKKVKVFTQPHYLHNFVQSTFNALSAEKVKG |
| | | | STLVVSGDGRYYSKDAIQIIIKMAAANGVRRVWVGQNGLLSTPAVSAVVRERVGA |
| | | | DGSKANGAFILTASHNPGGPNEDFGIKYNMGNGGPAPEGITDKIFENTKTIKEYFI |
| | | | AEGLPDVDISAIGVSNFSGPGGQFDVDVFDSASDYVKLMKSIFDFQSIKKLITSPQ |
| | | | FSFCFDALHGVGGAYAKRMFVEELGAKESSLLNCVPKEDFGGGHPDPNLTYAKEL |
| | | | VARMGLGTNPDSNPPEFGAAADGDADRNMILGKRFFVTPSDSVAIIAANAVQAIP |
| | | | YFSSGLKGVARSMPTSAALDVVAKSLNLKFFEVPTGWKFFGNLMDAGLCSICGEE |
| | | | SFGTGSDHIREKDGIWAVLAWLSILAHKNKDNLDGGKLVTVEDIVKQHWATFGR |
| | | | HYYTRYDYENVDAGAAKEVMAHLVDLQSSISGVNTTI |
| 692 | 4 | Amb_p | SIFDFQSIKKLITSPQFSFCFDALHGVGGAYAKRMFVEELGAKESSLLNCVPKEDFG |
| | | | GGHPDPNLTYAKELVARMGLGTNPDSNPPEFGAAADGDADRNMILGKRFFVTPSD |
| | | | SVAIIAANAVQAIPYFSSGLKGVARSMPTSAALDVVAKSLNLKFFEVPTGWKFFGN |
| | | | LMDAGLCSICGEESFGTGSDHIREKDGIWAVLAWLSILAHKNKDNLDGGKLVTVE |
| | | | DIVKQHWATFGRHYYTRYDYENVDAGAAKEVMAHLVDLQSSISGVNTTIKGIRSD |
| | | | VADVVSADEFEYKDPVDGSVSKNQGIRYLFEDGSRLVFRLSGTGSEGATIRLYIEQ |
| | | | YEKDSSKTGRDSQEALAPLVDVALKLSKMLEYTGRSAPTVIT |
| 693 | 4 | Bet_v | MVVFKVARVESTPFDGQKPGTSGLRKKVKVFIQPNYLENFVQSTFNALTPEKVRGA |
| | | | TLVVSGDGRYYSKDAIQIIIKMAAANGVRRVWVGQNGLLSTPAVSAVIRERVAVD |
| | | | GSRASGAFILTASHNPGGPHEDFGIKYNMENGGPAPEGLTDKIYENTKTIKEYFIAE |
| | | | DLPDVDITTTGVTRFGGPEGQFDVDVFDSASDYVKLMKSIFDFELIRKLLSSPKFTF |
| | | | CYDALHGVAGAYAKRIFVEELGAQESSLLNCTPKEDFGGGHPDPNLTYAKELVAR |
| | | | MGLGKSNSQDEVPEFGAAADGDADRNMILGKRFFVTPSDSVAIIAANAVQAIPYF |
| | | | SAGLKGVARSMPTSAALDVVAKHLNLKFFEVPTGWKFFGNLMDAGLCSVCGEESF |
| | | | GTGSDHIREKDGIWAVLAWLSILAHKNKENLGGEKLVTVEDIVRQHWATYGRHY |
| | | | YTRYDYENVDAAAAKALMAYLVKLQSSLSEVNEIVKGVRSDVAKVVDADEFEYKD |
| | | | PVDGSISKHQGIRYLFEDGSRLVFRLSGTGSEGATIRLYIEQYEKDPSKIGRDSQE |
| | | | ALAPLVEVALKLSKMQEFTGRGAPTVIT |
| 694 | 4 | Cyn_d | MVLFTVTKKATTPFEGQKPGTSGLRKKVTVFQQPNYLQNFVQATFNALPADQVKG |
| | | | ATIVVSGDGRYFSKDAVQIITKMAAANGVRRVWVGQNSLMSTPAVSCVIRDRVG |
| | | | SDGSKATGAFILTASHNPGGPTEDFGIKYNMGNGGPAPESVTDKIFSNTKTISEYLI |
| | | | SEDLPDVDISVVGVTSFSGPEGPFDVDVFDSSVDYIKLMKSIFDFEATKNLVTSPKF |
| | | | TFCYDALHGVAGAYAKQIFVEELGADESSLLNCVPKEDFGGGHPDPNLTYAKELVE |
| | | | RMGLGKSTSNVEPPEFGAAADGDADRNMILGKRFFVTPSDSVAIIAANAVQSIPYF |
| | | | SSGLKGVARSMPTSAALDVVAKNLNLKFFEVPTGWKFFGNLMDAGMCSICGEESF |
| | | | GTGSDHIREKDGIWAVLAWLSILAFKNKDNLRGDKLVSVEDIVRQHWATYGRHY |
| | | | YTRYDYENVDAGAAKELMANLVSMQSSLSDVNKLIKEIRSDVSDVVAADEFEYKD |
| | | | PVDGSVSKHQGIRYLFGDGSRLVFRLSGTGSVGATIRVYIEQYEKDSSKIGRESQ |
| | | | DALAPLVDVALKLSKMQEYTGRSAPTVIT |
| 695 | 4 | Que_a | MVFKVSRVETKPIDGQKPGTSGLRKKVKVFIQPHYLHNFVQSTFNALTPEKVRGAT |
| | | | LVVSGDGRYYSKDAIQIITKMSAANGVRRVWVGQNGLLSTPAVSAVIRERVGVDG |
| | | | SRASGAFILTASHNPGGPNEDFGIKYNMENGGPAPEGITDKIYENTKTIKEYFISED |
| | | | LPDVDISAVGVTSFAGPEGQFDVEVFDSASDYVKLMKSIFDFESIRKLISSPKFTFC |
| | | | YDALHGVAGAYAKRIFVEELGAQESSLLNCTPKEDFGGGHPDPNLTYAKELVARM |
| | | | GLGKSSSQGEPPEFGAAADGDADRNMILGKRFFVTPSDSVAIIAANAVESIPYFSA |
| | | | GLKGVARSMPTSAALDVVAKHLNLKFFEVPTGWKFFGNLMDAGLCSVCGEESFGT |
| | | | GSDHIREKDGIWAVLAWLSILAHKNKENLGEEKLVSVEDIVRQHWTTYGRHYYTR |
| | | | YDYENVDAGAAKELMAYLVKLQSSLPEVNEIVKGTRSDVSKVINADEFEYKDPVD |
| | | | GSISKHQGIRYLFEDGSRLVFRLSGTGSEGATIRLYIEQYEKDPSKTGRDSQDALA |
| | | | PLVEVALKLSKMQEFTARTAPTVIT |
| 696 | 5_64 | Amb_a | KCYPVVSEEYKKAVDKARKKLRGFIAEKRCAPLMLRLAWHSAGTYDVNTKTGGPF |
| | | | GTMRYKAELSHGANNGLDIAVRLLEPIKEQFPILSYGDFYQLAGVVAVEVTGGPDV |
| | | | PFHPGRVDKEEPPVEGRLPDATKGTDHLRDVFVKTMGLEDIDIVTLSGGHTLGAA |
| | | | HKERSGFEGPWTPNPLIFDNSYFTELLAGEKEGLLKLPTDKALLEDPVFRPLVDKYA |
| | | | ADEDAFFADYAVSHMKLSELGFADA |
| 697 | 5_64 | Amb_a | LAWHSAGTFDVQSKTGGPFGTMRHKAELAHGANNGLDIAVRLLEPLKEQFPEISY |
| | | | ADFYQLAGVVAVEVTGGPEVPFHPGREDKPEPPQEGRLPDATKGCDHLRDVFIKQ |
| | | | MGLTDQDIVALSGGHTLGRCHKERSGFEGPWTANPLVFDNSYFKELLSGEKEGLL |
| | | | QLPTDKALLSDPVFRPFVEKYAADEDAFFADYAEAHLKLSELGF |
| 698 | 5_64 | Amb_p | KSYPCVSEEYKKAVDKARRKLRGFIADKRCAPLMLRLAWHSAGTYDVKTKTGGPF |
| | | | GTMRYKAELSHGANNGLDIAVRLLEPIKEQFPNISYGDFYQLAGVVAVEIAGGPEV |
| | | | PFHPGREDKEEPPLEGRLPDATKGNDHLRDVFVKTMGLDDIDIVTLSGGHTLGAA |
| | | | HKERSGFEGPWTPNPLIFDNSYFTELLAGEKEGLLKLPTDKALLEDPVFRPLVEKYA |
| | | | ADEDAFFADYAVSHMKLSELGFAE |
| 699 | 5_64 | Amb_p | LAWHSAGTFDVQSKTGGPFGTMRHKAELAHGANNGLDIAVRLLEPLKEQFPEISY |
| | | | ADFYQLAGVVAVEVTGGPEVPFHPGREDKPEPPQEGRLPDATKGCDHLRDVFIKQ |
| | | | MGLTDQDIVALSGGHTLGRCHKERSGFEGPWTANPLVFDNSYFKELLSGEKEGLL |
| | | | QLPTDKALLSDPVFRPFVEKYAADEDAFFADYAEAHLKLSELGFADA |
| 700 | 5_64 | Bet_v | DCLWLLWRCSWHSAGTFDVETKTGGPFGTIRHPDELAHEANSGLDIAIRLLEPIKE |
| | | | QFPILSYADFYQLAGVVAVEVTGGPEIPFHPGRPDKTEPPPEGRLPDATKGSDHLR |
| | | | DIFGHMGLSDKDIVALSGGHTLGRCHKERSGFEGPWTNNPLIFDNSYFKELLSGE |
| | | | KEGLIQLPSDKALLEDPVFRPLVEKYAADEDAFFADYAEAHLKLSELGFADA |
| 701 | 5_64 | Cyn_d | KSYPAVSEDYLKAVDKAKRKLRGLIAEKNCAPLILRLAWHSAGTFDVATKSGGPYG |
| | | | TMKNPSEQAHAANAGLDIAVRLLEPIKEQFPILSYADFYQLAGVVAVEVTGGPDVP |
| | | | FHPGREDKPEPPPEGRLPDATKGSDHLRQVFATQMGLSDQDIVALSGGHTLGRCH |
| | | | KDRSGFEGAWTSNPLIFDNSYFKELLSGEKEGLLQLPSDKALLSDPSFRPLVEKYA |
| | | | ADEDAFFADYAEAHLKLSELGFAE |
| 702 | 5_64 | Cyn_d | MAKNYPTVSAEYQEAVEKARRKLRALIAEKSCAPLMLRLAWHSAGTFDVSTKTGG |
| | | | PFGTMKNPAEQAHGANAGLDIAVRMLEPVKEEFPILSYADLYQLAGVVAVEVTGGP |
| | | | EIPFHPGREDKPQPPPEGRLPDATKGTDHLRQVFGKQMGLSDQDIVALSGGHTLG |
| | | | RCHKERSGFEGPWTRNPLCFDNSYFTELLTGDKEGLLQLPSDKALLNDPVFRPLVE |
| | | | KYAADEKAFFEDYKEAHLRLSELGFADA |
| 703 | 5_64 | Que_a | MTKQYPSVSAEYQKTVEKARRKLRGLIAEKHCAPLMLRIAWHSAGTFDQKTKTGG |
| | | | PFGTMKQAAELSHGANNGLDIAVRLLEPIKEQFPTLSYADFYQLAGVVAVEITGGP |
| | | | EVPFHPGREDKPQPPPEGRLPDATKGSDHLRVVFGQQMGLSDQDIVALSGGHTL |
| | | | GRCHKERSGFEGPWTANPLIFDNSYFKELLSGEKEGLLQLPSDKALLADPVFRPLV |
| | | | EKYAADEDAFFADYAEAHLKLSELGFAEA |
| 704 | 6 | Amb_a | EKLNNLRSAVSSLTQISENEKSGFINLVSRYLSGEAEHVEWSKIQTPTDKIVVPYDT |
| | | | LSAVPEDAAETKSLLDKLVVLKLNGGLGTTMGCTGPKSVIEVRNGLTFLDLIVIQIE |
| | | | SLNKKYGCSVPLLLMNSFNTHEDTQKIIEKYAGSNIEIHTFNQSQYPRLVVDDFLPL |
| | | | PSKGETGKDGWYPPGHGDVFPSLMNSGKLDALLSQGKEYVFVANSDNLGAVVDL |
| | | | KILNHLIQNKNEYCMEVTPKTLADVKGGTLISYDGKVQLLEIAQVPDEHVNEFKSIE |
| | | | KFKIFNTNNLWVNLNAIKRLVQADALKMEIIPNPKEVNGVKVLQLETAAGAAIKFFD |
| | | | NAIGINVPRSRFLPVKASSDLLLVQSDLYTEKDGYVIRNPARTDPANPSIELGPEFK |
| | | | KVGDFLKRFKSIPSIIELASLKVSGDVWFGSNVVLKGKVVVAANSGEKLEIPDGAV |
| | | | LENKEVHSAGDI |
| 705 | 6 | Amb_p | YHHSRSKSINQSMAAADTEKLNNLRSAVSSLTQISENEKSGFINLVSRHLSGEAEH |
| | | | VEWSKIQTPTDKIVVPYDTLSAVPEDAAETKSLLDKLVVLKLNGGLGTTMGCTGPK |
| | | | SVIEVRNGLTFLDLIVIQIESLNKKYGCSVPLLLMNSFNTHEDTQKIIEKYAGSNIEI |
| | | | HTFNQSQYPRLVVDDFLPLPSKGETGKDGWYPPGHGDVFPSLMNSGKLDALLSQ |
| | | | GKEYVFVANSDNLGAVVDLKILNHLIQNKNEYCMEVTPKTLADVKGGTLISYDGKV |
| | | | QLLEIAQVPDAHVNEFKSIEKFKIFNTNNLWVNLNAIKRLVQADALKMEIIPNPKEV |
| | | | NGVKVLQLETAAGAAIKFFDNAIGINVPRSRFLPVKASSDLLLVQSDLYTEKDGYVI |
| | | | RNPARTDPANPSIELGPEFKKVGDFLKRFKSIPSIIELASLKVSGDVWFGSNVVLKG |
| | | | KVVVAANSGEKLEIPDGAVLENKEVHSAGDI |
| 706 | 6 | Amb_p | EKLNNLRSAVSSLTQISENEKSGFINLVSRHLSGEAEHVEWSKIQTPTDKIVVPYD |
| | | | TLSAVPEDAAETKSLLDKLVVLKLNGGLGTTMGCTGPKSVIEVRNGLTFLDLIVIQI |
| | | | ESLNKKYGCSVPLLLMNSFNTHEDTQKIIEKYAGSNIEIHTFNQSQYPRLVVDDFLP |
| | | | LPSKGETGKDGWYPPGHGDVFPSLMNSGKLDALLSQGKEYVFVANSDNLGAVVD |
| | | | LKILNHLIQNKNEYCMEVTPKTLADVKGGTLISYDGKVQLLEIAQVPDAHVNEFKSI |
| | | | EKFKIFNTNNLWVNLNAIKRLVQADALKMEIIPNPKEVNGVKVLQLETAAGAAIKFF |
| | | | DNAIGINVPRSRFLPVKASSDLLLVQSDLYTEKDGYVIRNPARTDPANPSIELGPEF |
| | | | KKVGDFLKRFKSIPSIIELASLKVSGDVWFGSNVVLKGKVVVAANSGEKLEIPDGA |
| | | | VLENKEVHSAGDI |
| 707 | 6 | Ant_o | PHPTSDRPSSILSSPSARTTHLATMADEKLAKLREAVAGLGQISDNEKSGFISLVS |
| | | | RYLSGDEEHIEWPKIHTPTDEVVVPYDTIDAPPEDLEATKALLNKLAVLKLNGGLGT |
| | | | TMGCTGPKSVIEVRNGFTFLDLIVLQIESLNKKYGSNVPLLLMNSFNTHDDTLKIVE |
| | | | KYANSSIDIHTFNQSQYPRVVADEFLPWPSKGKTDKDGWYPPGHGDIFPSLMNS |
| | | | GKLDLLLSQGKEYVFIANSDNLGAIVDMKILNHLIHKQNEYCMEVTPKTLADVKGG |
| | | | TLISYEGRVQLLEIAQVPDAHVDEFKSIEKFKIFNTNNLWVNLKAIKRLVEADALKM |
| | | | EIIPNPKEVEGVKVLQLETAAGAAIRFFDHAIGINVPRSRFLPVKATSDLQLVQSDL |
| | | | YTLVDGFVTRNSARTDPSNPSIELGPEFKKVGSFLGRFKSIPSIVELDSLKVSGDV |
| | | | WFGSGIVLKGKVTITAKPGVKLEIPDGAVLENKDIKGAEDL |
| 708 | 6 | Bet_v | EKLNKLKSAVDGLNQISENEKIGCINLVARYLSGEAQHVEWSKIQTPTDEIVVPYE |
| | | | SLAPTTDDPVETKKLLDKLVVLKLNGGLGTTMGCTGPKSVIEVRNGLTFLDLIVIQI |
| | | | ENLNSKYGCNVPLLLMNSFNTHDDTLKIVERYSGSKVEIHTFNQSQYPRLVVDDFS |
| | | | PLPSKGQTGKDGWYPPGHGDVFPSLKNSGKLDALLSQGKEYVFIANSDNLGAVV |
| | | | DLKILNHLVHNKNEYCMEVTPKTLADVKGGTLISYEGRVQLLEIAQVPDDHVNEFK |
| | | | SIEKFKIFNTNNLWVNLKAIKRLVETDALKMEIIPNPKEVDGIKVLQLETAAGAAIKF |
| | | | FDDAIGINVPRSRFLPVKATSDLLLVQSDLYTLEDGFVIRNEARKNPANPSIELGPE |
| | | | FKKVGNFLSRFKSIPSIIELDSLKVAGDVWFGTGVTLKGKVSIVAKPGVKLEIPDGA |
| | | | VLENKEINGPEDL |
| 709 | 6 | Bet_v | PFSFQFSFTSITMASEMATHLKPNGGAEFEKRHHGKTQSHVAFENTSTSVAASQM |
| | | | RNALNNLCDEVTDPAEKQRFETEMDNFFALFRRYLNDKAKGNEIEWSRIAPPKPEQ |
| | | | VVAYEDLPQQESVDFLNKLAVLKLNGGLGTSMGCVGPKSVIEVRDGMSFLDLSVR |
| | | | QIEYLNRTYGVNVPFVLMNSFNTDSDTANIIKKYEGHNIDIMTFNQSRYPRVLKDS |
| | | | LLPAPKSANSQISDWYPPGHGDVFESLYNSGILDKLLERGVEIVFLSNADNLGAVV |
| | | | DLKILQHMVDTKAEYIMELTDKTKADVKGGTIIDYEGQARLLEIAQVPKEHVNEFK |
| | | | SIKKFKYFNTNNIWMNLRAVKRIVENNELAMEIIPNGKSIPADKKGEADVSIVQLET |
| | | | AVGAAIRHFHNAHGVNVPRRRFLPVKTCSDLMLVKSDLYTLKHGQLIMDPNRFGP |
| | | | APLIKLGGDFKKVSSFQSRIPSIPKILELDHLTITGPVNLGRGVTCKGTVIIVASEGQ |
| | | | TIDIPPGSILENVVVQGSLRLLEH |
| 710 | 6 | Bet_v | LAGSLRMTIHSVVIQKLLSTNAHLGRRVAADHFKAYTYGIRNGMAIIDSDKTLIALR |
| | | | SACAFIGAMARQKARFMFVNTNPLFDEIFEQMTKRIGLYNPNQNSLWRTGGFLTN |
| | | | SFSPKRFRSRNKKLCFAPAQPPDCVVILDTERKSSVIFEAEKLQIPVVALVDSSMPL |
| | | | DVYKRIAYPVPANDSVQFVYLFCNLITKTFLLEQKRFGGTAREDSAAAIPSADDASK |
| | | | IENHREEVKRIEERESDSVGYAKDEVLVVPYESLTPVSGDGAEIKELLDKLVVLKFN |
| | | | GTLGTELGFDGPKSAIEVCNGLTFLDLIVNQIESLNSKYGCNVPLLLMNTIKTNDDS |
| | | | VKVLEKYPKSNIVMLKSFDGQTCEKESYPSDHDMEFLSLMKGGTLDVLLSQGKEYI |
| | | | LVVGSDNVAAGIDPKILKHLVQNKIEYCMEVTPTTSFGKDNDILNSSQQKFQLAKI |
| | | | ARNSAPHSMDKFKLVDTRSLWLNLRATKRLVDTDALNFENYSVSKGRETAAGSTI |
| | | | RFFDRAIGINVPQ |
| 711 | 6 | Bet_v | AMAAATLNTADAEKLNKLKSAVDGLNQISENEKIGCINLVARYLSGEAQHVEWSK |
| | | | IQTPTDEIVVPYESLAPTTDDPVETKKLLDKLVVLKLNGGLGTTMGCTGPKSVIEVR |
| | | | NGLTFLDLIVIQIENLNSKYGCNVPLLLMNSFNTHDDTLKIVERYSGSKVEIHTFNQ |
| | | | SQYPRLVVDDFSPLPSKGQTGKDGWYPPGHGDVFPSLKNSGKLDALLSQGKEYVF |
| | | | IANSDNLGAVVDLKILNHLVHNKNEYCMEVTPKTLADVKGGTLISYEGRVQLLEIA |
| | | | QVPDDHVNEFKSIEKFKIFNTNNLWVNLKAIKRLVETDALKMEIIPNPKEVDGIKVL |
| | | | QLETAAGAAIKFFDDAIGINVPRSRFLPVKATSDLLLVQSDLYTLEDGFVIRNEARK |
| | | | NPANPSIELGPEFKKVGNFLSRFKSIPSIIELDSLKVAGDVWFGTGVTLKGKVSIVA |
| | | | KPGVKLEIPDGAVLENKEINGPEDL |
| 712 | 6 | Cyn_d | PTPSSSSHLPVSSPLPDLSAHLAMADEKLAKLSEAVAGLAEISENEKSGFLSLVSRY |
| | | | LSGDEEHIEWAKIHTPTDEVVVPYDALETPPEDIEETKKLLDKLAVLKLNGGLGTTM |
| | | | GCTGPKSVIEVRNGFTFLDLIVLQIEALNKKYGSNVPLLLMNSFSTHDDTLKIVEKY |
| | | | ANSNIDIHTFNQSKYPRVVADEFLPWPSKGKTCKDGWYPPGHGDIFPSLMNSGKL |
| | | | DLLLSQGKEYVFIANSDNLGAIVDMKILNHUHKQNEYCMEVTPKTLADVKGGTLI |
| | | | SYEGRVQLLEIAQVPDAHVHEFKSIEKFKIFNTNNLWVNLKAIKRLVEADALKMEII |
| | | | PNPKEVDGVKVLQLETAAGAAIRFFDHAIGINVPRSRFLPVKATSDLQLVQSDLYTL |
| | | | VDGLVTRNEARTNPSNPSIELGPEFKKVGNFLGRFKSIPSIVELDSLKVSGDVWFG |
| | | | SGIVLKGKVSITAKPGVKLEIPDGAVIENKDISGPEDL |
| 713 | 6 | Cyn_d | MADEKLAKLSEAVAGLAEISENEKSGFLSLVSRYLSGDEEHIEWAKIHTPTDEVVV |
| | | | PYDALETPPEDIEETKKLLDKLAVLKLNGGLGTTMGCTGPKSVIEVRNGFTFLDLIVL |
| | | | QIEALNKKYGSNVPLLLMNSFSTHDDTLKIVEKYANSNIDIHTFNQSKYPRVVADE |
| | | | FLPWPSKGKTCKDGWYPPGHGDIFPSLMNSGKLDLLLSQGKEYVFIANSDNLGAI |
| | | | VDMKILNHLIHKQNEYCMEVTPKTLADVKGGTLISYEGRVQLLEIAQVPDAHVHEF |
| | | | KSIEKFKIFNTNNLWVNLKAIKRLVEADALKMEIIPNPKEVDGVKVLQLETAAGAAI |
| | | | RFFDHAIGINVPRSRFLPVKATSDLQLVQSDLYTLVDGLVTRNEARTNPSNPSIELG |
| | | | PEFKKVGNFLGRFKSIPSIVELDSLKVSGDVWFGSGIVLKGKVSITAKPGVKLEIPD |
| | | | GAVIENKDISGPEDL |
| 714 | 6 | Fra_e | LYSKMSTATLSAADKEKITKLQSAVSGLNQISENEKVGFVNLVTRYLSGEAQHVE |
| | | | WSKIQTPTDEVVVPYDTLTPVPEDPAETKKLLDKLVVLKLNGGLGTTMGCTGPKSV |
| | | | IEVRNGLTFLDLIVIQIETLNKKYGCSVPLLLMNSFNTHDDTLKIVEKYTNSNIEIHT |
| | | | FNQSQYPRLAIDNFTPLPCIKDAGKDGWYPPGHGDVFPSLVNSGKLEALLSQGKE |
| | | | YVFVANSDNLGAVVDLKILNHLISNKNEYCMEVTPKTLADVKGGTLISYEGKVQLL |
| | | | EIAQVSDEHVNEFKSIEKFKIFNTNNLWVNLNAIKRLVQADALKMEIIPNPKEVDGI |
| | | | KVLQLETAAGAAIRFFDRAIGINVPRSRFLPVKATSDLLLVQSDLYTLSDGFVTRNP |
| | | | ARTNPANPSIELGPEFKKVANFLSRFKSIPSIIELDSLKVTGDVWFGSGIALKGKVTI |
| | | | AAKPGVKLEIPDGAVIANKDINGPEDI |
| 715 | 6 | Fra_e | LYSKMSTATLSAADKEKITKLQSAVSGLNQISENEKVGFVNLVTRYLSGEAQHVE |
| | | | WSKIQTPTDEVVVPYDTLTPVPEDPAETKKLLDKLVVLKLNGGLGTTMGCTGPKSV |
| | | | IEVRNGLTFLDLIVIQIETLNKKYGCSVPLLLMNSFNTHDDTLKIVEKYTNSNIEIHT |
| | | | FNQSQYPRLAIDNFTPLPCIKDAGKDGWYPPGHGDVFPSLVNSGKLEALLSQGKE |
| | | | YVFVANSDNLGAVVDLKILNHLISNKNEYCMEVTPKTLADVKGGTLISYEGKVQLL |
| | | | EIAQVSDEHVNEFKSIEKFKIFNTNNLWVNLNAIKRLVQADALKMEIIPNPKEVDGI |
| | | | KVLQLETAAGAAIRFFDRAIGINVPRSRFLPVKATSDLLLVQSDLYTLSDGFVTRNP |
| | | | ARTNPANPSIELGPEFKKVANFLSRFKSIPSIIELDSLKVTGDVWFGSGIALKGKVTI |
| | | | AAKPGVKLEIPDGAVIANKEINGPQDI |
| 716 | 6 | Lol_p | LISYEGKVQLLEIAQVPDEHVNEFKSIEKFKIFNTNNLWVNLNAIKRLVQADALKME |
| | | | IIPNPKEVDGIKVLQLETAAGAAIKFFDRAIGINVPRSRFLPVKATSDLLLVQSDLYT |
| | | | LSDGFVTRNPARTNPANPSIELGPE |
| 717 | 6 | Lol_p | SPSPTSDDPPLPFPQKHLPPHVHATMADEKLAKLREAVAGLGQISDNEKSGFISLV |
| | | | SRYLSGDEEHIEWPKIHTPTDEVVVPYDTIDAPPEDLEATKALLNKLAVLKLNGGLG |
| | | | TTMGCTGPKSVIEVRNGFTFLDLIVLQIESLNKKYGSNVPLLLMNSFNTHDDTLKIV |
| | | | EKYANSSIDIHTFNQSQYPRVVADEFLPWPSKGKTDKDGWYPPGHGDIFPSLMNS |
| | | | GKLDLLLSQGKEYVFIANSDNLGAIVDMKILNHLIHKQNEYCMEVTPKTLADVKGG |
| | | | TLISYEGRVQLLEIAQVPDAHVDEFKSIEKFKIFNTNNLWVNLKAIKRLVEADALKM |
| | | | EIIPNPKEVEGVKVLQLETAAGAAIRFFDHAIGMNVPRSRFLPVKATSDLQLVQSDL |
| | | | YTLVDGFVTRNSARTDPSNPSIELGPEFKKVGSFLGRFKSIPSIVELDSLKVSGDV |
| | | | WFGSGIVLKGKVTITAKPGVKLEIPDGKVIENKDINGVEDL |
| 718 | 6 | Lol_p | THHHHHLTTSSHLKSPPVLSSSSASRSLLCLPARIAMAATAVAAGPDAKIEKFRDA |
| | | | VAKLDEISENEKAGCISLVSRYLSGEAEQIEWSKIQTPTDEVVVPYDTLAPAPEDLD |
| | | | AMKALLDKLVVLKLNGGLGTTMGCTGPKSVIEVRNGFTFLDLIVIQIESLNKKYGC |
| | | | DVPLLLMNSFNTHDDTQKIVEKYSNSNINIHTFNQSQYPRIVTEDFLPLPSKGKSG |
| | | | KDGWYPPGHGDVFPSLNNSGKLDTLLSQGKEYVFVANSDNLGAIVDIKILNHLIN |
| | | | NQNEYCMEVTPKTLADVKGGTLISYEGRVQLLEIAQVPDEHVNEFKSIEKFKIFNTN |
| | | | NLWVNLKAIKRLVEADALKMEIIPNPKEVDGVKVLQLETAAGAAIRFFEKAIGINGP |
| | | | RSRFLPVKATSDLLLVQSDLYTLVDGYVIRNPARVKPSNPSIELGPEFKKVASFLAR |
| | | | FKSIPSIVELDSLKVSGDVTFGSGVVLKGNVTIAAKSGVKLEIPDGAVLENKDINGP |
| | | | EDL |
| 719 | 6 | Ole_e | EMATATLSATDNEKISKLQSSVSGLNQISENEKAGFLNLVTRYLSGEAQHVEWSKI |
| | | | QTPTDEVVVPYDTLAPVPEDHAETKKLLSKLVVLKLNGGLGTTMGCTGPKSVIEVR |
| | | | NGLTFLDLIVIQIETLNKKYGCSVPLLLMNSFNTHDDTLKIVEKYANSNIEIHTFNQS |
| | | | QYPRLAVDNFTPLPCIKDAGKDGWYPPGHGDVFPSLMNSGKLEALLSQGKEYVFV |
| | | | ANSDNLGAVVDMKILNHLINNKNEYCMEVTPKTLADVKGGTLISYEGKVQLLEIAQ |
| | | | VPDEHVNEFKSIEKFKIFNTNNLWVNLNAIKRLVQADALKMEIIPNPKEVDGIKVLQ |
| | | | LETAAGAAIKFFDRAIGINVPRSRFLPVKATSDLLLVQSDLYTLSDGFVTRNPARTN |
| | | | PANPSIELGPEFKKVANFLSRFKSIPSIIDLDSLKVTGDVWFGSGITLKGKVTIAAKP |
| | | | GVKLEIPDGAVIANKEINGPEDI |
| 720 | 6 | Pla_l | KEMAAATLSQADAEKLSKLTSSVATLDGISENEKSGFISLVGRYLSGEAQHVEWS |
| | | | KIQTPTDEVVVPYDTMSPVPEDPAETKKLLDKLVVLKLNGGLGTTMGCTGPKSVIE |
| | | | VRNGLTFLDLIVVQIESLNAKYGCSVPLLLMNSFNTHDDTLKIVEKYSNSKIEIHTF |
| | | | NQSQYPRMVVEDFSPLPTKISGKDAWYPPGHGDVFPALMNSGKLDALIAQGKEYV |
| | | | FVANSDNLGAVVDLKILNHLVNNKNEYCMEVTPKTLADVKGGTLISYEGKVQLLEI |
| | | | AQVPDEHVNEFKSIEKFKIFNTNNLWVNLQSIKKLVQGDVLKMEIIPNPKEVEGIKI |
| | | | LQLETAAGAAIRFFDHAIGANVPRARFLPVKATSDLLLVQSDLYTLSDGFVLRNPAR |
| | | | TNPENPSIELGPEFKKVANFLGRFKSIPSIIGLDSLKVSGDVWFGAGITLKGKVTIA |
| | | | AKSGTKLEIPDGAVIADKEINGPEDI |
| 721 | 6 | Poa_p | DLQLVQSDLYTLVDGLVTRNEARTNPSNPSIELGPEFKKVGNFLGRFKSIPSIVELD |
| | | | SLKVSGDVWFGSGIILKGKVTIT |
| 722 | 6 | Poa_p | VNVAAFPHFPPATCSSLFSGINSQRHLLLLPPSTLLFPHIYLPLPSVRTRTHLAATMA |
| | | | DEKLAKLGEAVTGLPQISDNEKSGFISLVSRYLSGDEEHIEWPKIHTPTDEVVVPY |
| | | | DAIDAPPEDLEATKALLDKLAVLKLNGGLGTTMGCTGPKSVIEVRNGFTFLDLIVLQ |
| | | | IESLNKKYGSNVPLLLMNSFNTHDDTLKIVEKYANSSIDIHTFNQSQYPRVVADEFL |
| | | | PWPSKGKTDKDGWYPPGHGDIFPSLMNSGKLDLLLSQGKEYVFIANSDNLGAIVD |
| | | | MKILNHLIHKQNEYCMEVTPKTLADVKGGTLISYEGRVQLLEIAQVPDAHVDEFKS |
| | | | IEKFKIFNTNNLWVNLKAIKRLVEADALKMEIIPNPKEIDGVKVLQLETAAGAAIRFF |
| | | | DHAIGINVPRSRFLPVKATSDLQLVQSDLYTLVDGFVTRNSARTDPSNPSIELGPEF |
| | | | KKVGSFLGRFKSIPSIVELESLKVSGDVWFGSGIVLKGKVTITAKPGVKLEIPDGAV |
| | | | LENKDINGAEDL |
| 723 | 6 | Que_a | TMAAPTLSAADAEKLNSLKSSVAALPQISENEKNGFINLIARFLSGEAQHVDWSKI |
| | | | QTPTDEVVVPYDTLKPAPHDPAETKKLLDKLVVLKLNGGLGTTMGCTGPKSVIEVR |
| | | | NGLTFLDLIVIQIENLNKQYGCNVPLLLMNSFNTHDDTQKIVEKYSGANVEIHTFN |
| | | | QSQYPRLVVEDFSPLPSKGVTGKDGWYPPGHGDVFPSLRNSGKLDLLLSQGKEYV |
| | | | FIANSDNLGAVVDLKILNHLVHNKNEYCMEVTPKTMADVKGGTLISYEGRVQLLEI |
| | | | AQVPDEHVNEFKSIEKFKIFNTNNLWANLKAIKRLVEADALKMEIIPNPKEVEGIKV |
| | | | LQLETAAGAAIRFFDNAIGNNVPRSRFLPVKATSDLLLVQSDLYTLEDGFVIRNKAR |
| | | | TNPANPSIELGPEFKKVGNFLNRFKSIPSIVELDSLKVTGDVWFGANITLKGKVTIV |
| | | | AKPGAKLEIPDGAVLENKEINGPEDI |
| 724 | 6 | Que_a | TPKPPNETVTMTIHSVVIQKLLSTNAHLGRRVVADHLKPYAYGVRNGMAILDSDKT |
| | | | LISLRTACAFIGALARNNARFMFVNTNPLFDEIFDQMTKKIHLYNPNQNTLWRTGG |
| | | | FLTNSRSPKKFRSRNKKLCFAPPQPPDCVVILDTERKSSVVLEADRLQIPVVAIVDS |
| | | | SMPLDIYKRIAYPVPANDSVQFVYLFCNLITKTFLAEQKRFAKHDSIAVDDDSSKIE |
| | | | NTEEAKRVEESEKVGVSPKDEVVVVPYESLAPISQDRAEAKELLEKLVVLKFNGAL |
| | | | GKEMGFNGPKSVIEVCKGSTVLDLIVKQIESLNSKYGCNVPLLLMNTAKTNDDTVK |
| | | | VVEKYPNSNIVTLNTSDGQASENEAYPSDHDMVFLSLMNGGTLDVLLSQGKEYIL |
| | | | VVGSDNVAAVVDPNILNHLIQNKLEYCMEVTPTTLFDTNNSILNSHQQKFQLAEIA |
| | | | RNSNEHLADKFKLTDTRSLWVNLRAIKRLVDTDALKIENYTVSKGGKNDKILSPKT |
| | | | AAGSAIQFFDHAIGINVPQSRYLPMNATSDLLLLQSDLYTSNNGVLVRNSARTNPL |
| | | | NPSIILGPEFGKVSDLLSRFKSFPSIVELDSLKVTGDVWFGADVTLKGRVNIVAKPG |
| | | | MKLEIPDRAVLHNKDISDPIDI |
| 725 | 6 | Que_a | EKLNSLKSSVAALPQISENEKNGFINLIARFLSGEAQHVDWSKIQTPTDEVVVPYD |
| | | | TLKPAPHDPAETKKLLDKLVVLKLNGGLGTTMGCTGPKSVIEVRNGLTFLDLIVIQI |
| | | | ENLNKQYGCNVPLLLMNSFNTHDDTQKIVEKYSGANVEIHTFNQSQYPRLVVEDF |
| | | | SPLPSKGVTGKDGWYPPGHGDVFPSLRNSGKLDLLLSQGKEYVFIANSDNLGAVV |
| | | | DLKILNHLVHNKNEYCMEVTPKTMADVKGGTLISYEGRVQLLEIAQVPDEHVNEFK |
| | | | SIEKFKIFNTNNLWANLKAIKRLVEADALKMEIIPNPKEVEGIKVLQLETAAGAAIRF |
| | | | FDNAIGNNVPRSRFLPVKATSDLLLVQSDLYTLEDGFVIRNKARTNPANPSIELGPE |
| | | | FKKVGNFLNRFKSIPSIVELDSLKVTGDVWFGANITLKGKVTIVAKPGAKLEIPDGA |
| | | | VLENKEINGPEDI |
| 726 | 7 | Amb_a | DDKVTVESAEATLKYNVAIKCATITPDEARMKEFTLKSMWKSPNGTIRNILNGTVF |
| | | | REPILCKNIPRLIPGWTKPICIGRHAFGDQYKATDAVIKGPGKLKMVFVPEGEGENT |
| | | | ELEVYNFTGAGGVALSMYNTDESITAFAEASMNTAYLKKWPLYLSTKNTILKKYDG |
| | | | RFKDIFQEVYEKNWKSKFEAAGIWYEHRLIDDMVAYALKSDGGYVWACKNYDGD |
| | | | VQSDFLAQGFGSLGLMTSVLVCPDGKTIEAEAAHGTVTRHYRVHQKGGETSTNSI |
| | | | ASIFAWTRGLAHRAKLDDNAKLLDFTEKLEAACIGCVESGKMTKDLALIIHGSKLS |
| | | | REHYLNTEEFIDAVADELKARLSSN |
| 727 | 7 | Amb_p | GDEMTRVFWESIKNKLIFPFLDLDIKYYDLGLLNRDATDDKVTVESAEATLKYNVAI |
| | | | KCATITPDEARMKEFTLKSMWKSPNGTIRNILNGTVFREPILCKNIPRLIPGWTKPI |
| | | | CIGRHAFGDQYKATDAVIKGPGKLKMVFVPEGEGENTELEVYNFTGAGGVALSMY |
| | | | NTDESITAFAEASMNTAYLKKWPLYLSTKNTILKKYDGRFKDIFQEVYEKNWKSKF |
| | | | EAAGIWYEHRLIDDMVAYALKSDGGYVWACKNYDGDVQSDFLAQGFGSLGLMTS |
| | | | VLVCPDGKTIEAEAAHGTVTRHYRVHQKGGETSTNSIASIFAWTRGLAHRAKLDD |
| | | | NAKLLDFTEKLEAACIGCVESGKMTKDLALITHGSKLSREHYLNTEEFIDAVADELK |
| | | | ARLSSN |
| 728 | 7 | Amb_p | SVNKMGFEKIKVANPIVEMDGDEMTRVFWESIKNKLIFPFLDLDIKYYDLGLLNRD |
| | | | ATDDKVTVESAEATLKYNVAIKCATITPDEARMKEFTLKSMWKSPNGTIRNILNGT |
| | | | VFREPILCKNIPRLIPGWTKPICIGRHAFGDQYKATDAVIKGPGKLKMVFVPEGEGE |
| | | | NTELEVYNFTGAGGVALSMYNTDESITAFAEASMNTAYLKKWPLYLSTKNTILKKY |
| | | | DGRFKDIFQEVYEKNWKSKFEAAGIWYEHRLIDDMVAYALKSDGGYVWACKNYD |
| | | | GDVQSDFLAQGFGSLGLMTSVLVCPDGKTIEAEAAHGTVTRHYRVHQKGGETST |
| | | | NSIASIFAWTRGLAHRAKLDDNAKLLDFTEKLEAACIGCVESGKMTKDLALITHGS |
| | | | KLSREHYLNTEEFIDAVADELKARLSSN |
| 729 | 7 | Amb_p | YNFTGAGGVAIAMYNTDESIRAFAEASMNTAYQKKWPLYLSTKNTILKKYDGRFK |
| | | | DIFQEVYEANWKSKYEAAGISYAVFC |
| 730 | 7 | Ant_o | CRRPPTHLPRLAPLRSRSPRQAAPAEAAMAFEKIKVANPIVEMDGDEMTRVFWQSI |
| | | | KDKLIFPFLDLDIKYYDLGVLHRDATDDKVTVEAAEATLKYNVAIKCATITPDEDRV |
| | | | KEFNLKQMWRSPNGTIRNIINGTVFREPIICKNVPKLVPGWTKPICIGRHAFGDQY |
| | | | RATDAVLKGPGKLRLVFEGKDETVDLEVFNFTGAGGVALAMYNTDESIQGFAEAS |
| | | | MATAYEKKWPLYLSTKNTILKKYDGRFKDIFQAVYEAGWKSKYEAAGIWYEHRLID |
| | | | DMVAYALKSEGGYVWACKNYDGDVQSDFLAQGFGSLGLMTSVLMCPDGKTIEAE |
| | | | AAHGTVTRHYRVHQKGGETSTNSIASIFAWTRGLAHRAKLDDNARLLDFTQKLED |
| | | | ACVGTVESGKMTKDLALLVHGSSKVTRGDYLNTEEFIDAVAAELQSRLAAN |
| 731 | 7 | Bet_v | GDEMTRVFWKSIKDKLIFPFVELDIKYFDLGLPHRDATDDKVTIESAEATLKYNVAI |
| | | | KCATITPDEDRVKEFKLKQMWKSPNGTIRNILNGTVFREPIICKNIPRLVPSWNKPI |
| | | | CIGRHAFGDQYRATDTVIKGAGKLKLVFVPEGKEEKTELEVYNFTGAGGVALSMYN |
| | | | TDESIRSFAEASMNTAYQKKWPLYLSTKNTILKKYDGRFKDIFQEVYVANWKSKYE |
| | | | AAGIWYEHRLIDDMVAYALKSDGGYVWACKNYDGDVQSDFLAQGFGSLGLMTS |
| | | | VLVCPDGKTIEAEAAHGTVTRHFRVHQKGGETSTNSIASIFAWSRGLAHRAKLDE |
| | | | NPRLLDFTEKLEAACIGVVESGKMTKDLALIIHGPKLAREHYLNTEEFIDAVAAELR |
| | | | ARLSA |
| 732 | 7 | Bet_v | KVRQKPRMLSPRATTTLRLSAMSGAKMLTSSCSSSASSSMALRSPRLHLQFPSSG |
| | | | PKLSNGVVLRGNRVSFASSSTRFAHASLRCYASSAGSDRVRVENPIVEMDGDEMT |
| | | | RIIWKMIKDKLIFPYLDLDIKYFDLGISNRDATDDKVTVESAEAALKYNVAVKCATI |
| | | | TPDETRVKEFGLKSMWRSPNGTIRNILNGTVFREPIICCNIPRIITGWKKPICIGRH |
| | | | AFGDQYRATDTVIEGPGKLKMVFVPEDGSTPVELDVFDFKGPGVALAMYNVDESI |
| | | | RVFAESSMSLAFAKKWPLYLSTKNTILKKYDGRFKDIFQEVYEEKWKQMFEENSI |
| | | | WYEHRLIDDMVAYAIKSEGGYVWACKNYDGDVQSDLLAQGFGSLGLMASVLLSS |
| | | | DGKTLEAEAAHGTVTRHFRLHQKGQETSTNSIASIFAWTRGLEHRGKLDKNERLL |
| | | | DFVHKLEAACIETVEMGKMTKDLAILIHGSKVSREHYLNTEEFIDAVAQNLEVKLRE |
| | | | PAPVTL |
| 733 | 7 | Bet_v | ATLKYNVAIKCATITPDEDRVKEFNLKQMWKSPNGTIRNILNGTVFREPIICKNIPR |
| | | | LVPGWTKPICIGRHAFGDQYRATDTVIKGSGKLKLVFVPDGHYEKKEFEVFNFTGA |
| | | | GGVALSMYNTDESIRSFAEASMNTAYQKKWPLYL |
| 734 | 7 | Bet_v | ETSTNSIASIFAWTRGLAHRAKLDGNARLLDFTENLEAACVGVVESGKMTKDLALL |
| | | | IHGPKVTRSKYLNTEEFIDHVAEELRARLFTKAKL |
| 735 | 7 | Bet_v | FNIKGSSCLSTFAPLSPSIFVFVPIPARLSLFRAFREKMALEKIKVANPIVEMDGDEM |
| | | | TRVFWKSIKDKLIFPFVELDIKYFDLGLPHRDATDDKVTIESAEATLKYNVAIKCATI |
| | | | TPDEDRVKEFKLKQMWKSPNGTIRNILNGTVFREPIICKNIPRLVPSWNKPICIGRH |
| | | | AFGDQYRATDTVIKGAGKLKLVFVPEGKEEKTELEVYNFTGAGGVALSMYNTDESI |
| | | | RSFAEASMNTAYQKKWPLYLSTKNTILKKYDGRFKDIFQEVYVANWKSKYEAAGI |
| | | | WYEHRLIDDMVAYALKSDGGYVWACKNYDGDVQSDFLAQGFGSLGLMTSVLVC |
| | | | PDGKTIEAEAAHGTVTRHFRVHQKGGETSTNSIASIFAWSRGLAHRAKLDENPRL |
| | | | LDFTEKLEAACIGVVESGKMTKDLALIIHGPKLAREHYLNTEEFIDAVAAELRARLS |
| | | | A |
| 736 | 7 | Cyn_d | PTPFHRRRRLPTRLAARPFPISEASCAVTAAMAFEKIKVANPIVEMDGDEMTRVFW |
| | | | KSIKDKLIFPFLDLDIKYYDLGILHRDATDDKVTVEAAEATLKYNVAIKCATITPDET |
| | | | RVKEFNLKHMWRSPNGTIRNIINGTVFREPIICKNVPRLVPGWTKPICIGRHAFGD |
| | | | QYRATDAVLKGPGKLKLVFEGKEEQIDLEVFNFTGAGGVALSMYNTDESVRAFAA |
| | | | ASMTMAYEKKWPLYLSTKNTILKKYDGRFKDIFQEVYEADWKSKFEAAGIWYEHR |
| | | | LIDDMVAYALKSEGGYVWACKNYDGDVQSDFLAQGFGSLGLMTSVLVCPDGKTI |
| | | | EAEAAHGTVTRHFRVHQKGGETSTNSIASIFAWTRGLAHRAKLDDNARLLDFAQK |
| | | | LEAACVGTVESGKMTKDLALLVHGSSKVTRSDYLNTEEFIDAVAAELQSRLAAN |
| 737 | 7 | Cyn_d | RLASPLARLPLPAARVFRGVSLRCYAAAAAVAEQHRIKVDNPIVEMDGDEMTRVIW |
| | | | KMIKDKLILPYLDVDLKYYDLGILNRDATDDRVTVESAEATREYNVAVKCATITPDE |
| | | | TRVKEFNLKSMWRSPNGTIRNILNGTVFREPILCKNIPRILSGWKHPICIGRHAFGD |
| | | | QYRATDMIIDGPGKLKMVFVPDGGAEPVELDVYDFKGPGVALSMYNVDESIRAFA |
| | | | ESSMAMAFSKKWPLYLSTKNTILKTYDGRFKDIFQEVYEENWRGKFEENSIWYEH |
| | | | RLIDDMVAYAVKSEGGYVWACKNYDGDVQSDFLAQGFGSLGLMTSVLLSSDGKT |
| | | | LESEAAHGTVMRHFRLHQKGQETSTNSIASIFAWTRGLEHRAKLDKNERLLDFTR |
| | | | KLESACVETVESGKMTKDLALLIYGPKVTREFYLNTEEFIDAVAHQLREKIQIPAAV |
| 738 | 7 | Cyn_d | SPTQSRPAMAFNKIKVANPVVEMDGDEMTRVFWKSIKDKLIFPFVDLDIKYFDLGL |
| | | | PHRDATDDKVTVEAAEATLKYNVAIKCATITPDEARVKEFNLKSMWRSPNGTIRNI |
| | | | LNGTVFREPIICQNIPRLVPGWTKPICIGRHAFGDQYRATDAVIKGPGKLKLVYEGK |
| | | | EEQVELEVFNFTGAGGVALAMYNTDESIRSFAEASMATAYEKKWPLYLSTKNTILK |
| | | | KYDGRFKDIFQEVYEAEWRSKYEAAGIWYEHRLIDDMVAYALKSEGGYVWACKN |
| | | | YDGDVQSDFLAQGFGSLGLMTSVLVCPDGKTMEAEAAHGTVTRHYRVHQKGGET |
| | | | STNSIASIFAWTRGLAHRAKLDDNARLLDFTQKLEAACIGAVESGKMTKDLALLVH |
| | | | GSSNVTRSHYLNTEEFIDAVAEELRSRLGANSNL |
| 739 | 7 | Cyn_d | GDEMTRVFWKSIKDKLIFPFLDLDIKYYDLGILHRDATDDKVTVEAAEATLKYNVAI |
| | | | KCATITPDETRVKEFNLKHMWRSPNGTIRNIINGTVFREPIICKNVPRLVPGWTKPI |
| | | | CIGRHAFGDQYRATDAVLKGPGKLKLVFEGKEEQIDLEVFNFTGAGGVALSMYNT |
| | | | DESVRAFAAASMTMAYEKKWPLYLSTKNTILKKYDGRFKDIFQEVYEADWKSKFE |
| | | | AAGIWYEHRLIDDMVAYALKSEGGYVWACKNYDGDVQSDFLAQGFGSLGLMTSV |
| | | | LVCPDGKTIEAEAAHGTVTRHFRVHQKGGETSTNSIASIFAWTRGLAHRAKLDDN |
| | | | ARLLDFAQKLEAACVGTVESGKMTKDLALLVHGSSKVTRSDYLNTEEFIDAVAAEL |
| | | | QSRLAAN |
| 740 | 7 | Fra_e | YSVMIRVLQTAMAGALNLSSSYSAFKNPSLVSISNPKLFNGVLFKTRLCFSTRISNA |
| | | | SIRCFTSNAIDKVRVQNPIVEMDGDEMTRAIWKMIKDKLIFPYLELDVKYFDLGILN |
| | | | RDATDDKVTVESAEATLKYNVAIKCATITPDETRVKEFGLKAMWRSPNGTIRNILN |
| | | | GTVFREPILCSNIPRIVPGWNKPICIGRHAFGDQYRATDAIIKGPGKLKMVFVPENG |
| | | | EGPMELDVYDFKGPGVALAMYNVDQSIRAFAESSMAMAFAKKWPLYLSTKNTILK |
| | | | KYDGRFKDIFQEVYEEKWKEQFEEHSIWYEHRLIDDMVAYAVKSDGGYVWACKN |
| | | | YDGDVQSDLLAQGFGSLGMMTSVLLSGDGKTLEAEAAHGTVTRHYRLYQKGQET |
| | | | STNSIASIFAWTRGLEHRAKLDGNEKLLDFSHKLEAACIETVESGKMTKDL |
| 741 | 7 | Fra_e | NFFHREKRSRFSQMDLEKIKVDNPIVEMDGDEMTRVIWKSIKEKLILPFLELDIKYF |
| | | | DLGLPHREATNDKVTIESAEATLKYNVAIKCATITPDEARVKEFSLKHMWKSPNGT |
| | | | IRNILNGTVFREPIMCKNVPRLVPGWTKPICIGRHAFGDQYRATDLVIQGAGKLKM |
| | | | VFVPNSGDGSTELEVYNFTGSGGVALSMYNTDESIRAFAEASMNTAFQKRWPLYL |
| | | | STKNTILKKYDGRFKDIFQEVYEREWKSKFESAGIWYEHRLIDDMVAYALKSEGGY |
| | | | VWACKNYDGDVQSDFLAQGFGSLGLMTSVLVCPDGKTIEAEAAHGTVTRHYRVH |
| | | | EKGGETSTNSIASIFAWSRGLAHRAKLDNNARLLDYTKKLEAACIASVESGKMTK |
| | | | DLAILIHGPKVTRSRYLNTEEFIEAVAEELKARLPKKAKL |
| 742 | 7 | Fra_e | REKMAFEKIKVANPIVEMDGDEMTRVIWQFIKDKLILPFVELDIKYYDLGLPHRDAT |
| | | | DDKVTIESAEAALKYNVAIKCATITPDEARVKEFGLKQMWKSPNGTIRNILNGTVF |
| | | | REPILCKNVPRLVPGWTKPICIGRHAYGDQYRATDTVIKGAGKLKLVFVPEGKDEK |
| | | | TEIEVFNFTGEGGVALSMYNTDESIRSFAEASMNTAYQKKWPLYLSTKNTILKKYD |
| | | | GRFKDIFQEVYELNWKSKFEEAGIWYEHRLIDDMVAYALKSEGGYVWACKNYDG |
| | | | DVQSDFLAQGFGSLGLMSSVLVCPDGKTIEAEAAHGTVTRHYRVHQKGGETSTN |
| | | | SIASIFAWTRGLAHRAKLDDNAKLLDFTEKLEAACIGVVESGKMTKDLALIIHGSKL |
| | | | GRDKYLNTEEFIDSVANELKAKLSC |
| 743 | 7 | Lol_p | KWIKDKLIFPFLDLDIKYYDLGLPNRDATGDKVTIESAEATLKYNVAIKCATVTPDE |
| | | | GRVKEFNLKAMWRSPNGTIRNILNGTVFREPIICKNVPRLVPGWTKPICIGRHAFG |
| | | | DQYRATDVIIRGPGKLKLVFDGVEEQIELDVFNFNGAGGVALSMYNTDESIRAFAE |
| | | | SSMNVAYQKRWPLYLSTKNTILKKYDGRFKDIFQENYEKNWRGKFEKAGIWYEHR |
| | | | LIDDMVAYALKSEGGYVWACKNYDGDVQSDLIAQGFGSLGLMTSVLVCPDGRTV |
| | | | EAEAAHGTVTRHYRVHQKGGETSTNSIASIFAWSTGLAHRAKLDDNKRLLDFTQK |
| | | | LEAACVGTVESGKMTKDLALLIHGPTVSRDKYLNTVEFIDAVADELKTSLSVKSKL |
| 744 | 7 | Lol_p | LNALAKLVTPFSLLPVPPSPAPPAPFPISQASSSAVAAMAFEKIKVANPIVEMDGDE |
| | | | MTRVFWQSIKDKLIFPFLDLDIKYYDLGVLHRDATDDKVTVEAAEATLKYNVAIKC |
| | | | ATITPDEDRVKEFNLKQMWRSPNGTIRNIINGTVFREPIICKNVPKLVPGWTKPICI |
| | | | GRHAFGDQYRATDAVLKGPGKLRLVFEGKDETVDLEVFNFTGAGGVALAMYNTDE |
| | | | SIQGFAAASMAIAYEKKWPLYLSTKNTILKKYDGRFKDIFQAVYEADWKSKYEAAG |
| | | | IWYEHRLIDDMVAYALKSEGGYVWACKNYDGDVQSDFLAQGFGSLGLMTSVLMC |
| | | | PDGKTIEAEAAHGTVTRHFRVHQKGGETSTNSIASIFAWTRGLAHRAKLDDNARL |
| | | | HDFTLKLEEACVGTVESGKMTKDLALLVHGSSKVTRGDYLNTEEFIDAVAAELKSR |
| | | | LAAN |
| 745 | 7 | Ole_e | RRKMAFEKIKVANPIVEMDGDEMTRVIWQFIKDKLIFPFVELDIKYYDLGLPHRDAT |
| | | | DDKVTIESAEATLKYNVAIKCATITPDEARVKEFGLKQMWKSPNGTIRNILNGTVF |
| | | | REPILCKNVPRLVPGWTKPICIGRHAFGDQYRATDTVIKGPGKLKLVFVPEGKDEK |
| | | | TEIEVFNFTGEGGVALSMYNTDESIRSFAEASMNTAYQKKWPLYLSTKNTILKKYD |
| | | | GRFKDIFQEVYESNWKSKFEEAGIWYEHRLIDDMVAYALKSEGEYVWACKNYDG |
| | | | DVQSDFLAQGFGSLGLMTSVLVCPDGKTIEAEAAHGTVTRHYRVHQKGGETSTN |
| | | | STASIFAWTRGLAHRAKLDDNDKLLDFTEKLEAACIGVVESGKMTKDLALIIHGSK |
| | | | LGRDKYLNTEEFIDAVADELKAKLSC |
| 746 | 7 | Ole_e | KTELEVYNFTGAGGVAIAMYNTDESIRAFAEASMNTAYQKKWPLYLSTKNTILKKY |
| | | | DGRFKDIFQEVYEANWKSKYEAAGISYAVFC |
| 747 | 7 | Pla_l | LAILLHGPKVQRAQYLNTEEFIDAVAQELRDRLPKRAKL |
| 748 | 7 | Pla_l | LVSLTVTVTPLLELRFRFCFLKFANKKPFLTNSVFFCCLYISINSFTAEIPIPISLTISIH |
| | | | PSSTLFTLLVTTQHKQTKPNPMAFEKIKVANPIVEMDGDEMTRVIWTFIKDKLIFPF |
| | | | VELDIKYFDLGLPHRDATDDKVTVESAEATLKYNVAIKCATITPDEARVKEFGLKSM |
| | | | WRSPNGTIRNILNGTVFREPILCKNVPRLVPGWTKPICIGRHAFGDQYRATDAVIK |
| | | | GPGKLKMVFVPEGKDESTEFEVYNFTGEGGVALAMYNTDESIRSFADASMNVAFE |
| | | | KKWPLYLSTKNTILKKYDGRFKDIFQEVYEASWKSKFEEAGIWYEHRLIDDMVAYA |
| | | | LKSEGGYVWACKNYDGDVQSDFLAQGFGSLGLMTSVLVCPDGKTIEAEAAHGTV |
| | | | TRHFRVHQKGGETSTNSIASIFAWTRGLAHRAKLDDNAKLLEFTEKLEAACIGVVE |
| | | | AGKMTKDLALILHGPKLSRDTYLNTEEFLDAVAEELKAKLSC |
| 749 | 7 | Poa_p | RRPPHLPRLAAFPISEASIAAADAMAFEKIKVANPIVEMDGDEMTRVFWQSIKEKLI |
| | | | FPFLDLDIKYYDLGVLHRDATDDKVTVEAAEATLKYNVAIKCATITPDEDRVKEFNL |
| | | | KQMWRSPNGTIRNIINGTVFREPIICKNVPKLVPGWTKPICIGRHAFGDQYRATDA |
| | | | VLKGPGKLRLVFEGKDETVDLEVFNFTGAGGVALAMYNTDESIQGFAEASMAIAYE |
| | | | KKWPLYLSTKNTILKKYDGRFKDIFQAVYEADWKSKYEAAGIWYEHRLIDDMVAY |
| | | | ALKSEGGYVWACKNYDGDVQSDFLAQGFGSLGLMTSVLMCPDGKTIEAEAAHGT |
| | | | VTRHFRVHQKGGETSTNSIASIFAWTRGLAHRAKLDDNARLLDFTQKLEDACVGT |
| | | | VESGKMTKDLALLVHGSSKVTRGDYLNTEEFIDAVAAELQSRLAAN |
| 750 | 7 | Que_a | TAKQRLTIHQYKKSPQHLLISPSTIIARHQPLFVSLTHSRSLFKKMAFEKIKVANPIV |
| | | | EMDGDEMTRVFWKSIKDKLIFPFVDLDIKYFDLGLPYRDATDDKVTIESAEATLKY |
| | | | NVAIKCATITPDEARVKEFGLKQMWKSPNGTIRNILNGTVFREPIICKNVPRLVPG |
| | | | WTKPICIGRHAFGDQYRATDTVIKGAGKLKLVFVPEGKDEKTELEVYNFTGAGGVA |
| | | | IAMYNTDESIRAFAEASMNTAYQKKWPLYLSTKNTILKKYDGRFKDIFQEVYEANW |
| | | | KSKYEAAGIWYEHRLIDDMVAYAVKSEGGYVWACKNYDGDVQSDFLAQGFGSLG |
| | | | LMTSVLVCPDGKTIEAEAAHGTVTRHYRVHQKGGETSTNSIASIFAWSRGLSHRA |
| | | | KLDDNARLLDFTEKLEAACVGTVESGKMTKDLALLIHGSKVTREQYLSTEEFIDAV |
| | | | ATELKARLSA |
| 751 | 7 | Que_a | RTTALRLSAMSSGAKMLASTSSSSSSFLAVRNPSFSSTSTRLFNGGVLHRGNKNR |
| | | | VSFSSATRFANASLRCYASSAGFDRVQVQNPIVEMDGDEMTRIIWRMIKDKLIFPY |
| | | | LDLDIKYFDLGILNRDATDDRVTVESAEAALKYNVAVKCATITPDETRVKEFGLKS |
| | | | MWRSPNGTIRNILNGTVFREPILCRNIPKIIPGWKKPICIGRHAFGDQYRATDTVIE |
| | | | GPGKLKMVFVPDDGKTPVELDVFNFKGPGIALAMYNVDESIRAFAESSMTLAFAKK |
| | | | WPLYLSTKNTILKKYDGRFKDIFQEVYEEKWKQKFEENSIWYEHRLIDDMVAYVVK |
| | | | SEGGYVWACKNYDGDVLSDLLAQGFGSLGLMSSVLLSSDGKTLEAEAAHGTVTR |
| | | | HFRLHQKGQETSTNSIASIFAWTRGLEHRAKLDENEKLREFVHKLEAACIETVETG |
| | | | KMTKDLAILIHGSKVSREHYLNTEEFIDAVAQNLEAKIQEPVLA |
| 752 | 7 | Que_a | RTTALRLSAMSSGAKMLASTSSSSSSFLAVRNPSFSSTSTRLFNGGVLHRGNKNR |
| | | | VSFSSATRFANASLRCYASSAGFDRVQVQNPIVEMDGDEMTRIIWRMIKDKLIFPY |
| | | | LDLDIKYFDLGILNRDATDDRVTVESAEAALKYNVAVKCATITPDETRVKEFGLKS |
| | | | MWRSPNGTIRNILNGTVFREPILCRNIPKIIPGWKKPICIGRHAFGDQYRATDTVIE |
| | | | GPGKLKMVFVPDDGKTPVELDVFNFKGPGIALAMYNVDESIRAFAESSMTLAFAKK |
| | | | WPLYLSTKNTILKKYDGRFKDIFQEVYEEKWKQKFEENSIWYEHRLIDDMVAYVVK |
| | | | SEGGYVWACKNYDGDVLSDLLAQGFGSLGLMSSVLLSSDGKTLEAEAAHGTVTR |
| | | | HFRLHQKGQETSTNSIASIFAWTRGLEHRAKLDENEKLREFVHKLEAACIETVETG |
| | | | KMTKDLAILIHGSKVSREHYLNTEEFIDAVAQNLEAKIREPVLA |
| 753 | 7 | Que_a | GRHAFGDQYRATDIVIQESGKLKLVFVPNGHNEKKEFEVFNFTGAGGVALSMYNT |
| | | | DESIRAFAEASMNTAYQKKWPLYLSTKNTILKKYDGRFKDI |
| 754 | 7 | Que_a | GRFKDIFQEVYETQWKSKFEAAGIWYEHRLIDDMVAYAMKSEGGYVWACKNYDG |
| | | | DVQSDFLAQGFGSLGMMTSVLVCPDGKTIESEAAHGTVTRHYRVHQKGGETSTN |
| | | | SIASIFAWTRGLAHRAKLDSNARLLDFTEKLEAACVGTVESGKMTKDLALLIHGPK |
| | | | VTRSQYLNTEEFIDAVAEELRARLSTRAKL |
| 755 | 7 | Que_a | GDEMTRVFWKSIKDKLIFPFVDLDIKYFDLGLPYRDATDDKVTIESAEATLKYNVAI |
| | | | KCATITPDEARVKEFGLKQMWKSPNGTIRNILNGTVFREPIICKNVPRLVPGWTKPI |
| | | | CIGRHAFGDQYRATDTVIKGAGKLKLVFVPEGKDEKTELEVYNFTGAGGVAIAMYN |
| | | | TDESIRAFAEASMNTAYQKKWPLYLSTKNTILKKYDGRFKDIFQEVYEANWKSKYE |
| | | | AAGIWYEHRLIDDMVAYAVKSEGGYVWACKNYDGDVQSDFLAQGFGSLGLMTS |
| | | | VLVCPDGKTIEAEAAHGTVTRHYRVHQKGGETSTNSIASIFAWSRGLSHRAKLDD |
| | | | NARLLDFTEKLEAACVGTVESGKMTKDLALLIHGSKVTREQYLSTEEFIDAVATELK |
| | | | ARLSA |
| 756 | 8 | Amb_a | RGHNVFWDDPASQMAWVNKLSKEQLKEAMDKRVKSVVNKYKGQVIHWDVNNE |
| | | | NVHFNFFETKFGPDASTKIFQQVHQIDPDVILFLNDFNTLEQPGDTNATPDKYLKK |
| | | | FHEIRAGNPNAKMAIGLESHFDVPNIPHMRAVLDKMATAGVPIWLTEVDVAGTDP |
| | | | NQAHYLEQILREGYSHPAVQGIVMWASWTPKGCYRMCLTNNQFQNLPVGDTVDK |
| | | | LIKEWKTHASGTTAADGSFQTTLAHGDYKVTVTH |
| 757 | 8 | Amb_a | EVVAKERKKKVKITVECGGKPLPNAELSVQWVAKGFPLGNAMTKEILDMPEYEEW |
| | | | FTKRFKWATMENAMKWYSTEYNEGQEGFEVADKMLALAEKHNISVRGHNVFWD |
| | | | DQSHQMPWVEKLSVGKLKAAVAKHLKAVVSRYAGKVIHWDVVNENLHFSFFEDK |
| | | | LGKDASGEIFKEVAKLDSKPILFMNEFNTIEEPCDLAPLPTKYLAKLKQIQSYPGN |
| 758 | 8 | Amb_p | GYNERLSIGLEGHFQNVNIPYMRSAIDKVASSGLPIWITEVDVQTGPNQAMFFDQ |
| | | | VLREAHAHPSIHGIVVWSAWSPQGCYRMCLTDNNFNNLPTGDVVDRIIREFFSVE |
| | | | LTATTDVNGFYETSLIHGDYEVSFAH |
| 759 | 8 | Bet_v | VRIQAVDGQGNPISNTTVLLEQKKLSFPFGTAINKNILTNSDYQKWFTSRAFTVTV |
| | | | FENEMKWYANEPSQGEEEYDDADALLEFANQHGLDVRGHTV LWEDPQMIQGWV |
| | | | SSLSSSDLAEAVKKRINSIMSKYKGQVIAWDVVNENMHHSFFEDRLGGDASASFY |
| | | | NRAQKIDGSTTLFLNEYNTIEDNRDGSSNPHAYLQKLEEIQGFPGNSDLKMGIGLQ |
| | | | GHFSYPPDLSYVRASIDTLASTGLPIWITELDVKSSVGDEQTQAEYLEQILRELHAH |
| | | | PNVDGIMLWTAWLPSGCYRMCLTDNNFDNLATGDVVDKLMEEWGSKAFAGKTD |
| | | | ANGYFEASLFHGEYEVKISHPTEPSSDLSQSFVV |
| 760 | 8 | Cyn_d | FSFDEWDAHTRRSGDKTRRRTVRLVAKGADAKPMANANVSIELLRLGFPFGNTMT |
| | | | AEILSLPAYEKWFTSRFTHATFENEMKWYSTEWSQNQENYDVPDRMLKMAQKYG |
| | | | IKVRGHNVFWDDQNSQMRWVKPLNLDQLKSAMQKRLKNVVTRYAGKVIHWDVV |
| | | | NENLHFNFFESKLGSSASAQIYNQVGQIDRNAILFMNEFNVLEQPGDPNAVPSKYI |
| | | | AKMNQIRSYPGNSGLKMGVGLESHFSTPNIPYMRSTLDTLAKLKLPMWLTEVDVV |
| | | | KNPNQVKYLEQVLREGYAHPNVDGIIMWAAWHAKGCYVMCLTDNNFKNLPVGDL |
| | | | VDKLITEWKTHRTVATTDENGAVVLDLPLGEYKFTVHHPSLSGTTVDLMTVDGAS |
| | | | S |
| 761 | 8 | Que_a | IWVDSISLQPFTQEQWKSHQDQSIEKARKRKVRIHVVDEQGNPLPNASISIIQKK |
| | | | VSFPFGTAINKNILTNKAYQNWFSSRFTVTVFEDEMKWYTTEPSPGQEDYTAADAL |
| | | | FQFAKKHSIPVRGHNVLWDDPSKVQGWVSSLSPTDLAVAVKKRINSVMSRYKGQ |
| | | | VIAWDVVNENLHFSVFEDKLGSTASATFFNAAQEIDGTTTLFMNDYNIIEDSRDRS |
| | | | STPDKYIQKLKQIQRFPRNNNLKQGIGLESHFSIAPDLAYMRSSIDTLASTGLPVWI |
| | | | TELDIASALGQQVQARYLEQVLRELYAHPKINGIIMWSAWKPGGCYQMCLTDNSF |
| | | | NNLPTGNVVDKLLREWRSSLKGTADGDGFFEASLSHGDYELKISHPNVTSSSLAQ |
| | | | SQRFEVSSAD |
| 762 | 9 | Amb_a | PLEVQVYAEHAYQTTVARFSPNGEWVASADVSGMVRIWGTHNGFVLKNEFRVLS |
| | | | GRIDDLQWSGDGMRIVASGDGKGKSFVRAFMWDSGSNVGEFDGHSRRVLSCAF |
| | | | KPTRPFRIVTCGEDFLINFYEGPPFKFKLSHRDHSNFVNCVRFSPDGSKFITVSSDK |
| | | | QGLLYDGKTAEKKGELSSEDGHKGSIYAVSWSPDSKQVLTVSADKTAKIWTISED |
| | | | FNGTVAKTLCCPGSGGVEDMLVGCLWQNDYIVTVSLGGTIYLYSASDLDKDPTIL |
| | | | CGHMKNITSLVVLKTNPETILSSSYDGLISKWIRGVGYNGKLERKDKNQIKCLTAV |
| | | | DEEIISSGFDNKIWRIPLTGDECGDANIVDIGSQPIDLSVAIHKHELALISIEKGVVL |
| | | | LNGTQVLSTIDLGFTVSACAIAPDGTEAIVGGQDGKLHIYSVNGDSLTEEAVLEKH |
| | | | RGAITVIHYSPDVSMFASADANREAVVWDRVTREVKLKNMLYHTARINSLAWSPD |
| | | | NTMVATGSLDTCVIVYEISKPASSRITIKGANLGGVYAVSFVDDNTVVSSGEDACI |
| | | | RLWQISPQ |
| 763 | 9 | Amb_p | MANLVETYACIPSTERGRGILISGDPKTNAFLYCNGRSVIIRYLDRPLEVQVYAEHA |
| | | | YQTTVARFSPNGEWVASADVSGMVRIWGTHNGFVLKNEFRVLSGRIDDLQWSG |
| | | | DGMRIVASGDGKGKSFVRAFMWDSGSNVGEFDGHS |
| 764 | 9 | Amb_p | EFDGHSRRVLSCAFKPTRPFRIVTCGEDFLINFYEGPPFKFKLSHRDHSNFVNCVRF |
| | | | SPDGSKFITVSSDKQGLLYDGKTAEKKGELSSEDGHKGSIYAVSWSPDSKQVLTV |
| | | | SADKTAKIWTISEDFNGTVAKTLCCPGSGGVEDMLVGCLWQNDYIVTVSLGGTIY |
| | | | LYSASDLDKDPTILCGHMKNITSLVVLKTNPETILSSSYDGLISKWIRGVGYNGKLE |
| | | | RKDKNQIKCLTAVDEEIISSGFDNKIWRIPLTGDECGDANIVDIGSQPIDLSVAIHK |
| | | | HELALISIEKGVVLLNGTQVLSTIDLGFTVSACAIAPDGTEAIVGGQDGKLHIYSVN |
| | | | GDSLTEEAVLEKHRGAITVIHYSPDVSMFASADANREAVVWDRVTREVKLKNMLY |
| | | | HTARINSLAWSPDNTMVATGSLDTCVIVYEISKPASSRITIKGANLGGVYAVSFVD |
| | | | DNTVVSSGEDACIRLWQISPQ |
| 765 | 9 | Bet_v | MPQLAETYASVPTTERGRGILISGHPKSNTVLYTNGRSVIMINLDNPLDVSVYAEH |
| | | | AYPATVARYSPNGEWIASADVSGTVRIWGTRNEFVLKKEFKVLSGRIDDLQWSAD |
| | | | GQRIVACGDGKGKSLVRAFMWDSGTNVGEFDGHSRRVLSCAFKPTRPFRIVTCG |
| | | | EDFLVNFYEGPPFKFKQSHRDHSNFANCVRYSPDGNKFISVSSDKKGIIYDGKSG |
| | | | EKIGELSSEDGHKGSIYAVSWSPDGKQVFTASADKSAKVWEISEDGTGKVKKTLT |
| | | | SPVSGGVDDMLVGCLWQNDHLVTVSLGGTISLFSVTDLDKAPLLLSGHMKNVNS |
| | | | LAVLKSDPKVILSSSYDGLIIKWIQGIGYSGRLQRKENSQIKCFAAVEEEIVTSGFD |
| | | | NKIWRVSVHGDQCGDADSVDIGTQPKDLSLALLSPELALVSTDSGVVLLRGTKVL |
| | | | STINLGFSVTASAIAPDGSEAIVGGQDGKLHIYSITGDTLKEEAVLEKHRGAVSVIR |
| | | | YSPDVSMFASGDVNREAVVWDRVSREVKLKNMLYHTARINCLAWSPDSSIVATG |
| | | | SLDTCVIIYEVGKPASSRSTIKGAHLGGVYGLAFTDQYSVVSSGEDACVRVWRLTP |
| | | | E |
| 766 | 9 | Cyn_d | MAQLAETYACSPATERGRGILLAGDPKTDTIAYCTGRSVIIRRLDAPLDAWAYQDH |
| | | | AYPTTVARFSPNGEWVASADASGCVRVWGRYGDRALKAEFRPLSGRVDDLRWSP |
| | | | DGLRIVVSGDGKGKSFVRAFVWDSGSTVGEFDGHSKRVLSCDFKPTRPFRIVTCG |
| | | | EDFLANFYEGPPFKFKHSIRDHSNFVNCIRYSPDGSKFITVSSDKKGLIYDGKTGE |
| | | | KIGELSSEGSHTGSIYAVSWSPDSKQVLTVSADKTAKVWDIMEDATGKLNRTLVC |
| | | | TGIGGVDDMLVGCLWQNDHLVTVSLGGTFNVFSASNPDQEPVTFAGHLKTISSLV |
| | | | LFPQSNPRTILSTSYDGVIMRWIQGVGYGGRLMRKNNTQIKCFAAVEEELVTSGY |
| | | | DNKIFRIPLNGDQCGDAESVDVGGQPNAVNLAIQKPEFALVTTDSGIILLHNSKVI |
| | | | STTKVDYTITSSSVSPDGSEAVVGAQDGKLRIYSISGDTLTEEAVLEKHRGAITSIH |
| | | | YSPDVSMFASADANREAVVWDRATREVKLKNMLYHTARINCLAWSPDSRLVATG |
| | | | SLDTCAIVYEIDKPAASRITIKGAHLGGVRGLTFVDNDTLVTAGEDACIRDWKLVQ |
| | | | Q |
| 767 | 9 | Que_a | MSQLAETYACVPTTERGRGILISGNPKSNTITYTNGRSVIMINLDNPLDVSVYAEHA |
| | | | YPATVARYSPNGEWIASADVSGTVRIWGTRNEFVLKKEFKVLSGRIDDLQWSPDG |
| | | | MRIVACGDGKGKSLVRAFMWDSGTNVGEFDGHSRRVLSCAFKPTRPFRIVTCGE |
| | | | DFLVNFYEGPPFKFKLSHRDHSNFVNCVRFSPDGSKFISVSSDKKGLIYDAKTAEK |
| | | | MGELSSEDGHKGSIYAVSWSPDGKQVLTASADKSAKVWEISEDGNGKVKKTLAS |
| | | | PGSGGVDDMLVGCLWQNDHLVTVSLGGTISLFSATDLDKAPLLLSGHMKNVTSL |
| | | | AVLKSDPKMIWSTSYDGLIIKWIQGIGYSGRLQRKENSQIKCFAAVEEEIVTSGFD |
| | | | NKIWRISVHGDQCGDADSVDIGSQPKDLNLALLSPDLALVSTDSGVVLLRGAKIV |
| | | | STISLGFTVTASAISPDGTEAIVGGQDGKLHIYSVTGDTLNEEAVLEKHRGAISVIC |
| | | | YSPDVSMFASGDVNREAIVWDHDSREVKLKNMLYHTARINCLAWSPDSSMIATG |
| | | | SLDTCVIIYEVDKPASSRLTIKGAHLGGVYGLAFTDQYSVVSSGEDACVRVWKLTP |
| | | | Q |
| 768 | 10 | Amb_a | MANFTVNRVVTSPIEGQKPGTSGLRKKVKVFTQPHYLHNFVQSTFNALSAEKVKG |
| | | | STLVVSGDGRYYSKDAIQIIIKMAAANGVRRVWVGQNGLLSTPAVSAVVRERVGA |
| | | | DGSKANGAFILTASHNPGGPNEDFGIKYNMGNGGPAPEGITDKIFENTKTIKEYFI |
| | | | AEGLPDVDISAIGVSNFSGPGGQFDVDVFDSASDYVKLMKSIFDFQSIKKLITSPQ |
| | | | FSFCFDALHGVGGAYAKRMFVEELGAKESSLLNCVPKEDFGGGHPDPNLTYAKEL |
| | | | VARMGLGTNPDSNPPEFGAAADGDADRNMILGKRFFVTPSDSVAIIAANAVQAIP |
| | | | YFSSGLKGVARSMPTSAALDVVAKSLNLKFFEVPTGWKFFGNLMDAGLCSICGEE |
| | | | SFGTGSDHIREKDGIWAVLAWLSILAHKNKDNLDGGKLVTVEDIVKQHWATFGR |
| | | | HYYTRYDYENVDAGAAKEVMAHLVDLQSSISGVNTTI |
| 769 | 10 | Amb_a | AANAVEAIPYFSDGLKGVARSMPTSAALDVVAEALNLKFFEVPTGWKFFGNLMDA |
| | | | GLCSVCGEESFGTGSDHVREKDGIWAVLAWLSILAQKNKEKLNGEKLVTVEDIVR |
| | | | QHWATYG |
| 770 | 10 | Amb_p | SIFDFQSIKKLITSPQFSFCFDALHGVGGAYAKRMFVEELGAKESSLLNCVPKEDFG |
| | | | GGHPDPNLTYAKELVARMGLGTNPDSNPPEFGAAADGDADRNMILGKRFFVTPSD |
| | | | SVAIIAANAVQAIPYFSSGLKGVARSMPTSAALDVVAKSLNLKFFEVPTGWKFFGN |
| | | | LMDAGLCSICGEESFGTGSDHIREKDGIWAVLAWLSILAHKNKDNLDGGKLVTVE |
| | | | DIVKQHWATFGRHYYTRYDYENVDAGAAKEVMAHLVDLQSSISGVNTTIKGIRSD |
| | | | VADVVSADEFEYKDPVDGSVSKNQGIRYLFEDGSRLVFRLSGTGSEGATIRLYIEQ |
| | | | YEKDSSKTGRDSQEALAPLVDVALKLSKMLEYTGRSAPTVIT |
| 771 | 10 | Amb_p | GAFILTASHNPGGPNEDFGIKYNMGNGGPAPEGITDKIFENTKTIKEYFIAEGLPDV |
| | | | DISAIGVSNFSGPGGQFDVDVFDSASDYVKLMKSIFDFQ |
| 772 | 10 | Bet_v | MVVFKVARVESTPFDGQKPGTSGLRKKVKVFIQPNYLENFVQSTFNALTPEKVRGA |
| | | | TLVVSGDGRYYSKDAIQIIIKMAAANGVRRVWVGQNGLLSTPAVSAVIRERVAVD |
| | | | GSRASGAFILTASHNPGGPHEDFGIKYNMENGGPAPEGLTDKIYENTKTIKEYFIAE |
| | | | DLPDVDITTTGVTRFGGPEGQFDVDVFDSASDYVKLMKSIFDFELIRKLLSSPKFTF |
| | | | CYDALHGVAGAYAKRIFVEELGAQESSLLNCTPKEDFGGGHPDPNLTYAKELVAR |
| | | | MGLGKSNSQDEVPEFGAAADGDADRNMILGKRFFVTPSDSVAIIAANAVQAIPYF |
| | | | SAGLKGVARSMPTSAALDVVAKHLNLKFFEVPTGWKFFGNLMDAGLCSVCGEESF |
| | | | GTGSDHIREKDGIWAVLAWLSILAHKNKENLGGEKLVTVEDIVRQHWATYGRHY |
| | | | YTRYDYENVDAAAAKALMAYLVKLQSSLSEVNEIVKGVRSDVAKVVDADEFEYKD |
| | | | PVDGSISKHQGIRYLFEDGSRLVFRLSGTGSEGATIRLYIEQYEKDPSKIGRDSQE |
| | | | ALAPLVEVALKLSKMQEFTGRGAPTVIT |
| 773 | 10 | Cyn_d | MVLFTVTKKATTPFEGQKPGTSGLRKKVTVFQQPNYLQNFVQATFNALPADQVKG |
| | | | ATIVVSGDGRYFSKDAVQIITKMAAANGVRRVWVGQNSLMSTPAVSCVIRDRVG |
| | | | SDGSKATGAFILTASHNPGGPTEDFGIKYNMGNGGPAPESVTDKIFSNTKTISEYLI |
| | | | SEDLPDVDISVVGVTSFSGPEGPFDVDVFDSSVDYIKLMKSIFDFEATKNLVTSPKF |
| | | | TFCYDALHGVAGAYAKQIFVEELGADESSLLNCVPKEDFGGGHPDPNLTYAKELVE |
| | | | RMGLGKSTSNVEPPEFGAAADGDADRNMILGKRFFVTPSDSVAIIAANAVQSIPYF |
| | | | SSGLKGVARSMPTSAALDVVAKNLNLKFFEVPTGWKFFGNLMDAGMCSICGEESF |
| | | | GTGSDHIREKDGIWAVLAWLSILAFKNKDNLRGDKLVSVEDIVRQHWATYGRHY |
| | | | YTRYDYENVDAGAAKELMANLVSMQSSLSDVNKLIKEIRSDVSDVVAADEFEYKD |
| | | | PVDGSVSKHQGIRYLFGDGSRLVFRLSGTGSVGATIRVYIEQYEKDSSKIGRESQ |
| | | | DALAPLVDVALKLSKMQEYTGRSAPTVIT |
| 774 | 10 | Que_a | MVFKVSRVETKPIDGQKPGTSGLRKKVKVFIQPHYLHNFVQSTFNALTPEKVRGAT |
| | | | LVVSGDGRYYSKDAIQIITKMSAANGVRRVWVGQNGLLSTPAVSAVIRERVGVDG |
| | | | SRASGAFILTASHNPGGPNEDFGIKYNMENGGPAPEGITDKIYENTKTIKEYFISED |
| | | | LPDVDISAVGVTSFAGPEGQFDVEVFDSASDYVKLMKSIFDFESIRKLISSPKFTFC |
| | | | YDALHGVAGAYAKRIFVEELGAQESSLLNCTPKEDFGGGHPDPNLTYAKELVARM |
| | | | GLGKSSSQGEPPEFGAAADGDADRNMILGKRFFVTPSDSVAIIAANAVESIPYFSA |
| | | | GLKGVARSMPTSAALDVVAKHLNLKFFEVPTGWKFFGNLMDAGLCSVCGEESFGT |
| | | | GSDHIREKDGIWAVLAWLSILAHKNKENLGEEKLVSVEDIVRQHWTTYGRHYYTR |
| | | | YDYENVDAGAAKELMAYLVKLQSSLPEVNEIVKGTRSDVSKVINADEFEYKDPVD |
| | | | GSISKHQGIRYLFEDGSRLVFRLSGTGSEGATIRLYIEQYEKDPSKTGRDSQDALA |
| | | | PLVEVALKLSKMQEFTARTAPTVIT |
| 775 | 11 | Amb_a | QLQLLLKGASERGAKRIRVHVLTDGRDVVDGSSVGFAETLEKDLAELRGKGIDAQ |
| | | | VASGGGRMYVTMDRYENDWEVVKRGWDAQVLG |
| 776 | 11 | Amb_a | MGSTGFSWKLADHPKLPKGKLLAMIVLDGWGEASPDKFNCIHVADTPTMDSLKN |
| | | | GAPDKWRLVRAHGTAVGLPTEDDMGNSEVGHNALGAGRIYAQGAKLVDLALASG |
| | | | KIYEDEGFNYIKESFATNTLHLIGLMSDGGVHSRLDQLQLLLKGASQHGAKRIRVH |
| | | | VLTDGRDVLDGSSVGFAEILEAELSDLRSKGIDAQVASGGGRMYVTMDRYENDW |
| | | | EVVKRGWDAQVLGEAPHKFKNVVEAIKTLREAPGANDQYLPPFVIVDDSGKSVGP |
| | | | IVDGDAVVTFNFRADRMTMLAQALEYENFDKFDRVRVPKIRYAGMLQYDGELKLP |
| | | | SHYLVSPPLIERTSGEYLVHNGVRTFACSETVKFGHVTFFWNGNRSGYFNSELEEY |
| | | | VEIPSDSGITFNVQPKMKALEIGEKARDAILSGRFDQVRVNIPNGDMVGHTGDVE |
| | | | ATVVACKAADEAVKMIIDAVEQVGGIYVVTADHGNAEDMVKRNKKGEPILKDGEV |
| | | | QILTSHTLQPVPIAIGGPGLAAGVKFRKDV |
| 777 | 11 | Amb_p | EKFDKFDRVRFPKIRYAGMLQYDGELKLPSHYLVSPPLIERTSGEYLVHNGIRTFAC |
| | | | SETVKFGHVTFFWNGNRSGYFNKELEEYVEIPSDSGITFNVQPKMKALEIGEKARD |
| | | | AILSRKFDQVRVNIPNGDMVGHTGDIEATIVACKAADQAVKMILDAIEQVGGIYLV |
| | | | TADHGNAEDMVKRNKKGEPLLKDGEVQILTSHTLQPVPIAIGGPGLAAGVKFRKD |
| | | | VPSGGLANVAATVMNLHGFVAPDDYETTLIEVVD |
| 778 | 11 | Amb_p | DQLQLLLRGASQHGAKRIRVHVLTDGRDVLDGSSVGFAETLEAELSDLRSKGIDA |
| | | | QVASGGGRMYVTMDRYENDWEVVKRGWDAQVLGEAPHKFKNVVEAIKTLREAP |
| | | | GANDQYLPPFVIVDDSGKAVGPVVDGDAVVTFNFRADRMTMLAQALEYEKFDKFD |
| | | | RVRVPKIRYAGMLQYDGELKLPSHYLVSPPLIDRTSGEYLVNNGVRTFACSETVKF |
| | | | GHVTFFWNGNRSGYFNSELEEYVEIPSDSGITFNVQPKMKALEIGEKARDAILSGK |
| | | | FDQVRVNIPNGDMVGHTGDVEATVVACKAADEAVKMILDAVEQVGGIYVVTADH |
| | | | GNAEDMVKRNKKGEPLLKDGEVQILTSHTLQPVPIAIGGPGLAAGVKFRKDVPSG |
| | | | GLANVAATVMNLHGFVAPDDYETTLIEVVD |
| 779 | 11 | Bet_v | MGTSGFSWKLPEHPKLPKGKTVAVVVLDGWGEAKPDQYNCIHVAETPTMDSLKQ |
| | | | GAPEKWRLVRAHGKAVGLPTEDDMGNSEVGHNALGAGRIFAQGAKLVDSALASG |
| | | | KIYEGEGFKYIKECFENGILHLIGLLSDGGVHSRLDQLQLLLKGASERGAKRIRVHI |
| | | | LTDGRDVLDGSSVGFVETLENDLAKLREKGVDAQIASGGGRMYVTMDRYENDWE |
| | | | VIKRGWDAHVLGEAPYKFKSAVEAVKKLREELKVSDQYLPPFVIVDDNGKPVGPIV |
| | | | DGDAVVTINFRADRMVMIAKALEYENFDKIDRVRFPKIRYAGMLQYDGELKLPSHY |
| | | | LVEPPEIERTSGEYLVHNGVRTFACSETVKFGHVTFFWNGNRSGYFNSELEEYVEIP |
| | | | SDSGITFNVQPKMKALEIAEKTRDAILSGKFDQVRVNLPNGDMVGHTGDIEATVV |
| | | | ACKAADEAVKMILDAIEQVGGIYVVTADHGNAEDMVKRNKSGQPLLDKNGNLQV |
| | | | LTSHTLQPVPIAIGGPGLASGVRFRKDLPDGGLANVAATVINLHGFEAPSDYEPTLI |
| | | | ELVD |
| 780 | 11 | Cyn_d | SAMATAWTLPDHPKLPKGKTVAVVVLDGWGEANPDQYNCIHVAQTPVMDSLKNG |
| | | | APERWRLVKAHGTAVGLPSDDDMGNSEVGHNALGAGRIFAQGAKLVDSALASGK |
| | | | IYDGEGFNYIKESFENGTLHLIGLLSDGGVHSRLDQVQLLLKGASERGAKRIRVHIL |
| | | | TDGRDVLDGSSVGFVETLENDLSELREKGIDAQIASGGGRMNVTMDRYENDWGV |
| | | | VKRGWDAQVLGEAPHKFKSAVEAVKTLRAVPDANDQYLPPFVIVDESGKAVGPIV |
| | | | DGDAVVTFNFRADRMVMLAKALEYADFDKFDRVRVPKIRYAGMLQYDGELLLPKR |
| | | | YLVSPPEIDRTSGEYLVKNGVRTFACSETVKFGHVTFFWNGNRSGYFDESKEEYVE |
| | | | VPSDSGITFNVKPKMKAVEIAEKARDAILSGKFDQIRVNLPNGDMVGHTGDIEATV |
| | | | VACKAADEAVKIILDAVEQVGGIYLVTADHGNAEDMVKRNKAGKPLLDKSGAIQIL |
| | | | TSHTLQPVPVAIGGPGLHPGVKFRSDIETPGLANVAATVMNLHGFEAPADYEPTLIE |
| | | | VAD |
| 781 | 11 | Que_a | MGSSWKLADHPKLPKGKTVAVVVLDGWGEAKPDQYNCIHVAETPTMDSLKKGDP |
| | | | DKWRLVKAHGSAVGLPTEDDMGNSEVGHNALGAGRIFAQGAKLVDLALESGKIY |
| | | | DGEGFKYISECFEKGTLHLIGLLSDGGVHSRLDQLLLLLKGSSERGAKRIRVHILTD |
| | | | GRDVLDGSSVGFVETLENYLAELRGKGVDAQIASGGGRMYVTMDRYENDWEVVK |
| | | | RGWDAQVLGEAPFKFRNAVEGVKQLRQAPKASDQYLPPFVIADESGKPVGPIVDG |
| | | | DAVVTINFRADRMVMVAKAFEYEDFDKFDRVRVPKIRYAGMLQYDGELKLPSHYL |
| | | | VSPPEIDRTSGEYLVHNGIRTFACSETVKFGHVTFFWNGNRSGYFNEELEEYVEIPS |
| | | | DSGITFNVQPKMKALEIGEKVRDAILSGKFDQVRVNIPNGDMVGHTGDIEATVVA |
| | | | CKAADEAVKMILDAIEQVGGIYVVTADHGNAEDMVKRNKTGQPQLDKGGKIQILT |
| | | | SHTCQPVPIAIGGPGLAPGCRFRRDIPTGGLANVAATVMNLHGFEAPSDYEPTLVE |
| | | | VVD |
| 782 | 13 | Amb_a | MDEEYDVIVLGTGLKECILSGLLSVDGLKVLHMDRNDYYGGESTSLNLSQLWKRF |
| | | | KGGEAPPEELGASKDYNVDMVPKYMMANGTLVRVLIHTSVTKYLNFKAVDGSYVF |
| | | | NKGKVHKVPATDVEALKSPLMGLFEKRRARKFFIYIQDYDDNDPKSHEGMDVTKV |
| | | | PAKDLISKKYGLDDHTVDFIGHALALHRDDDYLEQPAIDLIKRVKLYAESLARFAG |
| | | | GSPYIYPLYGLGELPQAFARLSAVYGGTYMLNKPECKVEFEDGKVVGVTSEGETAK |
| | | | CKKVVCDPSYLPDKVQKVGKVARAICIMSHPIPNTNDAHSAQVILPQKQLGRKSD |
| | | | MYLFCCSYSHNVAPKGKFIAFVTTEAETDDPETELKPGIDLLGPVDQIFFDTYDRYE |
| | | | PVNQGEEDNCYISASYDATTHFESTVQDVIAMYSRITGKTLDLSVDLSAASAAGDE |
| 783 | 13 | Amb_p | MDEEYDVIVLGTGLKECILSGLLSVDGLKVLHMDRNDYYGGESTSLNLSQLWKRF |
| | | | KGGEAPPEELGASKDYNVDMVPKYMMANGTLVRVLIHTSVTKYLNFKAVDGSYVF |
| | | | NKGKVHKVPATDVEALKSPLMGLFEKRRARKFFIYIQDYDDNDPKSHEGMDVTKV |
| | | | PAKDLISKKYGLDDHTVDFIGHALALHRDDDYLEQPAIDLIKRVKLYAESLARFAG |
| | | | GSPYIYPLYGLGELPQAFARLSAVYGGTYMLNKPECKVEFEDGKVVGVTSEGETAK |
| | | | CKKVVCDPSYLPDKVQKVGKVARAICIMSHPIPNTNDAHSAQVILPQKQLGRKSD |
| | | | MYLFCCSYSHNVAPKGKFIAFVTTEAETDDPETELKPGIDLLGPVDQIFFDTYDRYE |
| | | | PVNQGEEDNCYISASYDATTHFESTVQDVIAMYSRITGKTLDLSVDLSAASAAGDE |
| 784 | 13 | Bet_v | MDEEYDVIVLGTGLKECILSGLLSVDGLKVLHMDRNDYYGGDSSSLNLTQLWKRF |
| | | | RGNDTPPEKLGSSREYNVDMIPKFMMANGKLVRVLIHTDVTKYLHFKAVDGSFVY |
| | | | NKGKIYKVPASDVEALTSSLMGLFEKRRARKFFLYVQDYEDNDPKSHEGLDLNKVT |
| | | | ARELITKYGLEDDTIGIIGHALALQIDDSYLDQPAMDFVKRMKLYAESLARFQGNS |
| | | | PYIYPLYGLGELPQAFARLSAVYGGTYMLNKPECKVEFGNDGKAFGVTSEGETAKC |
| | | | KKVVCDPSYLPDKVQKVGKVARAICIMSHPIPDTNDSHSVQVILPQKQLGRKSDM |
| | | | YLFCCSYAHNVAAKGKYIAFVSTEAETDKPEVELKAGIDLLGPVEEIFYDTYDRFVP |
| | | | TNKHEVDSCFISTSYDATSHFESTVDDVIQLYSKITGKALDLSVDL |
| 785 | 13 | Cyn_d | MDEEYDVIVLGTGLKECILSGLLSVDGLKVLHMDRNDYYGGESTSLNLTKLWKRF |
| | | | KGNDNPPEHLGISKQYNVDMIPKFMMANGALVRVLIHTSVTKYLNFKAVDGSFVY |
| | | | NNGKIHKVPATDVEALKSNLMGLFEKRRARKFFIYVQDYEEEDPKSHEGLDLHKVT |
| | | | TREVISKYGLEDDTVDFIGHALALHRDDNYLDEPAIHTVKRMKLYAESLARFQSAS |
| | | | PYIYPLYGLGELPQAFARLSAVYGGTYMLNKPECKVEFDENGKAYGVTSEGVTAKC |
| | | | KKVVCDPSYLPEKVKKVGKVARAICIMKHPIPHTKDSHSVQIILPKKQLKRKSDMY |
| | | | VFCCSYAHNVAPNGKFIAFVSTEAETDKPEIELKPGIDLLGPVEETFFDIYDRYEPTN |
| | | | NPEEDSCFLTNSYDATTHFETTVQDVLSMYNKITGKELDLSVDLNAASATEQE |
| 786 | 13 | Que_a | MDEEYDVVVLGTGLKECILSGLLSVDGLKVLHMDRNDYYGGESTSLNLIQLWKRF |
| | | | RGNDKPPAHLGSSRDYNVDMIPKFMMANGTLVRVLIHTDVTKYLYFKAVDGSFVY |
| | | | NKGKVHKVPATDMEALKSPLMGIFEKRRARKFFIYVQDYNETDPKTHDGMDLTRV |
| | | | TTRELIAKYGLDDNTVDFIGHALALHRDDRYLDEPALDTVKRMKLYAESLARFQGG |
| | | | SPYIYPLYGLGELPQAFARLSAVYGGTYMLNKPECKVEFNEVGQVLGVTSEGETAR |
| | | | CKKVVCDPSYLPNKVRKVGRVARAIAIMSHPIPNTNESHSVQVILPQKQLGRKSD |
| | | | MYLFCCSYSHNVAPKGKFIAFVSTEAETDHPETELKAGIDLLGPVDEIFFDIYDRYEP |
| | | | VNEPTLDNCFISTSYDATTHFESTVLDVLNMYTMITGKVLDLSVDLSAASAAE |
| 787 | 19 | Amb_a | MAKDPIRVLVTGAAGQIGYALVPMIARGIMLGPDQPVILHMLDIPPAAEALNGVKM |
| | | | ELVDAAFPLLKGVVATTDAVEACTGVNVAVMVGGFPRKEGMERKDVMSKNVSIYK |
| | | | SQASALEKYAAANCKVLVVANPANTNALILKEFAPSIPEKNITCLTRLDHNRALGQI |
| | | | SEKLNVQVSDVKNVIIWGNHSSTQYPDVTHATVTTPSGDKRVPELVNDDEWLKS |
| | | | GFIATVQQRGAAIIKARKLSSALSAASSACDHIRDWVCGTPAGTWVSMGVYSDG |
| | | | SYDVPAGLIYSFPVTCRNGEWTIVQGLSIDEFSRKKLDLTAEELSEEKALAYSCL |
| 788 | 19 | Amb_p | MAKDPIRVLVTGAAGQIGYALVPMIARGIMLGPDQPVILHMLDIPPAAEALNGVKM |
| | | | ELVDAAFPLLKGVVATTDAVEACTGVNVAVMVGGFPRKEGMERKDVMSKNVSIYK |
| | | | SQASALEKYAAANCKVLVVANPANTNALILKEFAPSIPEKNITCLTRLDHNRALGQI |
| | | | SEKLNVQVSDVKNVIIWGNHSSTQYPDVTHATVTTPSGDKRVPELVNDDEWLKS |
| | | | GFIATVQQRGAAIIKARKLSSALSAASSACDHIRDWVCGTPAGTWVSMGVYSDG |
| | | | SYDVPAGLIYSFPVTCRNGEWTIVQGLSIDEFSRKKLDLTAEELSEEKALAYSCL |
| 789 | 19 | Bet_v | MAKEPVRILVTGAAGQIGYALVPMIARGVVLGPDQPVILHMLDIPPAAEALNGVKM |
| | | | ELVDAAFPLLKGVIATTDVVEACTGVNIAIMVGGFPRKEGMERKDVMSKNVSIYKS |
| | | | QASALEKHAAANCKVLVVANPANTNALILKECAPSIPEKNISCLTRLDHNRALGQIS |
| | | | ERLNVPVCDVKNVIIWGNHSSTQYPDVSHATVKTPSGEKPVPELVADDAWLKGEF |
| | | | ITTVQQRGAAIIKARKLSSALSAASSACDHIRDWVLGTPEGTWVSMGVYSDGSYN |
| | | | VPAGLIYSFPVTCRNGEWKIVQGLSIDEFSRKKLDLTAEELSEEKTLAYSCL |
| 790 | 19 | Bet_v | MAKNPVRVLVTGAAGQIGYAIVPMVARGIMLGPDQPVILHLLDIEPAAEALNGVKM |
| | | | ELVDAAFPLLKGVVATTDVVEACKGVNVAVMVGGFPRKEGMERKDVMSKNVSIYK |
| | | | AQASALEEHAAEDCKVLVVANPANTNALILKEFAPSIPEKNISCLTRLDHNRALGQI |
| | | | SERLNVHVSDVKNVIIWGNHSSTQYPDVNHATVTTSGAEKPVRELVADDHWLNA |
| | | | EFITTVQQRGAAIIKARKLSSALSAASAACDHIRDWVLGTPKGTWVSMGVYSDGS |
| | | | YGIQPGLIYSFPVTCEKGQWSIVQGLKIDEFSRAKMDATAKELIEEKSLANSCL |
| 791 | 19 | Cyn_d | MAKEPMRVLVTGAAGQIGYALVPMIARGIMLGADQPVILHMLDIPPAAEALNGVK |
| | | | MELVDAAFPLLKGVVATTDVVEACTGVNVAVMVGGFPRKEGMERKDVMSKNVSI |
| | | | YKAQASALEAHAAPNCKVLVVANPANTNALILKEFAPSIPEKNITCLTRLDHNRALG |
| | | | QISERLNVQVSDVKNVIIWGNHSSTQYPDVNHATVKTPSGEKPVRELVADDEWL |
| | | | NGEFVKTVQQRGAAIIKARKLSSALSAASSACDHIRDWVLGTPEGTYVSMGVYSD |
| | | | GSYGVPAGLIYSYPVTCSGGEWKIVQGLPIDDLSRQKMDATAQELSEEKTLAYSCL |
| 792 | 19 | Que_a | MGKEPVRVLVTGAAGQIGYALVPMIARGVMLGPDQPVILHMLDIPPAAEALNGVK |
| | | | MELVDAAFPLLKGVVATTDVVEGCTGVNIAIMVGGFPRKEGMERKDVMSKNVSIY |
| | | | KSQASALEQHAAANCKVLVVANPANTNALILKEFAPSIPEKNITCLTRLDHNRALG |
| | | | QISERLNVQVSDVKNAIIWGNHSSTQYPDVNHATVKTPSGEKPVRELVADDAWL |
| | | | HGEFIATVQQRGAAIIKARKLSSALSAASSACDHIRDWVLGTPEGTWVSMGVYSD |
| | | | GSYNVPAGLIYSFPVTCRNGEWKIVQGLSIDELSRKKLDLTAEELTEEKALAYSCL |
| 793 | 20 | Amb_a | SQSRSFATAPPPPAVFVDKNTRVICQGITGKNGTFHTEQAIEYGTKMVGGVTPKK |
| | | | GGTEHLGLPVFNTVADAKAETKANASVIYVPPPFAAAAIMEALEAELDLIVCITEGIP |
| | | | QHDMVKVKAALLQQSKTRLIGPNCPGIIKPGECKIGIMPGYIHKPGRIGIVSRSGTL |
| | | | TYEAVYQTTVVGLGQSTCVGIGGDPFNGTNFVDCMEKFIADPQTEGIVLIGEIGGT |
| | | | AEEDAAALIKESGTEKPIVGFIAGLTAPPGRRMGHAGAIVSGGKGTAQDKIKTLKE |
| | | | AGVTVVESPAKIGSAMF |
| 794 | 20 | Amb_p | TRQYATASSQYAETIKNLRINGDTKVLFQGFTGKQGTFHAQQAIEYGTKVVGGTN |
| | | | PKKAGTEHLGLPVFKNVAEAMKETQASATAIFVPPPVAAASIEEAINAEVPLIVTITE |
| | | | GIPQHDMVRITDMLKTQSKSRMVGPNCPGIIAPGQCKIGIMPGFIHKRGRVGIVSR |
| | | | SGTLTYEAVNQTTQAGLGQSLVVGIGGDPFSGTNFIDCLNVFLKDEETDGIIMIGEI |
| | | | GGTAEEDAADFLKEYNTANKPVVSFIAGISAPPGRRMGHAGAIVSGGKGDANSKI |
| | | | TALEAAGVTVERSPAKLGSSLYDQFVKRDLI |
| 795 | 20 | Amb_p | CQTETKANASVIYVPPPFAAAAIMEALEAELDLIVCITEGIPQHDMVKVKAALLQQS |
| | | | KTRLIGPNCPGIIKPGECKIGIMPGYIHKPGRIGIVSRSGTLTYEAVYQTTVVGLGQ |
| | | | STCVGIGGDPFNGTNFVDCMEKFIADPQTEGIVLIGEIGGTAEEDAAALIKESGTEK |
| | | | PIVGFIAGLTAPPGRRMGHAGAIVSGGKGTAQDKIKTLKEAGVTVVESPAKIGSAM |
| | | | FEVFKQRGLV |
| 796 | 20 | Bet_v | AKLIGSIASRRASSIAAQTRQYGSAPHPSPAVFVDKNTRVICQGITGKNGTFHTEQ |
| | | | AIEYGTKMVGGVTPKKGGTEHLGLPVFNSVAEAKAETKANASVIYVPPPFAAAAIM |
| | | | EALEAELDLVVCITEGIPQHDMVRVKAAINTQSKTRLIGPNCPGIIKPGECKIGIMP |
| | | | GYIHKPGRVGIVSRSGTLTYEAVFQTTAVGLGQSTCVGIGGDPFNGTNFVDCIEKF |
| | | | IVDPQTEGIVLIGEIGGTAEEDAAALIKESGTQKPIVAFIAGLTAPPGRRMGHAGAI |
| | | | VSGGKGTAQDKIKTLREAGVTVVESPAKIGVAMLDVFKQRGLV |
| 797 | 20 | Cyn_d | AATRRASHLLGSTASRLLHARGFAAAAAAAPSPAVFVDKSTRVICQGITGKNGTFH |
| | | | TEQAIEYGTNMVGGVTPKKGGTEHLGLPVFNSVAEAKAETKANASVIYVPPPFAAA |
| | | | AIMEAMDAELDLVVCITEGIPQHDMVKVKAALNRQSKTRLIGPNCPGIIKPGECKI |
| | | | GIMPGYIHKPGRVGIVSRSGTLTYEAVFQTTAVGLGQSTCVGIGGDPFNGTNFVD |
| | | | CLEKFVNDPQTEGIVLIGEIGGTAEEDAAAFIQESKTEKPVVAFIAGLTAPPGRRMG |
| | | | HAGAIVSGGKGTAQDKIKALREAGVTVVESPAKIGSKMFEIFKERGMVE |
| 798 | 20 | Que_a | WTQTRQYAAAAAHPPPAVFVDKNTRVICQGITGKNGTFHTEQAIEYGTKMVGGVT |
| | | | PKKGGTEHLGLPVFNTVAEAKAETKANASVIYVPPPFAATAILEAMEAELDLVVCIT |
| | | | EGIPQHDMVRVKSALNRQSKTRLIGPNCPGIIKPGECKIGIMPGYIHKPGRVGIVS |
| | | | RSGTLTYEAVFQTTAVGLGQSTCVGIGGDPFNGTNFVDCIEKFLVDPQTEGIVLIG |
| | | | EIGGTAEEDAAALIKESGTEKPIVAFIAGLTAPPGRRMGHAGAIVSGGKGTAQDKI |
| | | | KTLREAGVTVVESPAKIGVTMHDVFKQKGLV |
| 799 | 22 | Amb_a | MALPNQQTVDYPSFKLVIVGDGGTGKTTFVKRHLTGEFEKKYEPTIGVEVHPLDFF |
| | | | TNCGKIRFYCWDTAGQEKFGGLRDGYYTHGQCAIIMFDVTARLTYKNVPTWH |
| 800 | 22 | Amb_a | QGSVPTFKLVLVGDGGTGKTTFVKRHLTGEFEKKYIATLGVEVHPLGFTTNLGPIQF |
| | | | DVWDTAGQEKFGGLRDGYYINGQCGIIMFDVTSRITYKNVPNWHRDLVRVCENIP |
| | | | IVLTGNKVDVKERKVKAKTITFHRKKNLQYYDISAKSNYNFEKPFLWLARKLVGNQ |
| | | | SLDFVAAPALAPPEVQVDQAVLDQYRQEMEAASALPLPDEDD |
| 801 | 22 | Amb_a | FDVTARLTYKNVPTWHRDLCRVCENIPIVLCGNKVDVKNRQVKAKQVTFHRKKNL |
| | | | QYYEISAKSNYNFEKPFLYLARKLAGDPNLHFVESPALAPPEVQIDMVAQQQHEAE |
| | | | LAVAANQPLPDDDDDAFE |
| 802 | 22 | Amb_p | QGSVPTFKLVLVGDGGTGKTTFVKRHLTGEFEKKYIATLGVEVHPLGFTTNLGPIQF |
| | | | DVWDTAGQEKFGGLRDGYYINGQCGIIMFDVTSRITYKNVPNWHRDLVRVCENIP |
| | | | IVLTGNKVDVKERKVKAKSITFHRKKNLQYYDISAKSNYNFEKPFLWLARKLVGNQ |
| | | | SLDFVAAPALAPPEVQVDQAVLDQYRQEMEAASALPLPDEDD |
| 803 | 22 | Amb_p | MALPNQQTVDYPSFKLVIVGDGGTGKTTFVKRHLTGEFEKKYEPTIGVEVHPLDFF |
| | | | TNCGKIRFYCWDTAGQEKFGGLRDGYYIHGQCAIIMFDVTARLTYKNVPTWHRDL |
| | | | CRVCENIPIVLCGNKVDVKNRQVKAKQVTFHRKKNLQYYEISAKSNYNFEKPF |
| 804 | 22 | Bet_v | MALPNQQTVDYPSFKLVIVGDGGTGKTTFVKRHLTGEFEKKYEPTIGVEVHPLDFF |
| | | | TNCGKIRFYCWDTAGQEKFGGLRDGYYIHGQCAIIMFDVTARLTYKNVPTWHRDL |
| | | | CRVCENIPIVLCGNKVDVRNRQVKAKQVTFHRKKNLQYYEISAKSNYNFEKPFLYL |
| | | | ARKLAGDPSLHFVESPALAPPEVQIDLAAQQQHEAELMAAASQPLPDDDDDTFE |
| 805 | 22 | Cyn_d | MALPNQQVVDYPSFKLVIVGDGGTGKTTFVKRHLTGEFEKKYEPTIGVEVHPLDFS |
| | | | TNCGKIRFYCWDTAGQEKFGGLRDGYYIHGQCAIIMFDVTSRLTYKNVPTWHRDL |
| | | | CRVCENIPIVLCGNKVDVKNRQVKAKQVTFHRKKNLQYYEISAKSNYNFEKPFLYL |
| | | | ARKLAGDQNLHFVEAVALKPPEVQIDMAMQQQHEAELVAAAAQ |
| 806 | 22 | Que_a | MALPNQQTVEYPSFKLVIVGDGGTGKTTFVKRHLTGEFEKKYEPTIGVEVHPLDFFT |
| | | | NCGKIRFYCWDTAGQEKFGGLRDGYYIHGQCAIIMFDVTARLTYKNVPTWHRDLC |
| | | | RVCENIPIVLCGNKVDVKNRQVKAKQVTFHRKKNLQYYEISAKSNYNFEKPFLYLA |
| | | | RKLAGDPALHFVESPALAPPEVQIDLAAQQQHEAELQQAASQPLPDDDDDTFE |
| 807 | 22 | Que_a | MALPNQQTVEYPSFKLVIVGDGGTGKTTFVKRHLTGEFEKKYEPTIGVEVHPLDFFT |
| | | | NCGKIRFYCWDTAGQEKFGGLRDGYYIHGQCAIIMFDVTARLTYKNVPTWHRDLC |
| | | | RVCENIPIVLCGNKVDVKNRQVKAKQVTFHRKKNLQYYEISAKSNYNFEKPFLYLA |
| | | | RKLAGDANLHFVESPALAPPEVQIDLAAQQQHEAELQQAASQPLPDDDDDTFE |
| 808 | 24 | Amb_a | MATKKSVSSLTEADLKGKRVFVRVDLNVPLDDTFKITDDTRIRAAVPTIKYLMSNG |
| | | | ARVILSSHLGRPKGVTPKFSLKPLVPRLSELLGIEVKMADDCVGPEVEKLVAEIPEG |
| | | | GVLLLENVRFYKEEEKNDPEFAKKLASLADLYVNDAFGTAHRAHASTEGVAKHLKP |
| | | | AVAGFLMQKELDYLVGAVSNPKKPFAAIVGGSKVSSKIGVIESLLEKVNILVLGGG |
| | | | MIFTFYKAQGLAVGSSLVEEDKLDLATTLLEKAKSKGVSLLLPSDVVIADKFAADAN |
| | | | SKVVPASSIPDGWMGLDIGPDSIKSFNEALDTTKTVIWNGPMGVFEFDKFAVGTE |
| | | | AIAKKLAELSGKGVTTIIGGGDSVAAVEKVGLADKMSHISTGGGASLELLEGKPLP |
| | | | GVLALDDA |
| 809 | 24 | Amb_p | SLTEADLKGKRVFVRVDLNVPLDDTFKITDDTRIRAAVPTIKYLMSNGARVILSSHL |
| | | | GRPKGVTPKFSLKPLVPRLSELLGIEVKMADDCVGPEVEKLVAEIPEGGVLLLENVR |
| | | | FYKEEEKNDPEFAKKLASLADLYVNDAFGTAHRAHASTEGVAKHLKPAVAGFLMQ |
| | | | KELDYLVGAVSNPKKPFAAIVGGSKVSSKIGVIESLLEKVNILVLGGGMIFTFYKAQ |
| | | | GLAVGSSLVEEDKLDLATTLLEKAKSKGVSLLLPSDVVIADKFAADANSKVVPASSI |
| | | | PDGWMGLDIGPDSIKSFNESLDTTKTVIWNGPMGVFEFDKFAVGTEAIAKKLAEL |
| | | | SGKGVTTIIGGGDSVAAVEKVGLADKMSHISTGGGASLELLEGKPLPGVLALDDA |
| 810 | 24 | Bet_v | MATKRSVSTLKEADLKGKRVFVRVDLNVPLDDNFNITDDTRIRAAVPTIKYLQAHG |
| | | | AKVILSSHLGRPKGVTPKYSLKPLVPRLSELLGTEVKMANDCVGEEVEKLVAEIPEG |
| | | | GVLLLENVRFHKEEEKNDPEFAKKLASLADLYVNDAFGTAHRAHASTEGVAKYLKP |
| | | | SVAGFLMQKELDYLVGAIANPKRPFAAIVGGSKVSSKIGVIESLLAKVDLLLLGGG |
| | | | MIFTFYKAQGYSVGSSLVEEDKLDLARSLIEKAKSKGVSLLLPTDVIIADKFAPDAN |
| | | | SKVVPASGIPDGWMGLDIGPDSVKTFNKALDTTKTIIWNGPMGVFEFEKFAAGTE |
| | | | AIAKKLAELSDKGVTTIIGGGDSVAAVEKVGLAEKMSHISTGGGASLELLEGKPLP |
| | | | GVLALDDA |
| 811 | 24 | Cyn_d | MATKRSVGTLGEADLKGKKVFVRADLNVPLDDAQKITDDTRIRASVPTIKFLLEKG |
| | | | AKVILASHLGRPKGVTPKYSLKPLVPRLSELLGIDVVMANDCIGEEVEKLAAALPEG |
| | | | GVLLLENVRFYKEEEKNDPEFAKKLASVADLYVNDAFGTAHRAHASTEGVTKYLKP |
| | | | AVAGFLMQKELDYLVGAVANPKKPFAAIVGGSKVSTKIGVIESLLAKVDILILGGG |
| | | | MIYTFYKAQGYAVGKSLVEEDKLDLATSLIEKAKAKGVSLLLPTDIVVADKFAADAE |
| | | | SKIVPATSIPDDWMGLDVGPDATKTFNEALDTTQTIIWNGPMGVFEFDKFAAGTE |
| | | | ATAKKLAELTSTKGVTTIIGGGDSVAAVEKAGLADKMSHISTGGGASLELLEGKPL |
| | | | PGVLALDEA |
| 812 | 24 | Que_a | MATKRSVSTLKQADLKGKRVFVRVDLNVPLDDNFNITDDTRIRAAVPTIKYLQSHG |
| | | | ARVILSTHLGRPKGVTPKYSLKPIVPRLSELLGVEVKMANDCIGEEVEKLVAETPEG |
| | | | GVLLLENVRFHKEEEKNDPEFSKKLASLADLYVNDAFGTAHRAHASTEGVAKFLKP |
| | | | AVAGFLMQKELDYLVGAVSNPKRPFAAIVGGSKVSSKIGVIESLLGKVNLLLLGGG |
| | | | MIFTFYKAQGYSVGSSLVEEDKLDLATTLIEKAKAKGVSLLLPTDVVIADKFAADAN |
| | | | SKVVPASAIPDGWMGLDIGPDSIKTFNEALDTTQTVIWNGPMGVFEFEKFAAGTE |
| | | | ATAKKLADLSAKGVTTIIGGGDSVAAVEKVGLADKMSHISTGGGASLELLEGKPLP |
| | | | GVLALDDA |
| 813 | 27 | Amb_a | GVFTDKDKAAAHLKGGAKKVVISAPSANAPMFVMGVNEKEYTPDITIVSNASCTT |
| | | | NCLAPLAKVIHDKFGIVEGLMTTVHSITATQKTVDGPSMKDWRGGRAASFNIIPSS |
| | | | TGAAKAVGKVLPALNGKLTGMAFRVPTVDVSVVDLTVRLEKKATYEQVKAAIKEES |
| | | | EGKLKGILGYVDEDVVSTDFVGDSRSSIFDAKAGIALNDNFLKLVSWYDNEWGY |
| 814 | 27 | Amb_a | MSCYKGKYADELIANAAYIGTPGKGILAADESTGTIGKRLSSINVENSESNRRALR |
| | | | ELLFCTPGALQYISGIILFEETLYQKTAAGKPFVELMKEANVLPGIKVDKGVVELAGT |
| | | | NGETTTTGLDGLAQRCAQYYEAGARFAKWRAVLKIGANEPSQLAINENANGLARY |
| | | | AIICQENGLVPIVEPEILVDGSHDINKCADVTERVLAACYKALNDHHVLLEGTLLKP |
| | | | NMVTPGSDSKKVAPEVVGEYTVRALQRTMPAAVPAVVFLSGGQSEEEATVNLNAI |
| | | | NQYKGKKPWSLTFSYGRALQQSTLKAWGGKEENVKKAQETFLIRCKANSEASLG |
| | | | KYEGGAAGEGANESLHVKDYKY |
| 815 | 27 | Amb_p | MSCYKGKYADELIANAAYIGTPGKGILAADESTGTIGKRLSSINVENSESNRRALR |
| | | | ELLFCTPGALQYISGIILFEETLYQKTAAGKPFVELMKEANVLPGIKVDKGVVELAGT |
| | | | NGETTTTGLDGLAQRCAQYYEAGARFAKWRAVLKIGANEPSQLAINENANGLARY |
| | | | AIICQENGLVPIVEPEILVDGSHDINKCADVTERVLAACYKALNDHHVLLEGTLLKP |
| | | | NMVTPGSDSKKVAPEVVGEYTVRALQRTMPAAVPAVVFLSGGQSEEEATVNLNAI |
| | | | NQYKGKKPWSLTFSYGRALQQSTLKAWGGKEENVKKAQETFLIRCKANSEASLG |
| | | | KYEGGAAGEGANESLHVKDYKY |
| 816 | 27 | Bet_v | MSAFKGKYHDELIANAAYIGTPGKGILAADESTGTIGKRLSSINVENVEENRRALR |
| | | | ELLFTAPNALQYLSGVILFEETLYQKTASGQLFAELLKENGVLPGIKVDKGTVVLAG |
| | | | TNGETTTQGLDGLAQRCQKYYEAGARFAKWRAVLNIGPNEPSQLSINENANGLAR |
| | | | YAIICQENGLVPIVEPEILVDGSHSIEKCADVTERVLAACYKALNDHHVLLEGTLLKP |
| | | | NMVTPGSDAPKVAPEVVAEHTVRALLRTVPAAVPAVVFLSGGQSEEEATINLNAM |
| | | | NKLKGKKPWTLSFSFGRALQSSTLKAWGGKLENVAKAQAALLARAKANSEATLGI |
| | | | YKGDAQLGEGASESLHVKGYKY |
| 817 | 27 | Cyn_d | MSAHVGKFADELIKNAAYIGTPGKGILAADESTGTIGKRFSSINVENIEENRRALRE |
| | | | LLFCAPGALQYLSGVILFEETLYQKTKDGKPFVDVLKEGGVLPGIKVDKGTIEVAGT |
| | | | DKETTTQGHDDLGKRCAKYYEAGARFAKWRAVLKIGPNEPSQLAIDLNAQGLARY |
| | | | AIICQENGLVPIVEPEILVDGPHDIERCAYVTEMVLAACYKALSEHHVLLEGTLLKPN |
| | | | MVTPGSDAKKVAPEVIAEYTVRALQRTVPAAVPAIVFLSGGQSEEEATLNLNAMNK |
| | | | LNTKKPWSLSFSFGRALQASTLKAWAGKEENVEKARAALLARCKANSEATLGTYK |
| | | | GDAAAGEGVSESLHVKDYKY |
| 818 | 27 | Que_a | MSAYQGKYADELCANAAYIGTPGKGILAADESTGTIGKRLSSINVENVEENRRALR |
| | | | ELLFTTPGALQYLSGVILFEETLYQKTHDGKPFVNLLKENGVLPGIKVDKGTVELAG |
| | | | TNGETTTQGLDGLAQRCQKYYEAGARFAKWRAVLKIGPTEPSQLAINENANGLAR |
| | | | YAIICQENGLVPIVEPEILVDGPHDILKCADVTERVLAAVYKALNDHHVLLEGTLLKP |
| | | | NMVTPGSEAPKVAPEVIAEHTVRALQRTMPAAVPAVVFLSGGQSEEQATVNLNAM |
| | | | NKYKGKKPWTLSFSFGRALQQSTLKAWGGKKENVQKAQAAFLARAKANSEATLG |
| | | | TYKGDATLGEGASESLHVKDYKY |
| 819 | 28 | Amb_p | AKKVIISAPSKDAPMFVVGVNAHEYTPDLDIVSNASCTTNCLAPLAKVINDRFGIVE |
| | | | GLMTTVHAMTATQKTVDGPSMKDWRGGRAASFNIIPSSTGAAKAVGKVLPALNG |
| | | | KLTGMAFRVPTVDVSVVDLTVRIEKAATYEQVKAAIKEESEGKLKGILGYVDEDVV |
| | | | STDFVGDSRSSIFDAKAGIALNDNFLKLVSWYDNEWGYSSRVIDLICHIASVK |
| 820 | 29 | Amb_a | MAEKSFKYVIIGGGVSAGYAAREFAKQGVQPGELAIISKEAVAPYERPALSKAYLFP |
| | | | EGAARLPGFHVCVGSGGEKLLPEWYTEKGIELILNTEIVKADLASKSLTSAAGDTY |
| | | | KYKILITATGSTVLKLTDFKVEGADAKNILYLREIDDADKLVEAIKAKKNGKAVVVG |
| | | | GGYIGLELSAVLKINNFDVKMVYPEPWCMPRLFTADIAAFYEGYYEKKGVGIIKGTV |
| | | | ASGFTKNDNGEVKEVKLKDGRVLEADIVVVGVGARPLTNLFKGQVEEDKGGIKTD |
| | | | AFFKTSVPDVYAVGDVATFPMKMYGDIRRVEHVDHSRKSAEQAVKAIFASEQGKD |
| | | | IEAYDYLPYFYSRSFDLSWQFYGDNVGDAVIFGDHDPASAKAKFGSYWIKDGKVV |
| | | | GAFLEGGAPEENQAIAKVAKTQPAASSLDVLAKEGLGFASKI |
| 821 | 29 | Amb_p | MGKVKIGINGFGRIGRLVARVALLSDDIELVAVNDPFISTEYMTYMFKYDSVHGPW |
| | | | KKHEIQVKDSNTLLFGDKPVTVFGMKNPEETPWGEAGAEYVVESTGVFTDKDKAA |
| | | | AHLKGGAKKVVISAPSANAPMFVMGVNEKEYTPDITIVSNASCTTNCLAPLAKVIH |
| | | | DKFGIVEGLMTTVHSITATQKTVDGPSMKDWRGGRAASFNIIPSSTGAAKAVGKV |
| | | | LPALNGKLTGMAFRVPTVDVSVVDLTVRLEKKATYEQVKAAIKEESEGKLKGILGY |
| | | | VDEDVVSTDFVGDSRSSIFDAKAGIALNDNFLKLVSWYDNEWGYSSRVIDLICHI |
| | | | ASVQ |
| 822 | 29 | Amb_p | MAEKSFKYVIIGGGVSAGYAAREFAKQGVQPGELAIISKEAVAPYERPALSKAYLFP |
| | | | EGAARLPGFHVCVGSGGEKLLPEWYTEKGIELILNTEIVKADLASKSLTSAAGDTY |
| | | | KYKILITATGSTVLKLTDFKVEGADAKNILYLREIDDADKLVEAIKAKKNGKAVVVG |
| | | | GGYIGLELSAVLKINNFDVKMVYPEPWCMPRLFTADIAAFYEGYYEKKGVGIIKGTV |
| | | | ASGFTKNDNGEVKEVKLKDGRVLEADIVVVGVGARPLTNLFKGQVEEDKGGIKTD |
| | | | AFFKASVPDVYAVGDVATFPMKMYGDIRRVEHVDHSRKSAEQAVKAIFASEQGKD |
| | | | IEAYDYLPYFYSRSFDLSWQFYGDNVGDAVIFGDHDPASAKAKFGSYWIKDGKVV |
| | | | GAFLEGGAPEENQAIAKVAKTQPAASSLDVLAKEGLGFASKI |
| 823 | 29 | Bet_v | MAEKSFKYVIVGGGVAAGYAAKEFAKQGLKPGELAIVSKEAVAPYERPALSKAYLF |
| | | | PESPARLPGFHVCVGSGGERLLPEWYKEKGIELILRTEIVKADLAAKILTSAAGETF |
| | | | KYQILITATGSSVIRLTDFGVQGADAKNIFYLREIDDADKLIEAFKAKKNGKAVVVG |
| | | | GGYIGLELGAVLKMNNYDVSMVYPEPWCMPRLFTSGIAAFYEGYYKNKGIEIIKGT |
| | | | VAVGFTSDSKGEVKEVKLKDGRVLEADIVVVGVGGRPLTTLFKGQVEEEKGGIKT |
| | | | DASFKTSVTGVYAVGDVATFPLKLYNELRRVEHVDHARKSAEQAVKAIKASEEGK |
| | | | TIEEYDYLPYFYSRSFDLSWQFYGDNVGDSVLFGDNNPASPKPKFGSYWIKDGKV |
| | | | VGAFLEGGNPEENKAIAKVARVQPPVENLDLLTKEGLSFAAKI |
| 824 | 29 | Cyn_d | MAKHFKYVILGGGVAAGYAAREFGKQGVKPGELAIISKEPVAPYERPALSKGYLFP |
| | | | QNAARLPGFHTCVGSGGERLLPEWYSEKGIELILSTEIVKVDLASKTLTSASEATFT |
| | | | YEILLIATGSSVIKLTDFGVQGAEYNNILYLRDIQDGEKLVAAMQAKKDGKAVVVG |
| | | | GGYIGLELSAALKMNNFDVTMVYPEPWCMPRLFTAGIAHFYEGYYASKGINLVKGT |
| | | | YAAGFDADSNGDVTAVKLKDGRVLEADIVIVGVGGRPLTGLFKGQVAEEKGGIKT |
| | | | DGFFETSVPDVYAIGDVATFPMKLYNDQRRVEHVDHARKSAEQAVRAIKAKESGE |
| | | | SIAEYDYLPYFYSRSFDVAWQFYGDNVGDDVLFGDNDPAAAKPKFGSYWVKDGK |
| | | | VVGVFLEGGSADEYQAIARVARAQPQVADVEALRKDGLDFAIKT |
| 825 | 29 | Que_a | MAAKSFKYVIVGGGVSAGYAAREFAKQGVKPGELAIISKEAVAPYERPALSKAYLFP |
| | | | ESPARLPGFHVCVGSGGERLLPEWYKEKGIELILSTEIVKADLAAKTLISAAGETFN |
| | | | YQILITATGSSVIRLTDFGVQGADAKNIYYLREVDDADKLVEAIKAKKNGKVVIVGG |
| | | | GYIGLELSAVMKINNLDVNMVYPEPWCMPRLFTADIAAFYEGFYKNKGIQIIKGTV |
| | | | AVGFTADSNGEVKEVKLKDGRVLEADIVVVGVGGRPLTTLFKGQVEEEKGGIKTD |
| | | | SFFKTSVPNVYAVGDVATFPLKLYKELRRVEHVDHSRKSAEQAVKAIKASEEGKTI |
| | | | EEYDYLPFFYSRSFDLSWQFYGDNVGDTVIFGDNNPETPKPKFGSYWIKDGKVLG |
| | | | AFLEGGTPEENKAIAKVARVQPPVENLDVLSKEGLSFACKI |
| 826 | 30 | Amb_a | AQGSQLVTPWNMSISSGHALLRDPRLNKGLAFTEREREVHYLTGLLPPTIATQELQ |
| | | | EKKAMQIIRQYEVPLQKYIAMIGLQERNERLFYKLLTDHVEELLPVVYTPTVGEACQ |
| | | | KFGSIFQRPQGLYISLKDKGKVLQVLRNWPERNIEVIVVTDGERILGLGDLGCQGM |
| | | | GIPVGKLSLYTALGGVRPSACLPITIDVGTNNEKLLNDEFYIGLKQNRSRGEEYDEL |
| | | | LEEFMKAVKINYGEKILIQFEDFANHNAFSLLNRYRTTHLVFNDDIQGTASVVLSGL |
| | | | LSALNLLGGTLSDHTFLFLGAGEAGTGIAELIALQISKKTDTSIEEARKKIWLVDSK |
| | | | GLVESSRTESLQHFKLPWAHEHEPVSNLLDAVEDIKPSVLIGTSGVGRQFTQEVIE |
| | | | AMSSINEKPLIMALSNPTSQAECTAEEAYTWSKGKAIFASGSPFDPVTYEDQVFVP |
| | | | GQANNAYIFPGFGLGLIMCGATRVHDDLLLAASEGLASQVTDEDYAKGIIFPPFSCI |
| | | | RKISAHIAAQVADKAYELGLASLLPRPNDLVQYAESCMYSPIYPNYR |
| 827 | 30 | Amb_p | AQGSQLVTPWNMSISSGHALLRDPRLNKGLAFTEREREVHYLTGLLPPTIATQELQ |
| | | | EKKAMQIIRQYEVPLQKYIAMIGLQERNERLFYKLLTDHVEELLPVVYTPTVGEACQ |
| | | | KFGSIFQRPQGLYISLKDKGKVLQVLRNWPERNIEVIVVTDGERILGLGDLGCQGM |
| | | | GIPVGKLSLYTALGGVRPSACLPITIDVGTNNEKLLNDEFYIGLKQNRSRGEEYDEL |
| | | | LEEFMTAVKINYGEKILIQFEDFANHNAFSLLNRYRTTHLVFNDDIQGTASVVLSGL |
| | | | LSALNLLGGTLSDHTFLFLGAGEAGTGIAELIALQISKKTDTSIEEARKKIWLVDSK |
| | | | GLVESSRTESLQHFKLPWAHEHEPVSNLLDAVEDIKPSVLIGTSGVGRQFTQEVIE |
| | | | AMSSINEKPLIMALSNPTSQAECTAEEAYTWSKGKAIFASGSPFDPVTYEDQVFVP |
| | | | GQANNAYIFPGFGLGLIMCGATRVHDDLLLAASEGLASQVTDEDYAKGIIFPPFSCI |
| | | | RKISAHIAAQVADKAYELGLASLLPRPNDLVQYAESCMYSPIYPNYR |
| 828 | 30 | Bet_v | MGKIKIGINGFGRIGRLVARVALQRDDVELVAVNDPFITTDYMTYMFKYDTVHGP |
| | | | WKHHELKVQDSKTLLFGDKPVTVFGIRNPEEIPWAEAGADFVVESTGVFTDKDKA |
| | | | AAHLKGGAKKVIISAPSKDAPMFVVGVNEKEYKPELNIVSNASCTTNCLAPLAKVI |
| | | | NDRFGIVEGLMTTVHSITATQKTVDGPSMKDWRGGRAASFNIIPSSTGAAKAVGK |
| | | | VLPALNGKLTGMAFRVPTVDVSVVDLTVRLEKKASYEEIKAAIKEESEGKLKGILGY |
| | | | TEEDVVSTDFVGDNRSSIFDAKAGIALNDNFVKLVAWYDNEWGYSSRVVDLIRHI |
| | | | ASVQ |
| 829 | 30 | Bet_v | GGGVQDVYGEDTATEDHFVTPWSVSVASGYSLLRDPHHNKGLAFTERERDAHFL |
| | | | RGLLPPTVASQELQVKKMMHNIRQYQVPLQKYMAMMDLQERNEKLFYKLLIDNVE |
| | | | ELLPIVYTPTVGEACQKYGSIFMRPQGLFISLKEKGKILEVLRNWPEKNIQVIVVTD |
| | | | GERILGLGDLGCQGMGIPVGKLSLYTALGGVRPSACLPITIDVGTNNEQLLNDEFYI |
| | | | GLRQRRATGQEYAELLHEFMTAVKQIYGEKVLIQFEDFANHNAFDLLAKYGTTHLV |
| | | | FNDDIQGTASVVLAGLVAAQKLVGGTLADHRYLFLGAGEAGTGIAELIALEISKQT |
| | | | NAPLEETRKKVFLVDSKGLIVSSRKESLQHFKKPWAHEHEPVKELVDAVKVIKPTV |
| | | | LIGTSGVGNKFTKEVVEAMASINERPIILALSNPTSQSECTAEEAYRWSQGRAIFAS |
| | | | GSPFAPVEYEGKVFVPGQANNAYIFPGFGLGLLMSGAIRVHDDMLLAASEALAAQV |
| | | | TQEDFDKGLIFPPFTNIRKISAQIAAKVAAKAYELGLATRLPQPIDLVKCAESCMYSP |
| | | | AYRSYR |
| 830 | 30 | Cyn_d | MAGGGVEDAYGEDRATEEQLVTPWAFSVASGYTLLRDPRHNKGLAFSEAERDAH |
| | | | YLRGLLPPAFASQELQEKKLMHNLRQYTVPLQRYIAMMDLQERNERLFYKLLIDNV |
| | | | EELLPVVYTPTVGEACQKYGSIYRRPQGLYISLKDKGKILEVLKNWPERSIQVIVVT |
| | | | DGERILGLGDLGCQGMGIPVGKLSLYTALGGVRPSACLPITIDVGTNNETLLNDEF |
| | | | YIGLRQRRATGEEYHELLEEFMTAVKQNYGEKVLIQFEDFANHNAFDLLAKYSKSH |
| | | | LVFNDDIQGTASVVLAGLLASLKVVGGSLADHTYLFLGAGEAGTGIADLIALEMSK |
| | | | HNEMPIDECRKKIWLVDSKGLIVESRKESLQHFKKPWAHEHEPLKTLLEAVESIKP |
| | | | TVLIGTSGVGRTFTKEVIEAMASFNEKPVIFSLSNPTSHSECTAEEAYTWTQGRAV |
| | | | FASGSPFDPVEYEGKVYVPGQSNNAYIFPGFGLGVVISGAIRVHDDMLLAASEALA |
| | | | EQVTEEHFGKGLIFPSFTNIRGISARIAAKVAAKAYELGLASHLPRPDDLVKYAESC |
| | | | MYTPAYRSYR |
| 831 | 30 | Que_a | AGGVRDVYGEDSATEDQFVTPWSVSVASGYSLLRDPHHNKGLAFTIRERDAHFLR |
| | | | GLLPPTVASQDLQVKKMMHNIRQYQVPLQKYMAMMDLQERNQRLFYKLLIDNVEE |
| | | | LLPIVYTPTVGEACQKYGSIFMRPQGLFISLKEKGKILEVLRNWPEKNIQVIVVTDG |
| | | | ERILGLGDLGCQGMGIPVGKLSLYTALGGIRPSACLPITIDVGTNNEKLLNDEFYIG |
| | | | LKQKRATGQEYAELLDEFMMAVKQNYGEKVLIQFEDFANHNAFDLLAKYGTTHLVF |
| | | | NDDIQGTASVVLAGLVAGQKLVGGTLADHRFLFLGAGEAGTGIAELIALEMSKQTK |
| | | | APLEETRKKIWLVDSKGLIVSSRKESLQQFKKPWAHEHEPIKELVDAVKAIRPTVLI |
| | | | GTSGVGRTFTKEVVEAMASINEKPIILALSNPTSQSECTAEEAYTWSQGRAIFASG |
| | | | SPFPPVEYDGKVFMPGQANNAYVFPGLGLGLIMSGAIRVHDDMLLAASEALAAQV |
| | | | SQENFDRGLLYPPFTNIRKISAHIAANVAAKAYELGLATRLPEPKDLVKYAESCMYS |
| | | | PAYRNYR |
| 832 | 32 | Que_a | MGKIKIGINGFGRIGRLVARVALERDDVELVAVNDPFITTDYMTYMFKYDTVHGQ |
| | | | WKHHELKVKDSKTLLFGDRPVATFGIRNPEEIPWGEAGAEFVVESTGVFTDKEKA |
| | | | AAHLKAGAKKVIISAPSKDAPMFVVGVNENDYKPELDIVSNASCTTNCLAPLAKVI |
| | | | HDRFGIVEGLMTTVHSITATQKTVDGPSMKDWRGGRAASFNIIPSSTGAAKAVGK |
| | | | VLPSLNGKLTGMAFRVPTVNVSVVDLTVRLEKKASYEEIKAAIKEESEGKLKGILGY |
| | | | TQEDVVSSDFVGDSRSSIFDAKAGIALNDNFVKLVSWYDNEWGYSSRVIDLIRHI |
| | | | ASVQ |
| 833 | 32 | Cyn_d | MAKIKIGINGFGRIGRLVARVALQSDDVELVAVNDPFITTDYMTYMFKYDTVHGQ |
| | | | WKHHDVKVKDSKTLLFGEKEVTVFGCRNPEETPWGEAGAEYVVESTGVFTDKDK |
| | | | AAAHLKGGAKKVVISAPSKDAPMFVCGVNEKEYKSDIHIVSNASCTTNCLAPLAKV |
| | | | INDKFGIVEGLMTTVHAITATQKTVDGPSAKDWRGGRAASFNIIPSSTGAAKAVG |
| | | | KVLPALNGKLTGMAFRVPTVDVSVVDLTVRLEKSATYDEIKAAIKAESEGDLKGILG |
| | | | YVEEDLVSTDFQGDNRSSIFDAKAGIALNDKFVKLVSWYDNEWGYSSRVIDLIRH |
| | | | MHST |
| 834 | 34 | Amb_a | SSGQVIRCKAAVAREAGKPLVIEEVEVAPPQKMEVRLKIHFTSLCHTDVYFWEAKG |
| | | | QHPLFPRILGHEAGGIVESVGEGVTELKPGDKVLPIFTGECGECRHCKSEESNMCD |
| | | | LLRINTDRGVMINDGKTRFSKDGQPIYHFLGTSTFSEYTVVHSGCVAKINPDAPLD |
| | | | KVCVLSCGISTGMGATLNVAKPKKGMSVAIFGLGAVGLAAAEGARIAG |
| 835 | 34 | Amb_a | WEAKGQNPVFPRILGHEAGGVVESVGEGVTDLQPGDHVLPVFTGECKECAHCKS |
| | | | EESNMCDLLRINTDR |
| 836 | 34 | Amb_p | TTTGQVIRCKAAVAWEAGKPLVMEEVEVAPPQKHEVRIKILFTSLCHTDVYFWEAK |
| | | | GQNPVFPRILGHEAGGVVESVGEGVTDLQPGDHVLPVFTGECKECAHCKSEESN |
| | | | MCDLLRINTDRGVMLHDQKSRFSINGKPIFHFVGTSTFSEYTVVHVGCLAKINPDA |
| | | | PLDKVCVLSCGISTGLGATLNVAKPKKGSSVAVFGLGAVGLAAAEGARIAGASRII |
| | | | GVDLNANRFELAKKFGVTEFVNPKDYKKPVQEVIAELTNGGVDRSVECTGHIDAMI |
| | | | SAFECVHDGWGVAVLVGVPHKDAVFKTNPMNLLNERTLKGTFFGNYKPRSDIPSV |
| | | | VEKYMNKELELEKFITHEVPFSEINKAFDLMLKGEGLRCIIRMD |
| 837 | 34 | Amb_p | GKPLVIEEVEVAPPQKMEVRLKIHFTSLCHTDVYFWEAKGQHPLFPRILGHEAGGI |
| | | | VESVGEGVTELKPGDKVLPIFTGECGECRHCKSEESNMCDLLRINTDRGVMINDG |
| | | | KTRFSKDGQPIYHFLGTSTFSEYTVVHSGCVAKINPDAPLDKVCVLSCGISTGMGA |
| | | | TLNVAKPKKGMSVAIFGLGAVGLAAAEGARIAGASRIIGIDLNPSRAKEAMKFGVT |
| | | | EFVNPKDHDKPIHEVIAAMTDGGVDRSVECTGNVKAMISAFECVHD |
| 838 | 34 | Amb_p | CVHDGWGVAVLVGVPNKDDEFKTLPINFLNERTLKGTFFGNYKPRTDIPGVVEKY |
| | | | MNKELEVEKFITHTIGFSEINKAFDYMLKGESLRCIIRMDA |
| 839 | 34 | Amb_p | SMSTTTGQVIRCKAAVAWEAGKPLVMEEVEVAPPQKHEVRIKILFTSLCHTDVYF |
| | | | WEAKGQNPVFPRILGHEAGGVVESVGEGVTDLQPGDHVLPVFTGECKECAHCKS |
| | | | EESNMCDLLRINTDRGVMLHDQKSRFSINGKPIFHFVGTSTFSEYTVVHVGCLAKI |
| | | | NPDAPLDKVCVLSCGISTGLGATLNVAKPKKGSSVAVFGLGAVGLAAAEGARIAG |
| | | | ASRIIGVDLNANRFELAKKFGVTEFVNPKDYKKPVQEVIAELTNGGVDRSVECTGH |
| | | | IDAMISAFECVHDGWGVAVLVGVPHKDAVFKTNPMNLLNERTLKGTFFGNYKPRS |
| | | | DIPSVVEKYMNKELELEKFITHEVPFSEINKAFDLMLKGEGLRCIIRMDA |
| 840 | 34 | Ant_o | SSVAIWVLFPSEIVISVPVDSRGERAMATAGKVIKCKAAVAWEAGKPLSIEEVEVA |
| | | | PPQAMEVRVKILFTSLCHTDVYFWEAKGQTPVFPRIFGHEAGGIVESVGEGVTDVA |
| | | | PGDHVLPVFTGECKECPHCKSAESNMCDLLRINTDRGVMISDGKSRFSIDGKPIY |
| | | | HFVGTSTFSEYTVMHVGCVAKINPEAPLDKVCVLSCGISTGLGASINVAKPPKGST |
| | | | VAIFGLGAVGLAAAEGARIAGASRIIGIDLNANRFEEARKFGCTEFVNPKDHSKPV |
| | | | QEVLIEMTNGGVDRSVECTGNVNAMIQAFECVHDGWGVAVLVGVPHKDAEFKTH |
| | | | PMKFLNERTLKGTFFGNFKPRTDLPNVVEMYMKKELEVEKFITHSVPFSEINKAFDL |
| | | | MARGEGIRCIIRMEN |
| 841 | 34 | Ant_o | HTDVYFWEAKGQTPVFPRILGHEAGGIVESVGEGVTELVPGDHVLPVFTGECKEC |
| | | | AHCKSEESNLCDLLRINVDRGVMIGDGQSRFTIDGKPIFHFVGTSTFSEYTVIHVG |
| | | | CLAKINPEAPLDKVCVLSCGISTGLGATLNVAKPKKDSTVAIFGLGAVGLAAMEGA |
| | | | KMAGASRIIGVDLNPAKYEQAKKFGCTDFVNPKDHTKPVQEVLVEMTNGGVDRA |
| | | | VECTGHIDAMIAAFECVHDGWGVAVLVGVPHKEAVFKTHPMNFLNERTLKGTFFG |
| | | | NYKPRTDLPEVVEMYMRKELDVEKFITHSVPFSQINTAFDLMLKGEGLRCVMRMG |
| | | | E |
| 842 | 34 | Bet_v | MATQGQVITCKAAVAWEPNKPLVIEDVQVAPPQAGEVRIKILFTALCHTDAYTWS |
| | | | GKDPEGLFPCILGHEAAGIVESVGEGVTEVQPGDHVIPCYQAECQECKFCKSGKT |
| | | | NLCGKVRSATGVGVMLSDRKSRFSVNGKPIYHFMGTSTFSQYTVVHDVSVAKIDP |
| | | | KAPLEKVCLLGCGVPTGLGAVWNTAKVEPGSIVAVFGLGTVGLAVAEGAKAAGAS |
| | | | RIIGIDIDSKKYDVAKNFGVTEFVNPKDHEKPIQQVLVDLTDGGVDYSFECIGNVS |
| | | | VMRAALECCHKGWGTSVIVGVAASGQEISTRPFQLVTGRVWKGTAFGGFKSRSQ |
| | | | VPWLVEKYLKKEIKVDEYITHNLTLEEINKAFDLMHEGGCLRCVL |
| 843 | 34 | Bet_v | TAGQVIKCKAAVAWEAGKPLVIEEVEVAPPQANEVRVKILFTSLCHTDVYFWEAKG |
| | | | QTPLFPRIFGHEAGGIVESVGEGVTDLKPGDHVLPVFTGECKECRHCKSEESNMC |
| | | | DLLRINTDRGVMLSDGKTRFSIKGQPIYHFVGTSTFSEYTVVHVGCLAKINPKAPL |
| | | | DKVCILSCGISTGLGATLNVAKPKKGQSVAVFGLGAVGLAAAEGARIAGASRIIGV |
| | | | DLNPDRFEEAKKFGVTEFVNPKDHNKPVQEVIAELTDGGVDRAVECTGSIQAMIS |
| | | | AFECVHDGWGVAVLVGVPSKDDAFKTHPMNLLNERTLKGTFFGNYKPRTDIPGVV |
| | | | EKYMNKELELEKFITHTVPFSEINKAFDYMLHGKSIRCIISMD |
| 844 | 34 | Bet_v | LTIYITAERDTDTDLSQSKQRSPSSSSSEIAMSSTAGQVIKCKAAVAWEAGKPLVI |
| | | | EEVEVAPPQANEVRVKILFTSLCHTDVYFWEAKGQTPLFPRIFGHEAGGIVESVGE |
| | | | GVTDLKPGDHVLPVFTGECKECRHCKSEESNMCDLLRINTDRGVMLSDGKTRFSI |
| | | | KGQPIYHFVGTSTFSEYTVVHVGCLAKINPKAPLDKVCILSCGISTGLGATLNVAKP |
| | | | KKGQSVAVFGLGAVGLAAAEGARIAGASRIIGVDLNPDRFEEAKKFGVTEFVNPKD |
| | | | HNKPVQEVIAELTDGGVDRAVECTGSIQAMISAFECVHDGWGVAVLVGVPSKDD |
| | | | AFKTHPMNLLNERTLKGTFFGNYKPRTDIPGVVEKYMNKELELEKFITHTVPFSEIN |
| | | | KAFDYMLHGKSIRCIISMDA |
| 845 | 34 | Cyn_d | SLEERLVDLGFLLEKQMATTGKVIKCKAAVAWEAGKPLSMEEVEVAPPQAMEVRIK |
| | | | ILFTSLCHTDVYFWEAKGQNPVFPRIFGHEAGGIVESVGEGVTDVAPGDHVLPVFT |
| | | | GECKECAHCKSAESNMCDLLRINTDRGVMIGDGKSRFSINGKPIYHFVGTSTFSE |
| | | | YTVMHVGCVAKINPEAPLDKVCVLSCGISTGLGASINVAKPPKGSTVAVFGLGAVG |
| | | | LAAAEGARIAGASRIIGVDLNPNRFEEARKFGCTEFVNPKDHKKPVQEVLAEMTNG |
| | | | GVDRSVECTGNINAMIQAFECVHDGWGVAVLVGVPHKDAEFKTHPMNFLNERTL |
| | | | KGTFFGNFKPRTDLPNVVELYMKKELEVEKFITHTVPFSEINKAFDLMAKGEGIRCII |
| | | | RMDH |
| 846 | 34 | Cyn_d | MATTGKVIKCKAAVAWEAGKPLSMEEVEVAPPQAMEVRIKILFTSLCHTDVYFWE |
| | | | AKGQNPVFPRIFGHEAGGIVESVGEGVTDVAPGDHVLPVFTGECKECAHCKSAES |
| | | | NMCDLLRINTDRGVMIGDGKSRFSINGKPIYHFVGTSTFSEYTVMHVGCVAKINPE |
| | | | APLDKVCVLSCGISTGLGASINVAKPPKGSTVAVFGLGAVGLAAAEGARIAGASRII |
| | | | GVDLNPNRFEEARKFGCTEFVNPKDHKKPVQEVLAEMTNGGVDRSVECTGNINA |
| | | | MIQAFECVHDGWGVAVLVGVPHKDAEFKTHPMNFLNERTLKGTFFGNFKPRTDLP |
| | | | NVVELYMKKELEVEKFITHTVPFSEINKAFDLMAKGEGIRCIIRMDH |
| 847 | 34 | Fra_e | LSMSNTAGLVIPCKAAVSWEAGKPLVIQQVEVAPPQAMEVRVQIKYTSLCHTDLYF |
| | | | WEAKGQTPLFPRIFGHEAAGIIESVGEGVSDLQVGDHVLPVFTGECGDCAHCKSQ |
| | | | ESNMCDLLRINTDRGVMLSDGNSRFSINGNPINHFLGTSTFSEYTVVHSGCLAKV |
| | | | NPLAPLDKICILSCGISTGLGATLNVAKPKKGSSVAIFGLGAVGLAAAEGARIAGAS |
| | | | RIIGIDLNPNRFDEAKKFGVTEFVNPKEHDRPVQQVIAEMTNGGVDRSVECTGNV |
| | | | NVMVSAFECVHDGWGVAVLVGVPNKDAVFMTKPINLLNERTLKGTFFGNYKPRTD |
| | | | LPSVVDMYMNKKLELDKFITHRLSFSEINKAFEYMVKGEGLRCIISMEDE |
| 848 | 34 | Fra_e | TLSKRKGTKMSSTAGQVIRCKAAVSWEAGKPLVIEEVDVAPPQKMEVRLKILFTSL |
| | | | CHTDVYFWEAKEQTPLFPRIFGHEAGGIVESVGEGVADLQPGDHVLPMFTGECKE |
| | | | CRHCKSTESNMCDLLRINTDRGVMINDGKTRFSKNGQPIYHFLGTSTFSEYTVVH |
| | | | VGCVAKINPAAPLEKVCVLSCGISTGLGATLNVARPTKGSTVAIFGLGAVGLAAAE |
| | | | GARISGASRIIGIDLNPNRFKDAKKFGVTEFVNPKDHDRPVQQVLVEMTDGGVDR |
| | | | SVECTGNVDAMISAFECVHDGWGVAVLVGVPNKDDTFKTRPVNLLNERTLKGTFF |
| | | | GNYKPRSDIPSVVEKYMNKELELDKFITHQVRFSEINKAFDLMLRGESLRCIINMEA |
| 849 | 34 | Fra_e | IPPTGFSISHQTSYIQITQFTEIKKQISDMSSTVGQVIKCKAAVAWEAGKPLVIEEV |
| | | | EVAPPQKMEVRLKILFTSLCHTDVYFWEAKAQDSVFPRIFGHEAAGIVESVGEGVT |
| | | | ELTPGDHVLPVFTGECKECAHCKSEESNMCSLLRINTDRGVMINDGQTRFSINGK |
| | | | PIYHFVGTSTFSEYTVVHVGCVAKINPLAPLDKVCVLSCGISTGLGATLNVAKPKKG |
| | | | SSVAIFGLGAVGLGAAEGARLAGASRIIGVDLNSGRFEEAKKFGVTEFVNPKDHKK |
| | | | PVQEVIAEMTDGGVDRSVECTGNVNAMISAFECVHDGWGVAVLVGVPHKDAEFK |
| | | | THPMNLLNERTLKGTFFGNYKPRSDLPSVVELYMNNELELEKFITHEVPFNEINKAF |
| | | | ELMLKGEGLRCIIRM |
| 850 | 34 | Lol_p | HTDVYFWEAKGQTPVFPRILGHEAGGIVESVGEGVTELVPGDHVLPVFTGECKEC |
| | | | AHCKSEESNLCDLLRINVDRGVMIGDGQSRFTINGKPIFHFVGTSTFSEYTVIHVG |
| | | | CLAKINPEAPLDKVCVLSCGISTGLGATLNVAKPKKGSTVAIFGLGAVGLAAMEGA |
| | | | KMAGASRIIGVDLNPAKYEQAKKFGCTDFVNPKDHTKPVQEVLVEMTNGGVDSA |
| | | | VECTGNINAMISAFECVHDGWGVAVLVGVPHKEAVFKTHPMNFLNERTLKGTFFG |
| | | | NYKPRTDLPEVVEMYM |
| 851 | 34 | Lol_p | GEGAMATAGKVIKCKAAVAWEAGKPLSIEEVEVAPPQAMEVRVKILFTALCHTDVY |
| | | | FWEAKGQTPVFPRIFGHEAGGIVESVGEGVTELAPGDHVLPVFTGECKECPHCKS |
| | | | AESNMCDLLRINTDRGVMLSDGKSRFSIDGKPIYHFVGTSTFSEYTVLHVGCVAKI |
| | | | NPEAPLDKVCVLSCGISTGLGASINVAKPPKGSTVAIFGLGAVGLAAAEGARIAGA |
| | | | SRIIGIDLNANRFEEARKFGCTEFVNPKDHNKPVQEVLIEMTNGGVDRSVECTGNI |
| | | | NAMIQAFECVHDGWGVAVLVGVPHKDAEFKTHPMNFLNERTLKGTFFGNFKPRT |
| | | | DLPNVVEMYMKKELEVEKFITHSVPFSEINKAFDLMAKGEGIRCIIRMEN |
| 852 | 34 | Ole_e | TFLHFRGKSSMSNTAGLVIPCKAAVSWEAGKPLVIQQVEVAPPQAMEVRVKIKYTS |
| | | | LCRTDLYFWEAKGQTPLFPRIFGHEAAGIIESVGEGVSDLQVGDHVLPVFTGECGD |
| | | | CAHCKSEESNMCDLLRINTDRGFMLSDGKSRFSINGNPINHFLGTSTFSEYTVVHS |
| | | | GCLAKVNPLAPLDKICVLSCGISTGLGATLNVAKPKKGSSVAIFGLGAVGLAAAEG |
| | | | ARIAGASRIIGIDRNPSRFDEAKKFGVTEFVNPKEHNRPVQQVIAEMTNGGVDRSV |
| | | | ECTGNINAMVSAFECVHDGWGVAVLVGVPNKDAVFMTKPINLLNERTLKGTFFGN |
| | | | YKPRTDLPSIVDMYMNKKLELDKFITHHLSFSEINKAFEYMVKGEGLRCIISMED |
| 853 | 34 | Ole_e | KKQISEMSSTVGQVIKCKAAVAWEAGKPLVIEEVEVAPPQKMEVRLKVLFTSLCHT |
| | | | DVYFWEAKAQNSAFPRIFGHEAAGIVESVGEGVTELAPGDHVLPVFTGECKECAH |
| | | | CKSEESNMCSLLRINTDRGVMINDGQTRFSINGKPIYHFVGTSTFSEYTVVHIGCV |
| | | | AKINPLAPLDKVCILSCGISTGLGATLNVAKPTKGSSVAIFGLGAVGLGAAEGARLA |
| | | | GASRIIGVDLNPSRFEEAKKFGVTEFVNPKDHKKPVQEVIAEMTDGGVDRSVECT |
| | | | GNVNAMISAFECVHDGWGVAVLVGVPHKDAEFKTHPMNLLNERTLKGTFFGNYK |
| | | | PRSDLPSVVEMYMNKELELEKFITHEVPFHEINKAFELMLKGEGLRCIIRME |
| 854 | 34 | Ole_e | FLFTFIDSMATKGQAITCKAAVAWEPNKPLVIEEVQVAPPQAGEVRIKILFTALCHT |
| | | | DAYTWSGKDPEGLFPCILGHEAAGVVESVGEGVIELQPGDHVIPCYQAECKECKF |
| | | | CKSGKTNLCGKVRVATGAGVMLSDRNSRFSINGKPIYHFMGTSTFSQYTVVHDVS |
| | | | VAKIDPKAPLEKVCLLGCGIPTGLGAVWNTAKVEQGSIVAVFGLGTVGLAVAEGAK |
| | | | AAGASRIIGIDIDSKKFDTAKKFGVTEFINPKDYDKPIQQVIVDLTDGGVDYSFECI |
| | | | GNVSVMRSALECCHKGWGTSVIVGVAASGQEISTRPFQLVTSRVWKGTAFGGFK |
| | | | SRSQVPWLVDKYMKKEIKVDEYISHNLTLAEINKAFDLMHDGVCLRVVLNMHA |
| 855 | 34 | Pla_l | ESVGEGVTELAPGDHVLPVFTGECGDCAHCKSQESNMCNLLRINVERGVMINDG |
| | | | KSRFSINGKPVYHFVGTSTFSEYTVVHVGCLAKINPAAPLDKVCVLSCGISTGLGAT |
| | | | LNVAKPKKGQSVAIFGLGAVGLGAAEGARLAGASRIIGVDLNSSRFEEAKKFGVTE |
| | | | FVNPKDYKKPVQEVIAEMTDGGVDRSVECTGNINAMISAFECVHDGWGVAVLVG |
| | | | VPHKDAEFKTHPMNVLNERTLKGTFFGNYKPRSDLPSVVEMYMNKELELEKFITHE |
| | | | VPFAEINKAFDLMLKGEGLRCIIKME |
| 856 | 34 | Pla_l | FPNQIYNSLNLNFQAAEGARVSGASRIIGIDLNPARFEQAKKFGVTECLNPKDHKK |
| | | | PIQEVIVEMTDGGVDRSVECTGNVTAMISAFECVHDGWGVAVLVGVPNKEDAFK |
| | | | TNPVNLLNERTLKGTFFGNYKPRSDIPVVVEKYMNKEMELDKFITHRVPFSEINKAF |
| | | | DYMIRGESLRCIISMEN |
| 857 | 34 | Pla_l | EIMSSTTGQVIRCKAAVSWEAGKPLVIEEVEVAPPQKMEVRIKILFTSLCHTDVYF |
| | | | WEAKGQTPLFPRIFGHEAGGIVESVGEGVTDIQPGDHVLPVFTGECKECRHCKSA |
| | | | ESNMCDLLRINTDRGVMIQDGKSRFSKDGKPIHHFLGTSTFSEYTVVHVGCVAKI |
| | | | NPEAPLDKVCVLSCGFSTGFGATVNVAKPPQGSTVAIFGLGAVGLAAAEGARV |
| 858 | 34 | Poa_p | AQRTMATAGKVIKCKAAVAWEAGKPLSIEEVEVAPPQAMEVRVKILFTSLCHTDVF |
| | | | FWEPKVQKPLFPRIFGHEAGGIVESVGEGVTDVAPGDHVLPVFTGECKECRHCKS |
| | | | AESNMCDLLRINTDRGVMISDGKSRFSIDGKPIYHFVGTSTFSEYTVMHVGCVAKI |
| | | | NPEAPLDKVCVLSCGISTGLGASINVAKPPKGSTVAIFGLGAVGLAAAEGARIAGA |
| | | | SRIIGVDLNANRFEEARKFGCTEFVNPKDHTKPVQEVLAEMTDGGVDRSVECTGN |
| | | | INAMIQAFECVHDGWGVAVLVGVPHKDAEFKTHPMNFLNERTLKGTFFGNFKPRT |
| | | | DLPNVVEMYMKKELEVEKFITHSVPFSEINKAFDLMAKGEGIRCIIRMEH |
| 859 | 34 | Que_a | YYNIERKMENGLRNPSETTGKVITCKAAITWGPGEPFVIEEVRVDPPQKMEVRIKIL |
| | | | FTSICHTDLSAWQGENEAQRAYPRILGHEASGIVESVGEGVMDIKKGDHVVPIFN |
| | | | GECGDCLYCKCEKTNMCERAGVNPFMTVMVNDGKSRFSCKEGKPIFHFLNTSTFS |
| | | | EYTVVESACVVKIDPDASLKTMTLLSCGVSTGVGAAWNIANVKAGSTVAIFGLGA |
| | | | VGLAVAEGARARGATKIIGVDINPNKFTKGRAMGITDTINPRDFEKPVHECIREMT |
| | | | GGGVDYSFECAGISEVLREAFLSTHEGWGLTVILGIHTSPKMLPLHPMELFTGRVII |
| | | | ASVFGGFKGKTQLPNFAKECMQGVVNLEEFITHELPFEKINEAFQLLIDGKSVRCM |
| | | | LHL |
| 860 | 34 | Que_a | RIFGHEAGGIVESVGEGVTDLKPGDHALPVFTGECKECRHCKSEESNMCDLLRINT |
| | | | DRGVMLNDGKSRFSINGQPIYHFVGTSTFSEYTVLHVGSVAKINPAAPLDKVCVLS |
| | | | CGISTGLGATLNVAKPKKGSTVAVFGLGAVGLAAAEGARIAGASRIIGVDLNAKRF |
| | | | DEAKKFGVTEFVNPKDHDKPVHEVLAEMTNGGVDRSIECTGSINAMISAFECVHD |
| | | | GWGVAVLVGVPNKDDAFKTHPMNILNERTIKGTFFGNYKPRSDLPSVVEKYMNKE |
| | | | LELEKFITHEVSFSEINKAFEYMLRGEGLRCIIRMDA |
| 861 | 34 | Que_a | KAAIAWEAGKPLVIEQVEVAPPQTMEVRIKIKYTSLCHTDLYFWEAKGQTPLFPRIF |
| | | | GHEAAGVVESVGEGVSDLQVGDHVLPVFTGECGDCRHCKSEESNMCDLLRINTD |
| | | | RGVMLNDGKSRFSINGTPINHFLGTSTFSEYTVVHSGCLTKISPLAPLDKVCILSCG |
| | | | ISTGLGATLNVAKPKKGSTVAVFGLGAVGLAAAEGARIAGASRIIGIDLSPKRYEEA |
| | | | KKFGVTEFVNPKDHDRPVQEVIAEMTNGGVDRSIECTGNINCMISAFECVHDGW |
| | | | GVAVLVGVPNKDAVFMTKPINVLNERTLKGTFFGNYKPRTDLPSVVDMYMNKKLE |
| | | | VEKFITHRVPFSDINKAFEYMLKGEGLRCIISMEE |
| 862 | 34 | Que_a | AMSSTAGQVIKCKAAVAWEAGKPLVIEEVELAPPQANEVRMKILFTALCHTDVYF |
| | | | WEAKGQTPMFPRIFGHEAGGIVESVGEGVTELKPGDHVLPIFTGECGKCSHCNSE |
| | | | ESNLCDTLRINTERGVLLNDGKTRFSKNGQPIYHFLGTSTFSEYTIAHVGCVAKINP |
| | | | AAPLDKVCVLSCGVSTGMGATLNVAKPKKGQSVAVFGLGAVGLAACEGARMAGA |
| | | | GKIIGVDLNPDRFNEAKKFGVTDFVNPKDHDKPVQEVIAEMTNGGVDRAVECTGS |
| | | | FQAMIQAFECVHDGWGVAVLVGVPNKDDAFKTHPLNFLNERTLKGTFFGNYKPRT |
| | | | DIPSQVEKYMKKELELEKFITHSVPFSEINKAFDYMLKGESIRCIIRMDA |
| 863 | 34 | Que_a | AMSSTAGQVIKCRAAVAWEAGKPLVIEEVEVAPPQANEVRMRILFTALCHTDVYF |
| | | | WEAKGQTPLFPRIFGHEAGGIVESVGEGVTELKPGDHVLPIFTGECGKCSHCNSEE |
| | | | SNLCDTLRINTERGVLLNDGKTRFSKNGQPIYHFLGTSTFSEYTIAHVGCVAKINPA |
| | | | APLDKVCVLSCGVSTGMGATLNVAKPKKGQSVAVFGLGAVGLAACEGARMAGAG |
| | | | KIIGVDLNPDRFNEAKKFGVTDFVNPKDHDKPVQEVIAEMTDGGVDRALECTGSI |
| | | | QAMISAFECVHDGWGVAVLVGVPNKDDSFQTHPVNFLNERTLKGTFFGNYKPRT |
| | | | DIPSVVEKYMNKELELEKFITHSVPFSEINKAFDYMLKGQSIRCIIRMGA |
| 864 | 34 | Que_a | MATQGQVITCKAAVAWEPNKPLVIEDVQVAPPQAGEVRIKILFTALCHTDAYTWS |
| | | | GKDPEGLFPCILGHEAAGIVESIGEGVTEVQPGDHVIPCYQAECRECKFCKSGKTN |
| | | | LCGKVRSATGVGVMLSDRKSRFSVNGKSIYHFMGTSTFSQYTVVHDVSVAKIDPK |
| | | | APLEKVCLLGCGVPTGLGAVWNTAKVESGSIVAIFGLGTVGLAVAEGAKTAGASRI |
| | | | IGIDIDSKKFDTAKKFGVTEFVNPKDHEKPIQQVIVDLTDGGVDYSFECIGNVSVM |
| | | | RAALECCHKGWGTSVIVGVAASGQEISTRPFQLVTGRVWKGTAFGGFKSRSQVP |
| | | | WLVEKYLKKEIKVDEYITHNLTLGEINEAFHLMHEGGCLRCVLKV |
| 865 | 39_59 | Amb_a | VVSPPFVFLTTVKSELRPEIQVAAQNCWVKKGGAFTGEVSAEMLANLGVPWVILG |
| | | | HSERRALLNESNEFVGDKVAYALSQGLKVIACVGETLEQREAGTTMDVVAAQTKA |
| | | | IADKISSWDNVVLAYEPVWAIGTGKVASPAQAQEVHAGLRKWFEENISAEVSATT |
| | | | RIIYGGSVSGSNCKELAGQPDVDGFLVGGASLKPEFINIIKAAEAK |
| 866 | 39_59 | Amb_p | VSTLNAGDLPSTDIVEVVVSPPFVFLTTVKSELRPEIQVAAQNCWVKKGGAFTGEV |
| | | | SAEMLANLGVPWVILGHSERRALLNESNEFVGDKVAYALSQGLKVIACVGETLEQ |
| | | | REAGTTMDVVAAQTKAIADKISSWDNVVLAYEPVWAIGTGKVASPAQAQEVHAG |
| | | | LRKWFEENISAEVAATTRIIYGGSVSGSNCKELAGQPDVDGFLVGGASLKPEFINII |
| | | | KAAEAK |
| 867 | 39_59 | Bet_v | MARKFFVGGNWKCNGTTEEVKKIVSTLNEAQVPSQDVVEVVVSPPFVFLPLVKTLL |
| | | | RPDIHVAAQNCWVKKGGAYTGEVSAEMLVNLGIPWVILGHSERRLILNESNEFVG |
| | | | DKVAYALEKGLKVIACVGETLEQRESGSTVEIVAAQTKAIAERVSNWANVVLAYEP |
| | | | VWAIGTGKVATPAQAQEVHSELRKWLQANTSPEVAATTRIIYGGSVNGANCKELA |
| | | | AKPDVDGFLVGGASLKPEFIDIIKSAEVKKSA |
| 868 | 39_59 | Bet_v | RKFFVGGNWKCNGTAEEVKKIVSTLNEAEVPSEDVVEVVVSPPFVFLPLVKSLLRS |
| | | | DFHVAAQNCWVRKGGAFTGEISAEMLVNLGIPWVILGHSERRALLSESNEFVGDK |
| | | | VAYALSQGIKVIACVGET |
| 869 | 39_59 | Cyn_d | GGNWKCNGTGEDVKKIVTVLNEAEVPSEDVVEVVVSPPFVFLQQVKGLLRPDFSV |
| | | | AAQNCWVRKGGAFTGEISAEMLVNQQLPWVILGHSERRALLGESNDFVADKVAY |
| | | | ALSQGLKVIACIGETLEQREAGTTMDVVAAQTKAIAEKISDWTNVVLAYEPVWAIG |
| | | | TGKVASPAQAQEVHDGLRKWLQSAVSPAVAESTRIIYGGSVNGGNCKELAAQPD |
| | | | VDGFLVGGASLKPEFVDIIKSATVKSSS |
| 870 | 39_59 | Cyn_d | MGRKFFVGGNWKCNGTTEQVDKIVKTLNEGQIPSTDVVEVVVSPPYVFIPVVKTQ |
| | | | LRPEIQVAAQNCWVKKGGAYTGEVSAEMLANLGVPWVILGHSERRALLGESNEFV |
| | | | GDKVAYALAQGLKVIACVGETLEQRESGSTMDVVAAQTKAIAERIQDWTNVVVAY |
| | | | EPVWAIGTGKVATPAQAQEVHASLREWLKTNVSPEVSESTRITYGGSVTAANCKE |
| | | | LAGQPDVDGFLVGGASLKPEFIDIINSATVKSA |
| 871 | 39_59 | Que_a | MARKFFVGGNWKCNGTTEEVKKIVSTLNEGQVPPPDVVEVVVSPPFVFLPLVKNLL |
| | | | RPDFHVAAQNCWVKKGGAFTGEVSAEMLVNLGIPWVILGHSERRQILNETNEFVG |
| | | | EKVAYALSKGLKVIACVGETLEQRESGTTVEVVAAQTKAIAERVSNWADVVLAYEP |
| | | | VWAIGTGKVATPAQAQEVHFELRKWFHANISPEVAATIRITYGGSVNGANSKELAV |
| | | | QPDVDGFLVGGASLKPEFIDIIKSAEVKKSA |
| 872 | 43 | Amb_p | AASQWLYVVPWGLRKILNYISRKYNNPPIYITENGMDDEDNDASSLHEMLDDKLR |
| | | | IAYYKGYLASVFLAIKDGVDVRGYFAWSLVDNFEWPLGYTKRFGLVYIDYKNGLTR |
| | | | HPKSSAYWFMKLLKGE |
| 873 | 43 | Bet_v | LLSVIVIQCVAHATELNVNDTGGLGRHNFPKGFVFGTATSAYQVEGMAHKDGRGP |
| | | | SIWDPFVKIPGNIANNATADVSVDQYHRYKEDVDIMAKFNFDAYRFSISWSRIFPN |
| | | | GRGKVNWKGVAYYNRLIDYLLKRGITPYANLYHYDLPLALEMKYKGLLSDQVVKDF |
| | | | ADYADFCFKTFGDRVKNWMTFNEPRVVAALGYDNGIFAPGRCSKAFGNCTAGNS |
| | | | ATEPYIAAHHLILSHAAAVQRYRQKYQEKQKGRIGILLDFVWYEPLTKSKDDNNAA |
| | | | QRARDFHVGWFIHPIVYGEYPRTMQDIVADRLPRFTKEEVKMVKGSIDFVGINQYT |
| | | | AFYMYDPHQPKPKDLGYQQDWNVGFAYEKNGVPIGPRANSNWLYIVPWGLYKAL |
| | | | TYIKEHYGNPTVILSENGMDDPGNVTLSKGLHDTTRINFYTGYLTQLKKAVDEGAN |
| | | | VFGFFAWSLLDNFEWRSGYTSRFGIVYVDYTNLKRYPKMSAYWFKRLLRRNQ |
| 874 | 43 | Cyn_d | TMALSAHGKVGENTNLTRESFPPGFVFGTASSAYQVEGNANKYGRGPCIWDTFLM |
| | | | HPGTTPDNATANVTVDEYHRYMDDVDNMVRVGFDAYRFSISWSRIFPSGVGKIN |
| | | | KDGVDYYHRLIDYMLANKITPYVVLHHFDLPQVLQDQYNGWLSPRVVGDFEKFAD |
| | | | FCFKTYGDRVKNWFTINEPRMMAVHGYSDAFFAPARCTGCKVGGNSATEPYIAGH |
| | | | HLLLSHAAAVKTYREKYQAQQKGKIGILLDFVWYEPLSDSMEDGYAAHRARMFTL |
| | | | GWFLHPITYGHYPPSMENIVRGRLPNFTFEQSEMVKGSADYIGINHYTTYYASHYI |
| | | | NDTEMSYRNDWSVKLSYSRNGVPIGKKAYSDWLYVVPWGIYKAVMWTKEKFNN |
| | | | PVIIIGENGIDQPGNETLPGALYDTFRIDYFEQYLRELKSAVNDGANVIGYFAWSLL |
| | | | DTFEWRLGFTSKFGLVYVDRQTFTRYPKDSARWFRKVIKREE |
| 875 | 43 | Que_a | SMSLDSGGLSRDKFPKGFVFGTATSAYQVEGMAHKDGRGPSIWDTFVKIPGIVAN |
| | | | NGTADVSVDQYHRYKEDIDIMKKLNFDAYRFSISWSRIFPDGTGKVNHKGVAYYN |
| | | | RLINYLLRRGITPYANLYHYDLPLALEKKYKGLLSDQVVKDFADYADFCFRTFGDRV |
| | | | KNWMTFNEPRVVAALGYDNGFFAPGRCSKPYGNCTAGNSATEPYIVAHHLILAHA |
| | | | AAVQRYREKYLEKQKGRIGILLDFVWYEPLTRSKADNYAAQRARDFHVGWFIHPIV |
| | | | YGEYPRTMQDIVGDRLPKFTKEEVKMVKGSMDFVGINQYTAYYMYDPHKSKPKVL |
| | | | GYQQDWNAGFAYNKKGVPIGPKANSYWLYNVPWGLYKAITYIKEHYGNPTVILSE |
| | | | NGMDDPGNVTISKGLHDTTRINFYKGYLTQLKKAVDEGANVVGYFAWSLLDNFE |
| | | | WRLGYTSRFGIVYVDFANLKRYPKMSAYWFKRLLKRNK |
| 876 | 47 | Amb_a | VSGGSLIKSLRKLVEEPYVGSVDWSKWHMFWVDERVVPKDHPDSNYLLAFDGFL |
| | | | SKVPIPPGNVHAINDALSAEAAADDYETHIKHLVHNGIISTSETTGFPKFDLMLLGM |
| | | | GPDGHVASLFPGHPLLAEKSKWVTFIKESPKPPP |
| 877 | 47 | Amb_p | GGSLIKSLRKLVEEPYVGSVDWSKWHMFWVDERVVPKDHPDSNYLLAFDGFLSK |
| | | | VPIPPGNVHAINDALSAEAAADDYETHIKHLVHNGIISTSATTGFPKFDLMLLGMGP |
| | | | DGHVASLFPGHPLLAEKSKWVTFIKESPKPPPERITFTFPVINSSANVALVVAGAGK |
| | | | AHPVHVALGNGQEPEPLPVQMVAPEGQLAWFLDKDAASKL |
| 878 | 47 | Bet_v | MAATTAEKGGDKKKVEVFDTEEDLAVSLAKYTADLSDKFSKERGAFTVVLSGGSLI |
| | | | KSLRKLLEPPYIDSVEWSKWHVFWVDERVVPKDHEDSNYKLAYDGFLSKIPIVPG |
| | | | HVYAINDALSAEGAADDYETCLKHLVKINVIDLSAASGFPKFDLMLLGMGPDGHV |
| | | | ASLFPGHPLLKENEKWVTFIKDSPKPPPERITFTFPVVNSSAYIALVVAGAGKAGVV |
| | | | QQALGNGQNSDKLPVQIVSPEGELTWFLDKDAASKL |
| 879 | 47 | Cyn_d | SATAAAAVAFLPPLTGRTSPPAYRVPANSRRGSVSNSRIFTSFAPSPILRAAAMATD |
| | | | GAAPAASDAGSKQKLLTFDSEEELAVSLAKYTAELSAKFAAERGAFTAVLSGGSLI |
| | | | KALRKLTEPPYLDSVDWSKWHVFWVDERVVPKDHEDSNYKLALDGFLSKVPIPTR |
| | | | QVYAINDALSAEGAADDYETCLKQLVKNGVIAMSAATGFPRFDLQLLGMGPDGHI |
| | | | ASLFPGHPLVNENQKWVTYIKDSPKPPPERITFTFPVINSSAYIAMVVTGAGKAAAV |
| | | | QKALSDKEISSDKLPVEMAVLQDGEFTWFTDKEAVSLLQNK |
| 880 | 47 | Que_a | MATKGEVKKEVFESGEDLAVALAKYTAQLSDKFCKERGAFSVVLSGGSLINSLRKL |
| | | | VEPPYIDSIEWSRWHIFWADERVVPKDHEDSNYKLAYDGFLSKVPIPPGNVYAIND |
| | | | ALSAEGAAEDYETCLRHLVKSNVVDISAASGFPKFDLQLLGMGPDGHVASLFPGH |
| | | | PLVKENEKWVAFIKDSPKPPPERITFTFPVINSSAYIALVVNGANKAGAVQNALGNS |
| | | | QNSEKLPVAMVSPEGELAWFLDTAAASKL |
| 881 | 49 | Amb_a | MGPGEWSPEMRKTYNLLDAVSRHTIQVYPRSWTAIMLTFDNAGMWSVRSNIWER |
| | | | HYLGEQFYISVTSPERSLRDEYNMPDNALRCGKVVGLPLPPSYAAA |
| 882 | 49 | Amb_p | MGPGEWSPELRKTYNLLDAVSRNSIQVYPRSWTAVMLTFDNAGMWNVRSNLWE |
| | | | RHYLGEQFYISVVSPARSLRDEYNMPEDDLRCGKVVGLPMPPSYLPA |
| 883 | 49 | Bet_v | IEPGRWSPVKRKNYNLLDAVSRHNIQVYPNSWAAIMTTLDNAGMWSLRSEMWER |
| | | | VYLGQQLYFSVLSPARSLRDEYNLPDNTPLCGIVPGLPLPPPY |
| 884 | 49 | Cyn_d | MGPGTWSPQSRKTYNLLDTVSRHTIQVYPRSWTAVMLTFDNAGMWNVRSNLWE |
| | | | RQYLGEQMYISVISPARSLRDEYNMPETSLRCGKVVGLPMPPSYLPA |
| 885 | 49 | Que_a | MGPGEWSPELRKTYNLLDAVSRNSIQVYPRSWTAVMLTFDNAGMWNVRSNLWE |
| | | | RHYLGEQFYISVVSPARSLRDEYNMPEDDLRCGKVVGLPMPPSYLPA |
| 886 | 54 | Amb_a | GVELARRDMATTTRVAAGVLLVLSALALVARAEDPYLFFEWKVTYGTKPVLGVPQK |
| | | | VILINGEFPGPRINCTSNNNIVVNVFNQLDHPLLFTWNGMQHRKNSWMDGMPGT |
| | | | QCPILPNTNFTYKWQPKDQIGSFYYFPSIGMQRAAGGYGGISVYSRLLIPVPFDQP |
| | | | PPENDHVVLIGDWYTKDHEVLARQLDAGKSVGRPAGVVINGKGGKDLEAAPLFTF |
| | | | EAGKTYRLRVCNTGIKASLNFRIQGHIMTLVELEGSHTLQDVYDSLDVHVGHCLSV |
| | | | LVDADQKPGDYYMVASTRFIHDAKSAKAIIRYAGSSAPPPAELPEPPAGWAWSIN |
| | | | QARSFRWNLTSSAARPNPQGSYHYGQINITRTIKVRVSRGHINGKLRYGFSGVSH |
| | | | RDPETPVKLAEYFNVTDGVFSYNQMGDVPPAVNGPLHVVPNVITAEFRTFIEIVFEN |
| | | | PEKSLDSVHLDGYAFFGVGMGPGEWSPEMRKTYNLLDAVSRHTIQVYPRSWTAI |
| | | | MLTFDNAGMWSVRSNIWERHYLGEQFYISVTSPERSLRDEYNMPDNALRCGKVV |
| | | | GLPLPPSYAAA |
| 887 | 54 | Amb_p | AMGRTTFVALFICLSAGALMVHAEDPYHFFEWNVTYGTIAPLGVPQQGILINGQFP |
| | | | GPKINCTSNNNIVVNVFNHLDEPFLLTWNGVQQRKNSWQDGTLGTMCPILPGKN |
| | | | FTYHFQVKDQIGSFYYFPTTGLHKASGAIGGLQVHSRDLIPVPFDNPADEYFLLLGD |
| | | | WYNKGHKSLKKLLDSGRSIGRPDGIQINGKSGKVGDEAAEPLFTMESGKTYRYRV |
| | | | CNVGMRTSINFRLQGHTLKLVEMEGSHTVQNVYDSLDLHAGQCLSVLITANQAPK |
| | | | DYYLVVSSRFAQHQLSSVAIIRYLNGNSPASLELPPSPPDNTEGIAWSINQFRSFR |
| | | | WNLTASAARPNPQGSYHYGQINITRTIKLANSRSYVDGKLRFGLNGVSHVDSETP |
| | | | LKLAEYFEASDKLFKYDIIKDEPPQDDTKVILAPNVLNATFRNFVEIIFENHERTIQT |
| | | | YHLDGYSFFAVAIEPGRWSPEKRKNYNLLDAVSRHSIQVYPNSWAAVMTTLDNAG |
| | | | MWSLRSEMWERVYLGQQLYFSVLSPARSLRDEYNLPDNTPLCGIVPGLPLPPPY |
| 888 | 54 | Bet_v | RGRKMGGVMFILMLCLTAGAMSGVRGEDPYLFFTWNVTYGTISPLGVPQQGILIN |
| | | | GQFPGPNINSTTNNNIVINVHNSLHEPFLLTWSGIQHRKNSWQDGVLGTMCPIPP |
| | | | GTNYTYHFQVKDQIGSYTYYPTTATHRAAGAFGGLRVNSRLLIPVPYADPEDDYTVL |
| | | | IGDWYAKSHQTLRKFLDSGRSLGRPDGVLINGKSGKDKPLFTMKAGKTYKYRICN |
| | | | VGVKNSLNFRIQGHTMKLVELEGSHTVQNTYQSLDVHVGQCLSVLVTADQKPKD |
| | | | YYVVASTRFTKSVLTGKGIIRYIGGKGPASPEIPEAPVGWAWSLNQFRTFRWNLTA |
| | | | SAARPNPQGSFHYGAINITRTIKLVNSASKVDGKHRYAVNGISHIDPPTPLKLAEYY |
| | | | GVADKVFKYDTIPDDPPAQGAPNITSAPVVLNMTFRNFVETIFENHEKSIQSWHLD |
| | | | GYSFFAVAIEPGRWTPERRRNYNLLDAVSRHTVQVFPKSWAAILLTFDNAGMWNI |
| | | | RSEIVERRYLGQQLYASILSPARSLRDEYNIPDNALLCGLVKNLPKPPPYV |
| 889 | 54 | Cyn_d | GVLLVLTALAVVHAEDPYLFFEWKVTYGTKSLLGVPQKVILINGEFPGPRINCSSNN |
| | | | NIVVNVFNQLDQPLLFTWNGMQHRKNSWMDGLPGTNCPIAPGTNFTYKWQPKD |
| | | | QIGSFFYFPSLGMQRAAGGYGPISVVSRLLIPVPFDPPADDHVVLIGDWYTKDHEV |
| | | | MARLLDSGRSIGRPAGVLINGKGGKDAAAAPIFTFEAGKTYRLRVCNTGIKSSLNF |
| | | | RIQGHDMKLVEMDGSHTVQDMFDSLDVHPGHCFSVLVDADQKPGDYYVVASTR |
| | | | FIHDPKSVSAVIRYAGSSTPPAPHVPEPPEGWAWSINQWRSFRWNLTASAARPNP |
| | | | QGSYHYGQINITRTIKLQISRGHIDGKLRYGFNGVSHVDADTPLKLAEYFNVTDGV |
| | | | FKYNQMGDAPPAVNGPLRVMPSVISAEFRTFIEVIFENPEKSMDSLHLDGYAFFAV |
| | | | GMGPGKWSPELRKTYNLLDAVSRHTIQVYPRSWTAIMLTFDNAGMWNVRSNIWE |
| | | | RHYLGEQVYVSVISPERSLRDEYNMPENALRCGKVIGLPLPPSYNPA |
| 890 | 54 | Que_a | AMGRMTFVELFLCLSAGALMVHAEDPYHFFEWNVTYGTIAPLGVPQQGILINGQFP |
| | | | GPKINCTSNNNIVVNVFNNLDEPFLLTWNGVQHRKNSWQDGTLGTMCPILPGKN |
| | | | FTYHFQVKDQIGSFYYFPTTGLHKASGAIGGLQVHSRDLIPVPFDNPADEYFVVLG |
| | | | DWYNKGHKSLKKLLDSGRSIGRPDGIQINGKSGKVGDKVAEPLFTMESGKTYRYR |
| | | | VCNVGMRTSVNFRLQGHTLKLVEMEGSHTVQNVYDSLDLHAGQCLSVLITANQA |
| | | | PKDYYLVVSSRFAQHQLSSVAIIRYLNGNSPASLELPPSPPDNTEGIAWSINQFRSF |
| | | | RWNLTASAARPNPQGSYHYGQINITRTIKLTNSRSYVDGKLRFGLNGVSHVDSET |
| | | | PLKLAEYFEASDKVFKYDLMKDEPPQENTKVTLAPNVLNATFRNFVEIIFENHERTI |
| | | | QTYHLDGYSFFAVAIEPGRWSPEKRKNYNLLDAVSRHSIQVYPNSWAAIMTTLDN |
| | | | AGMWSLRSEMWERVYLGQQLYFSVLSPARSLRDEYNMPDNTPLCGIVRGLPLPPP |
| | | | Y |
| 891 | 49_54 | Amb_p | NITRTIKLKITRGHLDGKLKYGFNGVSHVDADTPLKLAEYFNVTDGVFRYNQMGDS |
| | | | PPGVNGPLHAIPNVITAEFRTFIEIIFENPEKSMDSLHLDGYAFFAVGMGPGEWSPE |
| | | | LRKTYNLLDAVSRNSIQVYPRSWTAVMLTFDNAGMWNVRSNLWERHYLGEQFYI |
| | | | SVVSPARSLRDEYNMPEDDLRCGKVVGLPMPPSYLPA |
| 892 | 49_54 | Amb_p | LWERHYLGEQMYISVISPARSLRDEYNMPETSLRCGKVVGLPMPPSYLPA |
| 893 | 49_54 | Amb_p | AATAGGVLLLALLVLSTTQVARAEDPYLFFEWHVTYGTRTLLGVPQKVILINDEFPG |
| | | | PRINCSSNNNIVVNVFNQLEEPLLFTWNGMQQRKNSWQDGLPGTNCPVAPGTNY |
| | | | TFKWQAKDQIGSFFYFPSLGMQRAAGGYGMISVVSRLLIPVPFDPPADDHVVLIG |
| | | | DWYTKDHTVMASLLDAGKSPGRPAGVLINGKGGNDAASQPMFTFEAGKTYRLRV |
| | | | CNVGIKSSLNFRIQGHDMKLVEMEGSHTLQNTYDSLDVHVGQCLSVLVDADQKP |
| | | | ADYLMVASTRFIADATSVSAVIRYAGSNTPAAANVPEPPAGWAWSINQWRSFRW |
| | | | NLTASAARPNPQGSYHYGQINITRT |
| 894 | 49_54 | Amb_p | AATAGGVLLLALLVLSTTQVARAEDPYLFFEWHVTYGTRTLLGVPQKVILINDEFPG |
| | | | PRINCSSNNNIVVNVFNQLDQPLLFTWNGMQHRKNSWMDGLPGTNCPIAPGTNF |
| | | | T |
| 895 | 49_54 | Ant_o | PPPSYSHKPGDVFHGRLLIDPPIPPQLLHYNPSRERNLFHSVRRPLILMATTMRGTA |
| | | | ATAGGVLLLALLVLSTTQVARAEDPYLFFEWHVTYGTRTLLGVPQKVILINDEFPGP |
| | | | RINCSSNNNIVVNVFNQLEEPLLFTWNGMQQRKNSWQDGLPGTNCPVAPGTNYT |
| | | | FKWQAKDQIGSFFYFPSLGMQRAAGGYGMISVVSRLLIPVPFDPPADDFQVLVGD |
| | | | WYTKDHTVMASLLDAGKSPGRPAGVLINGKGGKDAASQPMFTFEAGKTYRLRVC |
| | | | NVGIKSSLNFRIQGHDMKLVEMEGSHTLQNTYDSLDVHVGQCLSVLVDADQKPA |
| | | | DYLMVASTRFIADATSVSAVIRYAGSNTPPAANVPEPPAGWAWSINQWRSFRWN |
| | | | LTASAARPNPQGSYHYGQINITRTIKLKITRGHLDGKLKYGFNGVSHVDADTPLKL |
| | | | AEYFNVTDGVFRYNQMGDSPPGVNGPLHAIPNVITAEFRTFIETIFENPEKSMDSLH |
| | | | LDGYAFFAVGMGPGEWSPELRKTYNLLDAVSRNSIQVYPRSWTAVMLTFDNAGM |
| | | | WNVRSNLWERHYLGEQFYISVVSPARSLRDEYNMPEDDLRCGKVVGLPMPPSYLP |
| | | | A |
| 896 | 49_54 | Ant_o | PPPSYSHKPGDVFHGRLLIDPPIPPQLLHYNPSRERNLFHSVRRPLILMATTMRGTA |
| | | | ATAGGVLLLALLVLSTTQVARAEDPYLFFEWHVTYGTRTLLGVPQKVILINDEFPGP |
| | | | RINCSSNNNIVVNVFNQLEEPLLFTWNGMQQRKNSWQDGLPGTNCPVAPGTNYT |
| | | | FKWQAKDQIGSFFYFPSLGMQRAAGGYGMISVVSRLLIPVPFDPPADDFQVLVGD |
| | | | WYTKDHTVMASLLDAGKSPGRPAGVLINGKGGKDAASQPMFTFEAGKTYRLRVC |
| | | | NVGIKSSLNFRIQGHDMKLVEMEGSHTLQNTYDSLDVHVGQCLSVLVDADQKPA |
| | | | DYLMVASTRFIADATSVSAVIRYAGSNTPPAANVPEPPAGWAWSINQWRSFRWN |
| | | | LTASAARPNPQGSYHYGQINITRTIKLKITRGHLDGKLKYGFNGVSHVDADTPLKL |
| | | | AEYFNATKGIFEYNLIGDTPPPEGTPIKLAPNVINTEWRTYIEVVFENPEKSIDSFHL |
| | | | NGYAFFAAGMGPGLWTPECRQTYNLLDTVSRHTIQVYPRSWTAVMLTFDNAGMW |
| | | | NLRSNLWERYYMGEQMYISCVSPARSLRDEYNMPENGLRCGNVIGLPLPPSYIPG |
| 897 | 49_54 | Bet_v | IDRGRKMGGVMFILMLCLTAGAMSGVRGEDPYLFFTWNVTYGTISPLGVPQQGILI |
| | | | NGQFPGPNINSTTNNNIVINVHNSLHEPFLLTWSGIQHRKNSWQDGVLGTMCPIP |
| | | | PGTNYTYHFQVKDQIGSYIYYPTTATHRAAGAFGGLRVNSRLLIPVPYADPEDDYTV |
| | | | LIGDWYAKSHQTLRKFLDSGRSLGRPDGVLINGKSGKDKPLFTMKAGKTYKYRIC |
| | | | NVGVKNSLNFRIQGHTMKLVELEGSHTVQNTYQSLDVHVGQCLSVLVTADQKPK |
| | | | DYYVVASTRFTKSVLTGKGIIRYIGGKGPASPEIPEAPVGWAWSLNQFRTFRWNLT |
| | | | ASAARPNPQGSFHYGAINITRTIKLVNSASKVDGKHRYAVNGISHIDPPTPLKLAEY |
| | | | YGVADKVFKYDTIPDDPPAQGAPNITSAPVVLNMTFRNFVEIIFENHEKSIQSWHL |
| | | | DGYSFFAVAIEPGRWTPERRRNYNLLDAVSRHTVQVFPKSWAAILLTFDNAGMWN |
| | | | IRSEIVERRYLGQQLYASILSPARSLRDEYNIPDNALLCGLVKNLPKPPPYSI |
| 898 | 49_54 | Bet_v | IDRGRKMGGVMFILMLCLTAGAMSGVRGEDPYLFFTWNVTYGTISPLGVPQQGILI |
| | | | NGQFPGPNINSTTNNNIVINVHNSLHEPFLLTWSGIQHRKNSWQDGVLGTMCPIP |
| | | | PGTNYTYHFQVKDQIGSYIYYPTTATHRAAGAFGGLRVNSRLLIPVPYADPEDDYTV |
| | | | LIGDWYAKSHQTLRKFLDSGRSLGRPDGVLINGKSGKDKPLFTMKAGKTYKYRIC |
| | | | NVGVKNSLNFRIQGHTMKLVELEGSHTVQNTYQSLDVHVGQCLSVLVTADQKPK |
| | | | DYYVVASTRFTKSVLTGKGIIRYIGGKGPASPEIPEAPVGWAWSLNQFRTFRWNLT |
| | | | ASAARPNPQGSFHYGAINITRTIKLVNSASKVDGKHRYAVNGISHIDPPTPLKLAEY |
| | | | YGVADKVFKYDTIPDDPPAQGAPNITSAPVVLNMTFRNFVEIIFENHEKSIQSWHL |
| | | | DGYSFFAVAIEPGRWTPERRRNYNLLDAVSRHTVQVFPKSWAAILLTFDNAGMWN |
| | | | IRSEIVERRYLGQQLYASILSPARSLRDEYNIPDNALLCGLVKNLPKPPPYVI |
| 899 | 49_54 | Bet_v | IFENHERTIQTYHLDGYSFFAVAIEPGRWSPVKRKNYNLLDAVSRHNIQVYPNSWA |
| | | | AIMTTLDNAGMWSLRSEMWERVYLGQQLYFSVLSPARSLRDEYNLPDNTPLCGIV |
| | | | PGLPLPPPYTA |
| 900 | 49_54 | Cyn_d | TIAQTPHYTFHSREHHITRARPASVCLPREHFGRRPAGIMAATMRAAAAGVLLVLT |
| | | | ALAVVHAEDPYLFFEWKVTYGTKSLLGVPQKVILINGEFPGPRINCSSNNNIVVNVF |
| | | | NQLDQPLLFTWNGMQHRKNSWMDGLPGTNCPIAPGTNFTYKWQPKDQIGSFFYF |
| | | | PSIAMQRSAGGYGLISVHSRDLIPVPFDIPADDFAVLAGDWYTKDHTVLAKHLDA |
| | | | GKGIGRPAGLIINGKNDKDAASAPMYNFEAGKTYRFRVCNVGIKASLNVRVPGHN |
| | | | LKLVEMEGSHTVQNMYDSLDVHVGQCLSFLVTADQKPADYFLVVSTRFIKEVSTIT |
| | | | ALIRYKGSSTPPSPKLPEGPSGWAWSINQWRSFRWNLTASAARPNPQGSYHYGQ |
| | | | INITRTIKLQISRGHIDGKLRYGFNGVSHVDADTPLKLAEYFNATDGVFQYNLISDV |
| | | | PPKAGTPIKLAPNVLSAEFRTFIEVVFENPEKSIDSFHIDGYAFFAAGMGPGTWSPQ |
| | | | SRKTYNLLDTVSRHTIQVYPRSWTAVMLTFDNAGMWNVRSNLWERQYLGEQMYI |
| | | | SVISPARSLRDEYNMPETSLRCGKVVGLPMPPSYLPA |
| 901 | 49_54 | Cyn_d | TIAQTPHYTFHSREHHITRARPASVCLPREHFGRRPAGIMAATMRAAAAGVLLVLT |
| | | | ALAVVHAEDPYLFFEWKVTYGTKSLLGVPQKVILINGEFPGPRINCSSNNNIVVNVF |
| | | | NQLDQPLLFTWNGMQHRKNSWMDGLPGTNCPIAPGTNFTYKWQPKDQIGSFFYF |
| | | | PSLGMQRAAGGYGPISVVSRLLIPVPFDPPADDHVVLIGDWYTKDHEVMARLLDS |
| | | | GRSIGRPAGVLINGKGGKDAAAAPIFTFEAGKTYRLRVCNTGIKSSLNFRIQGHDM |
| | | | KLVEMDGSHTVQDMFDSLDVHPGHCFSVLVDADQKPGDYYVVASTRFIHDPKSV |
| | | | SAVIRYAGSSTPPAPHVPEPPEGWAWSINQWRSFRWNLTASAARPNPQGSYHYG |
| | | | QINITRTIKLQISRGHIDGKLRYGFNGVSHVDADTPLKLAEYFNVTDGVFKYNQMG |
| | | | DAPPAVNGPLRVMPSVISAEFRTFIEVIFENPEKSMDSLHLDGYAFFAVGMGPGKW |
| | | | SPELRKTYNLLDAVSRHTIQVYPRSWTAIMLTFDNAGMWNVRSNIWERHYLGEQV |
| | | | YVSVISPERSLRDEYNMPENALRCGKVIGLPLPPSYNPAR |
| 902 | 49_54 | Cyn_d | TIAQTPHYTFHSREHHITRARPASVCLPREHFGRRPAGIMAATMRAAAAGVLLVLT |
| | | | ALAVVHAEDPYLFFEWKVTYGTKSLLGVPQKVILINGEFPGPRINCSSNNNIVVNVF |
| | | | NQLDQPLLFTWNGMQHRKNSWMDGLPGTNCPIAPGTNFTYKWQPKDQIGSFFYF |
| | | | PSLGMQRAAGGYGPISVVSRLLIPVPFDPPADDHVVLIGDWYTKDHEVMARLLDS |
| | | | GRSIGRPAGVLINGKGGKDAAAAPIFTFEAGKTYRLRVCNTGIKSSLNFRIQGHDM |
| | | | KLVEMDGSHTVQDMFDSLDVHPGHCFSVLVDADQKPGDYYVVASTRFIHDPKSV |
| | | | SAVIRYAGSSTPPAPHVPEPPEGWAWSINQWRSFRWNLTASAARPNPQGSYHYG |
| | | | QINITRTIKLQISRGHIDGKLRYGFNGVSHVDADTPLKLAEYFNATDGVFQYNLISD |
| | | | VPPKAGTPIKLAPNVLSAEFRTFIEVVFENPEKSIDSFHIDGYAFFAAGMGPGTWSP |
| | | | QSRKTYNLLDTVSRHTIQVYPRSWTAVMLTFDNAGMWNVRSNLWERQYLGEQM |
| | | | YISVISPARSLRDEYNMPETSLRCGKVVGLPMPPSYLPA |
| 903 | 49_54 | Cyn_d | TIAQTPHYTFHSREHHITRARPASVCLPREHFGRRPAGIMAATMRAAAAGVLLVLT |
| | | | ALAVVHAEDPYLFFEWKVTYGTKSLLGVPQKVILINGEFPGPRINCSSNNNIVVNVF |
| | | | NQLDQPLLFTWNGMQHRKNSWMDGLPGTNCPIAPGTNFTYKWQPKDQIGSFFYF |
| | | | PSIAMQRSAGGYGLISVHSRDLIPVPFDIPADDFAVLAGDWYTKDHTVLAKHLDA |
| | | | GKGIGRPAGLIINGKNDKDAASAPMYNFEAGKTYRFRVCNVGIKASLNVRVPGHN |
| | | | LKLVEMEGSHTVQNMYDSLDVHVGQCLSFLVTADQKPADYFLVVSTRFIKEVSTIT |
| | | | ALIRYKGSSTPPSPKLPEGPSGWAWSINQWRSFRWNLTASAARPNPQGSYHYGQ |
| | | | INITRTIKLQISRGHIDGKLRYGFNGVSHVDADTPLKLAEYFNVTDGVFKYNQMGD |
| | | | APPAVNGPLRVMPSVISAEFRTFIEVIFENPEKSMDSLHLDGYAFFAVGMGPGKWS |
| | | | PELRKTYNLLDAVSRHTIQVYPRSWTAIMLTFDNAGMWNVRSNIWERHYLGEQVY |
| | | | VSVISPERSLRDEYNMPENALRCGKVIGLPLPPSYNPAR |
| 904 | 49_54 | Fra_e | ITRTIKLKITRGHLDGKLKYGFNGVSHVDADTPLKLAEYFNVTDGVFRYNQMGDSP |
| | | | PGVNGPLHAIPNVITAEFRTFIEIIFENPEKSMDSLHLDGYAFFAVGMGPGEWSPEL |
| | | | RKTYNLLDAVSRNSIQVYPRSWTAVMLTFDNAGMWNVRSNLWERHYLGEQFYIS |
| | | | VVSPARSLRDEYNMPEDDLRCGKVVGLPMPPSYLPA |
| 905 | 49_54 | Fra_e | ITRTIKLKITRGHLDGKLKYGFNGVSHVDADTPLKLAEYFNVTDGVFRYNQMGDSP |
| | | | PGVNGPLHAIPNVITAEFRTFIEIIFENPEKSMDSLHLDGYAFFAVGMGPGEWSPEL |
| | | | RKTYNLLDAVSRNSIQVYPRSWTAVMLTFDNAGMWNVRSNLWERQYLGEQMYIS |
| | | | VISPARSLRDEYNMPETSLRCGKVVGLPMPPSYLPA |
| 906 | 49_54 | Fra_e | ISVVSRLLIPVPFDPPADDLQVLIGDWYTKDHAVMASLLDAGKSFGRPAGVLINGR |
| | | | GGKDATNPPMFTFEAGKTYRLRVCNVGIKSSLNFRIQGHDMKLVEMEGSHTLQNT |
| | | | YDSLDVHVGQCLSVLVDADQKPADYLMVASTRFMVEPSSVSAV |
| 907 | 49_54 | Fra_e | PPSYSHKPGDVFHGRLLIDPPIPPQLLHYNPSRERNLFHSVRRPLILMATTMRGTAA |
| | | | TAGGVLLLALLVLSTTQVARAEDPYLFFEWHVTYGTRTLLGVPQKVILINDEFPGPRI |
| | | | NCSSNNNIVVNVFNQLEEPLLFTWNGMQQRKNSWQDGLPGTNCPVAPGTNYTYK |
| | | | WQPKDQIGSFFYFPSIGMQRAVG |
| 908 | 49_54 | Fra_e | WKVTYGTKNIMGTPQKVILINDMFPGPTINCTSNNNIVINVFNMLDQPLLFTWHGI |
| | | | QQRKNSWQDGMPGTNCPV |
| 909 | 49_54 | Lol_p | PLSHFHRPPHATHRSTAAAALIDLHTSRPEEETRRARRDMTAGSRMRACAAAAVL |
| | | | ALALLAVAVRAEDPYLFFEWKVTYGTRSPMGVPQKMILINDAFPGPTINCTSNNNII |
| | | | VNVFNQIDKPLLFTWHGIQQRKNSWQDGMPGAMCPIMPGTNFTYKMQFKDQIGT |
| | | | FFYFPSIGMQRAAGGYGLISIHSRPLIPIPFDPPAADFSAMIGDWFTKDHTVLEKHL |
| | | | DTGKTIGRPAGLLINGKNEKDASNPPMYEVEAGKTYRFRICNVGIKASLNVRVQGH |
| | | | ITRLVEMEGSHTVQNEYDSIDVHVGQCLSVLVTANQKPGDYFFVASTRFIKEVNTI |
| | | | TAVIRYKGSNTPPSPKLPEAPSGWAWSINQWRSFRWNLTASAARPNPQGSYHYG |
| | | | QINITRTIKLMVTRGHLEGKLKYGFNGVSHVDADTPLKLAEYFNVSDKVFKYNQM |
| | | | GDSPPGVNGPMHVAPNVITAEFRTFIEVVFENPEKSMDSLHIDGYAFFAVGMGPG |
| | | | KWSPDLRKTYNLLDAVSRHTIQVYPRSWSAVMLTFDNAGMWNLRSNLWERYYM |
| | | | GEQLYVSCTSPARSLRDEYNMPENGLRCGKIVGLPLPAPYIIA |
| 910 | 49_54 | Lol_p | PLSHFHRPPHATHRSTAAAALIDLHTSRPEEETRRARRDMTAGSRMRACAAAAVL |
| | | | ALALLAVAVRAEDPYLFFEWKVTYGTRSPMGVPQKMILINDAFPGPTINCTSNNNII |
| | | | VNVFNQIDKPLLFTWHGIQQRKNSWQDGMPGAMCPIMPGTNFTYKMQFKDQIGT |
| | | | FFYFPSIGMQRAAGGYGLISIHSRPLIPIPFDPPAADFSAMIGDWFTKDHTVLEKHL |
| | | | DTGKTIGRPAGLLINGKNEKDASNPPMYEVEAGKTYRFRICNVGIKASLNVRVQGH |
| | | | ITRLVEMEGSHTVQNEYDSIDVHVGQCLSVLVTANQKPGDYFFVASTRFIKEVNTI |
| | | | TAVIRYKGSNTPPSPKLPEAPSGWAWSINQWRSFRWNLTASAARPNPQGSYHYG |
| | | | QINITRTIKLMVTRGHLEGKLKYGFNGVSHVDADTPLKLAEYFNVSDKVFKYNQM |
| | | | GDSPPGVNGPMHVAPNVITAEFRTFIEVVFENPEKSMDSLHIDGYAFFAVGMGPG |
| | | | KWSPDLRKTYNLLDAVSRHTIQVYPRSWSAVMLTFDNAGMWNVRSNLWERHYL |
| | | | GEQLYISVISPARSLRDEYNMPETALRCGKVVGLPLPPSYLPA |
| 911 | 49_54 | Lol_p | IPYPAATPTLLSFKRAELDSARQVFHPARLPPILMAATTMRATAAGGVLLLALLLVTT |
| | | | NVARAEDPYVFFEWHVTYGTKSLLGVPQKVILINGEFPGPRINCSSNNNIVVNVFN |
| | | | QLDQPLLFTWNGIQHRKNSWQDGMPGTNCPVVPGTNYTFKWQAKDQIGSFFYFP |
| | | | SIGMQRTVGGYGLISVVSRLLIPVPFDPPADDLQVLIGDWYNKDHTVMASLLDAG |
| | | | KSPGRPAGVLINGRGAKDAANPPMFTFEAGKTYRLRICNVGIKASLNFRIQGHDM |
| | | | RLVEMDGSHTVQDSFDSLDVHVGHCLSVLVDADQKPADYLMVASTRFMVEPSSV |
| | | | SAVIRYAGSNTPPAPNVPEPPAGWAWSLNQWRSFRWNLTASAARPNPQGSYHYG |
| | | | QINITRTIKLMVTRGHLEGKLKYGFNGVSHVDADTPLKLAEYFNVSDKVFKYNQM |
| | | | GDSPPGVNGPMHVAPNVITAEFRTFIEVVFENPEKSMDSLHIDGYAFFAVGMGPG |
| | | | KWSPDLRKTYNLLDAVSRHTIQVYPRSWSAVMLTFDNAGMWNVRSNLWERHYL |
| | | | GEQLYISVISPARSLRDEYNMPETALRCGKVVGLPLPPSYLPA |
| 912 | 49_54 | Lol_p | IPYPAATPTLLSFKRAELDSARQVFHPARLPPILMAATTMRATAAGGVLLLALLLVTT |
| | | | NVARAEDPYVFFEWHVTYGTKSLLGVPQKVILINGEFPGPRINCSSNNNIVVNVFN |
| | | | QLDQPLLFTWNGIQHRKNSWQDGMPGTNCPVVPGTNYTFKWQAKDQIGSFFYFP |
| | | | SIGMQRTVGGYGLISVVSRLLIPVPFDPPADDLQVLIGDWYNKDHTVMASLLDAG |
| | | | KSPGRPAGVLINGRGAKDAANPPMFTFEAGKTYRLRICNVGIKASLNFRIQGHDM |
| | | | RLVEMDGSHTVQDSFDSLDVHVGHCLSVLVDADQKPADYLMVASTRFMVEPSSV |
| | | | SAVIRYAGSNTPPAPNVPEPPAGWAWSLNQWRSFRWNLTASAARPNPQGSYHYG |
| | | | QINITRTIKLMVTRGHLEGKLKYGFNGVSHVDADTPLKLAEYFNVSDKVFKYNQM |
| | | | GDSPPGVNGPMHVAPNVITAEFRTFIEVVFENPEKSMDSLHIDGYAFFAVGMGPG |
| | | | KWSPDLRKTYNLLDAVSRHTIQVYPRSWSAVMLTFDNAGMWNLRSNLWERYYM |
| | | | GEQLYVSCTSPARSLRDEYNMPENGLRCGKIVGLPLPAPYIIA |
| 913 | 49_54 | Ole_e | IQVYPRSWSAVMLTFDNAGMWNVRSNIWERHYLGEQVYVSVISPERSLRDEYNM |
| | | | PENALRCGKVIGLPLPPSYNPAR |
| 914 | 49_54 | Ole_e | PRINCSSNNNIVVNVFNQLDQPLLFTWNGMQHRKNSWMDGLPGTNCPIAPGTNF |
| | | | TYKWQPKDQIGSFFYFPS |
| 915 | 49_54 | Ole_e | GANLFHSARRPLILMATTMRGTAATAGGVLLLALLVLSTTQVARAEDPYLFFEWHV |
| | | | TYGTRTLLGVPQKVILINDEFPGPRINCSSNNNIVVNVFNQLEEPLLFTWNGMQQR |
| | | | KNSWQDGLPGTNCPVAPGTNYTYKWQPKDQIGSFFYFPSIGMQRAVGGYGLISV |
| | | | VSRLLIPVPFDPPADDLQVLIGDWYTKDHAVMASLLDAGKSFGRPAGVLINGRGG |
| | | | KDATNPPMFTFEAGKTYRLRVCNVGIKSSLNFRIQGHDMKLVEMEGSHTLQNTYD |
| | | | SLDVHVGHCLSVLVDADQKPADYLMV |
| 916 | 49_54 | Pla_l | DQVFKYNQMGDSPPGVNGPMHITPNVITAEFRTFIEVVFENPEKSMDSLHLDGYAF |
| | | | FAVGMGPGKWKPELRKTYNLLDAVSRHSIQVYPRSWSAVMLTFDNAGMWNLRS |
| | | | NLWERYYMGEQLYVSCTSP |
| 917 | 49_54 | Pla_l | LILMATTMRGTAATAGGVLLLALLVLSTTQVARAEDPYLFFEWHVTYGTRTLLGVPQ |
| | | | KVILINDEFPGPRINCSSNNNIVVNVFNQLEEPLLFTWNGIQHRKNSWQDGLPGT |
| | | | NCPVAPGTNYTYKWQPKDQIGSFFYFPSIGMQRAVGGYGLISVVSRLLIPVPFDPP |
| | | | ADDHVVLIGDWYTKDHEVMARLLDSGRS |
| 918 | 49_54 | Poa_p | RSPPILMATTMRATAAAAILLLALLLLSTTNVARAEDPYVFFEWHVTYGTKNLLGVP |
| | | | QKVILINGEFPGPRINCSSNNNIVVNVFNQLDQPLLFTWNGIQHRKNSWQDGLPG |
| | | | TNCPVAPGTNYTYKWQPKDQIGSFFYFPSIGMQRAVGGYGLISVVSRLLIPVPFDP |
| | | | PADDLQVLIGDWYTKDHAVMASLLDAGKSFGRPAGVLINGRGGKDATNPPMFTF |
| | | | EAGKTYRLRVCNVGIKASLNFRIQGHDMRLVEMDGSHTLQDSYDSLDVHVGHCL |
| | | | SVLVDADQKPADYLMVASTRFIVDASSVSAVIRYVGSNTPPAPNVPEPPAGWAWS |
| | | | LNQWRSFRWNLTASAARPNPQGSYHYGQINITRTIKLMITRGHLDGKLKYGFNGV |
| | | | SHVDADTPLKLAEYFNVSDQVFKYNQMGDSPPGVNGPMHITPNVITAEFRTFIEVV |
| | | | FENPEKSMDSLHLDGYAFFAVGMGPGKWKPELRKTYNLLDAVSRHSIQVYPRSW |
| | | | SAVMLTFDNAGMWNVRSNLWERHYLGEQLYISVISPARSLRDEYNFPENALRCGK |
| | | | VVGLPLPPSYLPA |
| 919 | 49_54 | Que_a | ELRKTYNLLDAVSRHTIQVYPRSWTAIMLTFDNAGMWNVRSNIWERHYLGEQVYV |
| | | | SVISPERSLRDEYNM |
| 920 | 49_54 | Que_a | TTQVARAEDPYLFFEWHVTYGTRTLLGVPQKVILINDEFPGPRINCSSNNNIVVNVF |
| | | | NQLEEPLLFTWNGIQHRKNSWQDGLPGTNCPVAPGTNYTFKWQAKDQIGSFFYF |
| | | | PSLGMQRAAGGYGMISVVSRLLIPVPFDPPADDFQVLVGDWYTKDHTVMASLLDA |
| | | | GKSPGRPAGVLINGKGGQDAASQPMFTFEAGKTYRLRVCNVGIKSSLNFRIQGHD |
| | | | MKLVEMEGSHTLQNTYDSLDVHVGQC |
| 921 | 51 | Amb_a | PTMDKEELVQRAKLAEQAERYDDMAQAMKQVTETGVELTNEERNLLSVAYKNVV |
| | | | GARRSSWRVISSIEQKTEGVERKQQMAREYRERVEKELREICYDVLGLLDKYLIPK |
| | | | ASNAESKVFYLKMKGDYYRYLAEVATGDQKTSVVEESQKAYQEAFDVSKGKMQP |
| | | | THPIRLGLALNFSVFYYEILNSPDRACQLAKQAFDDAIAELDTLNEDSYKDSTLIMQ |
| | | | LLRDNLTLWTSDTQGDGDEPQEGGD |
| 922 | 51 | Amb_a | AQDIANADLPPTHPIRLGLALNFSVFYYEILNSPDRACNLAKQAFDEAIAELDTLGE |
| | | | DSYKDSTLIMQLLRDNLTLWTSDMQEDGADEIKEASSKQ |
| 923 | 51 | Amb_p | MSNNDKDRETHVYMAKLSEQAERYEEMVECMKSVAKLNVELTVEERNLLSVGYK |
| | | | NVIGARRASWRIMSSIEQKEESKGNESNVTLIKGYCKKVEDELSKICSDILEIIDKH |
| | | | LIPSSGSGEATVFYHKMKGDYYRYLAEFKTDQERKDAAEQSLKGYEAAAAAANTEL |
| | | | PSTHPIRLGLALNFSVFYYEIMNSPERACHLAKQAFDEAIADLDSLSEESYKDSTLI |
| | | | MQLLRDNLTLWTSDLPEDAGDENQPKGEEPKPAE |
| 924 | 51 | Amb_p | DSKVFYLKMKGDYHRYLAEFKTGAERKEAAESTLNAYKAAQDIANAELAPTHPIRL |
| | | | GLALNFSVFYYEILN |
| 925 | 51 | Amb_p | VFYYEILNSPDRACNLAKQAFDEAISELDSLGEESYKDSTLIMQLLRDNLTLWTSDT |
| | | | NEDGGDEIKEAPAPK |
| 926 | 51 | Bet_v | MAVTPSAREENVYMAKLAEQAERYEEMVEFMEKVTAAVESEELSVEERNLLSVAYK |
| | | | NVIGARRASWRIISSIEQKEESRGNEDHVATIRDYRSKIETELSNICDGILKLLDTR |
| | | | LIPSASSGDSKVFYLKMKGDYHRYLAEFKTGADRKEAAESTLTAYKAAQDIANTEL |
| | | | APTHPIRLGLALNFSVFHYEILNSPDRACNLAKQAFDEAIAELDTLGEESYKDSTLI |
| | | | MQLLRDNLTLWSSDMQDDGADEIKEAP |
| 927 | 51 | Cyn_d | MSPSEPTREESVYMAKLAEQAERYEEMVEFMERVARSAGGAGGGEELSVEERNLL |
| | | | SVAYKNVIGARRASWRIISSIEQKEEGRGNEAHAASIRAYRSKIEAELARICDGILA |
| | | | LLDSHLVPSAGAAESKVFYLKMKGDYHRYLAEFKSGTERKEAAESTMNAYKAAQD |
| | | | IALADLAPTHPIRLGLALNFSVFYYEILNSPDRACNLAKQAFDEAISELDSLGEESYK |
| | | | DSTLIMQLLRDNLTLWTSDTNEDGGDEIKEAAAPKESGD |
| 928 | 51 | Que_a | MSPTDSSREENVYMAKLAEQAERYEEMVEFMEKVAKTVDVEELTVEERNLLSVAY |
| | | | KNVIGARRASWRIISSIEQKEESRGNEDHVVIIKEYRGKIENELSKICDGILGLLET |
| | | | HLIPSASAAESKVFYLKMKGDYHRYLAEFKTGAERKEAAESTLLAYKSAQDIALAEL |
| | | | PPTHPIRLGLALNFSVFYYEILNSPDRACNLAKQAFDEAISELDTLGEESYKDSTLIM |
| | | | QLLRDNLTLWTSDITDDAGDEIKEASKRESGE |
| 929 | 52 | Bet_v | ALGCDGSVLIDSTLSNTAEKDSPANNPSLRGFEVIDNAKAKLEAICKGVVSCADIV |
| | | | AFAARDSIEITGGLGYDVPAGRRDGIVSLASETLTNLPPPTFNVDQLTQLFANKGFT |
| | | | QEEMVTL |
| 930 | 52 | Bet_v | GCDASILIDSTNKKPSEKDASPNQTIRGYEVIDKAKKRLEVTCPSTSCADIITLAVR |
| | | | DAVALAGGPNY |
| 931 | 52 | Cyn_d | MDARMVFPLFLVAVAAAPLASGQLSPDFYKTTCPDAEKIIFGVVEKRFKEDPGTAA |
| | | | GLLRLVFHDCFANGCDASILIDPLSNQASEKEAGPNISVKGYDVIDDIKTELEKKCP |
| | | | EVVSCADIVAVSARDAVKLTGGPAYEVPTGRRDAVVSNREDADNLPGPDIAVPKLL |
| | | | SDFSKKGFDVEEAVALLAGGHTIGSCKCFFIEADAAPIDPEYKKNISAACDGANRD |
| | | | RGSVPLDQITPNVFDGNYFALALAKKMPLTVDRLMGMDPKTEPVLKAMAAKPESF |
| | | | VPIFAKAMEKISALQVLTGKDGEIRKSCGEFNNPKPTSDGPSVIRISSLNPDHMGL |
| | | | SGPGARKVGGRADGMKANGAED |
| 932 | 52 | Que_a | LSNQASEKEAGPNISVKGYDVIDDIKTELEKKCPEVVSCADIVAVSARDAVKLTGG |
| | | | PAYEVPTGRRDAVVSNR |
| 933 | 53 | Amb_p | ERIHDANLTLHVGVLKNEFMNFGFDYFADPMVEIATYYSLLFCDGLVTEFPATAAAY |
| | | | FRSPCSDTSKN |
| 934 | 53 | Amb_p | AFCLGSADLTTSTTAATTFMAKVVTVSEIQNKSGIFSFDLSWSEIQTLKPDLSGPYA |
| | | | QAGLKRNPAAKNAGKFLTLSEFLELAKSSNVSGIMIEIEDAPYLATRGLGVVDAISS |
| | | | AL |
| 935 | 53 | Ant_o | EITLTKSYGDIAKDLSIIKPFASGIMVPKHFIQPLNKEDYLLPYTTLVKDARALGLEVF |
| | | | AAGFNNDMLTSYNYSYDPAAEYLQFIDNPDFSVDGVLTDFTPTASGAVACLAHTK |
| | | | GNALLPTAKALLPTENGERPLIITHNGASGVFPGCTDLAYQQAVRDGADIIDCAVR |
| | | | MTKDGVAFCLGSADLTTSTTAATTFMTKVVTVSEIQNRSGIFSFDLSWSEIQTLKP |
| | | | DLSGPYAQAGLKRNPAAKNAGKFLTLSEFLELAKSSNVSGIMIEIEDAPYLATRGLG |
| | | | VVDAVSSALVNASYDKESNHQRVLIQSDDSSVLSVFKKFPKFERILVIEPIISDASK |
| | | | PSIDEIKEFAHTVMVSRGSLVQVNGFFLTAFSDLAERIHDANLTLHVGVLKNEFMN |
| | | | FGFDYFADPMVEIATYYSLLFCDGLVTEFPATAAAYFRSPCSDTSKNLSYTILAANP |
| | | | GALEQMVPLGALPPALPPAPVLEPADVIDPPLPPVAVSSPPESTPNGDDQPSGASS |
| | | | NAGNCRLLVAGIAAAFLYLMSSH |
| 936 | 53 | Ant_o | IFTKRTAVCSSRMGSRYPLLFLILLLVHGANALPPVPEWLTLTGRRPLVIARGGFSG |
| | | | VFPDSSNLAFSNAVTYSLPDVVLFCDLQFSSDGVGFCLSNLNLDNSTLISKNEGFA |
| | | | SRGSTYQVNGQDIQGWFSLDFKAEELHNIPLIQNTLSRSQIFDGVPYLLSLDNVVK |
| | | | TVQPHEIWINVQYDSFLREHGLSSEDYILGLPKEFPVTWVSSPEVALLKSLSGKLR |
| | | | NNTKLIFRFLSEDLVEPTTKKTYGELLKDLKSITTFASGILVPKQFIWPMNKDMYLD |
| | | | PATSLVEDAHAIGLEVYASGFANDDSCISHNYSYDPSKEYLQFIDNSDFSVDGVLT |
| | | | DYPPTASAAVACLAHTKGNALAPPGTDTPGGGRPLIITHNGASGVFSDSTDLAYQ |
| | | | QAVKDGADIIDCWVRMTKDGVAFCLGSLDLNSSTTAATSFLGKMTTVNEIQNKS |
| | | | GIFSFDLTWNEIQTLKPNLIGPFSEASLDRNPAAKNAGKFMTLAGFLDYAKASNIS |
| | | | GILIGIEHAAFLETRGHDVVATVSNALIKSGYDKETKKCVLIQSEDPPVLSAFKKFP |
| | | | KFKRVFEIEFDIGDVSQPSVVQILEFANAVKLRRSSAARVDGFFLTGFTDALVDRLH |
| | | | AANIAVYVGVLKNEYMSLAFDYWADPMVEIATDTWAVGADGLVTEFPATAAAYFR |
| | | | SPCSDTSKNLSYTILAANPGALEQMVPLGALPPALPPAPVLEPADVIDPPLPPVAVS |
| | | | SPPESTPNGDDQPSGASSNAGNCRLLVAGIAAAFLYLMSSH |
| 937 | 53 | Cyn_d | LKNEFMNFGFDYFADPMVEIATYYSLLFCDGLVTEFPATAAAYFRSPCSDTSKNLSY |
| | | | TILAANPGALEQMVPLGALPPALPPAPVLEPADVIDPPLPPVAVSSPPESTPNGDDQ |
| | | | PSGASSNAGNCRLLVAGIAAAFLYLMSSH |
| 938 | 53 | Cyn_d | PRWGRRKAFPSFVLGVSCEGAPPDQMGASNPHMFLILLLLLHGASAAPNAPLPKW |
| | | | RTLSGRPPLVIAHGGFSGLFPDSSQFAYQFAMSTSLPDVALFCDLQFSSDGMGFC |
| | | | KSGLTLDNSTIISEVFPKMEKTYKVNGEDVRGWFSLDFTADQLVQNVTLIQNIFSR |
| | | | PSTFDGALGMYMVDDVVELRPPHIWLNVEYHSFFLEHKISTEDYLKALPKEFSFSYI |
| | | | SSPEVAFLKSVGGLLKQSKTKFVFRLLNENVVEPSTKKTYGELAKDLKFIKEFASGI |
| | | | LVPKTYIWPLNKDQYLAPSTSLVKDAHALGLEVYASGFANDVGLSYNYSYDPSAEY |
| | | | LQFIDNPDFAVDGLLTDFPPTASGAVACLAHSKGNPLPPPQRPRPLIISHNGASGVF |
| | | | PGSTDLAYQQAMKDGTDIIDCTVQMSKDGVAFCMPSADLGSCTTAGISFINKGST |
| | | | VHEIQNKSGIFSFDLSWSEIQTLKPDLVGPFAQAGLKRNPVAKNAGKFMTLPGFLD |
| | | | MAKASNVSGILINIEHAAYLATKGLGVVDAVTGALTKAGYDKETKQQVLIQSEDSS |
| | | | VLSAFKKSFPASKRVLSIDTEISDVAKPSVDDIKGVADGVRIHRSSVAQVTGYFLT |
| | | | HFTHVVDTLHAANLTVFIGVLKNEFMNLGFDYFADPMVEIVTYSDAVMADGLITEF |
| | | | PATAAAYFKSPCSDMNLNLSYSILPAQPGALVNIAVPGALPPVGAPAPLLEPADVLD |
| | | | PPLPPVRAVSTAAAPAPTGAADNTTSAASTTAGNRSSSLLVAGIVALLSLSFLQ |
| 939 | 53 | Fra_e | DLAYRQAMKDGADIIDCTVQMSKDGVAFCMPSADLGSCTTAGISFINKGSTVHEI |
| | | | QNKSGIFSFDLSWSEIQTLKPDLVGPFAQAGLKRNPVAKNAGKFMTLPGFLDMAK |
| | | | ASNVSGILINIEHAAYLATKGLGVVDAVTGALTKAGYDKETKQQVLIQSEDSSVLS |
| | | | AFKKSFPASKRVLSIDTEISDVAKPSVDDIKGVADGVRIHRSSV |
| 940 | 53 | Fra_e | NAGKLLTLPQFLDLAKTSNVSGILIDIEDAPYLATRGLGVVDAVSSALVNASYDKES |
| | | | NQQKVYIQSDDSSVLSVFKKFPRFQRVLVIDPVISDASKPSIDEIKEFADIVMVSR |
| | | | GSLVRVNGFFLTGYNDLVEKIHNANLTLHVGVLKNEFMNFGFDYFADPMVEIATYS |
| | | | SALVADGIVTEFPATAAAYFKSPCSDPSKNVSYTINAAQPGA |
| 941 | 53 | Fra_e | FFLTAFSDLAERIHDANLTLHVGVLKNEFMNFGFDYFADPMVEIATYYSLLFCDGLV |
| | | | TEFPATAAAYFRSPCSDTSKNLSYTILAANPGALEQMVPLGALPPALPPAPVLEPAD |
| | | | VIDPPLPPVAVSSPPESTPNGDDQPSGASSNAGNCRLLVAGIAAAFLYLMSSH |
| 942 | 53 | Lol_p | LVKDAHALGLEVYASGFANDDACMSHNYSYDPNAEYLNFIDNSDFSVDGFLTDYP |
| | | | PTASGAIACLAHTKGNALASIGNETTDGSRPLIITHDGASGVFPGSTDLAYQQAVK |
| | | | DGADIIDCWVRMSKDGVAFCLGSSDLNGSTTAATTFLGKMTNVDEIQNKSGIFSF |
| | | | DLSWNEIQTLKPNLIGPFSESAMDRNPAAKNAGKFMTLAAFLDYAKASNISGILIGI |
| | | | EGAAYLATRGL |
| 943 | 53 | Lol_p | YLATRGLDVVGAVSTALTKFGYDKETKQVVLIQSEDPPVLSAFKKFPKFKRVYEIEF |
| | | | DITDISKPSVVEISEMANAVKLRRSSAVQVDGFYLTGFTHALVDRLHAAKIEVYVG |
| | | | VLKNEFMSLAFDYWADPMKEIATDTWAVPADGLITDFPATAAAYFRSPCSDMEQN |
| | | | MSYYTISPAEVGTLVRMASYGLPPAPPPAPVLEPEDVHHQPLPLCPKEPMFRTFRCR |
| | | | MPPKGEYTMATDG |
| 944 | 53 | Lol_p | QFIDNPDFAVDGLLTDFPPTASGAVACLAHSKGNPLPPPQRPRPLIISHNGASGVFP |
| | | | GSTDLAYQQAMKDGTDIIDCTVQMSKDGVAFCMPSADLGSCTTAGTSFINKGST |
| | | | VHEIQNKSGIFSFDLSWSEIQTLKPDLVGPFAQAGLKRNPVAKNAGKFMTLPGFLD |
| | | | MAKASNVSGILINIEHAAYLATKGLGVVDAVTGALTKAGYDKETKQQVLIQSEDSS |
| | | | VLSAFKKSFPASKRVLSIDTEISDVAKPSVDDIKGVADGVRIHRSSVAQVTGYFLT |
| | | | HFTHVVDTLHAANLTVFIGVLKNEFMNLGFDYFAD |
| 945 | 53 | Lol_p | MGGRYPHMLLILILLHAANAALDEPVDKWKTLGGTPPLVIARGGFSGLFPESSPAA |
| | | | YQFAISTALPGVILHCDLQLSSDAKGFCRSGVRLDKSTLIEDIYPNRDKTYKIGPED |
| | | | VHAWFSVDFTEAELLNVTVKQTIYSRPSTFDGVMPMYRLEDVASLEPDGIWVNVE |
| | | | YNSFYKEHKISTEDFLLALPKEFPITYISSPDISFLKSIGGKLKGNTKLILRSLWENAT |
| | | | EPTLLKSYGDIMKDLSIIKPFASGILVPRHFIWPTNKDEYLLPSTSLVKDAHALGLEV |
| | | | YAAGFANDIFTSYNYSYDPAAEYLQFIDNPDFSVDGVLTDFTPTASGAIACLAHTKG |
| | | | NALLPIAKPLLATENGERPLIITHNGASGVFSGCTDLAYQQAVRDGADILDCSVRM |
| | | | TKDGVAFCLGSADLTTSTTAATTFMAKVVTVSEIQNKSGIFSFDLSWSEIQTLKPE |
| | | | LNGPYAQAGLKRNPAAKNAGKFWSLSEFLDFAKTSNVSGVLIEIEDAPYLATRGLG |
| | | | VVDAISSALVNASYDKESHQQRVLIQSDDSSVLSVFKKFPKFERVFVIDPVISDAS |
| | | | KPSIDEIKEFAHTVMVSRGALVRAHGFFLTGFNDMLVGKIHDANLTLHVGVLKNEF |
| | | | MNIGFDYFADPMVEIVTYYMGLVCDGIVTEFPATAAAYFRSPCSDLTKNMSYSILAA |
| | | | NPGGLEKMVPLGALPPALPPAPVLEPADVIDPPLPPVAVSSPPESTPEGDEDASAAS |
| | | | SNAANCLLVAGIAAFLYLSSH |
| 946 | 53 | Ole_e | PPPQRPRPLIISHNGASGVFPGSTDLAYQQAMKDGTDIIDCTVQMSKDGVAFCMP |
| | | | SADLGSCTTAGISFINKGSTVHEIQNKSGIFSFDLSWSEIQTLKPDLVGPFAQAGL |
| | | | KRNPVAKNAGKFMTLPGFLDMAKASNVSGILINIEHAAYLATKGLGVVDAVTG |
| 947 | 53 | Ole_e | VGVLKNEFMNFGFDYFADPMVEIATYYSLLFCDGLVTEFPATAAAYFRSPCSDLTKN |
| | | | MSYSILAANPGGLEKMVPLGALPPAL |
| 948 | 53 | Ole_e | AQAGLKRNPAAKNAGKFWSLSEFLDFAKTSNVSGVLIEIEDAPYLATRGLGVVDAI |
| | | | SSALVNASYDKESHQQRVLIQSDDSSVLSVFKKFPKFERVFVIDP |
| 949 | 53 | Pla_l | PSVDDIKGVADGVRIHRSSVAQVTGYFLTHFTHVVDTLHAANLTVFIGVLKNEFMN |
| | | | LGFDYFADPMVEIVTYSDAVMA |
| 950 | 53 | Pla_l | VRAHGFFLTGFNDMLVGKIHDANLTLHVGVLKNEFMNIGFDYFADPMVEIVTYYM |
| | | | GLVCDGIVTEFPATAAAYFRSPCSDLTKNMSYSILAANPGGLEKMVPLGALPPALPP |
| | | | APVLEPADVIDPPLPPVAVSSPPESTPNGDDQPSGASSNAGNCRLLVAGIAAAFLYL |
| | | | MSSH |
| 951 | 53 | Poa_p | QFIDNPDFAVDGLLTDFPPTASGAVACLAHSKGNPLPPPQRPRPLIISHNGASGVFP |
| | | | GSTDLAYQQAMKDGTDIIDCTVQMSKDGVAFCMPSADLGSCTTAGISFINKGSTV |
| | | | HEIQNKSGIFSFDLSWSEIQTLKPDLVGPFAQAGLKRNPVAKNAGKFMTLPGFLD |
| | | | MAKASNVSGILINIEHAAYLATKGLGVVDAVTGALTKAGYDKETKQQVLIQSEDSS |
| | | | VLSAFKKSFPASKRVLSIDTEISDVAKPSVDDIKGVADGVRIHRSSVAQVTGYFLT |
| | | | HFTHVVDTLHAANLTVFIGVLKNEFMNLGFDYFAD |
| 952 | 53 | Poa_p | SEIQTLKPNLIGPFSASGLDRNPAAKNAGKFMTLAGFLDYAKASNITGILIGIEHSA |
| | | | YLATRGLDVVDAVSSALIKSAYDKETKQRVFIQSEDPPVLSAFKKIPKFMRVFEIEF |
| | | | DIRDVSQPSVVEISEFANAVKLRRSSATQADGYYLTGFTTALVQRLHAANILVYVG |
| | | | VLKNEFMSLAFDYWADPMVEIATDTWSVFADGLVTEFPATAAAYFRSPCSNMERN |
| | | | LSYTIRPASPGILLDLAAYGALPPAPPPAPVLEPADIHRQPLPLCPTEPMFRTFRCRLA |
| | | | PKATGKSAEYTANLASDG |
| 953 | 53 | Poa_p | SEIQTLKPNLIGPFSASGLDRNPAAKNAGKFMTLAGFLDYAKASNITGILIGIEHSA |
| | | | YLATRGLDVVDAVSSALIKSAYDKETKQRVFIQSEDPPVLSAFKNIPKSNRVFEIEF |
| | | | DIGDVSQPSVVEITKFANVVKLRRSSAAKVDGFYLTGFTDAVKRLKDAKIEVHVGV |
| | | | LKNEFMSLAFDYWADPMVEIATDTWSVFADGLVTEFPATAAAYFRSPCSDMT |
| 954 | 53 | Poa_p | SEIQTLKPNLIGPFSASGLDRNPAAKNAGKFMTLAGFLDYAKASNITGILIGIEHSA |
| | | | YLATRGLDVVDAVSSALIKSAYDKETKQRVFIQSEDPPVLSAFKKIPKFMRVFEIEF |
| | | | DIRDVSQPSVVEISEFANAVKLRRSSATQADGYYLTGFTTALVQRLHAANILVYVG |
| | | | VLKNEFMSLAFDYWADPMVEIATDTWSVFADGLVTEFPATAAAYFRSPCSNMERN |
| | | | LSYTIRPASPGILLDLAAYGALPPAPPPAPVLEPTDVHRQPLPLCPTEPIFRTFRCRLP |
| | | | PKETGKNPEYTGSLAANG |
| 955 | 53 | Que_a | VADGVRIHRSSVAQVTGYFLTHFTHVVDTLHAANLTVFIGVLKNEFMNLGFDYFAD |
| | | | PMVEIVTYSDAVMADGLITEFPATAAAYFKSPCSDMNLNLSYSILPAQPGALVNIAV |
| | | | PGALPPVG |
| 956 | 53 | Que_a | KNEFMNIGFDYFADPMVEIVTYYMGLVCDGIVTEFPATAAAYFRSPCSDTSKNLSY |
| | | | TILAANPGALEQMVPLGALPPALPPAPVLEPADVIDPPLPPVAVSSPPESTPNGDDQ |
| | | | PSGASSNAGNCRLLVAGIAAAFLYLMSSH |
| 957 | 53 | Que_a | TAKALLPTENGERPLIITHNGASGVFPGCTDLAYQQAVRDGADIIDCAVRMTKDGV |
| | | | AFCLGSADLTTSTTAATTFMAKVVTVSEIQNKSGIFSFDLSWSEIQTLKPDLNGPY |
| | | | AQAGLKRNPAAKNAGKFWSLSEFLDFAKTSNVSGVLIEIEDAPYLATRGLGVVDAI |
| | | | SSALVNASYDKESHQQRVLIQSDDSSVLSVFKKFPKFERILVIEPIISDASKPSIDEI |
| | | | KEFADIVM |
| 958 | 53 | Que_a | IQTLKPDLVGPFAQAGLKRNPVAKNAGKFMTLPGFLDMAKASNVSGILINIEHAAY |
| | | | LATKGLGVVDAVTGALTKAGYDKETKQQVLIQSEDSSVL |
| 959 | 56 | Amb_a | ELLEFPNKDNRRLLHAVYRVGDLDRSIKFYTEAFGMKLLRKRDVPEEKYSNAFLGF |
| | | | GPEDSNFAVELTYNYGVDKYDIGTGFGHFAIATADVYKLAQDIKAKGGTITREAGP |
| | | | VKGGTSVIAFAKDPDGYLFELIERPNTPEPLCQVMLRVGDLDRSIKFYEKALGMKLC |
| | | | RKIDRPEQKYTLAMMGYAEEKETTVLELTYNYGVTEYTKGNAYAQVAVSTSDVYKS |
| | | | AQVVNHVIQELGGKITRQAGPLPGLGTKIVSFLDPDGWKTVLVDHEDFLKELHN |
| 960 | 56 | Amb_p | MAETLSAELLEFPNKDNRRLLHAVYRVGDLDRSIKFYTEAFGMKLLRKRDVPEEKY |
| | | | SNAFLGFGPEDSNFAVELTYNYGVDKYDIGTGFGHFAIATADVYKLAQDIKAKGGT |
| | | | ITREAGPVKGGTSVIAFAKDPDGYLFELIERPNTPEPLCQVMLRVGDLDRSIKFYEK |
| | | | ALGMKLCRKIDRPEQKYTLAMMGYAEEKETTVLELTYNYGVTEYTKGNAYAQVAVS |
| | | | TSDVYKSAQVVNHVIQELGGKITRQAGPLPGLGTKIVSFLDPDGWKTVLVDHEDF |
| | | | LKELH |
| 961 | 56 | Amb_p | CQVMLRVGDLDRSIAFHEKAFGMELLRRKDNPDYKYTIAMMGYGPEDKNAVLELT |
| | | | YNYGVTEYDKGNAYAQIAIGTDDVYKTAEAIKVFGGKITREPGPLPGISTKITACLD |
| | | | PDGWKTVFVDNVDFLKELE |
| 962 | 56 | Bet_v | MVRILPMASTIRPSLSSLKLPLLRFALSPHSPSRRLSMMHLGSAVPQSQFFGLKAVK |
| | | | LLRGEGNSMVVAAAGNAAQASTAATQENVLEWVKKDKRRMLHVVYRVGDLDRTI |
| | | | KFYTECLGMKLLRKRDIPEERYTNAFLGYGPEDSHFVIELTYNYGVDKYDIGTAFGH |
| | | | FGIAVEDVAKTVELIKAKGGKVTREPGPVKGGTTVIAFIEDFDGYKFELLERGPTPE |
| | | | PLCQVMLRVGDLDRSINFYEKAFGMELLRKRDNPEYKYTIAMMGYGPEDKSAVLEL |
| | | | TYNYGVTEYEKGNAYAQIAIGTDDVYKTAEAIKLSGGKITREPGPLPGISTKITACL |
| | | | DPDGWKAVFVDNVDFLKELE |
| 963 | 56 | Bet_v | MAEAAHVAPNAELLEWPKKDKRRFLHVVYRVGDLDRTIKFYTESFGMKLLRKRDIP |
| | | | EEKYSNAFLGFGPEQSNFVVELTYNYGVPSYDIGTGFGHFAISTPDVYKLVEDIRAG |
| | | | GGNVTREPGPVKGGQSVIAFVKDPDGYTFELIQRGPTPEPLCQVMLRVGDLDRAIK |
| | | | FYEKALGMRLLKKVDRPEYKYTIAMLGYAEEHETTVLELTYNYGVTEYTKGNAYAQI |
| | | | AIGTDDVYKSGEVVNLVIQELGGKITRQPGPIPGLNTKITSFLDPDGWKTVLVDNE |
| | | | DFLKELE |
| 964 | 56 | Cyn_d | GVTEYSKGNAYAQVAIGTNDVYKSAEAVDLATKELGGKILRQPGPLPGINTKIASF |
| | | | VDPDGWKVVLVDHADFLKELQ |
| 965 | 56 | Cyn_d | MRAFPATAGRGAVACAAAAPVPRRSLLLSTAAAGATLHSDSLRLATRSASGAGAIG |
| | | | ASADAAKAATFAGKDEAVAWAKSDNRRLLHVVYRVGDLDRTIKFYTECLGMKLLR |
| | | | KRDIPEDKYSNAFLGYGPEDSHFVVELTYNYGVDKYDIGEGFGHFGIAVDDVAKTV |
| | | | EFIRAKGGKVTREPGPVKGGKTVIAFVEDPDGYKFEILERPGTPEPLCQVMLRVGD |
| | | | LDRAISFYEKACGMELLRKRDNPEYKYTVAMLGYGPEDKNAVLELTYNYGVTEYAK |
| | | | GNAYGQIAIGTDDVYKTAEVAKLFGGQVVREPGPLPGINTKITSILDPDGWKSVFV |
| | | | DNIDFAKELE |
| 966 | 56 | Cyn_d | EPGPVKGGKSVIAFVEDPDGYKFELIERGPTPEPLCQVMLRVGDLDRAINFYEKAF |
| | | | GMELLRKRDNPQYKYTIAMMGYGPEDKNAVLELTYNYGVTEYDKGNAYAQIAIST |
| | | | DDVYKTAEVVRLNAGHITREPGPLPGINTKITACTDPDGWKTVFVDNIDFLKELEE |
| 967 | 56 | Cyn_d | MARLLLPLPFAAAAAASSSLHLAASRLRVPSVSVTRREGLFGGRLAGVSVPARLAR |
| | | | RGLSAGAEAGGGSAAQVVGPEEAMEWVKKDRRRLLHVVYRVGDLDKTIKFYTEC |
| | | | LGMKLLRKRDIPEERYTNAFLGYGPEDSHFVVELTYNYGVESYNIGTGFGHFGIAVE |
| | | | DVAKTVDLIKAKGGTVTREPGPVKGGKSVIAFVEDPDGYKFELIERGPTPEPLCQV |
| | | | MLRVGDLDRAINFYEKAFGMELLRKQDNPQYKKEYVLLTYY |
| 968 | 56 | Cyn_d | MATGSEAVLEWNKQDKKRMLHAVYRVGDLDRTIKCYTECFGMKLLRKRDVPDEK |
| | | | YTNAFLGFGPEDKNFALEL |
| 969 | 56 | Cyn_d | ELTYNYGVDKYEIGEGFGHFAIATEDISKLAEAVKSSCCCKITREPGPVKGGSTVIA |
| | | | FAQDPDGYMFELIQ RGPTPEPLCQVMLRVGDLERSIKFYEKALGMRLLRKKDVPEY |
| | | | KYTIAMLGYDDEDKTTVL |
| 970 | 56 | Que_a | SSYDIGTGFGHFAIATPDVYKLVEDIRAKGGVVTREPGPVKGGQSVIAFVKDPDGY |
| | | | VFELIQRGPTPEPLCQVMLRVGDLDRSIKFYEQALGMRVVKKVDRPEYKYTLAMLG |
| | | | YAEEHETTVLELTYNYGVTEYTKGNAYAQIAIGTDDVYKSAEVVNLVTQELGGKITR |
| | | | QPGPIPGLNTKITSFLDPDGWKTVLVDNEDFLKELHKE |
| 971 | 56 | Que_a | MAEAHAAPNAELLEWPKKDKRRFLHVVYRVGDLDRTIKFYTECFGMKLLRKRDIPE |
| | | | EKYSNAFLGFGSEETNFVVELTYNYGVTEYTKGNAYAQIAIGTDDVYKSAEVVNLV |
| | | | TQELGGKITRQPGPIPGLNTKITSFLDPDGWKTVLVDNEDFLKELH |
| 972 | 56 | Que_a | EDVAKTVELVKAKGGKVTREPGPVKGGSTVIAFVEDPDGYKFELLERGPTPEPLCQ |
| | | | VMLRVGDLDRSINFYEKAFGLELLRKRDNPEYKYTIAMMGYGPEDKNVVLELTYNY |
| | | | GVTEYDKGNAYAQIAIGTDDVYKTAEAIKLSGGKITREPGPLPGINTKITACLDPDG |
| | | | WKTVFVDNVDFIKELE |
| 973 | 56 | Que_a | MGVAAAGNAAQASTTATQENVLEWVKKDKRRMLHVVYRVGDLDRTIKFYTECLG |
| | | | MKLLRKRDIPEERYTNAFLGYGPEDSHFVIELTYNYGVDKYDIGTGFGHFGIAVEDV |
| | | | AKTV |
| 974 | 62 | Amb_a | RAERIVAEVVQAKQMMNPTTAAGVLRVFFHDCFVSGCDASVLIASTQFQKSEHDA |
| | | | EINHSLPGDAFDAVVRAKLALELECPGVVSCADILALASGVLVTMTGGPRYPIPLGR |
| | | | KDSLSSSPKDPDVELPHSNFTVDRLIQMFGAKGFTVQELVALSGAHTLGFSHCKEF |
| | | | ADRLYNFRSKGGKPEPFDPSMNPSYARGLKDVCKDYLKDPTIAAFNDIMTPGKFD |
| | | | NMYFVNLERGLGLLSTDEELWTDPRTKPLVQLYASNPTAFFTDFGKAMEKLSLFGV |
| | | | KTGKDGEVRRRCDAYN |
| 975 | 62 | Amb_p | AERDADINLSLPGDAFDIVTRIKTALELECPGVVSCSDILAIAARNLIKMTGGPKID |
| | | | VLFGRKDGLVSQASRVKGNLALPNMTMTHIINMFKLKGFTVQEMVALVGAHTIGF |
| | | | SHCKEFSSRIFSYSKTQPVDPKMNPKYADGLKRLCANYTKDHTMAAFNDVITPGK |
| | | | FDNMYYKNLQRGLGLLATDQAMADDPRTKPIVDLYAENEDAFFNDFAKAMQKVS |
| | | | MLDIKTDKNGEVRHRCDTFN |
| 976 | 62 | Amb_p | HGIAERDADINLSLPGDAFDIVTRIKTALELECPGVVSCSDILAIAARNLIKMTGGP |
| | | | KIDVLFGRKDGLVSQASRVKGNLALPNMTMTHIINMFKLKGFTVQEMVALVGAHT |
| | | | IGFSHCKEFSSRIFSYSKTQPVDPKMNPKYADGLKRLCANYTKDHTMAAFNDVITP |
| | | | GKFDNMYYKNLQRGLGLLATDQAMADDPRTKPIVDLYAENEDAFFNDFAKAMQK |
| | | | VSMLDIKTDKNGEVRHRCDTFNQQSGT |
| 977 | 62 | Ant_o | PGHSFPPFAPLHRLHENIVSNSPTLPSPSHFLDSHAPRRSSRRLLATSLQLGGTYRI |
| | | | NPRASHTHAGSTYQAAAMRRQSLLLLLAAATLLAATVSAQPGPTQPGPAQPVPTLP |
| | | | GPGPVPTLSPDFYSQTCPRAERIIAEVVQSKQMANPTTAAGVLRVFFHDCFVTGCD |
| | | | ASVLIAPTRFAKSEKDAEINHSLPGDAFDAVVRAKLALELECPGVVSCADILALASR |
| | | | VLVTMTGGPRYPIPLGRKDSLSSSPTAPDVELPHGNFTVGKIIELFLAKGFSIQEMV |
| | | | ALSGAHTLGFSHCQEFASRLYNYRDNGGKPAPFDPSMNPTYAKGLQAACQDYQK |
| | | | DPTIAAFNDIMTPGKFDNMYYINLQRGLGLLSTDEELWSDLRTKPFVQRYAANNTD |
| | | | FFEDFSKAMEKLSLYGVKTGAEGEIRRRCDAYNSGPITV |
| 978 | 62 | Bet_v | MAFPLLFILFLSIPFSEADLLSIDYYKKTCPDFDRIIRETVTSKQITNPTTAAGTLRAF |
| | | | FHDCVVNGCDASVLISSNSFNKAERDADLNLSLSGDAFDLIVRAKTALELACPNIV |
| | | | SCSDILAQATRDLITMVGGPYYKVILGRKDGLVSQASRVEGNIPRVNMSMNQIIK |
| | | | MFASKGFTVQEMVALTGSHTIGFSHCKEFADRIFNHSKTVPTDPETYPKFADALKK |
| | | | NCANYTKDPAMSAFNDVMTPGKFDNMYFQNLQRGLGLLASDHALIKNSRTKPIVD |
| | | | LFASNQTAFFEDFSQAMEKLGVYGIKTGQMGEVRHRCDAFN |
| 979 | 62 | Bet_v | ILISSTAFNSAERDADINHSLPGDAFDVVVRAKTALELACPNTVSCADILALATRDL |
| | | | VTMVGGPYYNVFLGRKDGLVSKSSYVEGKLPRPTMSISQIIELFASNGFSIQETVAL |
| | | | SGAHTIGFSHCKEFSSGIYNYSKYSQYDTQYNPRFAQALQKACADYQKNPTLSVF |
| | | | NDIMTPNKFDNMYFQNLPKGLGLLSSDHGLNSDPRTKPFVETYAADQNKFFEAFG |
| | | | KAMEKLSLYKVKTGRQGEIRHRCDEFN |
| 980 | 62 | Bet_v | CPGVVSCSDILAMAARDAVFWAGGPIYDIPKGRKDGRRSKIEDTINLPPPTFNASQ |
| | | | LIYMFGQHGFSAQEMVALSGAHTLG |
| 981 | 62 | Bet_v | ILISSTAFNSAERDADINHSLPGDAFDVVVRAKTALELACPNTVSCADILALATRDL |
| | | | VTMVGGPYYNVFLGRKDGLVSKSSYVEGKLPRPTMSISQIIELFASNGFSIQETVAL |
| | | | SGAHTIGFSHCKEFSSGIYNYSKYSQYDTQYNPRFAQALQKACADYQKNPTLSVF |
| | | | NDIMTPNKFDNMYFQNLPKGLGLLSSDHGLNSDPRTKPFVETYAADQNKFFEAFG |
| | | | KAMEKLSLYKVKTGRQGEIRHRCDEFN |
| 982 | 62 | Bet_v | MCPGVVSCSDILAMAARDAVFWAGGPIYDIPKGRKDGRRSKIEDTINLPPPTFNAS |
| | | | QLIYMFGQHGFSAQEMVALSGAHTLGV |
| 983 | 62 | Cyn_d | RHSIPSVGSRSSIALPPRTAIPSPRRISWTLTRAPRLQEGTHQEHYRISAMRLSLLL |
| | | | VLVAAFSAGAASQPLPPAGGKPLLTPDYYKQTCPRAERIIAEVIQSKQMANPTTAA |
| | | | GVLRVFFHDCFVGGCDASVLIASNQFAKSEHDADINQSLPGDAFDAVVRAKLALE |
| | | | MECPGVVSCADILSLASGVLVTMTGGPRYPVPLGRKDSLSSSPTAADADLPHSNF |
| | | | TVDRLIQMFGAKGFSVQELVALSGAHTLGFSHCKEFADRIFNYRDKAGKPEPFDPT |
| | | | MNPALAKGLQGACKDYLKDPTIAAFNDIMTPGKFDNMYFINLERGLGLLSTDEELW |
| | | | TDARTKPFVQLYASNSTKFFEDFGRAMEKLSLFGVKTGADGEIRRRCDTYNHGPM |
| | | | PK |
| 984 | 62 | Cyn_d | FSAGAASQPLPPAGGKPLLTPDYYKQTCPRAERIIAEVIQSKQMANPTTAAGVLRVF |
| | | | FHDCFVGGCDASVLIASNQFAKSEHDADINQSLPGDAFDAVVRAKLALEMECPGV |
| | | | VSCADILSLASGVLVTMTGGPRYPVPLGRKDSLSSSPTAADADLPHSNFTVDRLIQ |
| | | | MFGAKGFSVQELVALSGAHTLGFSHCKEFADRIFNYRDKAGKPEPFDPTMNPALA |
| | | | KGLQGACKDYLKDPTIAAFNDIMTPGKFDNMYFINLERGLGLLSTDEELWTDARTK |
| | | | PFVQLYASNSTKFFEDFGRAMEKLSLFGVKTGADGEIRRRCDTYN |
| 985 | 62 | Fra_e | RGFSVQEMVALSGAQTIRFFHCKEFSSILYNYSQTLESAPSYKRVMIYECIQLNAIK |
| | | | YKKVMIYECIQKPN |
| 986 | 62 | Lol_p | EHSRPLRSRHSLPSTSSEKHPLQVPRRPLSLAFLGPPRTSPALTSPAKLEGIKLTQR |
| | | | ATRAQDPRTKQQLAAMRRMSLLLLAAAAVLAAAVVAVHAGPPPPVKLSPDFYSQT |
| | | | CPRAERIIAEVVQSKQMANPTTAAGVLRVFFHDCFVSGCDASVLIAPTHYAKSEKD |
| | | | ADINHSLPGDAFDAVVRSKLALELECPGVVSCADILALASRVLITMTGGPRYPVPLG |
| | | | RKDSLSSNPAAPDVELPHSNFTVGRIIELFLAKGFTVQEMVALSGAHTLGFSHCQE |
| | | | FASRIYNYRDKGGKPAPFDPSMNPTYAKGLQAACQNYQKDPTIAAFNDIMTPGKF |
| | | | DNMYYVNIQRGLGLLSTDEDMWSDMRTKPFVQRYAANNADFFDDFSKAMEKLS |
| | | | MYGVKTGADGEIRRRCDAFNSGPITQ |
| 987 | 62 | Ole_e | PTYAKGLQAACQNYQKDPTIAAFNDIMTPGKFDNMYYVNIQRGLGLLSTDEDMWS |
| | | | DMRTKPFVQRYAANN |
| 988 | 62 | Pla_l | SSTAGEPLLLLGLIGPRTRPIFPVIIKNVGRKRLANVGAVASTSPLPRRQLLFMATTS |
| | | | FLLPFPNSASAVDEIDLIKEEIGKVITKIKAAGLLRLVFHDAGTFDQGDEAGGMNGS |
| | | | IVYELDRPENTGLAKSIKVLEKAKIQVGAVRPVSWADLIAVAGAEAVSICGGPNIPV |
| | | | KLGRIDAIVPDPEGRLPEESFAATAMKDNFQKKGFTTQELVALSGAHTLGGKGFGK |
| | | | PTVFDNSYYKILLDRPWSAGGMSSMIGLPSDRALVEDDECIRWISKYADDQVLFF |
| | | | EDFKNAYVKLVNTGAKWKR |
| 989 | 62 | Poa_p | PKSHTRVGSTYQPAAMRRLSLLLLAAAALLAAAVSAAPGPAPKLSPDFYSQTCPRA |
| | | | ERIIAEVVQSKQMANPTTAAGVLRVFFHDCFVSGCDASVLIAPTHYAKSEKDADIN |
| | | | HSLPGDAFDAVVRSKLALELECPGVVSCADILALASRVLVTMTGGPRYPVPLGRKD |
| | | | SLSSNPTAPDVELPHSNFTVGRIIELFVAKGFTVQEMVALSGAHTLGFSHCQEFAS |
| | | | RIYNYRDKGGKPAPFDPSMNPTYAKGLQAACQDYQKDPTIAAFNDIMTPGKFDNM |
| | | | YYVNIQRGLGLLSTDEDMWSDMRTKPFVQRYAANNTDFFDDFSKAMEKLSMYGV |
| | | | KTGADGEIRRRCDAFNSGPTTQ |
| 990 | 62 | Que_a | DFPFSLSLIFHTSFVLATLLLRFKSILIRSLSLVKMVIGKILGLILLMEMIVHGFRFEVV |
| | | | DGFRFDVVNGFRFGVVDGLSMEYYLLRCPLAELIVKIKVIKALQADPTLAASLVRLH |
| | | | FHDCFIEGCDGSVLLNSTKQNKAERDSPANLSLRGFELIDEIKEELEKQCPGIVSCA |
| | | | DILAMAARDAVCKAGGPLYDIPKGRMDGTRSKIEDTINLPAPTFNASQLINLFGQH |
| | | | GFSAQEMVALSGAHTLGVARCSSFKNRLVGGLDANLNADFAKTLFTTCSASDTAE |
| | | | QPFDETRNTFDNLYYRALQCKSGVLDSDQTLYASAETKGIVDSYASNKVMFFSDF |
| | | | KRAMVKMSMLNVKQGSQGEVRQNCYKIN |
| 991 | 62 | Que_a | KLSVDYYTKTCPDFDSIMRETVTSKQINSPTTAAGTLRLFFHDCMVDGCDASVLIS |
| | | | TNPFNKAERDADINLSLPGDAFDLVVRAKTALELSCPGIVSCADILAQATRDLITMV |
| | | | GGPFYKIRLGRKDGFESKAELVNGQVPQPNMSVNQLIKVFAAKGFSAQEMVALTG |
| | | | AHTIGFSHCKEFSHRIFNYSKTSPSDPEMYPKYAEALRKTCSNYLKDPGMSAFNDI |
| | | | MTPSKFDNMYYQNLQRGLGLLATDHALSKHPRTKPFVDLYASNQTKFFEDFSHAM |
| | | | EKLSVFGIKTGRKGEVRHKCDAFN |
| 992 | 65 | Bet_v | MELDLSPKLAKKVYGDNGGAYHAWSPSELPMLREGNIGAAKLALEKHGFALPRYS |
| | | | DSAKVAYVLQGNGVAGIVLPESEEKVLAIKKGDAIALPFGVVTWWYNKEDTELVVL |
| | | | FLGDTSKAHKAGEFTDFFLTGSNGIFTGFSTEFVGRAWDLDEKVVKTLVGKQSGN |
| | | | GIVKLDGKFEMPEPKKEHREGMALNCEEAPLDVDIKKGGRVVVLNTKNLPLVGEV |
| | | | GLGADLVRLDGGAMCSPGFSCDSALQVTYVVRGSGRVQVVGVDGRRVLETTLKA |
| | | | GNLFIVPRFFVVSKIASPDGMEWFSIITTPNPIFTHLAGKTSVWKALSPEVLKAAFN |
| | | | VDPDTEKLFRSKRTSDAIFFPP |
| 993 | 65 | Cyn_d | AKVAYVLQGAGTCGIVLPEATKEKVVAVKEGDALALPFGVVTWWHNLPESATELV |
| | | | VLFLGDTSKGHKPGQFTNFQLTGATGIFTGFSTEFVGRAWD |
| 994 | 65 | Cyn_d | FVGRAWDLTEADAAKLVSSQPASGIIKLGAGQKLPAPSAEDREGMALNCLEAPLD |
| | | | VDIKNGGRVVVLNTVNLPLVKEVGLGADLVRIDAHSMCSPGFSCDSAYQVTYIVR |
| | | | GSGRVQVVGPDGKRVLETRVEGGYLFIVPRFHVVSKIADESGMEWFSIIT |
| 995 | 65 | Cyn_d | MVNRTATAEVMSMDLSPKKPAKAYGSDGGSYYDWSPADLPMLGVASIGAAKLHL |
| | | | AAGGLALPSYSDSAKVAYVLQGTGTCGVVLPEATKEKVIPVKEGDALALPFGVVTW |
| | | | WHNAHAAATDLVVLFLGDTSKGHKAGQFTNFQLTGASGIFTGFSTEFVGRAWDL |
| | | | DQDAAAKLVSTQPGSGIVMVKDGHKMPAPRDEDRAGMVLNCLEAPLDVDIKGGG |
| | | | RVVVLNTQNLPLVKEVGLGADLVRIDAHSMCSPGFSCDSAYQVTYIVRGSGRVQV |
| | | | VGIDGTRVLETRAEGGCLFIVPRFFVVSKIADETGMEWFSIITTPNPIFSHLAGKTS |
| | | | VWKAISPAVLETSFNTTPEMEKLFRSKRLDSEIFFAP |
| 996 | 65 | Que_a | KTMEVDLSPKLAKKVYGDNGGSYHAWSPSELPMLREGNIGAAKLALEKNGFALPC |
| | | | YSDSSKVAFVLQGNGVAGIVLPESEEKVLAIKKGDAIALPFGAVTWWYNKEDTELV |
| | | | VLFLGDTSKAHKAGEFTEFFLTGSNGIFSGFSTEFVSRAWDLDENVVKTLVGKQS |
| | | | GNGIVKLDENFEMPEPKKEHRFGMAFNCEEAPLDVDIKKGGRVVLLNTNVLPMLG |
| | | | EAGLGGDLVRLDGSAMCSPGYSCDSALQVTYIVRGSGRVQVVGVDGRRVLESTL |
| | | | KAGNLFIVPRFFVVSKIASPEGMDWFTVITSPKSPTFTQLAGRTSVWKALSPSVLQ |
| | | | ASFDVDADTEKLFRSKRTSEAIFFPP |
| 997 | 65 | Que_a | KNGGRVVVLNTKNLPLVGEVGLGADLVRLDGHAMCSPGFSCDSALQVTYIVRGS |
| | | | GRVQVVGVDGRRVLET |
| 998 | 73 | Amb_a | SQDEAGTAAIKAVELDAILGGRAVQHREPQNFESDKFISYFKPCIAPLEGGVKSGF |
| | | | KKPVEEEFETRLYTCRGKRVVHLKQVPFSRSMLNHDDVFILDTKDKIFQFNGANSN |
| | | | IQERAKALEVIQFLKDKYHEGTCNVAIVDDGKLQAEGDSGEFWVIFGGFAPIGKKV |
| | | | LSDDDIIPDRTAGKLYSIAGGKVADQIADYSKSSFESDKCYLMDCGSEVFVWVGR |
| | | | ATQVDDRKAASQAAEEFLTSNKRPKATLITRLIQGYETHSFKSNFDSWPSSTAPAA |
| | | | ENRGKVAENRGKVSALLKQQGGGPKGKEKNTPTVEEAVPPLLEANGKLEVWSIDG |
| | | | GAKHPVASEDIGKFYNGDCYIVLYSYHSREKKEDFYLCHWIGKDSTEEDQNTAAK |
| | | | LTTSMFNSMKGRPVQGRIYQEKEPPQFIALFQPMVLFKGGLSSSYKSYIAEKGLTD |
| | | | ETYSPDNAAIIRISGTAVHNNKAVHLDPVPASLNSHECFVVHAGSHLYIWQGTQST |
| | | | YEQQEWAAKIAEFLKPGKTAKYQKEGTESATFWLGLGGKEDVSTNKVSFDTIRDP |
| | | | HLFAFSLSKGKFEVEEVYNFDQDDLLPEDMLILDTHAEVFVWIGHAVDPKEKKNAL |
| | | | EYGQKYIAWAESLDGLSPRVPLYRVPDGNEPNFFTTYFSWEPAKTMIHGNAFEKKV |
| | | | TILFGGHDEGAGNQGGGNTQRAAAMAALNSTFNSPGGGGKASGATKGSNANSQ |
| | | | RRAAVAALSGVIPDAKIDEPDSPEKPEEAPEEPVEPSEPIPEDNDSEPKVAIEEDEN |
| | | | GILTSKSTFSYEQVRVKSEDPAPDIDLKRREAYLSVEEFESVLGMTREEFYKLPKWK |
| | | | QDLTKKKVDLF |
| 999 | 73 | Amb_p | AVQHREPQNFESDKFISYFKPCIAPLEGGVKSGFKKPVEEEFETRLYTCRGKRVVHL |
| | | | KQVPFSRSMLNHDDVFILDTKDKIFQFNGANSNIQERAKALEVIQFLKDKYHEGTC |
| | | | NVAIVDDGKLQAEGDSGEFWVIFGGFAPIGKKVLSDDDIIPDRTAGKLYSIAGGKV |
| | | | ADQIADYSKSSFESDKCYLMDCGSEVFVWVGRATQVDDRKAASQAAEEFLTSNK |
| | | | RPKATLITRLIQGYETHSFKSNFDSWPSSTAPAAENRGKVAENRGKVSALLKQQG |
| | | | GGPKGKEKNTPTVEEAVPPLLEANGKLEVWSIDGGAKHPVASEDIGKFYNGDCYI |
| | | | VLYSYHSREKKEDFYLCHWIGKDSTEEDQNTAAKLTTSMFNSMKGRPVQGRIYQE |
| | | | KEPPQFIALFQPMVLFKGGLSSSYKSYIAEKGLTDETYSPDNASIIRISGTAVHNNK |
| | | | AVHLDPVPASLNSHECFVVHAGSHLYIWQGTQSTYEQQEWAAKIAEFLKPGKTAK |
| | | | YQKEGTESATFWLGLGGKEDVSTNKVSFDTIRDPHLFAFSLSKGKFEVEEVYNFD |
| | | | QDDLLPEDMLILDTHAEVFVWIGHAVDPKEKKNALEYGQKYIAWAESLDGLSPRV |
| | | | PLYRVPDGNEPNFFTTYFSWEPSKTMIHGNAFEKKVTILFGGHDEGAGNQGGGNT |
| | | | QRAAAMAALNSTFNSPGSGGKASGATKGSNANSQRRAAVAALSGVIPDAKIDEP |
| | | | DSPEKPEEAPEEPVEPSEPIPEDNDSEPKVAIEEDENGILTSKSTFSYEQVRVKSED |
| | | | PVPDIDLKRREAYLSVEEFESVLGMTREEFYKLPKWKQDLTKKKVDLF |
| 1000 | 73 | Bet_v | MSSSTKLDPAFQGAGQRVGTEIWRIENFQPVPLPKSENGKFYMGDCYIVLQTTQG |
| | | | RGGAYLFDIHFWIGKDSSQDESGTAAIKTVELDSALGGRAVQHRELQGHESDKFL |
| | | | SYFKPCIIPLEGGVASGFKTPEEEEFETRLYVCRGKRVVRMKQVPFARSSLNHDDV |
| | | | FILDTQDKIYQFNGANSNIQERAKALEVIQFLKEKYHVGKCDVAIVDDGKLDTESD |
| | | | SGEFWVLFGGFAPIGKKVASEDDIIPEATPAKLYSITDGQVKIIEGELSKSLLENNR |
| | | | CYLVDCGSEVFVWVGRVTQVEERKTAIQAAEEFVASQNRPKSTRITRLIQGYETHS |
| | | | FKSNFGSWPLGSATPGNEEGRGKVAALLKQQGVGVKGMTKSAPVNEEVPPLLEG |
| | | | GGKMEVWRINGSAKTPLPREDIGKFYSGDCYIVLYTYHSGDRKEDYFLCCWFGKD |
| | | | SIEEDQKMATRLANTMFNSLKGRPVQGRIFQGKEPPQFVALFQPMLVLKGGLSSG |
| | | | YKKIIADKGLVDETYTADSVALIQISGTSVHNNKAMQVDAVATSLNSMECFILQSG |
| | | | SSIFTWHGNQCTFEQQQLAAKVAEFLKPGVALKHAKEGTESSTFWFALGGKQSYT |
| | | | SKKVAQEIVRDPHLFTFSFNRGKFQVEEVHNFCQDDLLTEDILILDTHAEVFVWVG |
| | | | WSVDSKEKQNTFEIGQKYIEVAASLEGLSPQVPLYKVTEGNEPCFFTTYFQWDLTK |
| | | | AVVQGNSFQKKVALLFGIGHAVEDKSTGNQGGPTQRASALAALSSAFHPSSGKS |
| | | | GSMDKSNGSSQGPRQRAEALAALNSAFNSSSGTKTVAPRASAAGQGSQRAAAV |
| | | | AALSSVLTAEKKQSPDASPTRSSSSPPPESDAPEVPREVAEVKETEEVAPVSESNG |
| | | | EDSEPKQEQEEHDSGSSQTFSYDQLKAKSDNPVTGIDFKRREAYLSEEEFPTIFGI |
| | | | TKEAFYKLPKWKQDMQKRKFDLF |
| 1001 | 73 | Cyn_d | MSSAKAVLEPAFQGAGHKPGTEIWRIEDFKPVPLPKSDYGKFYRGDSYIVLQTTCN |
| | | | KGGAYLLDIHFWIGKDSSQDEAGTAAIKTVELDTMLGGRAVQHREPQGYESDKFL |
| | | | SYFKPCIIPLEGGFASGFKKPEEDKFETRLYICKGKRAIRVKEVPFARSQLNHDDVFI |
| | | | LDTEKKIYQFNGANSNIQERAKALEVIQHLKEKYHDGVCGVAIVDDGKLQAESDS |
| | | | GEFWVLFGGFAPIGKKTVSDDDVVLETTPPKLYSINNGQLKLEDTVLTKSILENTKC |
| | | | FLLDCGAELFVWVGRVTQVEDRKTASVAVENFILKQNRPKTTRITQVIQGYENHTF |
| | | | KSKFESWPVSNAAGNASAEEGRGKVAALLKQKGDVKGVSKSNAPVQDEVPPLLE |
| | | | SGDKLEVWCINENGKTCLEKEELGKFYSGDCYVVLYTYHSGDKREEFYLTYWIGK |
| | | | DSLPEDQEMALQTSNTIWNSLKGRPVLGRIYQGKEPPQFVALFQPMVILKGGISSG |
| | | | YKKFVEQKGLTDETYSADGIALVRISGTSVHNNKTLQVDSVSTSLSSTECFVLQSG |
| | | | KLMFTWIGNSSSFEQQQWAVKVAEFLKPGIAVKHCKEGTESSAFWSAIGGKRTYT |
| | | | SKNVAPDVFIRDPHLYTFSLRNGKMEVTEVFNFSQDDLLTEDMMIFDTHSEVFIWV |
| | | | GQCVETKDKQKAFEIGQKYVEHAVAFEGIAPDVPLYKVIEGNEPCFFRTYFSWDNT |
| | | | RSVIQGNSFEKKLSVLFGMRSEGGCKSSGDGGPTQRASALAALSSALNPSSQGK |
| | | | QSNERPTSSGDGGPTQRASAMAALTSALNPSSKPSSPQHQSRSGQGSQRAAAVA |
| | | | ALSNVLTAEGSSHSPHAEKTEVAPFSESEAEESPESFTDQDAQGGRTEPDVSHEQ |
| | | | TANENGGETTFSYDRLISKSTNPVGGIDYKRRETYLSDSEFETIFGMTKEEFYEQPR |
| | | | WKQELQKKKADLF |
| 1002 | 73 | Que_a | SSAKLDPAFQGAGQRVGTEIWRIENFQPVPLPKSEYGKFYMGDCYIVLQTAQGKG |
| | | | GAYTLDIHFWIGKDSSQDESGTAALKSVELDAVLGGRAVQHREIQGYESDKFLSY |
| | | | FKPCIIPLEGGVASGFKTPEEDVFETRLYVCRGKRVVRMKQVPFARSSLNHDDVFIL |
| | | | DTQNKIYQFNGANSNIQERAKALEVIQFLKEKYHVGTCDVAIVDDGKLDTESDSG |
| | | | EFWVLFGGFAPIGKKVTSEDDIIPEAAPAKLYSITDGQVKIVESGLSKSLLENNKCY |
| | | | LLDCGAEVFVWIGRVTQVEERKAAVQVAEEFLTGQNRPKSTRITRLIQGHETRSFK |
| | | | SNFDSWPSGSATPGNEEGRGKVAALLKQQGVGVKGMTKGAPVNEEVPPLLEGCG |
| | | | KMEVWRINGSAKTPLPKEDVGKFYSGDCYIVLYTYHSGDRKEDYLLCCWFGKDSI |
| | | | EEDQKMATRLASTMFNSLKGRPVQGRIFQGKEPPQFVALFQPMVVLKGGLSSGYK |
| | | | KFIADKGLTDETYTADSVALIQISGTSTHNNKAVQVDAAATSLNSMECFVLQSGS |
| | | | SIFSWHGNQSTFEQQQLAAKVSEFLRPGVALKHAKEGTESSSFWFPLGGKQSYTS |
| | | | KKVSQEIVRDPHLFTFSFNKGKFQVEEVYNFSQDDLLTEDILVFDTHAEVFVWVGQ |
| | | | SVDSREKQNAFEIGQKYIEMAASLEGLSSNVPLYKVTEGNEPCFFTTYFSWDQNK |
| | | | AVVQGNSFQKKIALLFGIGHVVEDKSSGNQGGPTQRASALAALSSAFHPSSGKPT |
| | | | QTDKSNGSNQGPRQRAEALAALNSAFNSSPGAKTSAPRPSGRGQGSQRAAAVAA |
| | | | LSSVLTAEKKSDESPTRSSSSPPPETNSPAETKSENDQSESEGPQEVAEIKESEEV |
| | | | APRSESNGGNSEPKQETVQENDSGSGRTFSYDQLKAKSDNPVTGIDFKRREAYLS |
| | | | DEEFQSVFGITKEAFNKLPRWKQDMQKKKVDLF |
| 1003 | 76 | Amb_a | QLQAFTKAYTDLESACSGLNVLVATYFADVPADAFKTLTTLPGVAGYTFDLVRGEK |
| | | | TLDLIKTSFPSGKYLFAGVVDGRNIWANDLAGSLSVL |
| 1004 | 76 | Amb_a | CSLLHTAVDLVNETKLDDEIKSWLAFAAQKVVEVNALAKALGGQKDEAFFSANAA |
| | | | AQASRKSSPRVNNEAVQKAAAGLKG |
| 1005 | 76 | Amb_a | KDEAYFSANAAAQASRKSSPRVTNEAVQKAAAALRGSDHRRATNVSARLDAQQK |
| | | | KLNLPILPTTTI |
| 1006 | 76 | Amb_a | KISEEEYVKAIKEEIFKVVQLQEELDIDVLVHGEPERNDMVEYFGEQLSGFAFTANG |
| | | | WVQSYGSRCVKPPIIYGDVSRPKAMTVFWSTM |
| 1007 | 76 | Amb_a | KISEEEYVKAIKEEIFKVVQLQEELDIDVLVHGEPERNDMVEYFGEQLSGFAFTANG |
| | | | WVQSYGSRCVKPPIIYGDVSRPKAMTVFWS |
| 1008 | 76 | Amb_p | DLEAYQLEAFTKAYSALESACSGLNVIVAIYFADVPAEAVKTLTSLPGVSGYTFDLV |
| | | | RGEKTLGLIKSNFPLGKYLFAVLFDGRNIWANDLAGSVAVLESLEGVVGKD |
| 1009 | 76 | Amb_p | MVHSSVLGFPRMGADRELKKANEAYWADKLSRDDLIKEGKRLRLEHWKIQKDAG |
| | | | VDVIPSNDFAFYDHLLDHIQLFNAIPERYSKHSLHKLDEYFAMGRGHQKDGVDVP |
| | | | SLEMVKWFDSNYHYVKPTLQDNQTFQLAENPKPVAEFLEAKEAGITTRPVLIGPVS |
| | | | FLALGKADRGQSVDPISLLEKLLPVYVELLQKLKEAGAEYVQIDEPVLVYDLPQKVK |
| | | | DAFKPAYEKLVSDSLPKLVLATYFGDIVHNFDVFPSLQGVAGIHIDLVRNPEQLESV |
| | | | AGKLGSNQVLSVGVVDGRNIWKTNFKRAIELVETAVQKLGKDRVLVATSSSLLHT |
| | | | PHSLDSEKKLPEEVKDWFSFAVQKVSEVVVIAKAVNDGPAAVREALEANAKSMQA |
| | | | RASSERTNNKAVKDRQASVTPEQHERKSAFPERYAQQKKHLSLPTFPTTTIGSFPQ |
| | | | TKEIRISRNKFTKGEITAEEYEKFIEKEIEEVVKIQDELGLDVYVHGEPERNDMVQYF |
| | | | GERLDGYVFTTKGWVQSYGSRCVRPPIIVGDISRPAPMTVKESKYAASVAKKPMK |
| | | | GMLTGPI |
| 1010 | 76 | Bet_v | MASHIVGYPRMGPKRELKFALESFWDGKTSAEDLQRVASDLRSSIWKQMADAGI |
| | | | KHIPSNTFSYYDQVLDTTALLGAVPPRYGWNGGEIGFDTYFSMARGNASVPAMEM |
| | | | TKWFDTNYHFIVPELGPDVKFSYASHKAVEEYKEAKALGVDTVPVLVGPVSYLLLS |
| | | | KPAKGVEKTFPLLSLLGKILPIYKEVISELKAAGATWIQFDEPTLVMDLDSHKLKAFT |
| | | | DAYSELESSLSGLNVIVETYFADVPAEAYKTLTALKGVTAFGFDLIRGTNTLDLIKGE |
| | | | FPKGKYLFAGVVDGRNIWANDLAASLGTLLALEGIVGKDKLVVSTSCSLLHTAVDL |
| | | | VNETKLDKEIKSWLAFAAQKVVEVNALAKALVGHKDEAFFSANAAALASRKSSPR |
| | | | VTNEAVQKAAAALKGSDHRRATNVSARLDAQQKKLNLPILPTTTIGSFPQTIELRR |
| | | | VRREYKANKISEEEYVKAIKEEINKVVKLQEELDIDVLVHGEPERNDMVEYFGEQLS |
| | | | GFAFTVNGWVQSYGSRCVKPPITYGDVSRPKPMTVFWSAAAQSMTARPMKGMLT |
| | | | GPV |
| 1011 | 76 | Bet_v | MASHVVGYPRMGPKRELKFALESFWDGKSSAEELKKVAADLRSSIWKQMADAGI |
| | | | KYIPSNTFSYYDQVLDTTAMLGAVPPRYGWSGGEIGFDVYFSMARGNASLPAMEM |
| | | | TKWFDTNYHFIVPELGPDVKFSYASHKAVDEFKEAKALGVDTVPVLVGPVSYLLLS |
| | | | KPAKGVEKSFSLLSLIDKILPVYKEVVTELKAAGATWIQFDEPSLILDLHAHQLQAF |
| | | | SHAYTELESSFSGLNVLIETYFADVSADAYKTLTSLKGVSGYGFDLVRGTQTLDLIK |
| | | | SGFPSGKYLFAGVVDGRNIWANDLASSLSILQTLEGTVGKDKIVVSTSCSLLHTAV |
| | | | DLVNETKLDKEIKSWLAFAAQKVVEVNALAKALSGHRDQAFFSANAAALASRKSS |
| | | | PRVTNEAVQKAAAALKGSDHRRATNVSARLDAQQKKLNLPILPTTTIGSFPQTIEL |
| | | | RRVRREYKANKISEEEYVKAIKEEINKVVKLQEELDIDVLVHGEPERNDMVEYFGE |
| | | | QLSGFAFTVNGWVQSYGSRCVKPPIIYGDVSRPKPMTVFWSAAAQSMTARPMKG |
| | | | MLTGPVTILNWSFVRNDQPRHETCYQIALAIKDEVEDLEKASINVIQIDEAA |
| 1012 | 76 | Cyn_d | MASHIVGYPRMGPKRELKFALESFWDGKSSAEDLEKVATDLRASIWKQMSEAGIK |
| | | | YIPSNTFSYYDQVLDTTAMLGAVPERYSWTGGEIGLSTYFSMARGNATVPAMEMT |
| | | | KWFDTNYHFIVPELGPTIKFTYASHKAVSEYKEAKALGIDTVPVLIGPVSYLLLSKPA |
| | | | KGVDKSFSLLSLLSSILPIYKEVVSELKAAGASWIQFDEPTLVKDLDAHELAAFTSA |
| | | | YAELESAFSGLNVLIETYFADIPAENYKTLTSLSGVTAYGFDLVRGSKTLDLVRSSFP |
| | | | SGKYLFAGAVDGRNIWADDLATSLSTLESLEAVVGKAKLVVSTSCSLMHTAVDLV |
| | | | NETKLDDEIKSWLAFAAQKVVEVNALAKALAGQKDEAYFAANAAAQASRRSSPRV |
| | | | TNEEVQKAAAALRGSDHRRATNVSARLDAQQKKLNLPVLPTTTIGSFPQTMDLRR |
| | | | VRREYKAKKISEEEYTNAIKEEISKVVKIQEELDIDVLVHGEPERNDMVEYFGEQLS |
| | | | GFAFTANGWVQSYGSRCVKPPITYGDVSRPNPMTVYWSKTAQSMTSRPMKGMLT |
| | | | GPV |
| 1013 | 76 | Cyn_d | MASHIVGYPRMGPKRELKFALESFWDGKSSAEDLEKVATDLRASIWKQMSEAGIK |
| | | | YIPSNTFSYYDQVLDTTAMLGAVPERYSWTGGEIGLSTYFSMARGNATVPAMEMT |
| | | | KWFDTNYHFIVPELGPTIKFTYASHKAVSEYKEAKALGIDTVPVLIGPVSYLLLSKPA |
| | | | KGVDKSFSLLSLLSSILPIYKEVVSELKAAGASWIQFDEPTLVKDLDAHELAAFTSA |
| | | | YAELESAFSGLNVLIETYFADIPAENYKTLTSLSGVTAYGFDLVRGSKTLDLVRSSFP |
| | | | SGKYLFAGAVDGRNIWADDLATSLSTLESLEAVVGKAKLVVSTSCSLMHTAVDLV |
| | | | NETKLDDEIKSWLAFAAQKVVEVNALAKALAGQKDEAYFAANAAAQASRRSSPRV |
| | | | TNEEVQKAAAALRGSDHRRATNVSARLDAQQKKLNLPVLPTTTIGSFPQTMDLRR |
| | | | VRREYKAKKISEEEYTNAIKEEISKVVKIQEELDIDVLVHGEPERNDMVEYFGEQLS |
| | | | GFAFTANGWVQSYGSRCVKPPITYGDVSRPNPMTVYWSKTAQSMTSRPMKGMLT |
| | | | GPVTILNWSFVRNDQPRFETCYQIALAIKKEVEDLEAAGIQVIQIDEAA |
| 1014 | 76 | Que_a | MASHIVGYPRMGPKRELKFALESFWDGKSSAEELQKVSADLRSSIWKQMADAGI |
| | | | KYIPSNTFAYYDQVLDTTAMLGAVPPRYGWNGGEIGFDTYFSMARGNASVPAMEM |
| | | | TKWFDTNYHFIVPELGPDVNFSYASHKAVSEYKEAKALGVDTVPVLVGPVSYLLLS |
| | | | KPAKGVDKNFSLLSLLEKILPIYKEVISELKAAGASWIQFDEPTIVLDLDSHKLKAFT |
| | | | DAYSELESSLSGLNVLIETYFADIPAEAFKTLTALKGVTAFGFDLVRGTKTLDLIKAE |
| | | | FPKGKYLFAGVVDGRNIWANDLAASLSTLHALEGIVGKDKLVVSTSCSLLHTAVDL |
| | | | VNETKLDKEIKSWLAFAAQKVVEVNALAKALAGHKDDAFFSDNAAAQASRKSSPR |
| | | | VTNESVQKAAAALKGSDHRRATNVSARLDAQQKKLNLPILPTTTIGSFPQTIELRR |
| | | | VRREYKAKKISEDEYVKAIKEEINKVVKLQEELDIDVLVHGEPERNDMVEYFGEQL |
| | | | SGFAFTVNGWVQSYGSRCVKPPIIYGDVSRPNPMTVFWSSAAQSMTARPMKGML |
| | | | TGPV |
| 1015 | 76 | Que_a | MASHIVGYPRMGPKRELKFALESFWDGKSSAEELQKVSADLRSSIWKQMADAGI |
| | | | KYIPSNTFAYYDQVLDTTAMLGAVPPRYGWNGGEIGFDTYFSMARGNASVPAMEM |
| | | | TKWFDTNYHFIVPELGPDVNFSYASHKAVSEYKEAKALGVDTVPVLVGPVSYLLLS |
| | | | KPAKGVDKNFSLLSLLEKILPIYKEVISELKAAGASWIQFDEPTIVLDLDSHKLKAFT |
| | | | DAYSELESSLSGLNVLIETYFADIPAEAFKTLTALKGVTAFGFDLVRGTKTLDLIKAE |
| | | | FPKGKYLFAGVVDGRNIWANDLAASLSTLHALEGIVGKDKLVVSTSCSLLHTAVDL |
| | | | VNETKLDKEIKSWLAFAAQKVVEVNALAKALAGHKDDAFFSDNAAAQASRKSSPR |
| | | | VTNESVQKAAAALKGSDHRRATNVSARLDAQQKKLNLPILPTTTIGSFPQTIELRR |
| | | | VRREYKAKKISEDEYVKAIKEEINKVVKLQEELDIDVLVHGEPERNDMVEYFGEQL |
| | | | SGFAFTVNGWVQSYGSRCVKPPIIYGDVSRPNPMTVFWSSAAQSMTARPMKGML |
| | | | TGPVTILNWSFVRNDQPRHETCYQIALSIKDEVEDLEKAGINVIQIDEAA |
| 1016 | 77 | Amb_a | MVKFTAEELRRIMDFKHNIRNMSVIAHVDHGKSTLTDSLVAAAGIIAQEVAGDVR |
| | | | MTDTRADEAERGITIKSTGISLYYEMTDEALKSFKGERNGNEYLINLIDSPGHVDFS |
| | | | SEVTAALRITDGALVVVDCIEGVCVQTETVLRQALGERIRPVLTVNKMDRCFLELQ |
| | | | VDGEEAYQTFQRVIENANVIMATYEDPLLGDVMVYPEKGTVAFSAGLHGWAFTLT |
| | | | NFAKMYASKFGVDEAKMMERLWGENYFDPKTKKWTTKSTGSATCKRGFVQFCYE |
| | | | PIKQIINTCMNDKKDQLWPMLTKLGVTMKSEEKELMGKALMKRVMQNWLPAATA |
| | | | LLEMMIFHLPSPHTAQRYRVENLYEGPLDDQYANAIRNCDPDGPLMLYVSKMIPAS |
| | | | DKGRFFAFGRVFAGRVSTGLKVRIMGPNYVPGEKKDLYVKSVQRTVIWMGKKQE |
| | | | TVEDVPCGNTVAMVGLDQFITKNATLTNEKEVDAHPIRAMKFSVSPVVRVAVQCK |
| | | | VASDLPKLVEGLKRLAKSDPMVVCTIEESGEHIIAGAGELHLEICLKDLQDDFMGG |
| | | | AEIVVSDPVVSFRETVLEKSSRTVMSKSPNKHNRLYMEARPMEDGLAEAIDEGRV |
| | | | GPRDDPKVRGKILSEEFGWD |
| 1017 | 77 | Amb_p | DFMGGAEIVVSDPVVSFRETVLEKSSRTVMSKSPNKHNRLYMEARPMEDGLAEAI |
| | | | DEGRVGPRDDPKVRGKILSEEFGWDKDLAKKIWCFGPETTGPNMVVDMCK |
| 1018 | 77 | Amb_p | AIDEGRVGPRDDPKVRGKILSEEFGWDKDLAKKIWCFGPETTGPNMVVDMCKGV |
| | | | QYLNEIKDSVVAGFQWASKEGALAEENMRGICFEVCDVVLHADAIHRGGGQVIPT |
| | | | ARRVIYASQLTAKPRLLEPVYLVEIQAPEQALGGIYSVLNQRRGHVFEEMQRPGTPL |
| | | | YNIKAYLPVVESFGFSGALRASTSGQAFPQCVFDHWDMMSSDPLEAGSQASTLVS |
| | | | QIRKRKGLKEQMTPLSEFEDKL |
| 1019 | 77 | Bet_v | MVKFTADELRRIMDYKHNIRNMSVIAHVDHGKSTLTDSLVAAAGIIAQESAGDVR |
| | | | MTDTRADEAERGITIKSTGISLYYEMTDESLKSYKGERHGNEYLINLIDSPGHVDFS |
| | | | SEVTAALRITDGALVVVDCVEGVCVQTETVLRQALGERIRPVLTVNKMDRCFLELQ |
| | | | VDGEEAYQTFQRVIENANVIMATYEDPLLGDVQVYPEKGTVAFSAGLHGWAFTLT |
| | | | NFAKMYASKFGVDESKMMERLWGENFFDPATKKWTTKNSGSPTCKRGFVQFCYE |
| | | | PIKQIINTCMNDQKDKLWPMLQKLGVTMKSDEKDLMGKALMKRVMQTWLPASTA |
| | | | LLEMMIFHLPSPSKAQRYRVENLYEGPLDDIYANAIRNCDPEGPLMLYVSKMIPASD |
| | | | KGRFFAFGRVFSGKVSTGLKVRIMGPNFVPGEKKDLYTKSVQRTVIWMGKKQETV |
| | | | EDVPCGNTVALVGLDQYITKNATLTNEKEVDAHPIRAMKFSVSPVVRVAVQCKVA |
| | | | SDLPKLVEGLKRLAKSDPMVVCTIEESGEHIIAGAGELHLEICLKDLQDDFMGGAEI |
| | | | IKSDPVVSFRETVLEKSCRTVMSKSPNKHNRLYMEARPLEEGLAEAIDDGRIGPRD |
| | | | DPKARSKILSEEFGWDKDLAKKIWCFGPETTGPNMVVDMCKGVQYLNEIKDSVV |
| | | | AGFQWASKEGALAEENMRGICFEVCDVVLHADAIHRGGGQVIPTARRVIYASQIT |
| | | | AKPRLLEPVYLVEIQAPEQALGGIYSVLNQKRGHVFEEMQRPGTPLYNIKAYLPVVE |
| | | | SFGFSSTLRAATSGQAFPQCVFDHWEMMSSDPLEPGSQASQLVADIRKRKGLKE |
| | | | QMTPLSEFEDK |
| 1020 | 77 | Cyn_d | EELRKIMDKKNNIRNMSVIAHVDHGKSTLTDSLVAAAGIIAQEVAGDVRMTDTRA |
| | | | DEAERGITIKSTGISLYYEMTDDSLKSFKGDRDGNEYLINLIDSPGHVDFSSEVTA |
| | | | ALRITDGALVVVDCIEGVCVQTETVLRQALGERIRPVLTVNKMDRCFLELQVDGEE |
| | | | AYQTFSRVIENANVIMATYEDKLLGDVQVYPEKGTVAFSAGLHGWAFTLTNFAKM |
| | | | YASKFGVDESKMMERLWGENFFDPSTKKWTTKNTGSPTCKRGFVQFCYEPIKQII |
| | | | NTCMNDQKDKLWPMLQKLNVTMKSDEKELMGKALMKRVMQTWLPASTALLEMM |
| | | | IFHLPSPSTAQKYRVENLYEGPLDDIYATAIRNCDPEGPLMLYVSKMIPASDKGRFF |
| | | | AFGRVFSGRVATGMKVRIMGPNYVPGQKKDLYVKSVQRTVIWMGKKQESVEDVP |
| | | | CGNTVAMVGLDQFITKNATLTNEKEVDACPIRAMKFSVSPVVRVAVQCKVASDLP |
| | | | KLVEGLKRLAKSDPMVLCTIEESGEHIIAGAGELHLEICLKDLQEDFMGGAEIIVSP |
| | | | PVVSFRETVLEKSCRTVMSKSPNKHNRLYMEARPLEEGLPEAIDEGRIGPRDDPKV |
| | | | RSKILSEEFGWDKDLAKKIWCFGPETTGPNMVVDMCKGVQYLNEIKDSVVAGFQ |
| | | | WASKEGALAEENMRGICFEVCDVVLHADAIHRGGGQVIPTARRVIYASQLTAKPR |
| | | | LLEPVYLVEIQAPENALGGIYGVLNQKRGHVFEEMQRPGTPLYNIKAYLPVIESFGF |
| | | | SSTLRAATSGQAFPQCVFDHWDMMSSDPLEAGSQAAQLVLDIRKRKGLKEQMTP |
| | | | LSEFEDKL |
| 1021 | 77 | Que_a | MVKFTADELRRIMDLKENIRNMSVIAHVDHGKSTLTDSLVAAAGIIAQEVAGDVR |
| | | | MTDTRADEAERGITIKSTGISLYYEMSNESLKSYKGERNGNEYLINLIDSPGHVDF |
| | | | SSEVTAALRITDGALVVVDCIEGVCVQTETVLRQALGERIRPVLTVNKMDRCFLEL |
| | | | QVDGEEAYTSFQKVIENANVIMATYEDPLLGDVQVYPEKGTVAFSAGLHGWAFTL |
| | | | TNFAKMYASKFGVDESKMMERLWGENFFDPATKKWTTKNTGSPTCKRGFVQFCY |
| | | | EPIKQIINTCMNDQKDKLWPMLAKLGVTMKSEEKELMGKPLMKRVMQNWLPASS |
| | | | ALLEMMIFHLPSPSTAQKYRVENLYEGPLDDSYASAIRNCDPEGPLMLYVSKMIPAS |
| | | | DKGRFFAFGRVFSGKVSTGLKVRIMGPNFVPGEKKDLYLKSVQRTVIWMGKKQET |
| | | | VEDVPCGNTVALVGLDQYITKNATLTNEKEVDAHPIRAMKFSVSPVVRVAVQCKV |
| | | | ASDLPKLVEGLKRLAKSDPMVVCSIEESGEHIIAGAGELHLEICLKDLQDDFMGGA |
| | | | EISKTDPIVSFRETVLDKSSRVVMSKSPNKHNRLYMEARPMEEGLAEAIDDGRIGP |
| | | | RDDPKVRSKILAEEFGWDKDLAKKIWCFGPETTGPNMVVDMCKGVQYLNEIKDS |
| | | | VVAGFQWASKEGALAEENMRGICFEVCDVVLHADAIHRGGGQVIPTARRVIYASQ |
| | | | LTAKPRLLEPVYMVEIQAPEQALGGIYSVLNRKRGHVFEEMQRPGTPLYNIKAYLPV |
| | | | KESFGFSQDLRAATSGQAFPQCVFDHWDIVSSDPLEAGSVAAQLVTDIRQRKGLK |
| | | | EQMTPLSDYEDKL |
| 1022 | 77 | Que_a | MVKFTADELRRIMDLKENIRNMSVIAHVDHGKSTLTDSLVAAAGIIAQEVAGDVR |
| | | | MTDTRADEAERGITIKSTGISLYYEMSNESLKSYKGERNGNEYLINLIDSPGHVDF |
| | | | SSEVTAALRITDGALVVVDCIEGVCVQTETVLRQALGERIRPVLTVNKMDRCFLEL |
| | | | QVDGEEAYTSFQKVIENANVIMATYEDPLLGDVQVYPEKGTVAFSAGLHGWAFTL |
| | | | TNFAKMYASKFGVDESKMMERLWGENFFDPATKKWTTKNTGSPTCKRGFVQFCY |
| | | | EPIKQIINTCMNDQKDKLWPMLAKLGVTMKSEEKELMGKPLMKRVMQNWLPASS |
| | | | ALLEMMIFHLPSPSTAQKYRVENLYEGPLDDSYASAIRNCDPEGPLMLYVSKMIPAS |
| | | | DKGRFFAFGRVFSGKVSTGLKVRIMGPNFVPGEKKDLYLKSVQRTVIWMGKKQET |
| | | | VEDVPCGNTVALVGLDQYITKNATLTNEKEVDAHPIRAMKFSVSPVVRVAVQCKV |
| | | | ASDLPKLVEGLKRLAKSDPMVVCSIEESGEHIIAGAGELHLEICLKDLQDDFMGGA |
| | | | EIIKSDPVVSFRETV |
| 1023 | 86 | Amb_p | DSKVFYLKMKGDYHRYLAEFKTGAERKEAAESTLNAYKAAQDIANAELAPTHPIRL |
| | | | GLALNFSVFYYEILN |
| 1024 | 86 | Amb_p | PNHRLLPSFVEPLIIMAREENVYMAKLSEQAERYEEMVQYMENVSNSLTDSEELTIE |
| | | | ERNLLSVAYKNVIGARRASWRIISSIEQKEESRGNQDHVSVIKDYRSKIEKELSDI |
| | | | CDGILKLLDSKLVPSAGSGDSKVFYL |
| 1025 | 86 | Amb_p | IIYKKTTKMASETKPDVSNSDKDEQVQRAKLAEQAERYDDMAAAMKLVTETGVEL |
| | | | SNEERNLLSVAYKNVVGARRSSWRVISSIEQKTEGSERKQQMAREYREKVEKELR |
| | | | EICYDVLNLLDKFLIPKATNAESKVFYLKMKGDYYRYLAEVATGDARTGVVEESQK |
| | | | AYQEAFDISKNKMQPTHPIRLGLALNFSVFYYEILNAPERACQLAKQAFDDAIAELD |
| | | | TLNEDSYKDSTLIMQLLRDNLTLWTSDTQADEDEPEEKKESK |
| 1026 | 86 | Amb_p | AFDQNTCTPFLVNNTHPASNNLRFCTLPPLYQLFSSLHITMGYEDSVYLAKLAEQAE |
| | | | RYEEMVENMKAVASADQELSVEERNLLSVAYKNVIGARRASWRIVTSIEQKEESK |
| | | | GNETQVTLIKEYRQKIEAELAKICEDILECLDGHLIPSAESGESKVFYHKMKGDYHR |
| | | | YLAEFASGEKRKVAATAAHEAYKTATDVAQTELTPTHPIRLGLALNFSVFYYEILNSP |
| | | | DRACHLAKQAFDDAIAELDSLSEESYRDSTLIMQLLRDNLTLWTSSDGNEGEAAG |
| | | | ATDAPKEEAKTTEDAPAASEPKADEQPPAAAPAPAA |
| 1027 | 86 | Amb_p | SPPTVYPSIRICTHPHSLPITQTHINSTITMATERESKTFLARLCEQAERYDEMVTYM |
| | | | KEVAKVAGELTVDERNLLSVAYKNVVGTRRASWRIISSIEQKEESKGNETQVTLIK |
| | | | EYRQKIEAELAKICEDILECLDGHLIPSAESGESKVFYHKMKGDYHRYLAEFASGEK |
| | | | RKVAATAAHEAYKTATDVAQTELTPTHPIRLGLALNFSVFYYEILNSPDRACHLAKQ |
| | | | AFDDAIAELDSLSEESYRDSTLIMQLLRDNLTLWTSSDGNEGEAAGATDAPKEEA |
| | | | KTTEDAPAASEPKADEQPPAAAPAPAA |
| 1028 | 86 | Amb_p | IFYLKMKGDYFRYLAEFKTGADRKEAAESTLLAYKSAQDIALSDLAPTHPIRLGLAL |
| | | | NFSVFYYEILNSPDRACNLAKQAFDEAIAELDTLGEDSYKDSTLIMQLLRDNLTLWT |
| | | | SDIADEAGDEIKESTKAEETQ |
| 1029 | 86 | Amb_p | IIPPFHFSPLSCLPNNLFSPHSSFVHRFIYKMSNEKERETHVYSAKLAEQAERYDEM |
| | | | VESMKNVAKLNVELTVEERNLLSVGYKNVIGARRASWRIMSSIEQKEESKGNENN |
| | | | VSLIKGYRKKVEDELSKICSDILDIIDKHLIPSSGSGEATVFYYKMKGDYFRYLAEFK |
| | | | TDEERKEAADQSLKGYEAASASASTDLPSTHPIRLGLALNFSVFYYEIMNSPEKAC |
| | | | HLAKQAFDEAIAELDTLSEESYKDSTLIMQLLRDNLTLWTSDLPEDGGDENPKGEE |
| | | | PKSAEPEKKQ |
| 1030 | 86 | Amb_p | VEKVSETDELTLEERNLLSVAYKNVIGARRASWRIISSIEQKEESRGNEDHVKVIK |
| | | | DYRAKIEAELTRICDGILKLLDSRLVPSASSGDSKVFYLKMKGDYHRYLAEFKTAGE |
| | | | RKDAAESTLTAYKSAQDIANTELAPTHPIRLGLALNFSVFYYEILNSPDRACSLAKQ |
| | | | AFDEAIAELDTLGEESYKDSTLIMQLLRDNLTLWTSDMQEDGADEIKEASGAKQS |
| | | | EDQEQQQQ |
| 1031 | 86 | Amb_p | QPLFPPLFSPLHTFPLNNLTPKPLTHLQTHPNLSDHHPNPNKMSLSDREQNVYMAK |
| | | | LAEQAERYDEMVEFMEKVSQTEELTVEERNLLSVAYKNVIGARRASWRIISSIEQK |
| | | | EESRGNEEHVKVIKEYRGKIESELTKVCDGILKLLDSRLIPKASSGDSKVFYLKMKG |
| | | | DYHRYLAEFKTAGERKDAAESTLTAYKSAQDIANTELAPTHPIRLGLALNFSVFYYEI |
| | | | LNSPDRACSLAKQAFDEAIAELDTLGEESYKDSTLIMQLLRDNLTLWTSDMQEDG |
| | | | ADEIKEASGAKQSEDQEQQQQ |
| 1032 | 86 | Ant_o | PLRIRASQRATMSPAEPTREESVYMAKLAEQAERYEEMVEFMERVAKATGGAGPG |
| | | | EELSVEERNLLSVAYKNVIGARRASWRIISSIEQKEESRGNDAHAATIRSYRSKIEA |
| | | | ELAKICDGILALLDSHLVPSAAAAESKVFYLKMKGDYHRYLAEFKSGAERKEAAES |
| | | | TMNSYKAAQDIALADLAPTHPIRLGLALNFSVFYYEILNSPDRACNLAKQAFDEAIS |
| | | | ELDSLGEESYKDSTLIMQLLRDNLTLWTSDTNEDGGDEIKEAPAPKESEGQ |
| 1033 | 86 | Ant_o | QTRGKMSTAEATREENVYMAKLAEQAERYEEMVEFMEKVAKTADVGELTVEERNL |
| | | | LSVAYKNVIGARRASWRIISSIEQKEESRGNEAYVASIKEYRTRIETELSKICDGILK |
| | | | LLDSHLVPSATAAESKVFYLKMKGDYHRYLAEFKAGTERKEAAENTLVAYKSAQDI |
| | | | ALADLPTTHPIRLGLALNFSVFYYEILNSPDRACNLAKQAFDEAIAELDSLGEESYK |
| | | | DSTLIMQLL |
| 1034 | 86 | Bet_v | LFGIAKMSPADSSREENVYMAKLAEQAERYEEMVEFMEKVAKTVDVEELSVEERN |
| | | | LLSVAYKNVIGARRASWRIISSIEQKEESRGNEDHVAVIKEYRGKIESELSKICDGI |
| | | | LSLLESHLIPSASSAESKVFYLKMKGDYHRYLAEFKTSAERKEAAESTLLAYKSAQD |
| | | | IALAELAPTHPIRLGLALNFSVFYYEILNSPDRACNLAKQAFDEAISELDTLGEESYK |
| | | | DSTLIMQLLRDNLTLWTSDITDDAGDEIKEASKRESAEGQQPPSQ |
| 1035 | 86 | Bet_v | SISEKMSTEKERETQVYLAKLAEQAERYEEMVECMKNVARLDLELTVEERNLLSVG |
| | | | YKNVIGARRASWRIMSSIEQKEESKGNEHNVKLIKGYRQRVEEELSKICYDILGIID |
| | | | KHLIPSSTSGEATVFYYKMKGDYYRYLAEFKIDQERKEAAEESLKGYEAASATANT |
| | | | DLPSTHPIRLGLALNFSVFYYEIMNSPERACHLAKQAFDEAIAELDTLSEESYKDSTL |
| | | | IMQLLRDNLTLWTSDLPEDGGEDNLKVEESKPTEAEH |
| 1036 | 86 | Bet_v | ALFSEKKKKKEKINDDLSTSLLLHSTENNSFFPTLQDSLSIVKFRFHLNVTQFTSLS |
| | | | PSLSLFAPMASSLTREQYVYMAKLSEQAERYEEMVEYMEKLVTGSTPAAELNVEER |
| | | | NLLSVAYKNVIGSLRAAWRIVSSIEQKEEGRKNEEHVVLVKEYRSKMESELSVVCA |
| | | | GILKLLDSHLVPSALSGESKVFYLKMKGDYHRYLAEFKVGDERKAAAEDTMLAYKA |
| | | | AQDIALADLAPTHPIRLGLALNYSVFYYEILNSSEKACSMAKQAFEEAIAELDTLGE |
| | | | DSYKDSTLIMQLLRDNLTLWTSDMQEQIDEA |
| 1037 | 86 | Bet_v | PPSQHPLSTPPPPTSPPHSRPPLPSTTPRNTPAEMATERESKTFLARLCEQAERYDE |
| | | | MVTYMKEVAKIGGELTVDERNLLSVAYKNVVGTRRASWRIISSIEQKEEAKGTEKH |
| | | | VGIIREYRQKIELELEKVCEDVLNVLDESLIPKAETGESKVFYHKMKGDYHRYLAEF |
| | | | ASGPKRKGAATAAHEAYKSATDVAQTELTPTHPIRLGLALNFSVFYYEILNSPDRAC |
| | | | HLAKQAFDDAIAELDSLSEESYRDSTLIMQLLRDNLTLWTSADGNEGEGAKEEKPE |
| | | | EEAQAPAAEAAAAPAEEKPEEAKPVEADS |
| 1038 | 86 | Cyn_d | SIEQKEEGRGNEDRVTLIKDYRGKIETELTKICDGILKLLESHLVPSSTAPESKVFYL |
| | | | KMKGDYYRYLAEFKTGTERKDAAENTMVAYKAAQDIALAELAPTHPIRLGLALNFS |
| | | | VFYYEILNSPDRACSLAKQAFDEAISELDTLSEESYKDSTLIMQLLRDNLTLWTSDI |
| | | | SEDPAEEIREAAPKSGEGQ |
| 1039 | 6 | Cyn_d | VFYLKMKGDYHRYLAEFKTGAERKEAADATLAAYQAAQDIAIKELPPTHPIRLGLAL |
| | | | NFSVFYYEILNSPDRACSLAKQAFDEAISELDTLGEESYKDSTLIMQLLRDNLTLWT |
| | | | SDMQDDGGDEMRDASKPEDEQ |
| 1040 | 6 | Cyn_d | PPRHPTAMRVPHPPHPGGRVLLKCPTPPVASPNRTDASHPPQEDPLRRANPVAFPV |
| | | | PGSPEEIPPPAAMSPSEPTREESVYMAKLAEQAERYEEMVEFMERVARSAGGAGG |
| | | | GEELSVEERNLLSVAYKNVIGARRASWRIISSIEQKEEGRGNEAHAASIRAYRSKIE |
| | | | AELARICDGILALLDSHLVPSAGAAESKVFYLKMKGDYHRYLAEFKSGTERKEAAE |
| | | | STMNAYKAAQDIALADLAPTHPIRLGLALNFSVFYYEILNSPDRACNLAKQAFDEAI |
| | | | SELDSLGEESYKDSTLIMQLLRDNLTLWTSDTNEDGGDEIKEAAAPKESGDAQ |
| 1041 | 6 | Cyn_d | MAKLAEQAERYEEMVEYMEKVAKTVDVEELTVEERNLLSVAYKNVIGARRASWRI |
| | | | VSSIEQKEESRKNEEHVNLIKEYRGKIEAELSNICDGILKLLDSHLVPSSTAAESKV |
| | | | FYLKMKGDYHRYLAEFKTGAERKESAESTMVAYKAAQDIALAELAPTHPIRLGLAL |
| | | | NFSVFYYEILNSPDKACNLAKQAFDEAISELDTLGEESYKDSTLIMQLLRDNLTLWT |
| | | | SDLTEEGAEDGKEASKGEAGEGQ |
| 1042 | 6 | Cyn_d | MAKLAEQAERYEEMVEYMEKVAKTVDVEELTVEERNLLSVAYKNVIGARRASWRI |
| | | | VSSIEQKEESRKNEEHVNLIKEYRGKIEAELSNICDGILKLLDSHLVPSSTAAESKV |
| | | | FYLKMKGDYHRYLAEFKTGAERKESAESTMVAYKAAQDIALAELAPTHPIRLGLAL |
| | | | NFSVFYYEILNSPDKACNLAKQAFDEAISELDTLGEESYKDSTLIMQLLRDNLTLWT |
| | | | SDLTEEGAEEGKEAPKGDAGEGQ |
| 1043 | 6 | Fra_e | FRQHTQNSPSKKRALSQSRSLSLNSMASNREENVYVAKLAEQAERYEEMVEYMEK |
| | | | VATAVEGDELTMEERNLLSVAYKNVIGARRASWRIISSIEQKEESRGNEGHVSTIK |
| | | | GYRSKIESELSSICDGILKLLDSKLIGSASSGDSKVFYLKMKGDYYRYLAEFKTGAE |
| | | | RKEAAENTLSSYKSAQDIANAELAPTHPIRLGLALNFSVFYYEILNSSDLACNLAKQ |
| | | | AFDEAIAELDSLGEESYKDSTLIMQLLRDNLTLWTSDMQDDGSEEIKEAPKPDNE |
| 1044 | 86 | Fra_e | VLFNILKMSPADSSREENVYMAKLAEQAERYEEMVEFMEKVAKTVSTEELTVEERN |
| | | | LLSVAYKNVIGARRASWRIISSIEQKEESRGNEDHVNVIKEYRSKIEAELSKICDGI |
| | | | LSLLESHLVPSASSAETKVFYLKMKGDYHRYLAEFKTGAERKEAAESTLVAYKSAQ |
| | | | DIALADLAPTHPIRLGLALNFSVFYYEILNSPDRACNLAKQAFDEAIAELDTLGEESY |
| | | | KDSTLIMQLLRDNLTLWTSDITDDAGDEIKEASKPETGEGHQ |
| 1045 | 86 | Fra_e | SRGNEDHVKVLKEYRAKIEAELSKISGGILSLLDSHLITSASTAESKVFYLKMKGDY |
| | | | HRYLAEFKTGAER |
| 1046 | 86 | Fra_e | REKKVKKERRIIFIFTISSDSSLTQEDIEMEKEREQQVYLARLAEQAERYDEMVEAM |
| | | | KSVAKLDVELTVEERNLVSVGYKNVIGARRASWRILSSIEQKEESKGHEQNVKRIK |
| | | | NYRQRVEDELTKICNDILSVIDEHLLPSSSTGESTVFYYKMKGDYYRYLGEFKTGD |
| | | | DRKEAADQSLKAYEAATSSASTDLPPTHPIRLGLALNFSVFYYEILNSPERACHLAK |
| | | | QAFDEAIAELDSLNEESYKDSTLIMQLLRDNLTLWTSDLPEEGGEQSKGDEAQRE |
| | | | VRFYDYNPVYNNIFKSLVST |
| 1047 | 86 | Lol_p | QTRGRMSTAEATREENVYMAKLAEQAERYEEMVEFM |
| 1048 | 86 | Lol_p | HAGPAPSAPGDLLKSPPLPAPASPTNTFTSSVPGSPQLPPYLPLAHPTMSPAEPTRE |
| | | | ESVYMAKLAEQAERYEEMVEFMERVAKATGGAGPGEELSVEERNLLSVAYKNVIG |
| | | | ARRASWRIISSIEQKEEGRGNDAHAATIRSYRTKIEAELAKICDGILALLDSHLVPS |
| | | | AGAAESKVFYLKMKGDYHRYLAEFKSGAERKEAAESTMNSYKAAQDIALADLAPT |
| | | | HPIRLGLALNFSVFYYEILNSPDRACNLAKQAFDEAISELDSLGEESYKDSTLIMQLL |
| | | | RDNLTLWTSDTNEDGGDEIKEAPAPKESGEGQ |
| 1049 | 86 | Lol_p | SWRIISSIEQKEESRGNEAYVASIKEYRTRIETELSKICDGILKLLDSHLVPSATAAE |
| | | | SKVFYLKMKGDYHRYLAEFKAGAERKEAAENTLVAYKSAQDIALADLPTTHPIRLGL |
| | | | ALNFSVFYYEILNSPDRACNLAKQAFDEAIAELDSLGEESYKDSTLIMQLLRDNLTL |
| | | | WTSDNADEGGDEIKEASKPEGEGH |
| 1050 | 86 | Lol_p | NPQKLKMAELSREENVYMAKLAEQAERYEEMVEFMEKVAKTVDSEELTVEERNLL |
| | | | SVAYKNVIGARRASWRIISSIEQKEESRGNEDRVTLIKDYRGKIETELTKICDGILK |
| | | | LLDSH |
| 1051 | 86 | Lol_p | MAKLAEQAERYEEMVEYMEKVAKTVDVEELTVEERNLLSVAYKNVIGARRASWRI |
| | | | VSSIEQKEEGRGNEEHVTLIKEYRGKIEAELSKICDGILKLLDSHLVPMSTAAESKV |
| | | | FYLKMKGDYHRYLAEFKASAERKEAAESTMVAYKAAQDIALAELAPTHPIRLGLALN |
| | | | FSVFYYEILNSPDKACNLAKQAFDEAISELDTLGEESYKDSTLIMQLLRDNLTLWTS |
| | | | DLTEEGGAEDGKEASKGEGAEGQ |
| 1052 | 86 | Lol_p | MAKLAEQAERYEEMVEYMEKVAKTVDVEELTVEERNLLSVAYKNVIGARRASWRI |
| | | | VSSIEQKEEGRGNEEHVTLIKEYRGKIEAELSKICDGILKLLDSHLVPMSTAAESKV |
| | | | FYLKMKGDYHRYLAEFKASAERKEAAESTMVAYKAAQDIALAELAPTHPIRLGLALN |
| | | | FSVFYYEILNSPDKACNLAKQAFDEAISELDTLGEESYKDSTLIMQLLRDNLTLWTS |
| | | | DITDDAGDEIKEASKPETGEGHQ |
| 1053 | 86 | Ole_e | RKREGSSSSLPYSQTHHSHRREDSEMEKEREQLVYLARLAEQAERYDEMVEAMK |
| | | | NVAKLDVELTVEERNLVSVGYKNVIGARRASWRILSSIEQKEESKGHEQNVKRIKS |
| | | | YRQRVEDELTKICNDILSVIDEHLLPSSSTGESTVFYHKMKGDYYRYLGEFKTGDD |
| | | | RKEAADQSLKAYEAATSAASTDLPPTHPIRLGLALNFSVFYYEILNSPERACHLAKQ |
| | | | AFDEAIAELDSLNEESYKDSTLIMQLLRDNLTLWTSDLPEEGGEQSKGDDAQGES |
| 1054 | 86 | Ole_e | SRSLSLNSMASNREENVYMAKLAEQAERYEEMVEYMEKVVTAVDGDELTVEERNL |
| | | | LSVAYKNVIGARRASWRIISSIEQKEESRGNEGHVSTIKGYRSKIESELSSICDGIL |
| | | | KLLDSKLIGSASSGDSKVFYLKMKGDYYRYLAEFKTGPERKEAAEHTLSSYKSAQD |
| | | | IANAELAPTHPIRLGLALNFSVFYYEILNSPELACNLAKQAFDEAIAELDTLGEESYK |
| | | | DSTLIMQLLRDNLTLWTSDMQDDGSEEIKEAPKPDNE |
| 1055 | 86 | Ole_e | VLYSTVKMSPADSSREENVYMAKLAEQAERYEEMVEFMEKVAKTVNAEEFSVEER |
| | | | NLLSVAYKNVIGARRASWRIISSIEQKEESRGNEDHVNVIKEYRVKIEAELCKICDG |
| | | | ILSLLESHLIPSASSAESKVFYLKMKGDYHRYLAEFKTGAERKEVAESTLLAYKSAQ |
| | | | DIALADLSPTHPIRLGLALNFSVFYYEILNSPDRACNLAKQAFDEAIAELDTLGEESY |
| | | | KDSTLIMQLLRDNLTLWTSDITDDAGDEIKDTSKPESGEEQQ |
| 1056 | 86 | Ole_e | IPSTPHISKPPNPFTLFPSDLIHILPSPCISFLFQKSGSPTIMAATAREENVYKAKLAE |
| | | | QAERYEEMVEFMEKVSESLTVNEELTVEERNLLSVAYKNVIGARRASWRIISSIEQ |
| | | | KEESRGNEDHVSTIKDYRSKIESELSNICDGILKLLESKLIVSASSGDSKVFYIKMK |
| | | | GDYHRYLAEFKTGAERKEAAESTLTAYKAAQDIANAELAPTHPIRLGLALNFSVFYY |
| | | | EILNSPDRACSLAKQAFDEAIAQLDTLGEESYKDSTLIMQLLRDNLTLWTSDMQD |
| | | | DGTDDIKEAPKRDDEQQGE |
| 1057 | 86 | Pla_l | TTSQPYRFEHLKMSREENVYMAKLAEQAERYEEMVEFMEKVAKTSDTDELTVEER |
| | | | NLLSVAYKNVIGARRASWRIISSIEQKEESRGNEDHVTIIKDYRGKIEAELSKICDG |
| | | | ILNLLETHLVPAASSAESKVFYLKMKGDYHRYLAEFKTGAERKEAAESTLLAYKSAQ |
| | | | DIALADLAPTHPIRLGLALNFSVFYYEILNSPDRACNLAKQAFDEAISELDTLGEESY |
| | | | KDSTLIMQLLRDNLTLWTSDTTDDAGDEIKETTKLVPGEGQE |
| 1058 | 86 | Pla_l | PHIIPLSLSHFPSKFTQSITPPIPNPPPMAAREDNVYMAKLAEQAERYEEMVEFMEK |
| | | | VSASLSDSDELTVEERNLLSVAYKNVIGARRASWRIISSIEQKEESRGNESHVSAI |
| | | | KSYRSKIENELSGICDGILKLLDTKLIGSAGNGDSKVFYLKMKGDYHRYLAEFKTG |
| | | | AERKEAAENTLSAYKAAQDIANAELAPTHPIRLGLALNFSVFYYEILNSPDRACNLA |
| | | | KQAFDEAIAELDTLGEESYKDSTLIMQLLRDNLTLWTSDMQDDNSEEIKEAPKPD |
| | | | NE |
| 1059 | 86 | Pla_l | PHIIPLSLSHFPSKFTQSITPPIPNPPPMAAREDNVYMAKLAEQAERYEEMVEFMEK |
| | | | VSASLSDSDELTVEERNLLSVAYKNVIGARRASWRIISSIEQKEESRGNEEHVSTI |
| | | | KDYRSKIEKELSDICDGILKLLDSRLIPSAATGDSKVFYLKMKGDYHRYLAEFKTGA |
| | | | NRKEAAESTLTAYKAAQDIANSELAPTHPIRLGLALNFSVFYYEILNSPDRACNLAK |
| | | | QAFDEAIAELDTLGEESYKDSTLIMQLLRDNLTLWTSDMQDEAADEVKEAPKAEE |
| | | | AEQQ |
| 1060 | 86 | Pla_l | PHIIPLSLSHFPSKFTQSITPPIPNPPPMAAREDNVYMAKLAEQAERYEEMVEFMEK |
| | | | VSASLSDSDELTVEERNLLSVAYKNVIGARRASWRIISSIEQKEESRGNESHVSAI |
| | | | KSYRSKIEDELSGICDGILKLLDTKLIGSAASGDSKVFYLKMKGDYHRYLAEFKTGA |
| | | | ERKEAAENTLSAYKAAQDIANAELAPTHPIRLGLALNFSVFYYEILNSPDRACNLAK |
| | | | QAFDEAIAELDTLGEESYKDSTLIMQLLRDNLTLWTSDMQDDTSEEIKEAPKPDNE |
| 1061 | 86 | Pla_l | CKWLKMSPAESSREDYVYLAKLAEQAERYEEMVEFMEKVAKSTESDELTVEERNL |
| | | | LSVAYKNVIGARRASWRIISSIEQKEESRGNEDHVKVIKEYRGKIETELNKICDGIL |
| | | | GLLDSHLVPSAASAESKVFYLKMKGDYYRYLAEFKIGAERKEAAENTLAAYKSAQD |
| | | | IALADLAPTHPIRLGLALNFSVFYYEILNSPDRACNLAKQAFDEAISELDTLGEESYK |
| | | | DSTLIMQLLRDNLTLWTSDTTDDAGDEIKESGKNDSGEGHE |
| 1062 | 86 | Pla_l | IVLFPSFPDPSAMTTEKERETHVYLAKLAEQAERYDEMVECMKQVAKLDVELSVDE |
| | | | RNLLSVGYKNVIGARRASWRIMSSIEQKEESKGNENNVKLIKDYRQKVEDELSKI |
| | | | CYDILEVIDKHLVPSSGSGEATVFYYKMKGDYFRYLAEFKTDQEKKEAAEQSLKGY |
| | | | EAASATANTDLPSTHPIRLGLALNFSVFYYEIMNSPERACHLAKQAFDEAIAELDTL |
| | | | SEESYKDSTLIMQLLRDNLTLWTSDLPEDGGDENGKAEETNTKPDENEKLLG |
| 1063 | 86 | Poa_p | PTRRHCHAGPAPSAPGDLLKSPPLLLRLPHKRVHLSPPSPDPLAHPSLFATMSPAEP |
| | | | TREESVYMAKLAEQAERYEEMVEFMERVAKATGGAGPGEELSVEERNLLSVAYKN |
| | | | VIGARRASWRIISSIEQKEEGRGNDAHAATIRSYRTQIEAELAKICEGILALLDSHL |
| | | | VPSAGAAESKVFYLKMKGDYHRYLAEFKSGAERKEAAESTMNAYKAAQDIALADL |
| | | | APTHPIRLGLALNFSVFYYEILNSPDRACNLAKQAFDEAISELDSLGEESYKDSTLIM |
| | | | QLLRDNLTLWTSDTNEEGGDDIKEAPAPKESGDGQ |
| 1064 | 86 | Poa_p | QTRGKMSTAEATREENVYMAKLAEQAERYEEMVEFMEKVAKTADVGELTVEERNL |
| | | | LSVAYKNVIGARRASWRIISSIEQKEESRGNEAYVASIKEYRTRIETELSKICDGILK |
| | | | LLDSHLVPSATAAESKVFYLKMKGDYHRYLAEFKAGAERKEAAENTLVAYKSAQDI |
| | | | ALADLPTTHPIRLGLALNFSVFYYEILNSPDRACNLAKQAFDEAIAELDSLGEESYK |
| | | | DSTLIMQLLRDNLTLWTSDNADEGGDEIKEASKPEGEGH |
| 1065 | 86 | Poa_p | RTRGKMSTAEATREENVYMAKLAEQAERYEEMVEFMEKVAKTADVGELTVEERNL |
| | | | LSVAYKNVIGARRASWRIISSIEQKEESRGNEAYVASIKEYRTRIETELSKICDGILK |
| | | | LLDSHLVPSATAAESKVFYLKMKGDYHRYLAEFKAGAERKEAAENTLVAYKSAQDI |
| | | | ALADLPTTHPIRLGLALNFSVFYYEILNSPDRACNLAKQAFDEAIAELDSLGEESYK |
| | | | DSTLIMQLLRDNLTLWTSDNADEGGDEIKEASKPEGEGH |
| 1066 | 86 | Que_a | LSSHPGGQRAWGSEHPSLYLSGHVLLNPQKQFQTLLSFSTFISFFISFHCILFVWLR |
| | | | LRLETERLAMAIDKERENHVYIAKLAEQAERYDEMVDAMTKVANMDVELSVEERN |
| | | | LLSVAYKNVVGARRASWRILSSLEQKEESKGNDLNVKRIKNYRHEIESELSRVCAD |
| | | | IIALIDEHLIPSCSVGESPVFFYKMKGDYYRYLAEFRADDERKETADLSMKAYQAAS |
| | | | TTAEAELPPTHPIRLGLALNFSVFYYEIMNSPERACALAKQAFDEAISELDSLSEESY |
| | | | KDSTLIMQLLRDNLTLWTSDIPENEVEEAPKLDSNAKAGGGEDAE |
| 1067 | 86 | Que_a | LFHFCSHTSFLSLTRTHTQRERNFSFFANQRAKMSPTDSSREENVYMAKLAEQAE |
| | | | RYEEMVEFMEKVAKTVDVEELTVEERNLLSVAYKNVIGARRASWRIISSIEQKEES |
| | | | RGNEDHVVIIKEYRGKIENELSKICDGILGLLETHLIPSASAAESKVFYLKMKGDYH |
| | | | RYLAEFKTGAERKEAAESTLLAYKSAQDIALAELPPTHPIRLGLALNFSVFYYEILNS |
| | | | PDRACNLAKQAFDEAISELDTLGEESYKDSTLIMQLLRDNLTLWTSDITDDAGDEI |
| | | | KEASKRESGEGQPPQQQ |
| 1068 | 86_51 | Amb_a | REENVYMAKLSEQAERYEEMVQYMENVSNSLTDSEELTIEERNLLSVAYKNVIGAR |
| | | | RASWRIISSIEQKEESRGNQDHVSVIKDYRSKIEKELSDICDGILKLLDSKLVPSAG |
| | | | SGDSKVFYLKMKGDYHRYLAEFKTGAERKEAAESTLNAYKAAQDIANAELAPTHPI |
| | | | RLGLALNFSVFYYEILNSPDRACGLAKQAFDEAIAELDTLGEDSYKDSTLIMQLLRD |
| | | | NLTLWTSDMQDEGADEIKEAKQSEE |
| 1069 | 86_51 | Amb_a | REQNVYMAKLAEQAERYDEMVEFMEKVSQTEELTVEERNLLSVAYKNVIGARRAS |
| | | | WRIISSIEQKEESRGNEEHVKVIKEYRGKIESELTKVCDGILKLLDSRLIPKASSGD |
| | | | SKVFYLKMKGDYHRYLAEFKTAGERKDAAESTLTAYKSAQDIANTELAPTHPIRLGL |
| | | | ALNFSVFYYEILNSPDRACSLAKQAFDEAIAELDTLGEESYKDSTLIMQLLRDNLTL |
| | | | WTSDMQEDGGDEIKEAASGKQS |
| 1070 | 86_51 | Amb_p | REQNVYMAKLAEQAERYDEMVEFMEKVSQTEELTVEERNLLSVAYKNVIGARRAS |
| | | | WRIISSIEQKEESRGNEEHVKVIKEYRGKIESELTKVCDGILKLLDSRLIPKASSGD |
| | | | SKVFYLKMKGDYHRYLAEFKTAGERKDAAESTLTAYKSAQDIANTELAPTHPIRLGL |
| | | | ALNFSVFYYEILNSPDRACSLAKQAFDEAIAELDTLGEESYKDSTLIMQLLRDNLTL |
| | | | WTSDMQEDGGDEIKEAASGKQS |
| 1071 | 86_51 | Amb_p | MSLSDREQNVYMAKLAEQAERYDEMVEFMEKVSQTEELTVEERNLLSVAYKNVIG |
| | | | ARRASWRIISSIEQKEESRGNEEHVKVIKEYRGKIESELTKVCDGILKLLDSRLIPK |
| | | | ASSGDSKVFYLKMKGDYHRYLAEFKTAGERKDAAESTLTAYKSAQDIANTELAPTH |
| | | | PIRLGLALNFSVFYYEILNSPDRACSLAKQAFDEAIAELDTLGEESYKDSTLIMQLLR |
| | | | DNLTLWTSDMQEDGADEIKEASGAKQSED |
| 1072 | 86_51 | Bet_v | MSPADSSREENVYMAKLAEQAERYEEMVEFMEKVAKTVDVEELSVEERNLLSVAY |
| | | | KNVIGARRASWRIISSIEQKEESRGNEDHVAVIKEYRGKIESELSKICDGILSLLES |
| | | | HLIPSASSAESKVFYLKMKGDYHRYLAEFKTSAERKEAAESTLLAYKSAQDIALAEL |
| | | | APTHPIRLGLALNFSVFYYEILNSPDRACNLAKQAFDEAISELDTLGEESYKDSTLIM |
| | | | QLLRDNLTLWTSDITDDAGDEIKEASKRESAEG |
| 1073 | 86_51 | Cyn_d | MSPSEPTREESVYMAKLAEQAERYEEMVEFMERVARSAGGAGGGEELSVEERNLL |
| | | | SVAYKNVIGARRASWRIISSIEQKEEGRGNEAHAASIRAYRSKIEAELARICDGILA |
| | | | LLDSHLVPSAGAAESKVFYLKMKGDYHRYLAEFKSGTERKEAAESTMNAYKAAQD |
| | | | IALADLAPTHPIRLGLALNFSVFYYEILNSPDRACNLAKQAFDEAISELDSLGEESYK |
| | | | DSTLIMQLLRDNLTLWTSDTNEDGGDEIKEAAAPKESGD |
| 1074 | 86_51 | Que_a | MSPTDSSREENVYMAKLAEQAERYEEMVEFMEKVAKTVDVEELTVEERNLLSVAY |
| | | | KNVIGARRASWRIISSIEQKEESRGNEDHVVIIKEYRGKIENELSKICDGILGLLET |
| | | | HLIPSASAAESKVFYLKMKGDYHRYLAEFKTGAERKEAAESTLLAYKSAQDIALAEL |
| | | | PPTHPIRLGLALNFSVFYYEILNSPDRACNLAKQAFDEAISELDTLGEESYKDSTLIM |
| | | | QLLRDNLTLWTSDITDDAGDEIKEASKRESGEG |
| 1075 | 87 | Amba | YFRYYSMYGHVEKLAEEIKKGAASVEGVEAKLWQVPETLNEDVLGKMSAPPKSDV |
| | | | PVITANDLSEADGFVFGFPTRFGMMSAQFKAFFDSTGGLWRTQQLAGKPAGIFYS |
| | | | TGSQGGGQETTALTAITQLVHHGMIFVPIGYTFGAGMFEMEKVKGGSPYGAGTYA |
| | | | GDGSRQPSELELQQAFHQGKHIATIAKKLKGAA |
| 1076 | 87 | Amba | SVEGVEAKLWQVPETLNDEVLGKMSAPPKSDAPIITPNELAEADGFIFGFPTRFGM |
| | | | MAAQFKAFFDATGGLWRTQQLAGKPAGIFYSTGSQGGGQETTPLTAITQLVHHG |
| | | | MIFVPIGYTFGAGMFEMEKVKGGSPYGAGT |
| 1077 | 87 | Amb_p | MAPKIAIVYYSMYGHIKKMADAELKGIQEAGGDAKLFQVAETLPQDVLDKMYAPP |
| | | | KDSSVPVLEDPAVLEEFDGILFGIPTRYGNFPAQFKTFWDKTGKQWQQGSFWGKY |
| | | | AGVFVSTGTLGGGQETTAITSMSTLVHHGFIYVPLGYKTAFSMLANLDEVHGGSP |
| | | | WGAGTFSAGDGSRQPSELELNIAQAQGKAF |
| 1078 | 87 | Amb_p | PIITPNELAEADGFIFGFPTRFGMMAAQFKAFFDATGGLWRTQQLAGKPAGIFYST |
| | | | GSQGGGQETTPLTAITQLVHHGMIFVPIGYTFGAGMFEMEKVKGGSPYGAGT |
| 1079 | 87 | Bet_v | MATKVYIVYYSMYGHVEKLAEEIKKGASSVEGVEAQLWQVPETLQEEVLGKMSAP |
| | | | PKSDVAIITPNELAEADGFVFGFPTRFGMMAAQFKAFLDATGGLWRTQQLAGKPA |
| | | | GLFYSTGSQGGGQETTALTAITQLVHHGMIFVPIGYTFGAGMFEMESVKGGSPYG |
| | | | AGTFAGDGSRQPTDLELKQAFHQGQYIATITKKLKGAA |
| 1080 | 87 | Cyn_d | MAAKVYIVYYSTYGHVGKLAEEIKKGASSVEGVEAKLWQVPETLSEEVLGKMGAP |
| | | | PKPDVPVITPQELAEADGILFGFPTRFGMMAAQMKAFFDATGGLWREQSLAGKPA |
| | | | GIFFSTGTQGGGQETTPLTAITQLTHHGMVFVPVGYTFGAKLFGMDQVQGGSPYG |
| | | | AGTFAADGSRWPSEVELEHAFHQGKYFAGIAKKLKGSA |
| 1081 | 87 | Cyn_d | MAVKVYVVFYSTYGHVAKLAEEIKKGAASVEGVEVKLWQVPETLSEEVLGKMGAP |
| | | | PKTDVPVITPQELAESDGSLFGFPTRFGMMAAQMKAFFDATGGLWREQSLAGKPA |
| | | | GIFFSTGTQGGGQE |
| 1082 | 87 | Cyn_d | QGGGQETTPLTAVTQLTHHGMVFVPVGYTFGAKMFDMESVHGGSPYGAGTFAGD |
| | | | GSRWPTEVELEHAFHQGKYFAGI |
| 1083 | 87 | Que_a | MATKVYIVYYSMYGHVEKLAEEIRKGAASVEGVEAKLWQVPETLPEEVLGKMSAPP |
| | | | KSDVPIITPDQLTDADGLVFGFPTRYGMMAAQFKAFLDATGGLWRSQQLAGKPAG |
| | | | LFYSTGSQGGGQETTALTAITQLVHHGMIFVPIGYTFGAGMFEMEKVKGGSPYGA |
| | | | GTFAGDGSRQPTELELEQAFHQGKYIAAITKKLKGGAA |
| 1084 | 87 | Que_a | LAGKPAGLFYSTGSQGGGQETTPLTAITQLVHHGMIFVPIGYTFGAGMFEMEKVRG |
| | | | GTPYGAGTYAGDGSRQPSE |
| 1085 | 89 | Amb_p | MTHPTLAIPELMRLLMDEEGLGWDEAWDVTSKYLNLFMTVILKSVTILILLFGPK |
| 1086 | 89 | Amb_p | VFIIFFVFLRKPTHIPLLISSCVILFLQVNGVAQLHNDILKAELCACYVSIWPTKFQNK |
| | | | TNGITPRR |
| 1087 | 89 | Amb_p | SLEGNEGFGRGDYFLVGKDFPSYIECQEKVDEAYRDQKRWTRMSILNTAGSYKFS |
| | | | SDRTIHEYARDIWNIQPLQLP |
| 1088 | 89 | Ant_o | KRIVKLVNDVGAVVNNDPDVNKYLKVVFIPNYNVSVAEVLIPGSELSQHISTAGME |
| | | | ASGTSNMKFSLNGCVIIGTLDG |
| 1089 | 89 | Ant_o | SFPKIVRLAQFLGRAIAVPSRPLQKAPTGSHLSPSPIRCPNSEALSPPPPHARRLRIP |
| | | | HHSAMSAADKVKPAANPAAEDAKAIAGNISYHAQYSPHFSPLAFGPEPAYFATAES |
| | | | VRDHLLQRWNDTYLHFHKTDPKQTYYLSMEYLQGRALTNAVGNLNITGAYAEAVK |
| | | | KFGYELEALAGQERDMALGNGGLGRLAACFLDSMATLNLPAWGYGLRYRYGLFKQ |
| | | | RITKEGQEEVAEDWLEKFSPWEIVRHDVVYPVRFFGHVEISPDGSRKVAGGEVLN |
| | | | ALAYDVPIPGYKTKNAISLRLWDAKASAEDFNLFQFNDGQYESSAQLHSRAQQIC |
| | | | AVLYPGDATEEGKLLRLKQQFFLCSASLQDIIFRFKERKSDRVSGKWSEFPSKVAV |
| | | | QMNDTHPTLAIPELMRLLMDEEGLGWDEAWEVTNKTVAYTNHTVLPEALEKWSQ |
| | | | AVMRKLLPRQMEIIEEIDKRFREMVISTRKDMEGKLDLMSVLDNSPQKPVVRMAN |
| | | | LCVVSAHTVNGVAELHSNILKEELFADYVSIWPNKFQNKTNGITPRRWLRFCNPEL |
| | | | SEIVTKWLKTDKWTSNLDLLTGLRKFADDEKLHTEWAAAKLASKKRLAKHVLDVT |
| | | | GVTIDPNSLFDIQIKRIHEYKRQLMNILGAVYRYKKLKEMSAEEKQKVTPRTVMVG |
| | | | GKAFATYTNAKRIVKLVTDVGAVVNNDPDVNKYLKVVFIPNYNVSVAEVLIPGSEL |
| | | | SQHISTAGMEASGTSNMKFSLNGCVIIGTLDGANVEIREEVGEDNFFLFGAKADQ |
| | | | VAGLRKDRENGLFKPDPRFEEAKNYIRSGTFGTYDYTPLLDSLEGNSGFGRGDYFL |
| | | | VGYDFPSYIDAQARVDEAYKNKKRWIKMSILNTAGSGKFSSDRTIAQYAKEIWGI |
| | | | TASPVP |
| 1090 | 89 | Bet_v | QIVMAAIREVNGSTGCTISAKVPAVAQPLAEEPAAIASNINYHAQFSPHFSPFKFEP |
| | | | EQAYYATAESVRDRLVQQWNETYVHFHKVDPKQTYYLSMEYLQGRALTNAIGNLK |
| | | | VQDAYGDALKKLGHKLEEITEEEKDAALGNGGLGRLASCFLDSMATLNLPAWGYG |
| | | | LRYKYGLFKQRFTKEGQEEIAEDWLEKFSPWEVVRHDIVYPVRFFGHVEVNPNESR |
| | | | KWVGGEVVQALAYDVPIPGYNTKNTISLRLWEAKACAEDFNLFQFNDGQYESAAQ |
| | | | LHSRAQQICAVLYPGDATENGKLLRLKQQFFLCSASLQDIIFRFKERRLGKGSWQ |
| | | | WSEFPSKVAVQLNDTHPTLAIPELMRLLMDDEGLGWDEAWDVTTRTVAYTNHTV |
| | | | LPEALEKWSQALMWKLLPRHMEIIGEIDKRFIAMIQKTQSDLESKLPSMRILDDNP |
| | | | QKPVVRMANLCVVSAHTVNGVAQLHSDILKSELFADYVSIWPTKFQNKTNGITPR |
| | | | RWLRFCSPELSNIITKWLKSEQWVTNLDLLAGLRQFADNVGFQDEWASAKMANK |
| | | | HRLAQYIERVTGVSIDPNSLFDIQVKRIHEYKRQLLNILGAIYRYKKLKEMSPEQRK |
| | | | NTTARTIMFGGKAFATYTNAKRIVKLVNDVGAVVNTDPEVNSYLKVVFVPNYNVSV |
| | | | AEMLIPGSELSQHISTAGMEASGTSNMKFALNGCLIIGTLDGANVEIREEIREENFF |
| | | | LFGATADEVPRLRKERENGLFKPDPRFEEAKQFIRSGAFGSYDYNPLLESLEGNSG |
| | | | YGRGDYFLVGHDFPSYMDAQAKVDEAYKDRKRWQKMSILSTAGSGKFSSDRTIA |
| | | | QYAKEIWKIGECRVP |
| 1091 | 89 | Bet_v | ASERERAMAASQFSATPIRPEALTQCNSLTRVFGFGSRSIRSKLLSIRTLSSRPSRR |
| | | | CFSVKNVSGETKQKLNPITEEGAPATHTSFTPDAASIASSIKYHAEFTPLFSPERFEL |
| | | | PKAFFATAQSVRDALLINWNATYDYYENLNQKQAYYLSMEFLQGRALLNAIGNLEL |
| | | | NGAYAEALRKLGHKLEDVASQEPDAALGNGGLGRLASCFLDSLATLNYPAWGYGL |
| | | | RYKYGLFKQRITKDGQEEVAEDWLEMGNPWEIVRNDVSYPVKFYGNVVSGSDGI |
| | | | RHWIGGEDIMAVAYDVPIPGYKTKTTINLRLWSTKALSKDFDLYTFNAGEHTKAYE |
| | | | ALANAEKICYILYPGDESMEGKALRLKQQYTLCSASLQDIIARFERRSGANVKWED |
| | | | IPKKVAVQMNDTHPTLCIPELMRILIDLKGLSWKEAWNITQRTVAYTNHTVLPEALE |
| | | | KWSLELMQKLLPRHVEIIEMIDEELIQTIVSEYGTADSELLEKKLKEMRILENVDLPA |
| | | | ELADLFVKPKESPIVVLKTKESPVVVLKTEESPVVVPSEELEKSEEAVEPVDEEDGS |
| | | | EEKGTQEKEMVLPEPVPEPPKMVRMANLCVVGGHAVNGVAEIHSEIVKDEVFNAF |
| | | | FKLWPEKFQNKTNGVTPRRWIRFCNPDLSKIITDWTGTEDWVLNTEKLAELRKFA |
| | | | DNEDLHTQWRAAKRSNKMKVVSFLKEKTGYSVSPDAMFDIQVKRIHEYKRQLMN |
| | | | ILGIVYRYKKMKEMSEEERRAKFVPRVCIFGGKAFSTYVQAKRIVKFITDVGATVNH |
| | | | DPEIGDLLKVVFVPDYNVSVAELLIPASELSQHISTAGMEASGTSNMKFAMNGCLL |
| | | | IGTLDGANVEIREEVGPDNFFLFGAKAHEIAGLRKERAEGKFVPDPCFEEVKEFVKS |
| | | | GAFGSNNYDELMGSLEGNEGFGCADYFLVGKDFPSYIECQENVDEAYQDQKRWT |
| | | | KMSILNTAGSYKFSSDRTIHEYAKDIWNIEPAQLP |
| 1092 | 89 | Cyn_d | SRPRPVYRIRRPPHVSPARLLEKPLPGSQTSSHSRSSIPRSWSVLVRRESPRLLDAI |
| | | | PQCREPAMPESKCGAAEKVAPAATPAAEKPADIAGNISYHATYSPHFAPLNFGPEQ |
| | | | AFYATAESVRDHLIQRWNETYLHFHKTDPKQTYYLSMEYLQGRALTNAVGNLGITG |
| | | | AYAEAVKKFGYELEALAAEEKDAALGNGGLGRLASCFLDSMATLNLPAWGYGLRY |
| | | | RYGLFKQRISKEGQEEIAEDWLDKFSPWEIPRHDVVFPVRFFGHVEILPNGTRKWV |
| | | | GGEVMKALAYDVPIPGYKTKNAISLRLWEAKATAEDFNLFQFNDGQYESSAQLHS |
| | | | RAQQICAVLYPGDATEEGKLLRLKQQFFLCSASLQDMIARFKERNPDRASGKWAE |
| | | | FPTKVAVQLNDTHPTLAIPELMRLLMDEEGLGWDEAWDITYRTVSYTNHTVLPEAL |
| | | | EKWSQIVMRKLLPRHMEIIEEIDKRFREMVISSHKEMEGKIDSMKVLDSSNPQKPV |
| | | | VRMANLCVVSSHTVNGVAELHSNILKQELFADYVSIWPSKFQNKTNGITPRRWLR |
| | | | FCNPELSELVTKWLKTDDWTSNLDLLTGLRKFADDEKLHAEWASAKLASKKRLAK |
| | | | YVLDVTGVEIDPTSLFDIQIKRIHEYKRQLLNILGVVYRYKKLKEMSAEERQKVTPR |
| | | | TVMLGGKAFATYTNAKRIVKLVNDVGAVVNNDPDVNKYLKVVFIPNYNVSVAEVLI |
| | | | PGSELSQHISTAGMEASGTSNMKFSLNGCVIIGTLDGANVEIREEVGEENFFLFGA |
| | | | KADQIAGLRKDRENGLFKPDPRFEEAKQLIRSGAFGSYDYEPLLDSLEGNSGFGRG |
| | | | DYFLVGYDFPSYIDAQNLVDKAYKDKKKWITMSILNTAGSGKFSSDRTIAQYAKEI |
| | | | WDIKASPVA |
| 1093 | 89 | Fra_e | GMFKPDPRFEEAKKFVRSGAFGTYDYNPLLDSLEGDSGYGRGDYFLVGHDFPSYM |
| | | | EAQARVDEAYKDRKRWIKMSILSTAGSGKFSSDRTISQYA |
| 1094 | 89 | Fra_e | RQIEKMATFSFYAATAVLSHRRSNSRLIDFSCRNGSCELFLTRRRVKSSFYVKSVS |
| | | | SEPKQEVIDPITEEGVHSYQSSFKPDAASIASSIKYHAEFTPLFSPEHFELPKAFYAT |
| | | | AQSVRDALIINWNATYDLYEKMNVKQAYYLSMEFLQGRALLNSIGNLELSGEYAEA |
| | | | LKKLGHSLESVASQEPDAALGNGGLGRLASCFLDSLATLNYPAWGYGLRYKYGLF |
| | | | KQRITKDGQEEVAENWLEMGNPWEIVRNDVSYPVKFYGKVLTGSDGKRRWIGGE |
| | | | DIVAVACDVPIPGYKTKTTINLRLWSTKVPSEQFDLYVFNAGEHTKACEAQANAEK |
| | | | ICYVLYPGDESTEGKILRLKQQYTLCSASLQDIIARFERRSGGNEIWEEFPEKVAVQ |
| | | | MNDTHPTLCIPELMRILMDLKGMSWEKAWSITQRTVAYTNHTVLPEALEKWSYEL |
| | | | MQKLLPRHVEIIEMIDEQLIQDIISEYGTSNPEMLEKKVNAMRILENVDLPPSLADLF |
| | | | AKPEEIII |
| 1095 | 89 | Fra_e | AKPEEIIIHETSDEVVLAHEDELEEKDPQEEKVVKPKQAPIPPKMVRMANLCVVGG |
| | | | HAVNGVAEIHSEIVRNEVFNDFFQLWPEKFQNKTNGVTPRRWIHFCNPDLSTIISK |
| | | | WIGTEDWVLNTEKLAELQKFADNEDLQIEWRAAKRSNKIKVASFLKDKTGYSVNP |
| | | | DAMFDIQVKRIHEYKRQLLNLLGIVYRYKKMKEMTAAERKEKFVPRVCIFGGKAFS |
| | | | TYIQAKRIVKFITDVGATINHDPDIADLLKVVFVPDYNVSVAELLIPASELSQHISTA |
| | | | GMEASGTSNMKFAMNGCLLIGTLDGANVEIRQEVGEDNFFLFGAQAHEIAALRKE |
| | | | RAEGKFVPDERFEEVKEFVKNGAFGPYNYDELMGSLEGNEGFGRADYFLVGKDFP |
| | | | SYIECQEKVDDAYRDQKRWTKMSILNTAGSSKFSSDRTIHEYAKDIWCIKPVELP |
| 1096 | 89 | Fra_e | AHLKTAPYYTMSATTVSLLTVGSSFSNPSVFSPCNFNRLLSTSLRPTKLHRSTHIFK |
| | | | LSNGFSSPLQASTTDNNDSITNVTTSGSSSTITFQNVDALDSTLFIIQARNKIGLLQ |
| | | | VITRVFKVLGLVVERATVEFEGDFFIKKFYIKNSEGKKIENVENLETIKKALMEAIEP |
| | | | GDASTGAEVRLGGRGWMRKAGLGFESLGDHRAKAEKMFRLMDGFLKNDPVSLQ |
| | | | KDIVYHVEYTVARSMFRFDDFEAYQALSHSVRDRLIERWHDTHHYFKKKDPKRLY |
| | | | FLSLEFLMGRSLSNSVINLGIRDQYVDALGQLGFEFEVLAEQEGDAALGNGGLARL |
| | | | SACQMDSLATLDFPAWGYGLRYQYGLFRQIIVDGFQHEQPDYWLNFGNPWEIER |
| | | | VQVSYAVKFYGTVEEEVSNGVNYKVWIPGETVEAVAYDNPIPGYGTRNAINLRLW |
| | | | AAKPSGQYDLESYNTGDYINAVVNRQKAEIISNVLYPDDRSYQGKELRLKQQYFFV |
| | | | SASVQDIIRRFKDAHENFEEFTEKVALQINDTHPSLAIVEVMRVLFDEEHLGWDKA |
| | | | WDIVCKIFSFTTHTVQPEGLEKIPVDLMGSLLPRHLQIIYDINYKFMEELKKKFGQD |
| | | | YSRHARMSIVEEGAVKSIRMANLSIVCCHMVNGVSKAHFELLKMRVFKDFYDLWP |
| | | | QKFQYKTNGVTQRRWIVVSNPSLCSVISKWLGTEAWVRNIDLLAGLQDYASDAEL |
| | | | QQEWGTVKKINKMRLAEYIETLSGVKVSLDAMFDVQIKRIHEYKRQLLNILGIIHRY |
| | | | DCIKNMNESDRRKVVPRVCIIGGKAAPGYEIAKKIIKLCHAVAEKINNDPVVGDLL |
| | | | KLIFIPDYNVSVAELVIPGSDLSQHISTAGHEASGTGSMKFLMNGCLLLATADGST |
| | | | VEIIEEIGADNMFLFGAKVNEVPALREQGASVRAPLQFVRVVRMVRDGYFGFKDYF |
| | | | ESLCDTLENGKDFYLLGADFASYLEAQAAADLTFVNQEKWTRMSILSTSGSGRFS |
| | | | SDRTIEEYAEQTWGIEPCKCPF |
| 1097 | 89 | Lol_p | RCANSEALSPPPPHALAQRIPHHTAMSAADKVKPAASPAAEDPAAIAGNISFHAQY |
| | | | SPHFSPLTFGPEPAYFATAESVRDHLLQRWNDTYLHFHKTDPKQTYYLSMEYLQGR |
| | | | ALTNAVGNLNITGAYAEAVKKFGYELEALAGQERDMALGNGGLGRLAACFLDSMA |
| | | | TLNLPAWGYGLRYRYGLFKQRITKEGQEEVAEDWLEKFSPWEIVRHDVVYPVRFF |
| | | | GHVEISPDGRRKAVGGEVLNALAYDVPIPGYKTKNAISLRLWDAKASAEDFNLFQF |
| | | | NDGQYESAAQLHSRAQQICAVLYPGDATEEGKLLRLKQQFFLCSASLQDIIFRFKE |
| | | | RKPDRASGKWSEFPSKVAVQMNDTHPTLAIPELMRILMDEEGLGWDEAWDVTNK |
| | | | TVAYTNHTVLPEALEKWSQAVMRKLLPRQMEIIEEIDKRFRELVISTRKDMEGKLD |
| | | | SMSVLDNSPQKPVVRMANLCVVAAHTVNGVAELHSNILKEELFADYLSIWPNKFQ |
| | | | NKTNGITPRRWLRFCNPELSEIVTKWLKTDQWTSNLDLLTGLRKFADDEKLHAEW |
| | | | AAAKLASKKRLAKHVLDVTGVTIDPNSLFDIQIKRIHEYKRQLMNILGAVYRYKKLK |
| | | | EMSAEEKQKVTPRTVMVGGKAFATYTNAKRIVKLVTDVGAVVNNDPDVNKYLKVV |
| | | | FIPNYNVSVAEVLIPGSELSQHISTAGMEASGTSNMKFSLNGCVIIGTLDGANVEIR |
| | | | EEVGQDNFFLFGAKADQVAGLRKDRENGLFKPDPRFEEAKQFVRSGAFGTYDYTP |
| | | | LLDSLEGNSGFGRGDYFLVGYDFPSYIDAQARVDEAYKDKKRWIKMSILNTAGSG |
| | | | KFSSDRTIAQYAKEIWGITASPVP |
| 1098 | 89 | Ole_e | FSPEHFELPKAFYATAQSVRDALIINWNATYDLYEKMNVKQAYYLSMEFLQGRALL |
| | | | NSIGNLELTGEYAEALKKLGHSLESVASQEPDAALGNGGLGRLASCFLDSLATLNY |
| | | | PAWGYGLRYKYGLFKQRITKEGQEEVAENWLEMGNPWEIVRNDVSYPVKFYGKVL |
| | | | TGLDGKRHWIGGEDIVAVACDVPIPGYKTKTTINLRLWSTKVPSEQFDLYAFNAGE |
| | | | HTKAREAQTNAEKICYILYPGDESTEGKILRLKQQYTLCTASLQDIIARFERRSGGN |
| | | | EIWEEFPEKVAVQMNDTHPTLCIPELMRILMDFKGMSWEKAWSITQRTVAYTNHT |
| | | | VLPEALEKWSYELMQKLLPRHVEIIEMIDEQLIQDIISEYGISNPEMLEKKVNAMRIL |
| | | | ENVDLPASLADLFAKPEEILIHETSDEVIHETSNEVIQETSDEVIHEISDEVVPAQED |
| | | | ELEGKDLQEEKVVKPEHAPIPPKMVRMANLCVVGGHAVNGVAEIHSEIVKKEVFN |
| | | | DFFQLWPEKFQNKTNGVTPRRWIHFCNPDLSTIISKWIGTDDWVLHTEKLAELQK |
| | | | FADNEDLQIEWRAAKRSNKIKVATFLKEKTGYLVSPDAMFDIQVKRIHEYKRQLLN |
| | | | ILGIVYRYKKMKEMTAAERKEKFVPRVCIFGGKAFATYIQAKRIVKFITDVGATINH |
| | | | DPDIGDLLKVVFVPDYNVSAAELLIPASELSQHISTAGMEASGTSNMKFAMNGCVL |
| | | | IGTLDGANVEIRQEVGEDNFFLFGAQAHEIAALRKERAEGKFVPDERFEEVKEFVRI |
| | | | GAFGPYNYDELMGSLEGNEGFGRADYFLVGKDFPSYIECQEKVDDAYRDQKRWT |
| | | | KMSVLNTAGSFKFSSDRTIHEYAKDIWSIKPMELS |
| 1099 | 89 | Pla_l | IPFTNHSLRIMAPGTEKATSDSTAPAVAKVPAVAHPLAEQPAEIASNISYHAQYSPH |
| | | | FSPLKFEPEQAYYATAESVRDRLIKQWNETYNLFNKANPKQTYYLSMEYLQGRALS |
| | | | NAVGNLDVQDAYASALQQLGHQLEEIVEQEKDAALGNGGLGRLASCFLDSMATL |
| | | | NLPAWGYGLRYRYGLFKQRIAKEGQEEIAEDWLEKFSPWEVVRHDVVFPVRFFGQ |
| | | | VAVLPSGARKLVGGETLQALAYDVPIPGYKTKNTNSLRLWEAKAGATDFDLFQFN |
| | | | DGQYESAAKLHSSAQQICAVLYPGDATESGKLLRLKQQFFLCSASLQDIIARFKER |
| | | | HATKEIKWSDFPSKVAVQLNDTHPTLAIPELMRLLMDEESLGWDEAWDITTRTIAY |
| | | | TNHTVLPEALEKWSQAVMWKLLPRHMEIITEIDKRFIQMIKSTRPDLEGKSSELCIL |
| | | | DNDPKKPVVRMANLCVVSAHTVNGVAQLHSDILKAELFVDYVSIWPTKFQNKTNG |
| | | | ITPRRWLKFCNPELSQIITKWLKTDQWVKNLDLLTNLRQFADNADLQSEWESAKL |
| | | | ASKKRLASYILRVTGETIDPNTLFDIQVKRIHEYKRQLLNILGAVYRYKKLKGMSPE |
| | | | DRKKTTPRTIMIGGKAFATYTNAKRIVKLVNDVGAVVNTDPEVNDLLKIVFVPNYN |
| | | | VSVAEVLIPGSELSQHISTAGMEASGTSNMKFALNGCLIIGTLDGANVEIREEIGED |
| | | | NFFLFGATADEVPRLRKEREEGKFKPDPRFEEAKQFIRSGAFGSYDYNPLLESLEGD |
| | | | TGYGRGDYFLVGHDFPAYMDAQERVDQAYKDRKRWAKMSILSTAGSGKFSSDRT |
| | | | IAQYASEIWKIKEHPVSSA |
| 1100 | 89 | Poa_p | GVLPVPPFGAPRLITSPATHAHRERSTQFPTAMSAADKVKPAASPAAEDPAAIAANI |
| | | | SYHAQYSPHFSPLAFGPEPAYFATAQSVRDHLLQRWNDTYLHFHKTDPKQTYYLS |
| | | | MEYLQGRALTNAVGNLDITGAYAEAVKKFGYELEALAGQERDMALGNGGLGRLAA |
| | | | CFLDSMATLNLPAWGYGLRYRYGLFKQRIAKEGQEEIAEDWLEKFSPWEIVRHDV |
| | | | VYPVRFFGHVEISPDGTRKSAGGEVLKALAYDVPIPGYKTKNAISLRLWDAKASAE |
| | | | DFNLFQFNDGQYESAAQLHSRAQQICAVLYPGDATEEGKLLRLKQQFFLCSASLQ |
| | | | DIIFRFKERKSDRVSGKWSEFPSKVAVQMNDTHPTLAIPELMRLLMDEEGLGWDE |
| | | | AWDVTNKTVAYTNHTVLPEALEKWSQSVMRKLLPRQMEIIEEIDKRFREMVISTRK |
| | | | DMEGKLDSMSVLDNSPQKPVVRMANLCVVSAHTVNGVAELHSNILKEELFADYVS |
| | | | IWPNKFQNKTNGITPRRWLKFCNPELSEIVTKWLKTDQWTSNLDLLTGLRKFADD |
| | | | EKLHAEWAAAKLASKKRLAKHVLDATGVTIDPTSLFDIQIKRIHEYKRQLMNILGA |
| | | | VYRYKKLKEMSAEEKQKVTPRTVMVGGKAFATYTNAKRIVKLVNDVGAVVNNDPD |
| | | | VNKYLKVVFIPNYNVSVAEVLIPGSELSQHISTAGMEASGTSNMKFSLNGCVIIGTL |
| | | | DGANVEIREEVGEDNFFLFGAKADQVAGLRKDRENGLFKPDPRFEEAKQYVRSGT |
| | | | FGTYDYTPLLDSLEGNSGFGRGDYFLVGYDFPSYIDAQARVDEAYKDKKRWTKMS |
| | | | ILNTAGSGKFSSDRTIAQYAKEIWGITASPVP |
| 1101 | 89 | Que_a | VRASEKERGENRYSKFAMAVSQFSAATSTGRSEALLTRSGLLGGGLGSRGSKSKV |
| | | | LLMRTWISRPVTVRRSFSVNSVSSDSNQTLKDPITQEEASTAHSSFTLDAASIASS |
| | | | IKYHAEFTPLFSPERFELPKAFFATAQSVRDALIINWNATYDYYEKLNVKQAYYLSM |
| | | | EFLQGRALLNAIGNLELTGAYAEALRNLGHKLEHVAIQEPDAALGNGGLGRLASCF |
| | | | LDSLATLNYPAWGYGLRYKYGLFKQRITKDGQEEVAEDWLEMGNPWEIVRNDVS |
| | | | YPVKFYGKVASGSDGKKHWIGGEDIKAVACDVPIPGYKTKTTINLRLWSTKALSE |
| | | | DFDLYAFNAGEHTKAYEALANAEKICYILYPGDESMEGKVLRLKQQYTLCSASLQD |
| | | | IIARFERRSGANVRWEEFPEKVAVQMNDTHPTLCIPELMRILIDLKGLSWKEAWNI |
| | | | TQRTVAYTNHTVLPEALEKWSLELMQKLLPRHVEIIEMIDEELIHTIVSEYGTEDYEL |
| | | | LEKKLKEMRILENVDLPSAFADLFVKLKPKESPVVVPSE |
| 1102 | 89 | Que_a | ALTNAIGNLNIQDAYGDALKKLGHELEEITEQEKDAALGNGGLGRLASCFLDSMAT |
| | | | LSLPAWGYGLRYKYGLFKQRITKEGQEEIAEDWLEKFSPWEVVRHDIIYPVRFFGS |
| | | | VEVNPNGSRNWVGGEVVQALAYDVPIPGYKTKNTISLRLWEAKACAEDFDLFQFN |
| | | | DSQYESAAELHSRAQQICAVLYPGDTKENGKLLRLKQQFFLCSASLQDIIFRFKER |
| | | | KLGKGSRQWSEFPSKVAVQMNDTHPTLAIPELMRLLMDEEGLGWDEAWDITTRT |
| | | | VAYTNHTVLPEALEKWSQAVMWKLLPRHMEIIGEIDKRFIAMIHKARPDLESKLPS |
| | | | MCILDNDPQKPVVRMANLCVVSAHTVNGVAQLHSDILKSELFADYVSLWPTKFQN |
| | | | KTNGITPRRWLRFCSPELSSIITKWLKTEEWIINLDLLTGLRQFADNADLQAEWAS |
| | | | AKMANKQRLAEYIERVTGVSIDPNSLFDIQVKRIHEYKRQLLNILGAIYRYKNLKEM |
| | | | SPEERKKTTSRTIMIGGKAFATYTNAKRIVKLVNDVGAVVNNDPEVNSYLKVVFVP |
| | | | NYNVSVAEILIPGSELSQHISTAGMEASGTSNMKFALNGCLIIGTLDGANVEIREEI |
| | | | GEENFFLFGATADEVPRLRKERENGKFKPDPRFEEAKEFIRSGAFGSYDFNPLLDSL |
| | | | EGNSGYGRGDYFLVGQDFPSYMDAQARVDEAYKDRKRWLKMSILSTAGSGKFSS |
| | | | DRTIAQYAKEIWNIEECRVP |
| 1103 | 89 | Que_a | CIAGDLGTFIPDSASIASSIKYHAEFTPSFSTEQFELPKAYFATAESVRDTLIINWNA |
| | | | TYDYYEMMNVKQAYYLSMEYLQGR |
| 1104 | 91 | Amb_a | MSNPRVYFDITIGGAPAGRIVMELFADQTPKTAENFRALCTGEKGTGRSGKPLHYQ |
| | | | GSSFHRVIPQFMLQGGDFTRGNGTGGESIYGEKFEDENFNLRHTGPGILSMANAG |
| | | | PGTNGSQFFICTVKTSWLDGKHVVFGQVVEGLDVVQAIEKVGSGSGSTSKQVTIA |
| | | | KSGQL |
| 1105 | 91 | Amb_a | AGRIVMELFADTTPRTAENFRALCTGEKGRGTSGKPLHYKGSSFHRVIPNFMCQG |
| | | | GDFTRGNGTGGESIYGNKFADENFIKKHTGPGILSMANAGPNTNGSQFFICTAKT |
| | | | EWLDGKHVVFGKV |
| 1106 | 91 | Amb_p | MANPKVFFDMTVGGAPAGRIVMELFADTTPRTAENFRALCTGEKGRGTSGKPLHY |
| | | | KGSSFHRVIPNFMCQGGDFTRGNGTGGESIYGNKFADENFIKKHTGPGILSMANA |
| | | | GPNTNGSQFFICTAKTEWLDGKHVVFGKVVEGMDVVKAIEKVGSGSGTCSKPVV |
| | | | VADCGQL |
| 1107 | 91 | Bet_v | MASNPKVFFDMEVGGQPVGRIVMELYADTTPRTAENFRALCTGEKGNGRSGKPLH |
| | | | YKKSSFHRVIPGFMCQGGDFTAGNGTGGESIYGAKFADENFIKKHTGPGILSMAN |
| | | | AGPGTNGSQFFICTAKTEWLDGKHVVFGQVVEGLDIVKAIEKVGSSSGRTSKPVV |
| | | | VADCGQL |
| 1108 | 91 | Cyn_d | MANPRVFFDMTVGGQPVGRIVMELYANEVPRTAENFRALCTGEKGTGKSGKPLHY |
| | | | KGSTFHRVIPDFMCQGGDFTRGNGTGGESIYGEKFPDEKFVRKHTGPGVLSMAN |
| | | | AGPNTNGSQFFICTVACPWLDGKHVVFGQVVEGMDVVKAIEKVGSRSGTTAKEV |
| | | | KIADCGQL |
| 1109 | 91 | Que_a | MASNPKVFFDMTIGGQPAGRIIMELYADVVPRTAENFRALCTGEKGAGRSGKPLH |
| | | | YKGSSFHRVIPGFMCQGGDFTAGNGTGGESIYGAKFADENFTKKHTGPGILSMAN |
| | | | AGPGTNGSQFFICTAKTEWLDGKHVVFGQIIEGMDVVKAVEKVGSSSGRTSKPVV |
| | | | VADCGQL |

TABLE 5
NTGA's with at least 1, 5 or 8 conserved peptides across GW, GT or Phl p and with co-release from
pollen together with major allergens

| Grass and | | | | | | | | Phl p | | | NTGA's or homolog | | |
|---|---|---|---|---|---|---|---|---|---|---|---|---|---|
| another | | GW | | | GT | | | Phl p, Amb p and | | | with “fast release” | | |
| pollen | | Phl p and Amb p | | | Phl p and Que a | | | Que a | | | from pollen | | |
| ≧1 | Th+ | ≧1 | ≧5 | ≧8 | ≧1 | ≧5 | ≧8 | ≧1 | ≧5 | ≧8 | GW | GT | Phl p |
| Col 1 | Col 2 | Col 3 | Col 4 | Col 5 | Col 6 | Col 7 | Col 8 | Col 9 | Col 10 | Col 11 | Col 12 | Col 13 | Col 14 |
| 1 | 1 | 1 | 1 | 1 | 1 | 1 | 1 | 1 | 1 | 1 | 1 | | |
| 2 | 2 | 2 | 2 | 2 | 2 | 2 | 2 | 2 | 2 | 2 | | | |
| 4 | 4 | 4 | 4 | 4 | 4 | 4 | 4 | 4 | 4 | 4 | 4 | | |
| 6 | 6 | 6 | 6 | 6 | 6 | 6 | 6 | 6 | 6 | 6 | 6 | | |
| 7 | 7 | 7 | 7 | 7 | 7 | 7 | 7 | 7 | 7 | 7 | 7 | | |
| 9 | 9 | 9 | | | 9 | | | 9 | | | | | |
| 10 | 10 | | | | 10 | | | | | | | | |
| 11 | 11 | 11 | | | 11 | | | 11 | | | | | |
| 16 | 16 | 16 | | | 16 | | | 16 | | | | | |
| 20 | 20 | 20 | 20 | | 20 | 20 | | 20 | 20 | | | | |
| 22 | 22 | 22 | 22 | | 22 | 22 | | 22 | 22 | | | | |
| 24 | 24 | 24 | 24 | 24 | 24 | 24 | 24 | 24 | 24 | 24 | 24 | 24 | 24 |
| 26 | 26 | 26 | 26 | 26 | 26 | 26 | | 26 | 26 | | 26 | | |
| 27 | 27 | 27 | | | 27 | 27 | | 27 | | | | | |
| 29 | 29 | 29 | 29 | | 29 | | | 29 | | | 29 | 29 | 29 |
| 30 | 30 | 30 | 30 | 30 | 30 | 30 | 30 | 30 | 30 | 30 | 30 | | |
| 32 | 32 | 32 | 32 | | 32 | 32 | | 32 | 32 | | | | |
| 34 | 34 | 34 | 34 | 34 | 34 | 34 | 34 | 34 | 34 | 34 | | | |
| 35 | 35 | 35 | | | 35 | | | 35 | | | | | |
| 39 | 39 | 39 | 39 | 39 | 39 | 39 | | 39 | 39 | | 39 | | |
| 40 | 40 | 40 | | | 40 | | | 40 | | | | | |
| 42 | 42 | 42 | 42 | | 42 | 42 | | 42 | 42 | | | | |
| 47 | 47 | 47 | | | 47 | | | 47 | | | 47 | | |
| 49 | 49 | 49 | | | 49 | | | 49 | | | | | |
| 51 | 51 | 51 | | | 51 | | | 51 | | | 39 | | |
| 53 | 53 | 53 | | | 53 | | | 53 | | | | | |
| 54 | 54 | 54 | | | 54 | | | 54 | | | | | |
| 56 | 56 | 56 | | | 56 | | | 56 | | | | 56 | |
| 59 | 59 | 59 | 59 | | 59 | 59 | | 59 | | | 59 | | |
| 62 | 62 | 62 | | | 62 | | | 62 | | | | | |
| 64 | 64 | 64 | | | 64 | | | 64 | | | 64 | | |
| 65 | 65 | | | | 65 | | | | | | | | |
| 72 | 72 | 72 | 72 | 72 | 72 | 72 | 72 | 72 | 72 | 72 | | | |
| 76 | 76 | | | | 76 | 76 | 76 | | | | | | |
| 77 | 77 | 77 | 77 | 77 | 77 | 77 | 77 | 77 | 77 | | | | |
| 79 | 79 | | | | | | | | | | | | |
| 83 | 83 | 83 | 83 | 83 | 83 | 83 | 83 | 83 | 83 | 83 | | | |
| 84 | 84 | 84 | 84 | | 84 | 84 | | 84 | 84 | | | | |
| 86 | 86 | 86 | 86 | 86 | 86 | 86 | 86 | 86 | 86 | 86 | 86 | | |
| 89 | 89 | 89 | | | 89 | 89 | 89 | 89 | | | | | |
| 90 | 90 | 90 | | | 90 | | | 90 | | | | | |
| 91 | 91 | 91 | | | 91 | | | 91 | | | 91 | 91 | 91 |
| 5/64 | 5/64 | 5/64 | 5/64 | 5/64 | 5/64 | 5/64 | 5/64 | | | | 5/64 | | |
| 39/59 | 39/59 | 39/59 | 39/59 | 39/59 | 39/59 | 39/59 | 39/59 | 39/59 | 39/59 | 39/59 | 39/59 | | |
| 49/54 | 49/54 | 49/54 | 49/54 | 49/54 | 49/54 | 49/54 | 49/54 | 49/54 | 49/54 | 49/54 | | | |
| 86/51 | 86/51 | 86/51 | 86/51 | 86/51 | 86/51 | 86/51 | 86/51 | 86/51 | 86/51 | 86/51 | 86/51 | | |

Table 5 lists NTGAs according to the number of PG+ peptides contained in their sequence. Column 1 shows NTGA's containing at least one TG+peptide; column 2 shows NTGA's containing at least one T cell epitope (Th+); columns 3, 4 and 5 show NTGA's containing at least one, five or eight peptide(s) conserved across Phl p and Amp p, respectively; columns 6, 7 and 8 show NTGA's containing at least one, five or eight peptide(s) conserved across Phl p and Que a, respectively and columns 9, 10 and 11 show NTGA's containing at least one, five or eight peptide(s) conserved across Phl p, Amp p and Que a, respectively.

Table 5 also shows which NTGA's or a homolog thereof that are released within a period overlapping with the release of major allergens from grass pollen and weed pollen (GW); from grass pollen and tree pollen (GT) or from both grass pollen, weed pollen and tree pollen (Phl p).

TABLE 6
No of PG+ or PP peptides per NTGA

| | | | PG+ & | | | | | | | | | | PG+ & | |
|---|---|---|---|---|---|---|---|---|---|---|---|---|---|---|
| NTGA | PG+ | PP | th+ | PP & Th+ | NTGA | PG+ | PP | PG+ & th+ | PP & Th+ | NTGA | PG+ | PP | th+ | PP & Th+ |
| 6 | 23 | 15 | 22 | 14 | 39 | 7 | 4 | 2 | 1 | 18 | 3 | 0 | 0 | 0 |
| 89 | 22 | 0 | 13 | 0 | 11 | 7 | 3 | 5 | 1 | 40 | 3 | 3 | 2 | 2 |
| 30 | 18 | 12 | 1 | 1 | 5 | 6 | 3 | 0 | 0 | 51 | 3 | 2 | 2 | 1 |
| 1 | 17 | 12 | 5 | 4 | 22 | 6 | 5 | 4 | 3 | 16 | 2 | 2 | 2 | 2 |
| 72 | 16 | 10 | 2 | 1 | 9 | 5 | 2 | 3 | 1 | 31 | 2 | 0 | 0 | 0 |
| 2 | 15 | 12 | 15 | 12 | 53 | 5 | 1 | 3 | 1 | 35 | 2 | 1 | 2 | 1 |
| 13 | 15 | 10 | 0 | 0 | 21 | 5 | 5 | 0 | 0 | 64 | 2 | 1 | 1 | 1 |
| 83 | 14 | 6 | 11 | 4 | 27 | 5 | 4 | 5 | 4 | 69 | 2 | 0 | 0 | 0 |
| 86 | 14 | 11 | 1 | 1 | 19 | 5 | 4 | 0 | 0 | 48 | 1 | 0 | 0 | 0 |
| 77 | 14 | 6 | 4 | 2 | 32 | 5 | 5 | 4 | 4 | 50 | 1 | 0 | 0 | 0 |
| 4 | 12 | 9 | 4 | 3 | 36 | 5 | 5 | 0 | 0 | 62 | 1 | 0 | 1 | 0 |
| 24 | 11 | 8 | 11 | 8 | 60 | 5 | 1 | 0 | 0 | 33 | 1 | 1 | 0 | 0 |
| 34 | 10 | 7 | 3 | 1 | 42 | 5 | 5 | 1 | 1 | 79 | 1 | 0 | 1 | 0 |
| 7 | 9 | 6 | 9 | 6 | 73 | 4 | 2 | 0 | 0 | 43 | 1 | 0 | 0 | 0 |
| 29 | 8 | 2 | 1 | 1 | 38 | 4 | 0 | 0 | 0 | 55 | 1 | 0 | 0 | 0 |
| 76 | 8 | 0 | 5 | 0 | 56 | 4 | 1 | 1 | 1 | 3 | 1 | 0 | 0 | 0 |
| 20 | 8 | 5 | 2 | 1 | 65 | 4 | 0 | 2 | 0 | 28 | 1 | 0 | 0 | 0 |
| 59 | 8 | 3 | 5 | 1 | 47 | 3 | 1 | 1 | 0 | 44 | 1 | 1 | 0 | 0 |
| 84 | 8 | 5 | 4 | 3 | 90 | 3 | 3 | 3 | 3 | 57 | 1 | 1 | 0 | 0 |
| 49/54 | 8 | 7 | 4 | 4 | 58 | 3 | 3 | 0 | 0 | 87 | 1 | 1 | 0 | 0 |
| 26 | 8 | 7 | 1 | 1 | 10 | 3 | 0 | 3 | 0 | 91 | 1 | 1 | 1 | 1 |
| | | | | | | | | | | Sum | 397 | 224 | 175 | 99 |

PG+; TG (Phl p) peptides with a mismatch of less than 3 to a corresponding peptide in at least one other non-grass pollen species
PP: TG (Phl p) peptides with a mismatch of less than 3 to a corresponding peptide within the non-grass pollen species Phl p, Amb p, Ole e, Fra e and Que a.

Table 6 shows NTGA's ranked according to the number of PG+ peptides contained in the NTGA. The table also shows number of PP peptides per NTGA and the number of peptides (Th+) recognized by T cells of a grass allergic donor population (n=20).

TABLE 7
NTGA's ranked according to the number of peptides and PP peptides per NTGA

| | # of TG | # | | | | | | | | # of TG | | % | # | | # | | |
|---|---|---|---|---|---|---|---|---|---|---|---|---|---|---|---|---|---|
| NTGA | peptides | any hit | % hit | # PG | % PG | # PP | % PP | # Th+ | NTGA | peptides | # any hit | hit | PG | % PG | PP | % PP | # Th+ |
| 1 | 21 | 18 | 86 | 18 | 86 | 12 | 57 | 0 | 89 | 32 | 32 | 100 | 29 | 91 | 0 | 0 | 4 |
| 30 | 24 | 24 | 100 | 22 | 92 | 12 | 50 | 0 | 38 | 18 | 17 | 94 | 8 | 44 | 0 | 0 | 0 |
| 86 | 16 | 16 | 100 | 15 | 94 | 11 | 69 | 0 | 48 | 16 | 15 | 94 | 3 | 19 | 0 | 0 | 0 |
| 6 | 29 | 28 | 97 | 26 | 90 | 11 | 38 | 9 | 78 | 15 | 2 | 13 | 0 | 0 | 0 | 0 | 0 |
| 13 | 15 | 15 | 100 | 15 | 100 | 10 | 67 | 0 | 8 | 14 | 12 | 86 | 1 | 7 | 0 | 0 | 0 |
| 72 | 23 | 23 | 100 | 19 | 83 | 10 | 43 | 0 | 76 | 14 | 12 | 86 | 9 | 64 | 0 | 0 | 0 |
| 4 | 18 | 18 | 100 | 15 | 83 | 9 | 50 | 0 | 50 | 12 | 7 | 58 | 1 | 8 | 0 | 0 | 0 |
| 2 | 26 | 26 | 100 | 19 | 73 | 9 | 35 | 5 | 62 | 12 | 10 | 83 | 3 | 25 | 0 | 0 | 2 |
| 24 | 15 | 14 | 93 | 13 | 87 | 8 | 53 | 2 | 47 | 9 | 9 | 100 | 5 | 56 | 0 | 0 | 0 |
| 26 | 9 | 9 | 100 | 8 | 89 | 7 | 78 | 0 | 52 | 9 | 7 | 78 | 2 | 22 | 0 | 0 | 1 |
| 49/54 | 10 | 9 | 90 | 8 | 80 | 7 | 70 | 3 | 79 | 8 | 8 | 100 | 1 | 13 | 0 | 0 | 0 |
| 34 | 12 | 10 | 83 | 10 | 83 | 7 | 58 | 0 | 43 | 7 | 6 | 86 | 1 | 14 | 0 | 0 | 0 |
| 7 | 12 | 12 | 100 | 11 | 92 | 6 | 50 | 4 | 55 | 6 | 3 | 50 | 1 | 17 | 0 | 0 | 0 |
| 77 | 14 | 14 | 100 | 14 | 100 | 6 | 43 | 0 | 81 | 6 | 5 | 83 | 2 | 33 | 0 | 0 | 0 |
| 83 | 21 | 18 | 86 | 17 | 81 | 6 | 29 | 0 | 3 | 5 | 4 | 80 | 3 | 60 | 0 | 0 | 0 |
| 32 | 5 | 5 | 100 | 5 | 100 | 5 | 100 | 0 | 10 | 5 | 5 | 100 | 3 | 60 | 0 | 0 | 0 |
| 42 | 5 | 5 | 100 | 5 | 100 | 5 | 100 | 0 | 18 | 5 | 5 | 100 | 4 | 80 | 0 | 0 | 0 |
| 21 | 8 | 6 | 75 | 5 | 63 | 5 | 63 | 0 | 28 | 5 | 5 | 100 | 2 | 40 | 0 | 0 | 0 |
| 22 | 8 | 7 | 88 | 7 | 88 | 5 | 63 | 0 | 31 | 5 | 5 | 100 | 3 | 60 | 0 | 0 | 0 |
| 84 | 10 | 10 | 100 | 9 | 90 | 5 | 50 | 0 | 46 | 5 | 1 | 20 | 0 | 0 | 0 | 0 | 1 |
| 20 | 13 | 13 | 100 | 9 | 69 | 5 | 38 | 0 | 63 | 5 | 3 | 60 | 0 | 0 | 0 | 0 | 0 |
| 19 | 5 | 5 | 100 | 5 | 100 | 4 | 80 | 0 | 65 | 5 | 5 | 100 | 5 | 100 | 0 | 0 | 0 |
| 36 | 5 | 5 | 100 | 5 | 100 | 4 | 80 | 0 | 66 | 5 | 3 | 60 | 0 | 0 | 0 | 0 | 0 |
| 40 | 5 | 4 | 80 | 3 | 60 | 3 | 60 | 0 | 67 | 5 | 5 | 100 | 3 | 60 | 0 | 0 | 0 |
| 5 | 8 | 8 | 100 | 7 | 88 | 3 | 38 | 0 | 68 | 5 | 4 | 80 | 0 | 0 | 0 | 0 | 0 |
| 27 | 8 | 8 | 100 | 5 | 63 | 3 | 38 | 0 | 69 | 5 | 4 | 80 | 3 | 60 | 0 | 0 | 0 |
| 58 | 8 | 7 | 88 | 3 | 38 | 3 | 38 | 0 | 70 | 5 | 5 | 100 | 1 | 20 | 0 | 0 | 0 |
| 11 | 9 | 9 | 100 | 8 | 89 | 3 | 33 | 0 | 71 | 5 | 3 | 60 | 0 | 0 | 0 | 0 | 0 |
| 90 | 9 | 9 | 100 | 5 | 56 | 3 | 33 | 0 | 82 | 5 | 3 | 60 | 1 | 20 | 0 | 0 | 0 |
| 39 | 10 | 10 | 100 | 7 | 70 | 3 | 30 | 0 | 85 | 5 | 5 | 100 | 0 | 0 | 0 | 0 | 0 |
| 16 | 5 | 5 | 100 | 3 | 60 | 2 | 40 | 0 | 92 | 5 | 2 | 40 | 0 | 0 | 0 | 0 | 0 |
| 51 | 5 | 5 | 100 | 3 | 60 | 2 | 40 | 0 | 93 | 5 | 5 | 100 | 0 | 0 | 0 | 0 | 0 |
| 59 | 13 | 13 | 100 | 11 | 85 | 2 | 15 | 0 | 61 | 4 | 2 | 50 | 0 | 0 | 0 | 0 | 0 |
| 29 | 15 | 14 | 93 | 13 | 87 | 2 | 13 | 0 | 74 | 4 | 2 | 50 | 1 | 25 | 0 | 0 | 0 |
| 9 | 16 | 14 | 88 | 8 | 50 | 2 | 13 | 0 | 12 | 3 | 0 | 0 | 0 | 0 | 0 | 0 | 0 |
| 35 | 5 | 5 | 100 | 4 | 80 | 1 | 20 | 0 | 14 | 2 | 2 | 100 | 0 | 0 | 0 | 0 | 0 |
| 44 | 5 | 5 | 100 | 3 | 60 | 1 | 20 | 0 | 15 | 2 | 0 | 0 | 0 | 0 | 0 | 0 | 0 |
| 56 | 5 | 5 | 100 | 4 | 80 | 1 | 20 | 0 | 17 | 2 | 1 | 50 | 0 | 0 | 0 | 0 | 0 |
| 57 | 5 | 5 | 100 | 2 | 40 | 1 | 20 | 0 | 23 | 2 | 0 | 0 | 0 | 0 | 0 | 0 | 0 |
| 60 | 5 | 5 | 100 | 5 | 100 | 1 | 20 | 0 | 75 | 2 | 1 | 50 | 0 | 0 | 0 | 0 | 0 |
| 64 | 5 | 5 | 100 | 3 | 60 | 1 | 20 | 0 | 80 | 2 | 0 | 0 | 0 | 0 | 0 | 0 | 0 |
| 87 | 5 | 4 | 80 | 3 | 60 | 1 | 20 | 0 | 25 | 1 | 0 | 0 | 0 | 0 | 0 | 0 | 0 |
| 91 | 5 | 5 | 100 | 1 | 20 | 1 | 20 | 2 | 37 | 1 | 0 | 0 | 0 | 0 | 0 | 0 | 0 |
| 33 | 9 | 1 | 11 | 1 | 11 | 1 | 11 | 0 | 41 | 1 | 1 | 100 | 0 | 0 | 0 | 0 | 0 |
| 53 | 13 | 12 | 92 | 5 | 38 | 1 | 8 | 3 | 45 | 1 | 0 | 0 | 0 | 0 | 0 | 0 | 0 |
| 73 | 29 | 27 | 93 | 17 | 59 | 1 | 3 | 0 | 88 | 1 | 0 | 0 | 0 | 0 | 0 | 0 | 0 |

Table 7 shows NTGA's ranked according to the number of PP peptides (# PP) contained in the NTGA. A PP peptide refers in this analysis to a peptide with a mismatch of less than 3 to a corresponding peptide within the non-grass pollen species Amb p, Pla I, Ole e, Fra e and Que a. The table also shows the number of PG peptides per NTGA: PG refers to a peptide with less than 3 mismatches to a corresponding peptide in another grass pollen species. Table also shows the number of peptides (# Th+) recognized by T cells of a grass allergic donor population (n=20) per NTGA.

TABLE 8
Table 8 - List of Pollen species

| | | | | | | | Major |
|---|---|---|---|---|---|---|---|
| | | Common | Latin name of | | | | Taxonomic |
| Category | ID | Name | species | Genus | Family | Order | group |
| Weed | | Giant ragweed | Ambrosia | Ambrosia | Asteraceae | Asterales | Magnoliopsidae |
| | | | trifida | | | | |
| Weed | | Short | Ambrosia | Ambrosia | Asteraceae | Asterales | Magnoliopsidae |
| | | Ragweed | artemisiifolia | | | | |
| Weed | Ambp | Western | Ambrosia | Ambrosia | Asteraceae | Asterales | Magnoliopsidae |
| | | ragweed | psilostachya | | | | |
| Herb | | Mugwort | Artemisia | Artemisia | Asteraceae | Asterales | Magnoliopsidae |
| | | | vulgaris | | | | |
| Herb | | Sunflower | Helianthus | Helianthus | Asteraceae | Asterales | Magnoliopsidae |
| | | | annuus | | | | |
| Tree | | Common | Alnus | Alnus | Betulaceae | Fagales | Magnoliopsidae |
| | | Alder | glutinosa | | | | |
| Tree | Bet v | European | Betula | Betula | Betulaceae | Fagales | Magnoliopsidae |
| | | white birch | Verrucosa | | | | |
| Tree | | Common | Carpinus | Carpinus | Betulaceae | Fagales | Magnoliopsidae |
| | | Hornbeam | betulus | | | | |
| Tree | | European | Castanea | Castanea | Betulaceae | Fagales | Magnoliopsidae |
| | | Chestnuts | sativa | | | | |
| Tree | | Common | Corylus | Corylus | Betulaceae | Fagales | Magnoliopsidae |
| | | Hazel | avellana | | | | |
| Tree | | European Hop- | Ostrya | Ostrya | Betulaceae | Fagales | Magnoliopsidae |
| | | hornbeam | carpinifolia | | | | |
| Tree | | Hazel- | Ostryopsis | Ostryopsis | Betulaceae | Fagales | Magnoliopsidae |
| | | hornbeam | | | | | |
| Tree | | American | Fagus | Fagus | Fagaceae | Fagales | Magnoliopsidae |
| | | Beech | grandifolia | | | | |
| Tree | | European | Fagus | Fagus | Fagaceae | Fagales | Magnoliopsidae |
| | | beech | sylvatica | | | | |
| Tree | Que a | White Oak | Quercus alba | Quercus | Fagaceae | Fagales | Magnoliopsidae |
| Tree | Fra e | European Ash | Fraxinus | Fraxinus | Oleaceae | Lamiales | Magnoliopsidae |
| | | | Excelsior | | | (Oleales) | |
| Tree | | Common | Ligustrum | Ligustrum | Oleaceae | Lamiales | Magnoliopsidae |
| | | Privet | vulgare | | | (Oleales) | |
| Tree | | Lilac | Syringa | Lilac | Oleaceae | Lamiales | Magnoliopsidae |
| | | | vulgaris | | | (Oleales) | |
| Tree | Ole e | European | Olea Europaea | Olea | Oleaceae | Lamiales | Magnoliopsidae |
| | | Olive | | | | (Oleales) | |
| Herb | Pla l | English | Plantago | Plantago | Plantaginaceae | Lamiales | Magnoliopsidae |
| | | plantain | lanceolata | | | (Oleales) | |
| Grass | Ant o | Sweet vernal | Anthoxanthum | Anthoxanthum | Poaceae | Poales | Liliopsida |
| | | grass | odoratum | | | | |
| Grass | Cyn d | Bermuda | Cynodon | Cynodon | Poaceae | Poales | Liliopsida |
| | | grass | dactylon | | | | |
| Grass | | Orchard Grass | Dactylis | Dactylis | Poaceae | Poales | Liliopsida |
| | | | glomerata L. | | | | |
| Grass | | Meadow | Festuca | Festuca | Poaceae | Poales | Liliopsida |
| | | fescue | pratensis | | | | |
| Grass | | Velvet Grass | Holcus lanatus | Holcus | Poaceae | Poales | Liliopsida |
| Grass | | Barley | Hordeum | Hordeum | Poaceae | Poales | Liliopsida |
| | | | vulgare | | | | |
| Grass | Lol p | Rye grass | Lollium | Lollium | Poaceae | Poales | Liliopsida |
| | | | Perenne | | | | |
| Grass | | Rice | Oryza sativa | Oryza | Poaceae | Poales | Liliopsida |
| Grass | | Bahia grass | Paspalum | Paspalum | Poaceae | Poales | Liliopsida |
| | | | notatum | | | | |
| Grass | | Canary Grass | Phalaris | Phalaris | Poaceae | Poales | Liliopsida |
| | | | aquatica | | | | |
| Grass | Phl p | Timothy grass | Phleum | Phleum | Poaceae | Poales | Liliopsida |
| | | | Pratense | | | | |
| Grass | Poa p | Kentucky blue | Poa pratensis | Poa | Poaceae | Poales | Liliopsida |
| | | grass | | | | | |
| Grass | | Rye | Secale Cereale | Secale | Poaceae | Poales | Liliopsida |
| Grass | | Johnson grass | Sorghum | Sorghum | Poaceae | Poales | Liliopsida |
| | | | halepense | | | | |
| Grass | | Wheat | Triticum | Triticum | Poaceae | Poales | Liliopsida |
| | | | aestivum | | | | |
| Grass | | Maize | Zea mays | Zea | Poaceae | Poales | Liliopsida |

Table 8 lists pollen species of the plant families Asteraceae, Betulaceae, Fagaceae, Oleaceae, Plantaginacea and Poaceae Pollen species used for the present conservation analysis are highlighted in grey colour.

TABLE 9

| Locus HLA DP | Locus HLA DQ | Locus HLA DR | |
|---|---|---|---|
| DPA1*01- | DQA1*0101-DQB1*0501 | DRB1*0101 | DRB1*0802 |
| DPB1*0401 | | | |
| DPA1*0103- | DQA1*0102-DQB1*0602 | DRB1*0301 | DRB1*0901 |
| DPA1*0201- | DQA1*0301-DQB1*0302 | DRB1*0401 | DRB1*1101 |
| DPA1*0201- | DQA1*0401-DQB1*0402 | DRB1*0404 | DRB1*1302 |
| DPA1*0301- | DQA1*0501-DQB1*0201 | DRB1*0405 | DRB1*1501 |
| | DQA1*0501-DQB1*0301 | DRB1*0701 | DRB3*0101 |
| | | | DRB4*0101 |
| | | | DRB5*0101 |

Table 9 shows a panel of 25 MHC II molecules (alleles) for which peptide binding affinities were predicted.

TABLE 10

Grass

| | | Phl p | | Ant o | | Cyn d | | Poa p | | Lol p | |
|---|---|---|---|---|---|---|---|---|---|---|---|
| | | | % | | % | | % | | % | | % |
| Anti- | | # of | Reac- | # of | Reac- | # of | Reac- | # of | Reac- | # of | Reac- |
| gen | Sequence | mm | tivity | mm | tivity | mm | tivity | mm | tivity | mm | tivity |
| NTGA | AVMLTFDNAG | 0 | 4 | 0 | 9 | 0 | 18 | 0 | 25 | 0 | 36 |
| 49 | MWNVR | | | | | | | | | | |
| NTGA | IGSFFYFPSIG | 0 | 0 | 2 | 3 | 2 | 9 | 1 | 2 | 0 | 6 |
| 54 | MQRT | | | | | | | | | | |
| NTGA | NPMTVFWSK | 0 | 0 | 1 | 0 | 2 | 1 | 0 | 0 | 1 | 1 |
| 76 | MAQSMT | | | | | | | | | | |
| NTGA | NGSQFFLCTA | 0 | 4 | 2 | 11 | 3 | 38 | 2 | 27 | 0 | 44 |
| 91 | KTAWL | | | | | | | | | | |
| NTGA | NGSQFFLCTA | 0 | 100 | 2 | 0 | 3 | 1 | 2 | 1 | 0 | 1 |
| 91 | KTAWL | | | | | | | | | | |
| NTGA | QYAKEIWGIT | 0 | | 1 | 2 | >3 | 10 | 1 | 22 | 1 | 24 |
| 89 | ANPVP | | | | | | | | | | |
| NTGA | FPIVQRFLEGA | 0 | 38 | 2 | 89 | 1 | 22 | 1 | 95 | 1 | 37 |
| 2 | SSID | | | | | | | | | | |
| NTGA | FPIVQRFLEGA | 0 | 100 | 2 | 100 | 1 | 39 | 1 | 100 | 1 | 100 |
| 2 | SSID | | | | | | | | | | |
| Lol p | LIEKINAGFKA | 1 | 24 | 3 | 4 | 0 | 1 | 1 | 41 | 0 | 39 |
| 51 | AVAA | | | | | | | | | | |
| Lol p | LIEKINAGFKA | 1 | 49 | 3 | 75 | 0 | 9 | 1 | 79 | 0 | 77 |
| 51 | AVAA | | | | | | | | | | |
| Lol p | NAGFKAAVAA | 2 | 0 | 0 | 36 | 0 | 3 | 2 | 23 | 0 | 35 |
| 51 | AAVVP | | | | | | | | | | |
| Lol p | NAGFKAAVAA | 2 | 0 | 0 | 2 | 0 | 10 | 2 | 22 | 0 | 24 |
| 51 | AAVVP | | | | | | | | | | |
| Lol p | NAGFKAAVAA | 2 | 70 | 0 | 100 | 0 | 100 | 2 | 100 | 0 | 100 |
| 51 | AAVVP | | | | | | | | | | |
| Lol p | SDAKTLVLNI | 2 | 36 | 2 | 24 | 0 | 18 | 2 | 74 | 0 | 47 |
| 3 | KYTRP | | | | | | | | | | |
| Lol p | SDAKTLVLNI | 2 | 100 | 2 | 100 | 0 | 29 | 2 | 100 | 0 | 100 |
| 3 | KYTRP | | | | | | | | | | |
| Lol p | SDAKTLVLNI | 2 | 0 | 2 | 35 | 0 | 2 | 2 | 37 | 0 | 52 |
| 3 | KYTRP | | | | | | | | | | |
| Lol p | SDAKTLVLNI | 2 | 36 | 2 | 100 | 0 | 55 | 2 | 100 | 0 | 100 |
| 3 | KYTRP | | | | | | | | | | |
| Lol p | MRNVFDDVV | 0 | 6 | 1 | 34 | >3 | 66 | 0 | 75 | 0 | 88 |
| 2 | PADFKV | | | | | | | | | | |
| Lol p | MRNVFDDVV | 0 | 69 | 1 | 68 | >3 | 10 | 0 | 91 | 0 | 82 |
| 2 | PADFKV | | | | | | | | | | |
| Lol p | MRNVFDDVV | 0 | 69 | 1 | 73 | >3 | 2 | 0 | 36 | 0 | 73 |
| 2 | PADFKV | | | | | | | | | | |
| Lol p | MRNVFDDVV | 0 | 18 | 1 | 55 | >3 | 1 | 0 | 67 | 0 | 72 |
| 2 | PADFKV | | | | | | | | | | |
| Lol p | MRNVFDDVV | 0 | 100 | 1 | 100 | >3 | 23 | 0 | 100 | 0 | 100 |
| 2 | PADFKV | | | | | | | | | | |
| Lol p | NVFDEVIPTAF | 2 | 75 | 3 | 0 | 3 | 0 | 2 | 42 | 0 | 96 |
| 3 | TVGK | | | | | | | | | | |
| Lol p | NVFDEVIPTAF | 2 | 81 | 3 | 60 | 3 | 0 | 2 | 100 | 0 | 100 |
| 3 | TVGK | | | | | | | | | | |
| Lol p | DAYVATLTEA | 2 | 91 | 1 | 95 | 0 | 1 | 2 | 100 | 0 | 100 |
| 5 1 | LRVIA | | | | | | | | | | |
| Lol p | DAYVATLTEA | 2 | 100 | 1 | 90 | 0 | 14 | 2 | 100 | 0 | 100 |
| 5 1 | LRVIA | | | | | | | | | | |
| Lol p | DAYVATLTEA | 2 | 16 | 1 | 58 | 0 | 9 | 2 | 36 | 0 | 34 |
| 5 1 | LRVIA | | | | | | | | | | |
| Lol p | DAYVATLTEA | 2 | 56 | 1 | 68 | 0 | 11 | 2 | 75 | 0 | 69 |
| 5 1 | LRVIA | | | | | | | | | | |
| Lol p | AFKIAATAAN | 2 | 1 | 2 | 48 | 2 | 0 | 2 | 12 | 0 | 5 |
| 5 2 | AAPTN | | | | | | | | | | |
| Lol p | AFKIAATAAN | 2 | 31 | 2 | 61 | 2 | 17 | 2 | 100 | 0 | 100 |
| 5 2 | AAPTN | | | | | | | | | | |
| Lol p | AFKIAATAAN | 2 | 21 | 2 | 68 | 2 | 0 | 2 | 65 | 0 | 100 |
| 5 2 | AAPTN | | | | | | | | | | |
| Poa p | DINVGFKAAV | 1 | 0 | >3 | 1 | 3 | 0 | 1 | 8 | 3 | 3 |
| 1 | AAAAG | | | | | | | | | | |
| Poa p | DINVGFKAAV | 1 | 6 | 3 | 8 | 3 | 3 | 1 | 7 | 3 | 6 |
| 1 | AAAAG | | | | | | | | | | |
| Poa p | DINVGFKAAV | 1 | 15 | 3 | 35 | 3 | 48 | 1 | 95 | 3 | 67 |
| 1 | AAAAG | | | | | | | | | | |
| Poa p | DINVGFKAAV | 1 | 12 | 3 | 18 | 3 | 13 | 1 | 21 | 3 | 47 |
| 1 | AAAAG | | | | | | | | | | |
| Poa p | EPIAAYHFDLS | 1 | 4 | 1 | 44 | 1 | 100 | 0 | 100 | 1 | 48 |
| 1 | GKAF | | | | | | | | | | |
| Poa p | EPIAAYHFDLS | 1 | 100 | 1 | 100 | 1 | 100 | 0 | 100 | 1 | 100 |
| 1 | GKAF | | | | | | | | | | |
| Poa p | FKAAVAAAAG | 2 | 2 | 2 | 62 | 2 | 31 | 0 | 26 | 1 | 68 |
| 5 | APPAD | | | | | | | | | | |
| Dac g | GSDEKNLALS | 1 | 32 | 0 | 58 | >3 | 8 | 1 | 67 | 0 | 66 |
| 2 | IKYNK | | | | | | | | | | |
| Dac g | NLALSIKYNK | 1 | 57 | 0 | 0 | >3 | 76 | 1 | 100 | 0 | 76 |
| 2 | EGDSM | | | | | | | | | | |
| Dac g | DIYNYMEPYV | 3 | 30 | >3 | N/A | >3 | 83 | >3 | 23 | >3 | 100 |
| 4 | SKVDP | | | | | | | | | | |
| Lol p | KASNPNYLAIL | 2 | | 3 | 80 | 3 | 12 | 2 | 100 | 0 | 100 |
| 1 | VKYV | | | | | | | | | | |
| Phl p | NFRFMSKGG | 0 | 51 | 0 | 56 | 0 | 3 | 0 | 68 | 2 | 57 |
| 3 | MRNVFD | | | | | | | | | | |
| Phl p | INVGFKAAVA | 0 | 14 | >3 | 5 | 2 | 17 | 0 | 23 | 2 | 30 |
| 5 | AAASV | | | | | | | | | | |
| Phl p | INVGFKAAVA | 0 | 1 | >3 | 0 | 2 | 0 | 0 | 13 | 2 | 9 |
| 5 | AAASV | | | | | | | | | | |
| Phl p | INVGFKAAVA | 0 | 44 | >3 | 35 | 2 | 3 | 0 | 72 | 2 | 60 |
| 5 | AAASV | | | | | | | | | | |
| Phl p | EEWEPLTKKG | 0 | 100 | 1 | 100 | 1 | 7 | 0 | 100 | 2 | 100 |
| 3 | NVWEV | | | | | | | | | | |
| Phl p | NVWEVKSSK | 0 | 100 | 2 | 100 | 2 | 4 | 0 | 100 | 2 | 100 |
| 3 | PLVGPF | | | | | | | | | | |
| Phl p | NVWEVKSSK | 0 | 45 | 2 | 32 | 2 | 10 | 0 | 58 | 2 | N/A |
| 3 | PLVGPF | | | | | | | | | | |
| Phl p | NVWEVKSSK | 0 | 25 | 2 | N/A | 2 | 4 | 0 | 55 | 2 | N/A |
| 3 | PLVGPF | | | | | | | | | | |
| Phl p | NVWEVKSSK | 0 | 77 | 2 | 98 | 2 | N/A | 0 | 100 | 2 | N/A |
| 3 | PLVGPF | | | | | | | | | | |
| Phl p | NVWEVKSSK | 0 | 6 | 2 | 4 | 2 | 7 | 0 | 12 | 2 | 1 |
| 3 | PLVGPF | | | | | | | | | | |
| Phl p | NVWEVKSSK | 0 | 10 | 2 | 49 | 2 | 19 | 0 | 37 | 2 | 41 |
| 3 | PLVGPF | | | | | | | | | | |
| Phl p | AFKVAATAAN | 0 | 53 | 2 | 88 | 0 | 9 | 0 | N/A | 2 | N/A |
| 5 | AAPAN | | | | | | | | | | |
| Phl p | AFKVAATAAN | 0 | 17 | 2 | 39 | 0 | 3 | 0 | N/A | 2 | N/A |
| 5 | AAPAN | | | | | | | | | | |
| Phl p | AFKVAATAAN | 0 | 100 | 2 | 100 | 0 | 3 | 0 | 100 | 2 | 100 |
| 5 | AAPAN | | | | | | | | | | |
| Phl p | AFKVAATAAN | 0 | 1 | 2 | 8 | 0 | 6 | 0 | 19 | 2 | 20 |
| 5 | AAPAN | | | | | | | | | | |
| Phl p | AFKVAATAAN | 0 | 30 | 2 | 45 | 0 | 16 | 0 | 59 | 2 | 62 |
| 5 | AAPAN | | | | | | | | | | |
| Phl p | STWYGKPTG | 0 | 32 | 1 | 51 | 1 | 1 | 0 | 44 | 0 | 64 |
| 1 | AGPKDN | | | | | | | | | | |
| Phl p | STWYGKPTG | 0 | 35 | 1 | 64 | 1 | N/A | 0 | 100 | 0 | N/A |
| 1 | AGPKDN | | | | | | | | | | |
| Phl p | STWYGKPTG | 0 | 5 | 1 | | 1 | 95 | 0 | 19 | 0 | 40 |
| 1 | AGPKDN | | | | | | | | | | |
| Phl p | SGIAFGSMAK | 0 | 10 | >3 | 15 | >3 | 73 | 3 | 58 | 2 | |
| 1 | KGDEQ | | | | | | | | | | |
| Phl p | SGIAFGSMAK | 0 | 3 | >3 | 11 | >3 | 1 | 3 | 14 | 2 | 6 |
| 1 | KGDEQ | | | | | | | | | | |
| Phl p | SGIAFGSMAK | 0 | 41 | >3 | 100 | >3 | 100 | 3 | 91 | 2 | 64 |
| 1 | KGDEQ | | | | | | | | | | |
| Phl p | SGIAFGSMAK | 0 | 43 | >3 | 100 | >3 | 8 | 3 | 100 | 2 | 38 |
| 1 | KGDEQ | | | | | | | | | | |
| Phl p | GELELQFRRV | 0 | 4 | 1 | 22 | 1 | 0 | 1 | 77 | 0 | 21 |
| 1 | KCKYP | | | | | | | | | | |
| Phl p | GELELQFRRV | 0 | 5 | 1 | 0 | 1 | 3 | 1 | 8 | 0 | 0 |
| 1 | KCKYP | | | | | | | | | | |
| Phl p | GELELQFRRV | 0 | 0 | 1 | 13 | 1 | 13 | 1 | 0 | 0 | 25 |
| 1 | KCKYP | | | | | | | | | | |
| Phl p | GELELQFRRV | 0 | 98 | 1 | 100 | 1 | 14 | 1 | 100 | 0 | 100 |
| 1 | KCKYP | | | | | | | | | | |
| Phl p | GELELQFRRV | 0 | 88 | 1 | 91 | 1 | 14 | 1 | 92 | 0 | 85 |
| 1 | KCKYP | | | | | | | | | | |
| Phl p | LAKYKANWIE | 0 | 6 | >3 | 14 | >3 | 47 | 2 | 22 | 2 | 33 |
| 13 | IMRIK | | | | | | | | | | |
| | | Weed | | | | Tree | | | | | |
| | | Amb p | | Pla l | | Ole e | | Que a | | Bet v | |
| | | | % | | % | | % | | % | | % |
| Anti- | | # of | Reac- | # of | Reac- | # of | Reac- | # of | Reac- | # of | Reac- |
| gen | Sequence | mm | tivity | mm | tivity | mm | tivity | mm | tivity | mm | tivity |
| NTGA | AVMLTFDNAG | 0 | 2 | 0 | 9 | 0 | 3 | 0 | 0 | 3 | 0 |
| 49 | MWNVR | | | | | | | | | | |
| NTGA | IGSFFYFPSIG | 2 | 0 | 1 | 2 | 1 | 1 | 2 | 1 | >3 | 2 |
| 54 | MQRT | | | | | | | | | | |
| NTGA | NPMTVFWSK | >3 | 0 | 2 | 0 | 2 | 0 | 2 | 0 | 3 | 0 |
| 76 | MAQSMT | | | | | | | | | | |
| NTGA | NGSQFFLCTA | 2 | 9 | 3 | 1 | 2 | 4 | 2 | 2 | 2 | 1 |
| 91 | KTAWL | | | | | | | | | | |
| NTGA | NGSQFFLCTA | 2 | 90 | 3 | 2 | 2 | 5 | 2 | 90 | 2 | 1 |
| 91 | KTAWL | | | | | | | | | | |
| NTGA | QYAKEIWGIT | >3 | 83 | >3 | 15 | >3 | 44 | >3 | 32 | >3 | 73 |
| 89 | ANPVP | | | | | | | | | | |
| NTGA | FPIVQRFLEGA | >3 | 4 | >3 | 1 | >3 | 4 | >3 | 0 | >3 | 5 |
| 2 | SSID | | | | | | | | | | |
| NTGA | FPIVQRFLEGA | >3 | 71 | >3 | 39 | >3 | 100 | >3 | 0 | >3 | 0 |
| 2 | SSID | | | | | | | | | | |
| Lol p | LIEKINAGFKA | >3 | 0 | 0 | 2 | >3 | 2 | 0 | 0 | >3 | 0 |
| 51 | AVAA | | | | | | | | | | |
| Lol p | LIEKINAGFKA | >3 | 11 | 0 | 15 | >3 | 12 | 0 | 3 | >3 | 9 |
| 51 | AVAA | | | | | | | | | | |
| Lol p | NAGFKAAVAA | >3 | 0 | 0 | 0 | >3 | 5 | 0 | 0 | >3 | 0 |
| 51 | AAVVP | | | | | | | | | | |
| Lol p | NAGFKAAVAA | >3 | 83 | 0 | 15 | >3 | 44 | 0 | 32 | >3 | 73 |
| 51 | AAVVP | | | | | | | | | | |
| Lol p | NAGFKAAVAA | >3 | 100 | 0 | 88 | >3 | 100 | 0 | 0 | >3 | 0 |
| 51 | AAVVP | | | | | | | | | | |
| Lol p | SDAKTLVLNI | 2 | 11 | 3 | 14 | 2 | 23 | 2 | 0 | >3 | 6 |
| 3 | KYTRP | | | | | | | | | | |
| Lol p | SDAKTLVLNI | 2 | 33 | 3 | 24 | 2 | 53 | 2 | 11 | >3 | 23 |
| 3 | KYTRP | | | | | | | | | | |
| Lol p | SDAKTLVLNI | 2 | 14 | 3 | 8 | 2 | 16 | 2 | 0 | >3 | 0 |
| 3 | KYTRP | | | | | | | | | | |
| Lol p | SDAKTLVLNI | 2 | 100 | 3 | 86 | 2 | 80 | 2 | 12 | >3 | 71 |
| 3 | KYTRP | | | | | | | | | | |
| Lol p | MRNVFDDVV | >3 | 56 | >3 | 81 | >3 | 66 | >3 | 28 | >3 | N/A |
| 2 | PADFKV | | | | | | | | | | |
| Lol p | MRNVFDDVV | >3 | 11 | >3 | 6 | >3 | 20 | >3 | 6 | >3 | 2 |
| 2 | PADFKV | | | | | | | | | | |
| Lol p | MRNVFDDVV | >3 | 2 | >3 | 1 | >3 | 9 | >3 | 2 | >3 | 3 |
| 2 | PADFKV | | | | | | | | | | |
| Lol p | MRNVFDDVV | >3 | 1 | >3 | 2 | >3 | 1 | >3 | 1 | >3 | 1 |
| 2 | PADFKV | | | | | | | | | | |
| Lol p | MRNVFDDVV | >3 | 55 | >3 | 25 | >3 | 25 | >3 | 0 | >3 | 0 |
| 2 | PADFKV | | | | | | | | | | |
| Lol p | NVFDEVIPTAF | 3 | 2 | 3 | 0 | 3 | 8 | 3 | 0 | >3 | 2 |
| 3 | TVGK | | | | | | | | | | |
| Lol p | NVFDEVIPTAF | 3 | 49 | 3 | 11 | 3 | 37 | 3 | 0 | >3 | 0 |
| 3 | TVGK | | | | | | | | | | |
| Lol p | DAYVATLTEA | >3 | 10 | 0 | 4 | 0 | 29 | 0 | 10 | >3 | 4 |
| 5 1 | LRVIA | | | | | | | | | | |
| Lol p | DAYVATLTEA | >3 | 23 | 0 | 18 | 0 | 62 | 0 | 8 | >3 | 26 |
| 5 1 | LRVIA | | | | | | | | | | |
| Lol p | DAYVATLTEA | >3 | 6 | 0 | 4 | 0 | 6 | 0 | 0 | >3 | 0 |
| 5 1 | LRVIA | | | | | | | | | | |
| Lol p | DAYVATLTEA | >3 | 8 | 0 | 8 | 0 | 5 | 0 | 0 | >3 | 0 |
| 5 1 | LRVIA | | | | | | | | | | |
| Lol p | AFKIAATAAN | >3 | 2 | >3 | 2 | 2 | 1 | 2 | 0 | >3 | 1 |
| 5 2 | AAPTN | | | | | | | | | | |
| Lol p | AFKIAATAAN | >3 | 24 | >3 | 9 | 2 | 35 | 2 | 4 | >3 | 0 |
| 5 2 | AAPTN | | | | | | | | | | |
| Lol p | AFKIAATAAN | >3 | 11 | >3 | 4 | 2 | 15 | 2 | 0 | >3 | 3 |
| 5 2 | AAPTN | | | | | | | | | | |
| Poa p | DINVGFKAAV | >3 | 1 | 3 | 0 | >3 | 2 | 3 | 2 | >3 | 0 |
| 1 | AAAAG | | | | | | | | | | |
| Poa p | DINVGFKAAV | >3 | 2 | 3 | 0 | >3 | 2 | 3 | 0 | >3 | 0 |
| 1 | AAAAG | | | | | | | | | | |
| Poa p | DINVGFKAAV | >3 | 2 | 3 | 3 | >3 | 11 | 3 | 0 | >3 | 2 |
| 1 | AAAAG | | | | | | | | | | |
| Poa p | DINVGFKAAV | >3 | 19 | 3 | 10 | >3 | 16 | 3 | 0 | >3 | 0 |
| 1 | AAAAG | | | | | | | | | | |
| Poa p | EPIAAYHFDLS | 1 | 1 | 1 | 1 | 1 | 2 | 1 | 0 | >3 | 0 |
| 1 | GKAF | | | | | | | | | | |
| Poa p | EPIAAYHFDLS | 1 | 19 | 1 | 17 | 1 | 52 | 1 | 2 | >3 | 0 |
| 1 | GKAF | | | | | | | | | | |
| Poa p | FKAAVAAAAG | >3 | 15 | 2 | 2 | >3 | 39 | 2 | 6 | >3 | 22 |
| 5 | APPAD | | | | | | | | | | |
| Dac g | GSDEKNLALS | >3 | 5 | >3 | 0 | >3 | 10 | >3 | 0 | >3 | 0 |
| 2 | IKYNK | | | | | | | | | | |
| Dac g | NLALSIKYNK | >3 | 76 | >3 | 41 | >3 | 78 | >3 | 0 | >3 | 0 |
| 2 | EGDSM | | | | | | | | | | |
| Dac g | DIYNYMEPYV | >3 | 37 | >3 | 17 | >3 | 82 | >3 | 0 | >3 | 0 |
| 4 | SKVDP | | | | | | | | | | |
| Lol p | KASNPNYLAIL | 3 | 12 | 0 | 6 | 0 | 41 | 0 | 2 | >3 | 4 |
| 1 | VKYV | | | | | | | | | | |
| Phl p | NFRFMSKGG | 0 | 6 | 0 | 0 | 0 | 6 | 0 | 0 | >3 | 0 |
| 3 | MRNVFD | | | | | | | | | | |
| Phl p | INVGFKAAVA | >3 | 39 | 2 | 0 | >3 | 19 | 2 | 0 | >3 | 1 |
| 5 | AAASV | | | | | | | | | | |
| Phl p | INVGFKAAVA | >3 | 0 | 2 | 0 | >3 | 0 | 2 | 0 | >3 | 0 |
| 5 | AAASV | | | | | | | | | | |
| Phl p | INVGFKAAVA | >3 | 14 | 2 | 9 | >3 | 13 | 2 | 2 | >3 | 1 |
| 5 | AAASV | | | | | | | | | | |
| Phl p | EEWEPLTKKG | 1 | 49 | 1 | 23 | 1 | 13 | 1 | 1 | >3 | 3 |
| 3 | NVWEV | | | | | | | | | | |
| Phl p | NVWEVKSSK | 2 | 0 | 2 | 1 | 2 | 4 | 2 | 0 | >3 | 1 |
| 3 | PLVGPF | | | | | | | | | | |
| Phl p | NVWEVKSSK | 2 | 10 | 2 | 0 | 2 | 3 | 2 | N/A | >3 | N/A |
| 3 | PLVGPF | | | | | | | | | | |
| Phl p | NVWEVKSSK | 2 | N/A | 2 | 8 | 2 | 6 | 2 | 3 | >3 | 2 |
| 3 | PLVGPF | | | | | | | | | | |
| Phl p | NVWEVKSSK | 2 | N/A | 2 | N/A | 2 | N/A | 2 | 15 | >3 | 5 |
| 3 | PLVGPF | | | | | | | | | | |
| Phl p | NVWEVKSSK | 2 | 9 | 2 | 7 | 2 | 1 | 2 | 1 | >3 | 4 |
| 3 | PLVGPF | | | | | | | | | | |
| Phl p | NVWEVKSSK | 2 | 39 | 2 | 12 | 2 | 15 | 2 | 0 | >3 | 0 |
| 3 | PLVGPF | | | | | | | | | | |
| Phl p | AFKVAATAAN | >3 | 0 | 3 | 0 | 0 | 3 | 0 | 0 | >3 | 0 |
| 5 | AAPAN | | | | | | | | | | |
| Phl p | AFKVAATAAN | >3 | 1 | 3 | 0 | 0 | 8 | 0 | 0 | >3 | 2 |
| 5 | AAPAN | | | | | | | | | | |
| Phl p | AFKVAATAAN | >3 | 5 | 3 | 16 | 0 | 16 | 0 | 3 | >3 | 3 |
| 5 | AAPAN | | | | | | | | | | |
| Phl p | AFKVAATAAN | 3 | 32 | 3 | 19 | 0 | 21 | 0 | 2 | >3 | 8 |
| 5 | AAPAN | | | | | | | | | | |
| Phl p | AFKVAATAAN | >3 | 5 | 3 | 13 | 0 | 3 | 0 | 0 | >3 | 3 |
| 5 | AAPAN | | | | | | | | | | |
| Phl p | STWYGKPTG | 1 | 3 | 1 | 1 | 1 | 1 | 0 | 1 | >3 | 0 |
| 1 | AGPKDN | | | | | | | | | | |
| Phl p | STWYGKPTG | 1 | N/A | 1 | 0 | 1 | 12 | 0 | N/A | 3 | N/A |
| 1 | AGPKDN | | | | | | | | | | |
| Phl p | STWYGKPTG | 1 | 100 | 1 | 71 | 1 | 100 | 0 | 0 | >3 | 0 |
| 1 | AGPKDN | | | | | | | | | | |
| Phl p | SGIAFGSMAK | 3 | 5 | 2 | 5 | >3 | 2 | 2 | 0 | >3 | 1 |
| 1 | KGDEQ | | | | | | | | | | |
| Phl p | SGIAFGSMAK | 3 | 2 | 2 | 1 | >3 | 1 | 2 | 0 | >3 | 0 |
| 1 | KGDEQ | | | | | | | | | | |
| Phl p | SGIAFGSMAK | 3 | 45 | 2 | 14 | >3 | 36 | 2 | 18 | >3 | 41 |
| 1 | KGDEQ | | | | | | | | | | |
| Phl p | SGIAFGSMAK | 3 | 20 | 2 | 4 | >3 | 16 | 2 | 5 | >3 | 3 |
| 1 | KGDEQ | | | | | | | | | | |
| Phl p | GELELQFRRV | 0 | 0 | 0 | 1 | 1 | 1 | 0 | 0 | >3 | 0 |
| 1 | KCKYP | | | | | | | | | | |
| Phl p | GELELQFRRV | 0 | 0 | 0 | 0 | 1 | 2 | 0 | 2 | 3 | 3 |
| 1 | KCKYP | | | | | | | | | | |
| Phl p | GELELQFRRV | 0 | 4 | 0 | 0 | 1 | 0 | 0 | 8 | 3 | 33 |
| 1 | KCKYP | | | | | | | | | | |
| Phl p | GELELQFRRV | 0 | 16 | 0 | 11 | 1 | 37 | 0 | 0 | 3 | 1 |
| 1 | KCKYP | | | | | | | | | | |
| Phl p | GELELQFRRV | 0 | 2 | 0 | 13 | 1 | 27 | 0 | 0 | 3 | 3 |
| 1 | KCKYP | | | | | | | | | | |
| Phl p | LAKYKANWIE | >3 | 83 | >3 | 11 | >3 | 17 | >3 | 0 | >3 | 22 |
| 13 | IMRIK | | | | | | | | | | |
| | | | | | | | | Tree | | | |
| | | | | | | | | Fra e | | Controls | |
| | | | | | | Anti- | | # of | % | Relevant | Irrelevant |
| | | | | | | gen | Sequence | mm | Reactivity | Pool | Pool |
| | | | | | | NTGA | AVMLTFDNAG | 0 | 0 | 100 | 0 |
| | | | | | | 49 | MWNVR | | | | |
| | | | | | | NTGA | IGSFFYFPSIG | 1 | 1 | 63 | 1 |
| | | | | | | 54 | MQRT | | | | |
| | | | | | | NTGA | NPMTVFWSK | 2 | 0 | 79 | 1 |
| | | | | | | 76 | MAQSMT | | | | |
| | | | | | | NTGA | NGSQFFLCTA | 2 | 3 | 100 | 0 |
| | | | | | | 91 | KTAWL | | | | |
| | | | | | | NTGA | NGSQFFLCTA | 2 | 0 | 89 | 23 |
| | | | | | | 91 | KTAWL | | | | |
| | | | | | | NTGA | QYAKEIWGIT | >3 | 12 | 100 | 7 |
| | | | | | | 89 | ANPVP | | | | |
| | | | | | | NTGA | FPIVQRFLEGA | >3 | 1 | 16 | 100 |
| | | | | | | 2 | SSID | | | | |
| | | | | | | NTGA | FPIVQRFLEGA | >3 | 100 | 0 | 100 |
| | | | | | | 2 | SSID | | | | |
| | | | | | | Lol p | LIEKINAGFKA | >3 | 2 | 83 | 14 |
| | | | | | | 51 | AVAA | | | | |
| | | | | | | Lol p | LIEKINAGFKA | >3 | 15 | 90 | 20 |
| | | | | | | 51 | AVAA | | | | |
| | | | | | | Lol p | NAGFKAAVAA | >3 | 1 | 77 | 0 |
| | | | | | | 51 | AAVVP | | | | |
| | | | | | | Lol p | NAGFKAAVAA | >3 | 12 | 100 | 7 |
| | | | | | | 51 | AAVVP | | | | |
| | | | | | | Lol p | NAGFKAAVAA | >3 | 0 | 100 | 0 |
| | | | | | | 51 | AAVVP | | | | |
| | | | | | | Lol p | SDAKTLVLNI | 3 | 21 | 88 | 0 |
| | | | | | | 3 | KYTRP | | | | |
| | | | | | | Lol p | SDAKTLVLNI | 3 | 43 | 100 | 30 |
| | | | | | | 3 | KYTRP | | | | |
| | | | | | | Lol p | SDAKTLVLNI | 3 | 4 | 81 | 4 |
| | | | | | | 3 | KYTRP | | | | |
| | | | | | | Lol p | SDAKTLVLNI | 3 | 93 | 80 | 37 |
| | | | | | | 3 | KYTRP | | | | |
| | | | | | | Lol p | MRNVFDDVV | >3 | N/A | 81 | 22 |
| | | | | | | 2 | PADFKV | | | | |
| | | | | | | Lol p | MRNVFDDVV | >3 | 18 | 94 | 6 |
| | | | | | | 2 | PADFKV | | | | |
| | | | | | | Lol p | MRNVFDDVV | >3 | 2 | 98 | 4 |
| | | | | | | 2 | PADFKV | | | | |
| | | | | | | Lol p | MRNVFDDVV | >3 | 4 | 100 | 1 |
| | | | | | | 2 | PADFKV | | | | |
| | | | | | | Lol p | MRNVFDDVV | >3 | 0 | 100 | 0 |
| | | | | | | 2 | PADFKV | | | | |
| | | | | | | Lol p | NVFDEVIPTAF | 3 | 4 | 100 | 17 |
| | | | | | | 3 | TVGK | | | | |
| | | | | | | Lol p | NVFDEVIPTAF | 3 | 55 | 100 | 28 |
| | | | | | | 3 | TVGK | | | | |
| | | | | | | Lol p | DAYVATLTEA | 0 | 12 | 100 | 49 |
| | | | | | | 5 1 | LRVIA | | | | |
| | | | | | | Lol p | DAYVATLTEA | 0 | 1 | 96 | 46 |
| | | | | | | 5 1 | LRVIA | | | | |
| | | | | | | Lol p | DAYVATLTEA | 0 | 3 | 67 | 0 |
| | | | | | | 5 1 | LRVIA | | | | |
| | | | | | | Lol p | DAYVATLTEA | 0 | 0 | 100 | 4 |
| | | | | | | 5 1 | LRVIA | | | | |
| | | | | | | Lol p | AFKIAATAAN | 2 | 3 | 44 | 2 |
| | | | | | | 5 2 | AAPTN | | | | |
| | | | | | | Lol p | AFKIAATAAN | 2 | 28 | 100 | 20 |
| | | | | | | 5 2 | AAPTN | | | | |
| | | | | | | Lol p | AFKIAATAAN | 2 | 21 | 100 | 22 |
| | | | | | | 5 2 | AAPTN | | | | |
| | | | | | | Poa p | DINVGFKAAV | >3 | 0 | 53 | 5 |
| | | | | | | 1 | AAAAG | | | | |
| | | | | | | Poa p | DINVGFKAAV | >3 | 0 | 100 | 1 |
| | | | | | | 1 | AAAAG | | | | |
| | | | | | | Poa p | DINVGFKAAV | >3 | 4 | 95 | 15 |
| | | | | | | 1 | AAAAG | | | | |
| | | | | | | Poa p | DINVGFKAAV | >3 | 1 | 100 | 2 |
| | | | | | | 1 | AAAAG | | | | |
| | | | | | | Poa p | EPIAAYHFDLS | 1 | 0 | 95 | 11 |
| | | | | | | 1 | GKAF | | | | |
| | | | | | | Poa p | EPIAAYHFDLS | 1 | 0 | 68 | 2 |
| | | | | | | 1 | GKAF | | | | |
| | | | | | | Poa p | FKAAVAAAAG | >3 | 38 | 100 | 23 |
| | | | | | | 5 | APPAD | | | | |
| | | | | | | Dac g | GSDEKNLALS | >3 | 4 | 87 | 19 |
| | | | | | | 2 | IKYNK | | | | |
| | | | | | | Dac g | NLALSIKYNK | >3 | 0 | 100 | 0 |
| | | | | | | 2 | EGDSM | | | | |
| | | | | | | Dac g | DIYNYMEPYV | >3 | 49 | 100 | 49 |
| | | | | | | 4 | SKVDP | | | | |
| | | | | | | Lol p | KASNPNYLAIL | 0 | 12 | 92 | 12 |
| | | | | | | 1 | VKYV | | | | |
| | | | | | | Phl p | NFRFMSKGG | 0 | 3 | 100 | 15 |
| | | | | | | 3 | MRNVFD | | | | |
| | | | | | | Phl p | INVGFKAAVA | >3 | 2 | 100 | 4 |
| | | | | | | 5 | AAASV | | | | |
| | | | | | | Phl p | INVGFKAAVA | >3 | 0 | 70 | 3 |
| | | | | | | 5 | AAASV | | | | |
| | | | | | | Phl p | INVGFKAAVA | >3 | 21 | 60 | 0 |
| | | | | | | 5 | AAASV | | | | |
| | | | | | | Phl p | EEWEPLTKKG | 1 | 76 | 100 | 32 |
| | | | | | | 3 | NVWEV | | | | |
| | | | | | | Phl p | NVWEVKSSK | 2 | 0 | 100 | 3 |
| | | | | | | 3 | PLVGPF | | | | |
| | | | | | | Phl p | NVWEVKSSK | 2 | N/A | 100 | 0 |
| | | | | | | 3 | PLVGPF | | | | |
| | | | | | | Phl p | NVWEVKSSK | 2 | N/A | 68 | 1 |
| | | | | | | 3 | PLVGPF | | | | |
| | | | | | | Phl p | NVWEVKSSK | 2 | N/A | 100 | 1 |
| | | | | | | 3 | PLVGPF | | | | |
| | | | | | | Phl p | NVWEVKSSK | 2 | 0 | 94 | 3 |
| | | | | | | 3 | PLVGPF | | | | |
| | | | | | | Phl p | NVWEVKSSK | 2 | 0 | 100 | 1 |
| | | | | | | 3 | PLVGPF | | | | |
| | | | | | | Phl p | AFKVAATAAN | 0 | N/A | 100 | 0 |
| | | | | | | 5 | AAPAN | | | | |
| | | | | | | Phl p | AFKVAATAAN | 0 | N/A | 100 | 1 |
| | | | | | | 5 | AAPAN | | | | |
| | | | | | | Phl p | AFKVAATAAN | 0 | 0 | 100 | 13 |
| | | | | | | 5 | AAPAN | | | | |
| | | | | | | Phl p | AFKVAATAAN | 0 | 13 | 99 | 13 |
| | | | | | | 5 | AAPAN | | | | |
| | | | | | | Phl p | AFKVAATAAN | 0 | 27 | 100 | 22 |
| | | | | | | 5 | AAPAN | | | | |
| | | | | | | Phl p | STWYGKPTG | 1 | 1 | 57 | 0 |
| | | | | | | 1 | AGPKDN | | | | |
| | | | | | | Phl p | STWYGKPTG | 1 | N/A | 25 | 1 |
| | | | | | | 1 | AGPKDN | | | | |
| | | | | | | Phl p | STWYGKPTG | 1 | 0 | 17 | 16 |
| | | | | | | 1 | AGPKDN | | | | |
| | | | | | | Phl p | SGIAFGSMAK | 2 | 2 | 26 | 0 |
| | | | | | | 1 | KGDEQ | | | | |
| | | | | | | Phl p | SGIAFGSMAK | 2 | 0 | 24 | 1 |
| | | | | | | 1 | KGDEQ | | | | |
| | | | | | | Phl p | SGIAFGSMAK | 2 | 0 | 100 | 23 |
| | | | | | | 1 | KGDEQ | | | | |
| | | | | | | Phl p | SGIAFGSMAK | 2 | 11 | 100 | 25 |
| | | | | | | 1 | KGDEQ | | | | |
| | | | | | | Phl p | GELELQFRRV | 0 | 1 | 88 | 0 |
| | | | | | | 1 | KCKYP | | | | |
| | | | | | | Phl p | GELELQFRRV | 0 | 0 | 53 | 2 |
| | | | | | | 1 | KCKYP | | | | |
| | | | | | | Phl p | GELELQFRRV | 0 | 46 | 92 | 0 |
| | | | | | | 1 | KCKYP | | | | |
| | | | | | | Phl p | GELELQFRRV | 0 | 5 | 100 | 0 |
| | | | | | | 1 | KCKYP | | | | |
| | | | | | | Phl p | GELELQFRRV | 0 | 12 | 100 | 8 |
| | | | | | | 1 | KCKYP | | | | |
| | | | | | | Phl p | LAKYKANWIE | 2 | 14 | 31 | 19 |
| | | | | | | 13 | IMRIK | | | | |

Table 10 shows individual peptide data for the cross reactivity experiments. Each peptide was used to stimulate cells and cross reactivity was tested for extracts from each pollen species. The number of mismatches (# of mm) for each peptide compared to the pollen species and the reactivity of the extracts as a percentage of the reactivity compared to the peptide are shown. Peptides are SEQ ID NO's 246, 258, 315, 1110-1177 in order of appearance, e.g. peptide NGSQFFLCTAKTAWL of NTGA 91 has SEQ ID NO: 1110.

### EXAMPLES

### Example 1

This example includes a description of transcriptomic analysis of various pollen species and conservation analysis.

A set of 93 proteins from Timothy grass (TG) pollen and the assembly of 822 peptides (15 mers) predicted to promiscuously bind HLA class II molecules shown in Table 9 and the immune reactivity in allergic donors have been reported in PCT application WO 2013/119863. Promiscuous binders were determined by predicting the binding affinity to a panel of 25 HLA class II molecules using a consensus prediction approach (Wang P, et al. (2008) and Wang P, et al. (2010). Peptides with predicted binding scores in the top 20% for a given allele were considered potential binders. Peptides predicted to bind 13 or more of the HLA molecules in Table 9 at this threshold were considered promiscuous binders, and selected for synthesis (after eliminating peptides overlapping by more than 9 contiguous residues). If less than 5 peptides from a given protein met this threshold, the top 5 peptides were chosen, and up to 4 peptides in proteins where length was prohibitive. In total, this resulted in the selection of 822 TG peptides from a total of 21,506 distinct 15-mers encoded in 620 ORFs derived from the transcriptomic analysis. Immune reactivity was determined by the production of IL-5 or IFNg from cultured PBMCs of the allergic donors in response to stimulation with a peptide and IL-5 and IFNg were measured by ELISPOT as described in Oseroff C et al, 2010.

In short, T cell immune reactivity was determined using PBMCs isolated from study participants and stored in liquid nitrogen until further use. For experimental testing, PBMCs were thawed and expanded in vitro with TG pollen extract (50 μg/mL) or the peptide pool (5 pg/mL). The TG extract and peptide pools had each been previously titrated to determine optimal stimulation concentrations.

Cytokine production by cultured PBMCs in response to antigen stimulation was measured by ELISPOT. Cells (1×10⁵ cells/well) were plated and incubated with peptide (10 μg/mL), the peptide pool (5 μg/mL), or the TG extract (50 μg/mL). Phytohaemagglutinin (10 μg/mL) and medium alone were used as positive and negative controls, respectively. Samples were considered to produce a cytokine if 100 spot-forming cells (SFCs)/10⁶ PBMCs were detected, with P .05 and a stimulation index of 2 or more. Criteria for individual peptides were the same except that a minimum of 20 SFCs were required for a sample to be counted as positive.

To study the conservation of the 822 TG peptides in other pollen species, RNA-sequencing were performed on pollen samples of four additional grass pollen species (Kentucky blue grass (Poa pratensis, Poa p), Sweet vernal grass (Anthoxanthum odoratum, Ant o), Rye grass (Lollium Perenne, Lol p), Bermuda grass (Cynodon dactylon, Cyn d)) and five non-grass pollen species (Western ragweed (Ambrosia psilostachya, Amb p), Short ragweed (Ambrosia artemisiifolia, Amb a), White oak (Quercus alba, Que a), European white birch (Betula verrucosa, Bet v), European Ash (Fraxinus Excelsior Fra e), European Olive (Olea Europaea, Ole e), English plantain (Plantago lanceolata Pla I),). RNA-seq was run at UCSD, using an Illumina HiSeq 2000. RNA-seq was run at UCSD, using an Illumina HiSeq 2000. The table below shows the number of reads assembled for each of the different pollens (top), with over 500 million reads over two replicate runs per allergen. Sequences were assembled into transcripts using Trinity (bottom), resulting in over 50 thousand transcripts per allergen with minimum lengths of 200 nucleotides. The transcripts include related variants, such as isoforms, and homologs.

Sequencing was performed on an Illumina Genome Analyzerllx (GAIIx). Briefly, adaptor-ligated cDNA was loaded into an Illumina flow cell. DNA was then bridge-amplified within the flow cell to generate millions of DNA clusters by using specific reagents and enzymes (Illumina Paired-End Cluster Generation Kit). The flow cell was loaded onto the GAIIx equipped with a paired-end module, and 72 sequencing cycles were performed to generate sequence in both directions by using Illumina Sequencing Kit v4. Replicate samples were run in seven of the eight lanes on the flow cell, producing 280 million raw sequence reads of 72 by in length. Reads went through several preprocessing steps using the FastX toolkit (2) before they were assembled into contigs: (i) the 3′ terminal base was removed; (ii) low-complexity reads were removed; (iii) portions of reads downstream of a low-quality score were removed; and (iv) portions of reads corresponding to adapter sequencers were removed. The remaining reads were assembled into contigs by using Velvet (Version 1.0.15) (3). Because of the excessive memory requirements inherent to de novo sequence assembly, the reads for each lane were considered separately and were each run with five different values for the word size parameter (k=21, 23, 25, 27, 29). We and others (4) have observed that different sets of contigs are obtained for each value for k. The contigs were further merged with Oases (Version 0.18.1; D. R. Zerbino, European Bio-informatics Institute, Hinxton, United Kingdom) into putative transcripts.

Table showing pollen RNA-seq reads for various pollen species

Grass Pollen Species
Raw read counts (millions)

| | Sweet vernal | Bermuda | Rye | Kentucky |
|---|---|---|---|---|
| | grass | grass | grass | blue grass |
| | Ant o | Cyn d | Lpl p | Poa p |
| 1st run | 394 | 354 | 332 | 363 |
| 2nd run | 360 | 309 | 319 | 309 |
| Total | 754 | 663 | 651 | 672 |

Transcripts after Trinity assembly

| | | | | |
|---|---|---|---|---|
| Count | 317,874 | 112,527 | 122,266 | 128,174 |
| min length | 201 | 201 | 201 | 201 |
| median length | 544 | 842 | 631 | 635 |
| max length | 11,515 | 14,364 | 9,631 | 10,100 |

Non-grass pollen species
Raw read counts (millions)

| | Short | Western | European | European | English | White |
|---|---|---|---|---|---|---|
| | Ragweed | ragweed | Ash | Olive | plantain | Oak |
| | Amb a | Amp p | Fra e | Ole e | Pla a | Que |
| 1st run | 528 | 328 | 410 | 385 | 303 | 329 |
| 2nd run | | 299 | 346 | 350 | 287 | 307 |
| Total | 528 | 627 | 756 | 735 | 590 | 635 |

Transcripts after Trinity assembly

| | | | | | | |
|---|---|---|---|---|---|---|
| count | 95,759 | 121,659 | 81,401 | 74,333 | 57,102 | 54,280 |
| min length | 201 | 201 | 201 | 201 | 201 | 201 |
| median length | 352 | 390 | 722 | 710 | 696 | 634 |
| max length | 10975 | 8,325 | 9,838 | 8,133 | 8,090 | 14,807 |

### Example 2

This example includes a description of how to identify which of the TG peptides that are conserved across a grass pollen and various non-grass pollen species.

The degree of conservation of the known 15-mer peptides deriving from TG pollen proteins was determined across the different pollens. For the purpose of this analysis, peptides that have a homologous hit with 0, 1 or 2 mismatches are considered as being conserved. Any substitution of an amino acid sequence within the 15mer TG peptide is considered to constitute a mismatch. All 15mer peptides (overlapping by 10 aa) of the representative/construct sequence were created in silico and compared against the protein sequences of non-TG species. All peptides with 2 or less mismatches to the TG construct peptides were run through the IEDB MHC class II peptide binding predictor for 20 common class II alleles.

In total 499 of the 822 TG peptides have a mismatch of less than 3 (0, 1, or 2 mismatces) to a homologous peptide in another grass pollen species. A fraction (397 peptides) of the 499 TG peptides had a mismatch of less than 3 to a homologous peptide in at least one of the non-grass pollen species (Amb p, Ole e, Pla I, Fra e and Que e), these peptides for the purpose of this application are named “pan-grass plus” peptides (PG+) and are conserved across each of the grass pollen species investigated and at least to one non-grass pollen species with less than 3 mismatches compared to the PG+ sequence. A fraction (224 peptides) of the 397 peptides had a mismatch of less than 3 to a corresponding peptide found in each of the non-grass species investigated, these peptides for the purpose of this application are named “pan-pollen” peptides (PP peptides).

Table 3 lists the 397 PG+ peptides and indicates for each non-grass pollen species whether a matching peptide with either less than 3, less than 2 or zero mismatches could be detected. The immune reactivity of the TG peptide was assessed as the number of TG grass allergic donors (n=20) having in vitro T cell response against the TG peptide.

### Example 3

This example includes a description of how to identify PG+ peptides having high correlation between immune reactivity and conservation across grass and non-grass pollen species.

Some PG+ peptides were conserved across several grass pollen and non-grass pollen species and produced a T cell response in a higher fraction of the donors. For example, PG+ peptides recognized by two or more grass allergic donors (n=20), i.e. NTGA's numbered 2, 6, 7, 24, 49/54, 89 and 91 (Table 7).

Furthermore, some highly conserved PG+ peptides produce high immune reactivity (high SFC counts in ELISPOT). Those peptides are derived from NTGA's numbered 2, 6, 7, 22, 24, 27, 49, and 90.

The degree of conservation of 36 peptides for which there was found 3 or more donors reacting to T cells (either for IFN-g or for IL-5) was determined. On average, these peptides were found conserved in 6.6±0.43 (average ±standard error of the mean) pollen species in addition to Timothy grass (Phl p). In contrast, peptides that were unreactive in all donors were found to be conserved in only 2.3±0.11 other pollen species. This shows that conservation and immune reactivity most likely are correlated.

### Example 4

This example includes a description of proteins with high number of conserved peptides.

Tables 5 and 6 shows NTGAs ranked according to the number of PG+ peptides or PP peptides contained in the NTGA sequence. For example it was found that NTGA's containing at least 5 PG+ peptides conserved across grass, weed and tree pollen (GWT) were proteins numbered 1, 2, 4, 5, 6, 7, 13, 20, 21, 22, 24, 26, 30, 32, 34, 36, 39, 42, 72, 77, 83, 84, 86, 39/59, 49/54, 86/51 (Table 5) and those containing at least 8 PG+ peptides conserved across grass, weed and tree pollen (GWT) were proteins numbered 1, 2, 4, 5, 6, 7, 13, 24, 30, 34, 72, 83, 86, 39/59, 49/54, 86/51. The top 20 list of NTGAs ranked according to their number of PG+ peptides are NTGA's numbered 6, 89, 30, 1, 72, 2, 13, 83, 86, 77, 4, 24, 34, 7, 29, 76, 20, 59, 84, 49/54.

Table 6 shows the proteins ranked according to the number of PP peptides contained in the NTGA. The top 20 list of pan-pollen NTGA's ranked according to the number of PP peptides are NTGA's numbered 30, 86, 6, 13, 72, 4, 2, 24, 26, 49/54, 34, 7, 77, 83, 32, 42, 21, 22, 84. A fraction of those proteins contains highly T cell reactive sequences (2, 6, 7 and 53).

### Example 5

This example includes a description of the full length sequences of NTGA's and their homologs in other pollen species.

Full length sequence of NTGA's were assembled using multiple sequence alignments of transcripts from the different pollens, thereby identifying with more confidence the full length sequence of selected antigens of interest based on conserved start- and stop-codons. For example this made it possible to distinguish between multiple variants of TG transcripts identified in the initial assembly, and then pick high confidence candidate sequences that are starting points for protein synthesis.

In order to identify the correct coding region of each transcript, there was identified the closest homologous sequence in the rice (Oryza sativa japonica) proteome (via Blast). Rice was chosen since it is a species closely related to Timothy grass with a completely sequenced and annotated genome. Homologous rice sequences were identified for 180 Timothy grass sequences. Subsequently, homologous sequences were identified (via Blast) in the translated transcriptomes of Cyn d, Amb a, Amb p, Que a, and Bet v. of all identified sequences, the one(s) sharing the largest number of conserved peptides with the Phl p sequence was selected as homolog. In addition, there was found evidence of the presence of the NTGA's upon extracting pollen in a buffered aqueous solution for at least 2 hours hours and detecting the NTGA's by mass spectrometry analysis of the trypsin-treated extract and comparing mass signals to protein databases. Table 2 shows Phl p amino acid sequences of the identified NTGA's in Phl p grass pollen and Table 4 shows amino acid sequences of proteins with high identity and similarity to the Phl p sequence that are found in non-grass pollen species or in grass pollen species other than Phl p.

During the work with assembling the full length sequences it was found that PG+ peptides of NTGA's 5 and 64 derives from the same full length sequence, thus hereinafter named NTGA 5/64. Likewise, PG+ peptides of NTGA's 86 and 51 derive from the same full length sequence, and the full length protein is hereinafter named 86/51. PG peptides of NTGA's 49 and 54 derive from the same full length sequence, thus hereinafter named NTGA 49/54. PG+ peptides of NTGA's 39 and 59 derive from the same full length sequence, thus hereinafter named NTGA 39/59.

### Example 6

This example includes a description of the identification of conserved regions of NTGA's of Table 2 across homologs thereof shown in Table 4.

Multiple sequence alignments were generated for each set of homologous sequences. For each Phl p reference sequence (e.g. NTGA 6 disclosed in Table 2), the degree of conservation of each 15 mer peptide contained in this sequence across the other species was determined. For the purpose of this analysis, it was defined that peptides that have a homologous hit with 0, 1 or 2 mismatches are considered as being conserved. Any substitution of an amino acid sequence within the 15 mer Phl p peptide is considered to constitute a mismatch. A conserved region (e.g. conserved stretch) was then defined as the region resulting from merging all conserved 15 mer peptides in a Phl p sequence.

A region was defined as conserved across “grass & weed & tree” if conserved across at least one weed species (Ambrosia artemisiifolia and/or Ambrosia psilostachya) and at least across one tree species (Quercus alba and/or Betula verrucosa). Table 3 shows for each NTGA tested, the amino acid sequences of the conserved regions found across “grass & weed & tree” (GWT sequences) .

### Example 7

This example includes a description of how to examine release patterns of immunogens from pollen (Screening for co-release of NTGA's with major allergens from various pollen species) and detecting polypeptides of the invention by Mass Spectrometry

Raw pollen or defatted pollen of various pollen sources, Glass bottles (100 ml) for extraction, PD-10 columns with PE bed support combined with 10 ml syringe with silicone tubing, PBS buffer, pH 7.2 containing the following salts:

| | | M_{w} | Conc. | Conc. |
|---|---|---|---|---|
| Salt | | (g/mol) | g/L | mM |
| Sodium chloride | NaCl | 58.44 | 8.0 | 137 |
| Potassium chloride | KCl | 74.55 | 0.2 | 2.7 |
| Na-phosphate | Na₂HPO₄, 2H₂O | 175.98 | 1.44 | 8.2 |
| K-phosphate | KH₂PO₄ | 136.09 | 0.2 | 1.5 |

Phosphate conc.: 8.2 + 1.5 = 9.7 mM phosphate
NaCl: μ = ½ * (137 * 12 + 137 * 12) = 137 mM
KCl: μ = ½ * (2.7 * 12 + 2.7 * 12) = 2.7 mM
Na2HPO4: μ = ½ * ((8.2 * 2 * 12) + (8.2 * 22)) = 24.6 mM
KH2PO4: μ = ½ * ((1.5 * 12) + (1.5 * 12)) = 1.5 mM
Total ionic strength: μ = 165.8 mM ≈ 0.17 M

Extraction Procedure (at room temperature, 21-24° C.):

5.0 g of pollen are weighed into a glass bottle and 50 ml of PBS is added and the bottle is immediately rotated, first 5 minutes by hand and thereafter rotated in a sample rotator during the entire extraction.

5 ml of slurry is taken out after 20 sec, transferred to a column with a bed filter and dragged through the filter with a syringe. The syringe is immediately transferred to a filter unit and the extract is pushed through the combined filters into a labelled test tube. The tube is stored in an ice bath until the sample is pipetted in aliquots for further analysis and frozen. About 5 ml of the suspension is taken out at various time points.

Samples are analysed for NTGA and major allergens by MS (Mass Spectrometry) using the following materials and methods:

Buffers/solutions for reduction, alkylation and digestion of the sample:

Sample buffer: 8 M urea in 0.4 M NH₄HCO₃

DTT (45 mM): Make it fresh from the frozen stock 1.0 M: 45 μl 1 M DTT+955 μl water Iodoacetamide (IAA): Make fresh solution, Iodoacetamid 100 mM,

Trypsin: Sigma T6567, Dissolve one vial in 20 μl of 1 mM HCl. This results in a solution containing 1 μg/μl trypsin. After reconstitution in 1 mM HCl frozen aliquots can be stored for up to 4 weeks.

Enzymatic digestion with trypsin in solution for mass spectrometry: Dilute the dried sample in 5 μl of water, add 15 μl of sample buffer (8 M Urea in 0.4 M NH₄HCO₃), add 5 μl 45 mM DTT, incubate at 56° C. for 15 min, cool it to room temperature, add 5 μl of 100 mM Iodoacetamide, incubate in the dark in room temperature for 15 min, add 90 μl of water to lower the concentration of urea <1-2 M, add 1 μg trypsin, incubate at 37° C. over night.

Chromatography: Reverse phase chromatography (Ultimate 3000 HPLC, Dionex) was performed using a C18 pre- and analytical column. The eluting peptides were sprayed directly into an ESI-QTOF mass spectrometer (MaXis, Bruker). After washing the trap column with 0.05% v/v formic acid for 5 min with a flow rate of 30 μl/min, the peptides were eluted with an acetonitrile gradient at a flow rate of 2 μl/min using solvent A: 0.05% v/v formic acid and solvent B: 80% v/v acetonitrile/0.04% v/v formic acid and the gradient: 4-50% B in 200 minutes; 50-80% B in10 minutes; 100% B in 10 min, 4% B in 5 min.

Spectra were acquired in the mass range 50-2599 m/z and a spectra rate of 1.5 Hz. The instrument was tuned and calibrated using ESI-L Low concentration Tunning Mix from Agilent Technology.

Data acquisition and instrument control were carried out with Bruker Compass HyStar 3.2. Data processing was performed using DataAnalysis 4.0 (Bruker). Protein identification was performed using the program Biotools3.2 (Bruker) and two different data bases, i.e. Swiss prot and NCBInr. The MS/MS data sets for the tryptic digest were analysed using the following parameters; peptide tolerance 10 ppm and fragment tolerance 0.05 Da.

Procedure: The extraction samples were all evaporated (50 μl) and re-suspended in 5 μl of water. The sample is then reduced, alkylated and digested with trypsin. Resulting peptides are separated and identified by reversed phase chromatography followed by MS/MS.

Results: The release of major allergens from the various pollen species investigated is initiated almost instantly after hydration of pollen with buffer and the release continues with high rate within a time range of at least 30 to 60 minutes (data not shown). Table 5 shows which NTGA's and the Amb a homolog thereof that starts release within a period overlapping with the release of major allergens from grass pollen (Phl p) and weed pollen (Amb a), respectively (GW release). Likewise, the NTGAs and its Que a homolog that starts release within 30 minutes from grass pollen (Phl p) and tree pollen (Que a) is also shown (GT release). Finally, NTGA's or its Amb a and its Que a homolog released from grass pollen (Phl p) and weed pollen (Amb a) and tree pollen (Que a) is also shown (GWT release).

It was found that at least the NTGA's 1, 4, 6, 7, 24, 26, 29, 30, 39, 47, 51, 59, 64, 86, 91, 5/64, 39/59, 51/86 start release within 30 minutes from Phl p grass pollen and the corresponding Amb a homolog starts release within 30 minutes from Amb a pollen after hydration. At least NTGA's 24, 29, 56, 91 start release within 30 minutes from Phl p grass pollen and the corresponding Que a homolog starts release within 30 minutes from Que a pollen after hydration. At least NTGA's 24, 29 and 91 start release within 30 minutes from Phl p grass pollen as well as weed pollen (Amb a) and Oak pollen (Que a). I was also found that the release of NTGA's 1, 3, 4, 6, 5/64, 20, 24, 26, 30, 39/59, 47, 62, 76, 86/51, 89 and 91 was started within 30 minutes from both Phl p grass pollen and Cyn d pollen. NTGA's 8, 9, 10, 19, 22, 32, 34, 40, 42, 43, 54, 65 and 77 has not been tested.

### Example 8

This example describes how to determine that T cells responding to a particular PG+ peptide (Phl p sequence) also recognizes a sequence of a corresponding peptide identified in a non-grass pollen species.

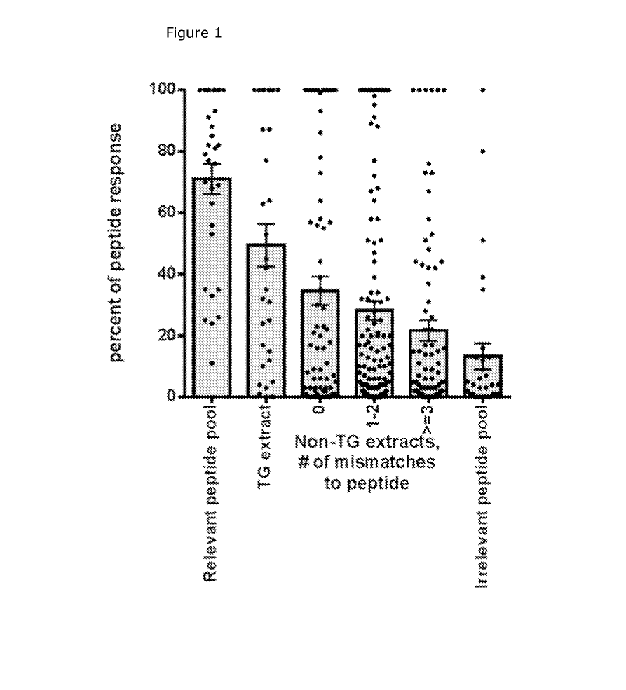

PBMCs from Phl p reactive donors were expanded with individual PG+ peptides as well as peptides derived from major allergens of Phl p for 14 days (peptides shown in Table 10). For each peptide, the mismatch to a corresponding sequence in a non-grass pollen species or a pollen species other than Phl p were determined. Cytokine IL-5 responses were measured in response to the peptide itself, Phl p extract and extracts of the other pollen species. Reponses to extracts and peptide pools were expressed as the relative fraction of the response to the peptide itself and plotted as a function of conservation of the peptide in the different extracts (FIG. 1). The data points for each peptide are contained in Table 10. A clear hierarchy of responses was observed, with non-Phl p extracts in which the peptide is completely conserved (zero mismatches) showing the highest response, followed by non-Phl p extracts with 1-2 mismatches, and lowest responses with non-Phl p extracts with 3 or more mismatches. The exact same hierarchy was observed when analyzing peptides from the major allergens and the NTGA-derived peptides separately. Thus, Phl p epitopes conserved in other pollen species, including pollen of Amb a and Que a and other non-grass pollen, were indeed able to induce cross-reactive T cell immune responses.

### Example 9

This example describes how to determine the ability of a NTGA or a corresponding sequence found in a non-grass pollen species to relieve an allergic immune response in mice.

Initially, the sensitization pattern of an immunogen of the invention (NTGA 86/51) was investigated in BALB/c mice sensitized to Phl p extract (FIG. 2). For the purposes of these studies, the immunogen were expressed in E. Coli using standard expression protocols.

Initially, the sensitization pattern of an immunogen of the invention (NTGA 86/51) was investigated in BALB/c mice sensitized to Phl p extract (FIG. 2). For the purposes of these studies, the immunogen were expressed in E. Coli using standard expression protocols.

Mice were sensitized by one intraperitoneal injection with Phl p extract adsorbed to aluminium hydroxide. Eleven days later the mice were euthanized and splenocytes were stimulated in vitro with Phl p extract, Phl p 1, Phl p 5, NTGA 86/51. The cells were incubated for 6 days at 37° C. under 5% CO2 and incorporated 3H-thymidine was counted and used as a measure for T cell proliferation.

The results show that the in vitro T-cell response towards NTGA 86/51 is much weaker compared to the response to Phl p 5. This correlates well with the human situation, where Phl p 5 is considered to be a major T-cell allergen. In line with this, the results also show that the response towards NTGA 86/51 is much weaker compared to the response towards the Phl p extract that was used for intraperitoneal sensitization.

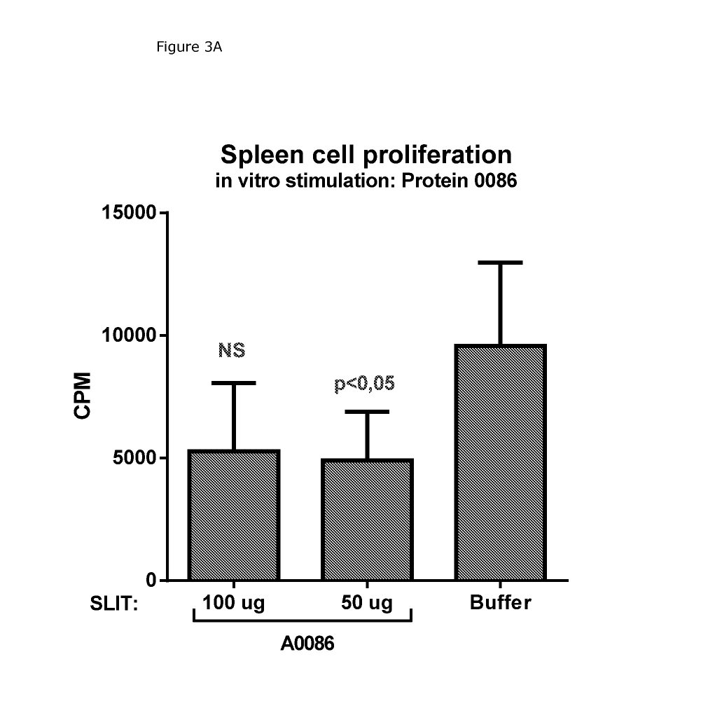

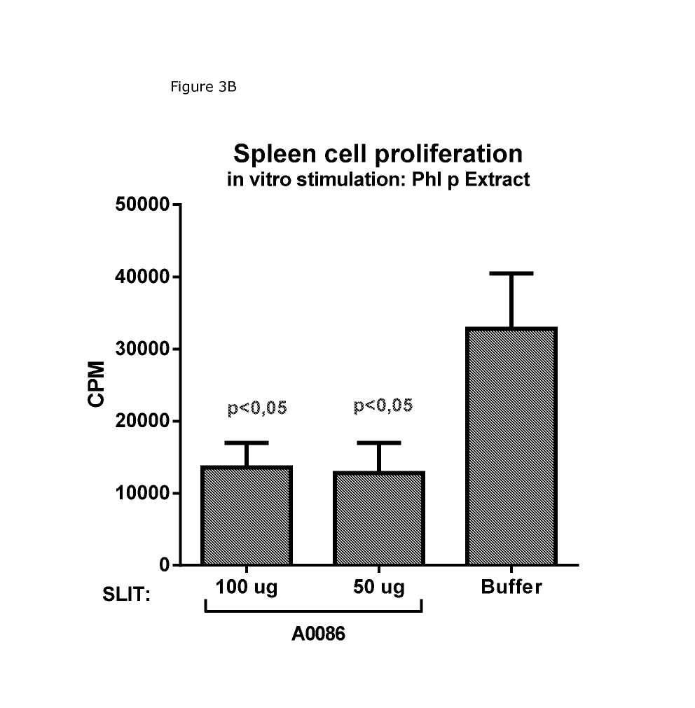

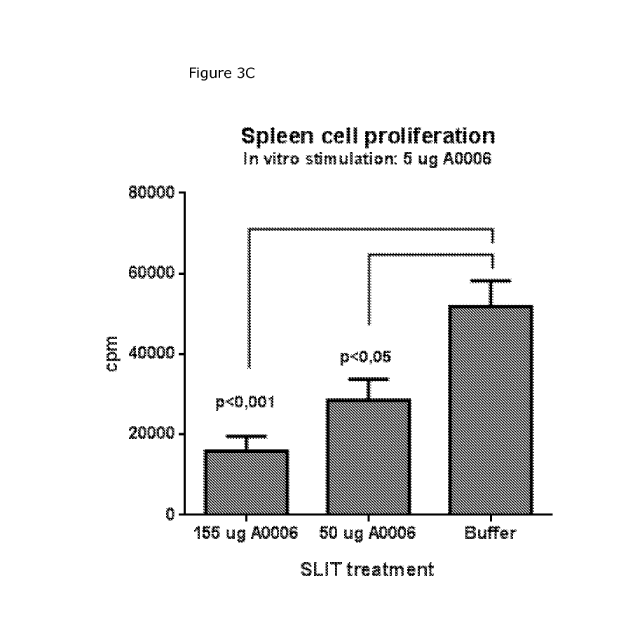

Then the tolerance induction of NTGA 86/51 was investigated in a prophylactic mice model using sublingual administration (FIG. 3)

The ability of NTGA 86/51 and NTGA 6 to induce prophylactic tolerance was investigated by SLIT treating naive BALB/c mice with NTGA 86/51 or NTGA 6 for two weeks (Monday-Friday) followed by one Phl p extract sensitization or sensitization to the immunogen itself (NTGA 86/51 or NTGA 6) as described above. Eleven days after the sensitization, splenocytes were harvested and stimulated in vitro with NTGA 86/51 as well as Phl p extract.

The result is presented in FIGS. 3A-C and show that prophylactic SLIT treatment with NTGA 86/51 is capable of inducing tolerance towards itself (3A) as well as towards the Phl p extract (3B), as shown by the reduced proliferation in splenocytes from the NTGA 86/51-treated mice compared to Buffer (sham) treated mice. In addition, it was shown that NTGA 6 is capable of inducing tolerance towards itself (3C)

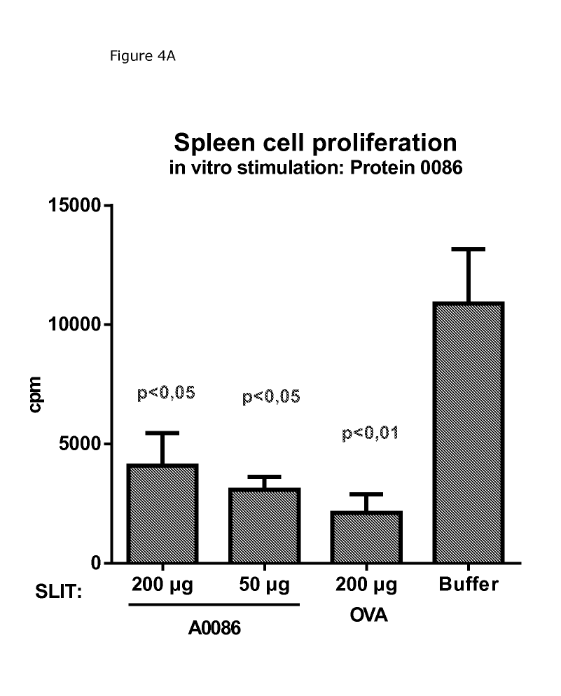

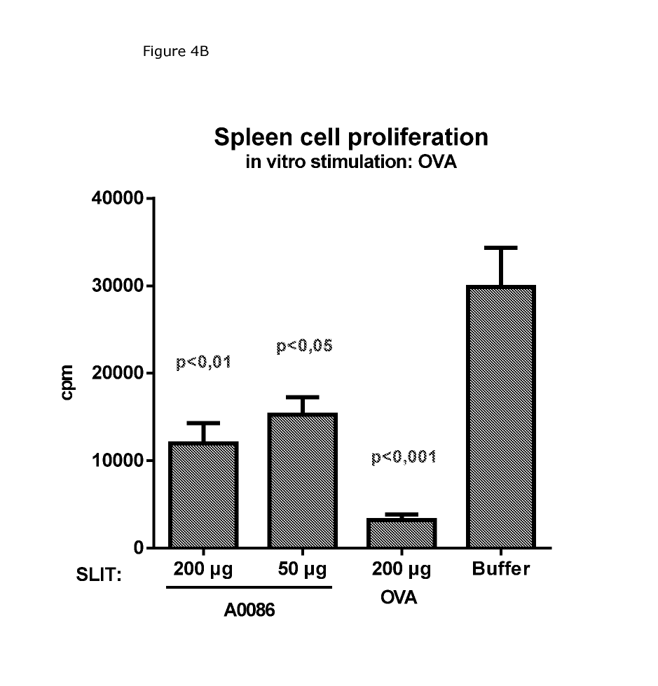

Bystander tolerance induction by prophylactic SLIT treatment with A0086 (FIG. 4). The ability of NTGA 86/51 to induce bystander tolerance, i.e. to induce tolerance against a non-related protein was investigated by SLIT treating the mice for two weeks with NTGA 86/51 followed by an IP sensitization with NTGA 86/51 together with the unrelated protein ovalbumin (OVA). Following this splenocytes were stimulated in vitro either with NTGA 86/51to confirm the ability of this protein to induce tolerance towards itself, or with OVA to investigate if NTGA 86/51 can induce bystander tolerance towards an unrelated protein.

As shown in FIG. 4A, prophylactic SLIT treatment with NTGA 86/51 is capable of inducing direct tolerance (towards NTGA 86/51 itself), as demonstrated by reduced proliferation of splenocytes from NTGA 86/51 treated mice compared to buffer treated mice. Furthermore, FIG. 4B shows that SLIT treatment with OVA is also able to downregulate the NTGA 86/51 specific in vitro response, demonstrating bystander tolerance induction by OVA SLIT. Likewise, SLIT treatment with NTGA 86/51 is also able to induce bystander tolerance, as measured by the decreased OVA specific in vitro proliferation of splenocytes from A NTGA 86/51-SLIT treated mice compared to buffer treated mice.

The mechanism behind tolerance induction towards major allergens using proteins that are not themselves major allergens is believed to be induction of regulatory T-cells specific for the proteins used for SLIT treatment. At challenge it is therefore important that these proteins are present in the pollen grains in sufficient amounts to re-activate the regulatory T-cells, in order for the tolerance to spill over to the major allergens. When targeting multiple pollen allergies by one immunogen, it is crucial that this immunogen or one highly conserved thereto is present in all the pollen species of interest in sufficient amounts (pan-pollen immunogen). Furthermore, it may be important that the epitopes recognized by the regulatory T-cells induced during SLIT treatment is sufficiently conserved across the immunogens - otherwise the regulatory T-cells will not be re-activated and tolerance will not occur.

Whether an immunogen of the invention can relieve an immune response triggered by a pollen allergen in mice that are sensitized to the pollen allergen when starting SLIT treatment can be investigated in a therapeutic mice model. For example, BALB/cJ mice or HLA-transgenic mice may be IP sensitized with model allergen adsorbed to aluminium hydroxide (e.g. an extract of a grass pollen species, e.g. cyn d, Poa p, Phl p or a model allergen like OVA). Subsequently, the mice might be treated by sublingual immunotherapy (SLIT) with an immunogen of the invention for a period of about 4 weeks, followed by about 2 weeks of intranasal challenge with model allergen together with the immunogen or model allergen alone to induce an allergic immune response in the airways. Mice are then sacrificed one day after the last challenge and blood, bronchoalveolar fluid (BAL), spleen and cervical lymph nodes may be collected for analysis. Clinically relevant readouts, such as sneezes, airway hyper-reactivity and presence of eosinophils, might be obtained on the last day of intranasal challenge. For example, sneezed may be observed in an 8 min-period after intranasal administration of model allergen and the numbers of sneezes be counted during this period. Airway hyper-reactivity may be determined using a whole body pletysmograph, airflow obstruction might be induced by increasing concentrations of aerosolized metacholine. Pulmonary airflow obstruction may be measured by enhanced pause (penh) in a period of 6 minutes after administration of metacholine. Differential counting of bronchial fluid (BAL) is performed after centrifugation of BAL fluid and removal of supernatant. The pellet was re-suspended in PBS and the fraction of eosinophils might be determined by an automated cell counter (Sysmex).

The results may show that an immunogen of the invention is able to reduce the number of sneezes, number of eosinophils, airway obstruction, T cell proliferation of spleen cells or cervical lymph nodes and may be shown to depend on the co-exposure of model allergen and immunogen at the target organ (airways).

Whether SLIT treatment with pan pollen immunogens is capable of inducing tolerance that can be re-activated by a non-identical, but highly conserved immunogen from a different pollen source can be addressed in several different in vivo models, as outlined below.

Experiment 1:
1. SLIT treatment with immunogen A
2. IP Sensitization with immunogen B (contains conserved regions overlapping with A)
3. in vitro stimulation with immunogen B

Where results verify that the specific in vitro proliferation to immunogen B is down-regulated in mice SLIT-treated with immunogen A, then cross-species tolerance induction has been demonstrated for this immunogen, since the two immunogens are sufficiently similar in order for cross-species tolerance induction to occur.

Experiment 2:
1. SLIT treatment with immunogen A
2. IP Sensitization with extract of pollen source containing immunogen B (pollen extract containing the homologous immunogen B)
3. In vitro stimulation with extract of pollen source containing immunogen B and immunogen B

Where results verify that the specific in vitro proliferation to immunogen B extract is down-regulated in mice SLIT-treated with immunogen A, then cross-species tolerance induction has been demonstrated for this immunogen. Furthermore, it has been demonstrated that pollen source B contains sufficient amounts of immunogen B to re-activate the tolerance induced by SLIT treatment with immunogen A.

In the above-mentioned mice model, Balb/cJ mice have been suggested. However, in vivo studies may instead be carried out in humanized mice models using transgenic mice, e.g. “HLA-DRB1*0401 transgenic mice” that may be obtained from Taconic. Also, in the above-mentioned mice models, the immune response against an allergen of a grass pollen (phl p grass extract) have been investigated, but other models may investigate the immune reponse against non-grass pollen allegens, e.g. allergens of weed or tree pollen, or there may be used model allergens like OVA protein.

Furthermore, the T cell responses in mice or humans may be evaluated by in-vitro T cell proliferation assays or ELISPOT assays. The production of IL-5 and IFN-y from cultured PBMCs (Peripheral blood monocytes) obtained from mice or human in response to stimulation with an immunogen disclosed herein. Such assays are well known in the art. The assays may be able to analyze various different cytokines or cellular mediators associated with the immune response, e.g the cytokines IL-2, IL-4, IL-5, IL-9, IL-10, IL-12, IL-13, IL-17, IL-22, IL-31 and IFN-gamma.

1. A method for relieving an allergic immune response against a pollen allergen of a plant genus selected from any one of Ambrosia, Betula, Fraxinus, Olea, Plantago and Quereus in a subject in need thereof, comprising administering an effective amount of an immunogenic molecule, wherein said molecule comprises or consists of
a polypeptide comprising an amino acid sequence having at least 85% sequence identity to a sequence selected from any one of SEQ ID NOs: 413 and 808-812.

2-41. (canceled)

42. The method according to claim 1, wherein one or more cysteine residues of the sequence of any one of SEQ ID NOs: 413 and 808-812 are substituted with serine, 2-aminobutyric acid or arginine.

43. The method according to claim 1, wherein the allergic immune response is atopic dermatitis, allergic conjunctivitis, allergic rhinitis, or allergic asthma.

44. The method according to claim 1, wherein the subject has exhibited a symptom of, or suffers from, an allergic reaction, allergic response, allergic disorder or allergic disease.

45. The method according to claim 1, wherein the method relieves one or more symptoms of an allergic response or delays the onset of symptoms, slows the progression of symptoms, or induce disease modification.

46. The method according to claim 45, wherein the symptom(s) of an allergic reaction is selected from any of nasal symptoms in the form of itchy nose, sneezing, runny nose, blocked nose; conjunctival symptoms in the form of itchy eyes, red eyes, watery eyes; and
respiratory symptoms in the form of decreased lung function.

47. The method according to claim 1, wherein relieving an allergic response is observed by the patient's need for less concomitant treatment with corticosteroids or HI antihistamines to suppress the symptoms.

48. The method according to claim 1, wherein the treatment comprises immunotherapy.

49-50. (canceled)

51. A molecule comprising or consisting of
a polypeptide comprising an amino acid sequence having at least 85% sequence identity to a sequence selected from any one of SEQ ID NOs: 413 and 808-811.

52-54. (canceled)

55. A composition comprising a molecule according to claim 51 and a pharmaceutically acceptable ingredient or carrier.

56. The composition according to claim 55 that is lyophilized.

57. The composition according to claim 55 that is sterile.

58. The composition according to claim 55, comprising a single dose of the molecule in the range of 5 to 500 microgram.

59. The composition according to claim 55, which is a unit dosage form.

60. The composition according to claim 55, which is a solid dosage form.

61. The molecule according to claim 51, which comprises or consists of a polypeptide comprising an amino acid sequence having at least 95% sequence identity to SEQ ID NO: 413.

62. The molecule according to claim 51, which comprises or consists of a polypeptide comprising an amino acid sequence having at least 95% sequence identity to SEQ ID NO: 811.

63. The molecule according to claim 51, which comprises or consists of a polypeptide comprising an amino acid sequence having at least 90% sequence identity to SEQ ID NO: 808, 809, 810, or 812.

64. The molecule according to claim 51 wherein one or more cysteine residues of the sequences of any one of SEQ IDF NOs: 413, 808-812 are substituted with serine, 2-aminobutyric or arginine.

65. A method for relieving an allergic immune response against a pollen allergen of a plant genus selected from any one of Ambrosia, Betula, Fraxinus, Olea, Plantago and Quercus in a subject in need thereof, comprising administering an effective amount of an immunogenic molecule, wherein said molecule comprises or consists of a polypeptide of 15 to 30 amino acid residues in length and which includes at least one amino acid sequence with 0, 1 or 2 mismatches to a sequence selected from any one of SEQ ID NOs:143-153.
